(19) United States
(12) Patent Application Publication
(10) Pub. No.: US 2016/0363591 A1
(43) Pub. Date: Dec. 15, 2016

(54) DIPEPTIDYL PEPTIDASE-4 (DPP4/CD26) AS A PERIPHERAL BIOMARKER OF IL-13 ACTIVATION IN ASTHMATIC LUNG

(71) Applicant: MEDIMMUNE, LLC, Gaithersburg, MD (US)

(72) Inventors: Katie Streicher, Gaithersburg, MD (US); Yihong Yao, Gaithersburg, MD (US); Koustubh Ranade, Gaithersburg, MD (US); Meina Liang, Gaithersburg, MD (US); Inna Vainshtein, Gaithersburg, MD (US); Edward Piper, Cambridge (UK); Richard May, Cambridge (UK); Lars Nordenmark, Molndal (SE)

(21) Appl. No.: 15/114,467

(22) PCT Filed: Jan. 26, 2015
(86) PCT No.: PCT/US15/12885

§ 371 Date: Jul. 27, 2016

Related U.S. Application Data
(60) Provisional application No. 61/931,878, filed on Jan. 27, 2014.
(60) Provisional application No. 61/990,932, filed on May 9, 2014.

(51) Int. Cl. G01N 33/573; A61K 31/56; A61K 39/395; C07K 16/24

(52) U.S. Cl. CPC G01N 33/573; C12Y 304/14005; C07K 16/244; A61K 31/56; A61K 39/3955; G01N 2333/948; G01N 2800/122; G01N 2800/202; C07K 2317/56; C07K 2317/76; A61K 2039/505

(57) ABSTRACT

13 antagonist, (iv) to prognosticate the outcome of treating an IL-13 mediated condition or disorder with a specific IL-13 antagonist. The disclosure further provides assay kits for the detection of DPP4, as well as computer implemented diagnostic methods.

### REFERENCE TO SEQUENCE LISTING SUBMITTED ELECTRONICALLY

The content of the electronically submitted sequence listing in ASCII text file (Name: IL13-400WO_Sequence_Listing_ascii.txt; Size: 174,694 bytes; and Date of Creation: Jan. 15, 2015) filed with the application is incorporated herein by reference in its entirety.

### BACKGROUND

Bronchial asthma is a common persistent inflammatory disease of the lung characterized by airways hyper-responsiveness, mucus overproduction, fibrosis, and raised serum IgE levels. Airways hyper-responsiveness (AHR) is the exaggerated constriction of the airways to non-specific stimuli such as cold air. Both AHR and mucus overproduction are thought to be responsible for the variable airway obstruction that leads to the shortness of breath characteristic of asthma attacks (exacerbations) and which is responsible for the mortality associated with this disease.

Current British Thoracic Society (BTS) and Global Initiative for Asthma (GINA) guidelines suggest a stepwise approach to the treatment of asthma (Society, B. T., Thorax, 2003. 58 Suppl 1:1-94; GINA, Global Strategy for Asthma Management and Prevention. 2002, National Institute of Health). Mild to moderate asthma can usually be controlled by the use of inhaled corticosteroids, in combination with beta-agonists or leukotriene inhibitors. However, due to the documented side effects of corticosteroids, patients tend not to comply with the treatment regime, which reduces the effectiveness of treatment (Milgrom, H. et al. Ann Allergy Asthma Immunol, 2002. 88:429-31; Fish, L. and C. L. Lung, Ann Allergy Asthma Immunol, 2001. 86:24-30; Bender, B. G. J. Allergy Clin. Immunol, 2002. 109:S554-9). Asthma presents significant heterogeneity in response to various treatments, thereby highlighting the need to develop more effective therapies for this disease or identify biomarkers that predict response to specific therapies.

Atopic dermatitis is a common chronic inflammatory skin disease that is often associated with other atopic disorders such as allergic rhinitis and asthma (Bieber, New England Journal of Medicine, 2008, 358: 1483-1494). Upregulation of IL-13 mRNA has been observed in subacute and chronic lesions of atopic dermatitis (Tazawa et al., Arch. Dermatol. Res., 2004, 295:459-464; Purwar et al, J. Invest. Derm., 2006, 126, 1043-1051; Oh et al., J Immunol., 2011, 186:7232-42).

Chronic Obstructive Pulmonary Disease (COPD) includes patient populations with varying degrees of chronic bronchitis, small airway disease and emphysema and is characterized by progressive irreversible lung function decline that responds poorly to current asthma based therapy. The underlying causes of COPD remain poorly understood. Zheng et al (J Clin Invest, 106: 1081-93, 2000) have demonstrated that overexpression of IL-13 in the mouse lung caused emphysema, elevated mucus production and inflammation, reflecting aspects of human COPD. Furthermore, AHR, an IL-13 dependent response in murine models of allergic inflammation, has been shown to be predictive of lung function decline in smokers (Tashkin et al., Am J Respir Crit Care Med, 153(6 Pt 1): 1802-1 1, 1996). A link has also been established between an IL-13 promoter polymorphism and susceptibility to develop COPD (Van Der Pouw Kraan et al., Genes Immun. 3: 436-9, 2002). The signs are therefore that IL-4/IL-13 pathway, and in particular IL-13, plays an important role in the pathogenesis of COPD. See, e.g., Chen et al. PLoS One 8:e68222, 2013; Dente et al., Respiration 84:98-100, 2012; Grubek-Jaworska et al., Respiration 84:101-107, 2012; Walsh, Curr. Opin. Investig. Drugs 11:1305-12, 2010, all of which are herein incorporated by reference in their entireties.

Interleukin (IL)-13 is a 114 amino acid cytokine with an unmodified molecular mass of approximately 12 kDa (McKenzie, A. N., et al. J Immunol, 1993. 150:5436-44; Minty, A., et al. Nature, 1993. 362:248-50). IL-13 levels have been shown to correlate with disease severity in asthmatics and rodent models of allergic inflammation (see U.S. Pat. Appl. Publ. No. 2012-0052060, published Mar. 1, 2012, and incorporated herein by reference in its entirety). IL-13 may also play a role in the pathogenesis of inflammatory bowel disease, and has been associated with fibrotic conditions, such as idiopathic pulmonary fibrosis (IPF). Anti-IL-13 antibodies are currently been developed as therapies for treatment of patients with moderate to severe asthma. However, only a subset of asthma patients appear to have IL-13 driven disease. Thus, there is a need to identify such patients and predict the outcome of the treatment with IL-13 antagonists such as anti-IL-13 antibodies using a simple biomarker or combination of biomarkers.

### BRIEF SUMMARY

The present disclosure provides a method of treating a patient having an interleukin-13 (IL-13)-mediated disease or disorder, comprising administering an IL-13 antagonist to the patient if the level of DPP4 (dipeptidyl peptidase-4) in a sample taken from the patient is above a predetermined DPP4 threshold level, or is above the DPP4 level in one or more control samples. Also provided is a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12%, (v) wall area % (WA %) of subsegmental airways above about 68% as measured via CT scan of the lungs, or (vi) combinations thereof.

In addition, the present disclosure provides a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents high periostin (≧median serum periostin or about 23 ng/mL). Also provided is a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents a high eosinophil cell count (blood eosinophil count ≧300 cells/μL). Also provided is a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents with high Th2 (high Th2 defined as IgE >100 IU/mL and blood eosinophils ≧0.14×10⁹/L). Also provided is a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents with FEV1 reversibility to a short-acting β2 agonist ≧12%. Also provided is a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents with one or more of: i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12% and (v) wall area % (WA %) of subsegmental airways above about 68% as measured via CT scan of the lungs.

In addition, the present disclosure provides a method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents a level of at least one additional biomarker in a sample taken from the patient which is above a predetermined biomarker threshold level, or is above the biomarker level in one or more control samples, wherein said additional biomarker is selection from the group consisting of POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C2ORF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1 (SEQ ID NO:36), ALOX15 (SEQ ID NO:37), and combinations thereof.

In some aspects of the methods disclosed herein, a sample is obtained from the patient and submitted for measurement of the level of DPP4 in the sample. In other aspects, the patient's DPP4 level is measured in an immunoassay. In some aspects, the immunoassay employs one or more anti-DPP4 antibodies or antigen binding fragments thereof which recognize human DDP4.

Also provided is a method of treating a patient having an IL-13-mediated disease or disorder comprising (a) submitting a sample taken from the patient for measurement of the DPP4 level in the sample, wherein the patient's DPP4 level is measured in an immunoassay employing one or more anti-DPP4 antibodies or antigen binding fragments thereof which recognize human DPP4; and, (b) administering an IL-13 antagonist to the patient if the patient's DPP4 level in the sample is above a predetermined DPP4 threshold level, or is above the DPP4 level in one or more control samples. Also provided is a method of treating a patient having an IL-13-mediated disease or disorder comprising (a) measuring the DPP4 level in a sample obtained from a patient having an IL-13-mediated disease or disorder, wherein the patient's DPP4 level in the sample is measured in an immunoassay employing one or more anti-DPP4 antibodies or antigen binding fragments thereof which recognize human DPP4; (b) determining whether the patient's DPP4 level in the sample is above a predetermined DPP4 threshold level, or is above the DPP4 level in one or more control samples; and, (c) advising a healthcare provider to administer an IL-13 antagonist to the patient if the patient's DPP4 level is above a predetermined DPP4 threshold level, or is above the DPP4 level in one or more control samples.

In some aspects of the above disclosed methods, the IL-13-mediated disease or disorder is a pulmonary disease or disorder, an inflammatory bowel disease or disorder, or a chronic inflammatory skin disease or disorder. In other aspects the pulmonary disease or disorder is asthma or allergic rhinitis. In some aspects the chronic inflammatory skin disease or disorder is atopic dermatitis. In some aspects the pulmonary disease or disorder is COPD. In some aspects, COPD is stable COPD or acute exacerbation of COPD (AECOPD).

The present disclosure also provides a method of treating a patient diagnosed with a pulmonary disease or disorder comprising administering an IL-13 antagonist to the patient if the DPP4 level in a sample taken from the patient is above a predetermined DPP4 threshold level, or is above the DPP4 level in one or more control samples. In some aspects, the patient's DPP4 level is measured in an immunoassay employing one or more anti-DPP4 antibodies or antigen binding fragments thereof which recognize human DPP4.

Also provided is a method of treating a patient diagnosed with a pulmonary disease or disorder, an inflammatory bowel disease or disorder, or a chronic inflammatory skin disease or disorder comprising (a) submitting a sample taken from the patient for measurement of the DPP4 level in the sample, wherein the patient's DPP4 level is measured in an immunoassay employing one or more anti-DPP4 antibodies or antigen binding fragments thereof which recognize human DPP4; and (b) administering an IL-13 antagonist to a patient if the patient's DPP4 level in the sample is above a predetermined DPP4 threshold level, or is above the DPP4 level in one or more control samples.

Also provided is a method of determining whether to treat a patient diagnosed with a pulmonary disease or disorder, an inflammatory bowel disease or disorder, or a chronic inflammatory skin disease or disorder with an IL-13 antagonist therapeutic regimen comprising (a) measuring, or instructing a clinical laboratory to measure the DPP4 level in a sample obtained from a patient diagnosed with a pulmonary disease or disorder, an inflammatory bowel disease or disorder, or a chronic inflammatory skin disease or disorder, wherein the patient's DPP4 level is measured in an immunoassay employing one or more anti-DPP4 antibodies or antigen binding fragments thereof which recognize human DPP4; and (b) treating, or instructing a healthcare provider to treat the patient with an IL-13 antagonist therapeutic regimen if the patient's DPP4 level in the sample is above a predetermined DPP4 threshold level, or is above the DPP4 level in one or more control samples.

Also provided is a method of selecting a patient diagnosed with a pulmonary disease or disorder, an inflammatory bowel disease or disorder, or a chronic inflammatory skin disease or disorder as a candidate for treatment with an IL-13 antagonist therapeutic regimen comprising (a) measuring, or instructing a clinical laboratory to measure the DPP4 level in a sample obtained from a patient diagnosed with a pulmonary disease or disorder, an inflammatory bowel disease or disorder, or a chronic inflammatory skin disease or disorder, wherein the patient's DPP4 level is measured in an immunoassay employing one or more anti-DPP4 antibodies or antigen binding fragments thereof which recognize human DPP4; and (b) treating, or instructing a healthcare provider to treat the patient with an IL-13 antagonist therapeutic regimen if the patient's DPP4 level in the sample is above a predetermined DPP4 threshold level, or is above the DPP4 level in one or more control samples. In some aspects, the pulmonary disease or disorder is asthma, IPF, COPD (stable COPD or AECOPD), chronic rhinosinusitis, or allergic rhinitis. In some aspects, the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids. In some aspects, the chronic inflammatory skin disease or disorder is atopic dermatitis.

In some aspects of the above disclosed methods, the IL-13 antagonist comprises one or more of an anti-IL-13 antibody or antigen-binding fragment thereof, an IL-13 mutein, an IL-4 mutein, an anti-IL-13Rα1 antibody or antigen-binding fragment thereof, or an anti-IL-4Rα antibody or antigen-binding fragment thereof. In some aspects, the patient has been treated with one or more additional medications, either before, during, or after administration of an IL-13 antagonist. In some aspects, the one or more additional medications comprises a steroid. In other aspects, the one or more additional medications further comprises a bronchodilator. In some aspects, the steroid is fluticasone or budesonide. In some aspects, the bronchodilator is salbutamol or salmeterol. In other aspects, the one or more additional medications are administered by inhalation, by oral administration, by injection, or by a combination thereof. In some aspects, inhalation administration is conducted using a metered dose inhaler (MDI) or a dry powder inhaler (DPI). In some aspects, the steroid is administered at a high dose.

In some aspects of the methods disclosed herein, the IL-13 antagonist is an anti-IL-13 antibody, or antigen-binding fragment thereof. In some aspects, the antibody or fragment thereof binds to the same IL-13 epitope as tralokinumab or competitively inhibits binding of tralokinumab to IL-13, or both. In other aspects, the antibody or fragment thereof comprises tralokinumab or an antigen-binding fragment thereof. In some aspects, the antibody or fragment thereof consists of tralokinumab or an antigen-binding fragment thereof. In some aspects, the antibody or fragment thereof binds to the same IL-13 epitope as lebrikizumab or competitively inhibits binding of lebrikizumab to IL-13, or both. In some aspects, the antibody or fragment thereof comprises lebrikizumab or an antigen-binding fragment thereof. In other aspects, the antibody or fragment thereof consists of lebrikizumab or an antigen-binding fragment thereof.

In some aspects of the methods disclosed herein, the sample taken from the patient comprises one or more of whole blood, serum, plasma, skin, saliva, sputum, bronchoalveolar lavage fluid, lung epithelial cells, urine, or nasal polyps. In some aspects, the sample taken from the patient is blood serum. In some aspects, the methods disclosed herein further comprise determining, submitting a sample taken from the patient for determination, or instructing a clinical laboratory to determine (i) the level of the patient's IgE levels, (ii) the patient's eosinophil count, (iii) the patient's Fraction of Exhaled Nitric Oxide (FE_{NO}), (iv) the patient's Eosinophil/Lymphocyte and Eosinophil/Neutrophil (ELEN) index, (v) the patient's EOS index, (vi) wall area % (WA %) of subsegmental airways above about 68% as measured via CT scan of the lungs, or (vii) a combination of two or more thereof. In some aspects, the methods disclosed herein further comprises determining, submitting a sample taken from the patient for determination, or instructing a clinical laboratory to determine the expression level or activity of isoforms 1, 2, 3, or 4 of human periostin, a patient's blood eosinophil cell count, the level of the patient's IgE levels, pre- or post-bronchodilator FEV1 reversibility, or combinations thereof.

In some aspects, the methods disclosed herein further comprise determining, submitting a sample taken from the patient for determination, or instructing a clinical laboratory to determine the expression level or activity of sCTLA-3 (soluble CTLA-3; also known as Cytotoxic T-Lymphocyte-Associated serine Esterase 3, granzyme A, or granzyme 1; Uniprot: P12544), sCD28 (soluble CD28; also known as cluster of differentiation 28 or Tp44; Uniprot: P10747), CCL5 (chemokine C-C motif ligand 5; also known as RANTES; Uniprot: P13501), CCL11 (C-C motif chemokine 11; also known as eosinophil chemotactic protein or eotaxin-1; Uniprot: P51671), CCL22 (C-C motif chemokine 22; Uniprot: O00626), or combinations thereof.

In some aspects, the methods disclosed herein further comprise determining, submitting a sample taken from the patient for determination, or instructing a clinical laboratory to determine the expression level or activity of POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C2ORF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1 (SEQ ID NO:36), ALOX15 (SEQ ID NO:37), and combinations thereof.

In some aspects, the IL-13 antagonist is administered at a fixed dose. In specific aspects, tralokinumab is administered at a fixed dose of about 300 mg/dose. In some aspects, the IL-13 antagonist is administered in two or more doses. In other aspects, the IL-13 antagonist is administered week, biweekly or monthly. In certain aspects, the IL-13 antagonist is administered biweekly.

In some aspects, the IL-13 antagonist is administered intravenously, intramuscularly, subcutaneously, or a combination thereof. In other aspects, the predetermined DPP4 threshold level is at least about 250 ng/ml, at least about 275 ng/ml, at least about 300 ng/ml, at least about 325 ng/ml at least about 350 ng/mL, at least about 375 ng/mL, at least about 400 ng/mL, at least about 425 ng/mL, at least about 450 ng/mL, at least about 475 ng/mL, at least about 500 ng/mL, at least about 525 ng/mL, at least 550 ng/mL, at least about 575 ng/mL, or at least about 600 ng/mL, as measured in serum using an ELISA. In some aspects, the ELISA is a QUANTIKINE® assay. In some aspects, the predetermined DPP4 threshold level is about 365 ng/mL.

In some aspects, the one or more control samples are obtained from normal healthy individuals; patients with a non-IL-13-mediated subset of asthma; asthma patients naïve for corticosteroid treatment; asthma patients treated with corticosteroids; a pre-determined standard amount of isolated DPP4; or a combination thereof. In some aspects, the one or more control samples comprise one or more of whole blood, serum, plasma, saliva, sputum, bronchoalveolar lavage fluid, lung epithelial cells, urine, or a combination thereof.

In some aspects of the methods disclosed herein, administration of the IL-13 antagonist results in (a) AER (Acute Exacerbation Rate) reduction; (b) FEV₁ (Forced Expiratory Volume in one second) increase; (c) improved ACQ-6 (Asthma Control Questionnaire, 6-item version) results; (d) improved AQLQ (Asthma Quality of Life Questionnaire) results; or, (e) a combination thereof. In some aspects, the AER reduction is at least 5%, at least 10%, at least 15%, at least 20%, at least 25%, at least 30%, at least 35%, at least 40%, or at least 45% compared to the AER observed in a population of patients treated with a placebo. In other aspects, the mean AER reduction is about 28% compared to the mean AER observed in a population of patients treated with a placebo. In some aspects, the FEV₁ increase is at least 3%, at least 5%, at least 7%, at least 9%, at least 11%, at least 13%, at least 15%, at least 17%, or at least 19% compared to the FEV₁ observed in a population of patients treated with a placebo. In other aspects, the mean FEV₁ increase is about 10% compared to the mean FEV₁ observed in a population of patients treated with a placebo.

### BRIEF DESCRIPTION OF THE DRAWINGS/FIGURES

FIG. 1 presents a scheme used to identify genes that are regulated by IL-13 in the lung. Protein levels in normal and asthmatic sera of the candidate genes identified in the screen were determined. Normal human bronchial epithelial cells or human bronchial epithelial cells from the EPIAIRWAY™ model were stimulated with IL-13 for 24 hours, harvested and lysed, and the resulting transcriptional alterations were analyzed by whole genome array (WGA) and TAQMAN® PCR.

FIG. 2 shows genes up-regulated by IL-13 stimulation of normal human bronchial epithelial cells from the EPIAIRWAY™ model. Shown are log 2 fold change (fc) values for each gene following IL-13 stimulation. A number of genes including CCL26/Eotaxin-3, dipeptidyl peptidase-4 (DPP4), fetuin B (FETUB) and periostin (POSTN) were found to be up-regulated by IL-13 treatment. Total RNA was prepared from normal bronchial epithelial cells grown in a multilayered, highly differentiated, air-liquid interface model (EPIAIRWAY™, Mattek Corp.) either unstimulated or stimulated with 100 ng/mL IL-13 for 24 hours. RNA was reverse transcribed to cDNA and assayed by whole genome microarray.

FIG. 3 shows genes up-regulated by IL-13 stimulation of normal human bronchial epithelial cells. Shown are log 2 fold change (fc) values for each gene following IL-13 stimulation. A number of genes including CCL26/Eotaxin-3, dipeptidyl peptidase-4 (DPP4), fetuin B (FETUB) and periostin (POSTN) were found to be up-regulated by IL-13 treatment. Total RNA was prepared from normal bronchial epithelial cells grown in a monolayer, either unstimulated or stimulated with 100 ng/mL IL-13 for 24 hours. RNA was reverse transcribed to cDNA and assayed by whole genome microarray.

FIG. 4 shows TAQMAN® qPCR confirmation of microarray data following IL-13 stimulation presented in FIG. 2 and FIG. 3. Shown are mean±SD linear fold change values comparing gene expression in unstimulated cells/tissues with gene expression following IL-13 stimulation. Four genes shown to be elevated by IL-13 stimulation (DPP4, POSTN, CCL26 and FETUB) were analyzed by TAQMAN® qPCR (Fluidigm). Results corresponding to unstimulated (Unstim) samples as well as dipeptidyl peptidase-4 (DPP4), periostin (POSTN), CCL26/Eotaxin-3 (CCL26), and fetuin B (FETUB) are shown. Total RNA was isolated from either bronchial epithelium (2 donors; left column for each sample), normal EPIAIRWAY™ tissue (3 donors; middle column for each sample), or asthma EPIAIRWAY™ tissue (4 donors; right column for each sample) following no stimulation or stimulation with IL-13 for 24 hours. Samples were reverse-transcribed to cDNA and pre-amplified.

FIG. 5 shows that DPP4 protein levels were elevated in serum from severe asthma patients. Serum from healthy volunteers (n=38) and asthma patients identified as either moderate (n=13) or severe (n=12) were obtained from commercial sources. DPP4 levels (ng/mL) were measured using the QUANTIKINE® ELISA from R&D Systems. Median DPP4 values in each population are indicated with black bars. A trend to increased DPP4 was observed in asthma patients with moderate disease, with a statistically significant increase (* p value <0.05) in DPP4 expression in asthma patients with severe disease.

FIG. 6 shows that DPP4 protein levels were lower in serum from asthma patients taking oral and inhaled steroids. Serum from asthma patients identified as taking various medications (No medication, n=8; ADVAIR® only, n=18; Albuterol and inhaled steroids, n=27; or oral and inhaled steroids, n=40) were obtained from commercial sources. DPP4 levels (ng/mL) were measured using the QUANTIKINE® ELISA from R&D Systems. Median DPP4 values in each population are indicated with black bars. Of the patients evaluated, the median DPP4 level was lower in patients taking oral and inhaled steroids.

FIG. 7 shows the design of a Tralokinumab Phase 2b Study (CD-RI-CAT-354-1049) and a summary of key questions, dosing frequency, entry criteria, and key primary and secondary endpoints (EP). Q2W=every 2 weeks; Q4W=every 4 weeks; Wk=Week; SC═Sub-cutaneous administration.

FIG. 8 shows the change from baseline in pre-bronchodilator FEV1 over time (ITT Population). Relative increases in FEV1 were observed in both treatment groups during the first 17 weeks of the study compared to placebo. These increases were maintained through to Week 53 in the tralokinumab 300 mg Q2W group but were lost in the tralokinumab 300 mg Q2/4W group. FEV1=forced expiratory flow in one second; ITT=intent to treat; MMRM=mixed-effect model repeated measure; SEM=standard error of the mean; CAT-354=tralokinumab.

FIG. 9 presents forest plots showing annual Acute Exacerbation Rate Reductions (AERR) and change from baseline in pre-bronchodilator at Week 53 by FEV1 reversibility and serum periostin level. FIG. 9A is a forest plot showing the percentage reduction in annual % AERR by FEV1 reversibility and serum periostin level for CAT-354 Q2W or CAT-354 Q4W cohorts compared to placebo (PBO). FIG. 9B is a forest plot showing the difference vs placebo in change from baseline in pre-bronchodilator FEV1 by FEV1 reversibility and serum periostin level for CAT-354 Q2W or CAT-354 Q4W cohorts. CAT-354=tralokinumab; FEV1=forced expiratory volume in one second; Q2W=every 2 weeks; Q2/4W=2 injections Q2W for 12 weeks followed by Q4W for 38 weeks.

FIG. 10 presents forest plots showing ACQ and AQLQ(S) at Week 53 By FEV1 reversibility and serum periostin level. FIG. 10A is a forest plot showing the difference vs placebo for ACQ by FEV1 reversibility and periostin for CAT-354 Q2W or CAT-354 Q4W cohorts. FIG. 10B is a forest plot showing the difference vs placebo for AQLQ(S) by FEV1 reversibility and periostin for CAT-354 Q2W or CAT-354 Q4W groups. ACQ-6=Asthma Control Questionnaire 6; AQLQ(S)=Asthma Quality of Life Questionnaire-Standardized Version; CI=confidence interval; FEV1=forced expiratory volume in 1 second; ITT=intent-to-treat; Q2W=every 2 weeks; Q2/4W=2 injections Q2W for 12 weeks followed by Q4W for 38 weeks.

FIG. 11 is a forest plot showing the effect of subgroup analysis on annual Acute Exacerbation Rate (AER). In the ITT population, a reduction in the primary endpoint, the annual AER, was not observed in either tralokinumab treatment cohort compared to placebo; however, trends towards reductions in AER in patients receiving tralokinumab were observed in a number of pre-specified subgroups including: the presence of FEV1 reversibility to SABA ≧12% at baseline, high periostin, high eosinophil, and high Th2 subgroups. Reductions in AER were not observed in the subgroups receiving chronic OCS in either tralokinumab treatment cohort. AERR=asthma exacerbation rate reduction; CAT-354=tralokinumab; Eos=eosinophil; FEV1=forced expiratory volume in one second; ITT=intent to treat; OCS=oral corticosteroid; PBO=placebo; Q2W=every 2 weeks; Q2/4W=2 injections Q2W for 12 weeks followed by Q4W for 38 weeks; Th2=T helper 2.

FIG. 12 is a forest plot showing the effect of subgroup analysis on pre-bronchodilator (pre-BD) FEV1. In the ITT population, a statistically significant increase from baseline in pre-bronchodilator FEV1 at Week 53 compared to placebo was observed in the tralokinumab 300 mg Q2W cohort. Within the tralokinumab 300 mg Q2W cohort at Week 53, increases in pre-bronchodilator FEV1 compared to placebo were closely matched in both the high and low periostin subgroups and were numerically higher in the high reversible, high eosinophil and high Th2 subgroups than in the corresponding low subgroups. No increase in pre-bronchodilator FEV1 was observed in the subgroups receiving chronic OCS in either tralokinumab treatment cohort. CAT-354=tralokinumab; Eos=eosinophil; FEV1=forced expiratory volume in one second; ITT=intent to treat; OCS=oral corticosteroid; PBO=placebo; Q2W=every 2 weeks; Q2/4W=2 injections Q2W for 12 weeks followed by Q4W for 38 weeks; Th2=T helper 2.

FIG. 13 shows the asthma exacerbation rate reduction and mean percent change from baseline in pre-bronchodilator FEV1 at Week 53 for patients by baseline serum periostin level (Tralokinumab Q2W vs Placebo). FIG. 13A is a continuous representation of AER reduction (95% CI) by serum periostin level showing the median periostin value used in the analysis and the effect of changing the median periostin value (baseline periostin cutpoint) on AER Reduction at week 53. FIG. 13B is a continuous representation of percent change from baseline in pre-bronchodilator FEV1 (95% CI) by serum periostin level showing the median periostin value used in the analysis and the effect of changing the median periostin value (baseline periostin cutpoint) on the percent change from baseline in pre-bronchodilator FEV1 at Week 53. Q2W=every 2 weeks; AER=Asthma Exacerbation Rate; FEV1=forced expiratory volume in 1 second; CI=confidence interval.

FIG. 14A shows the percent change from baseline in pre-bronchodilator FEV1 over time, when periostin >=median (ITT population). Relative increases in FEV1 were observed in the 300 mg Q2W group through to Week 53. FEV1=forced expiratory flow in one second; ITT=intent to treat; Q2W=every 2 weeks; Q2/4W=2 injections Q2W for 12 weeks followed by Q4W for 38 weeks; CAT-354=tralokinumab.

FIG. 14B shows the percent change from baseline in pre-bronchodilator FEV1 over time, when periostin <median (ITT population). No significant increases in FEV1 were observed in the 300 mg Q2W group or the Q2/4W through to Week 53. FEV1=forced expiratory flow in one second; ITT=intent to treat; Q2W=every 2 weeks; Q2/4W=2 injections Q2W for 12 weeks followed by Q4W for 38 weeks; CAT-354=tralokinumab.

FIG. 15 presents data showing that DPP4 (DPP4-high classifier; i.e., DPP4>=median) outperforms periostin (periostin-high classifier; i.e., periostin level >=median) in the Intention to Treat (ITT) population, 300 mg tralokinumab Q2W group compared to placebo in various endpoints including acute exacerbation rate reduction, percentage change from baseline in FEV1, ACQ-6 change from baseline, and AQLQ change from baseline. FEV1=forced expiratory flow in one second; ACQ-6=Asthma Control Questionnaire 6; AQLQ(S)=Asthma Quality of Life Questionnaire-Standardized Version; CI=confidence interval; Q2W=every 2 weeks.

FIG. 16 compares acute exacerbation rate reduction, percentage change from baseline in FEV1, ACQ-6 change from baseline, and AQLQ change from baseline in the Intention to Treat (ITT) population, 300 mg tralokinumab Q2W group, stratified according to 4 classifiers: periostin-high (periostin level >=Median), periostin-low (periostin level <Median), DPP4-high (DPP4 level >=Median), and DPP4-low (DPP4 level <Median). FEV1=forced expiratory flow in one second; ACQ-6=Asthma Control Questionnaire 6; AQLQ=Asthma Quality of Life Questionnaire; CI=confidence interval; Q2W=every 2 weeks.

FIG. 17A-17I show the percent change from baseline for different endpoints depending of the DPP4 classifier user (i.e., DPP4 level >=Median or DPP4 level <Median) in the tralokinumab 300 mg Q2W group or the Q2/4W through to Week 53. Q2W=every 2 weeks; Q2/4W=2 injections Q2W for 12 weeks followed by Q4W for 38 weeks; CAT-354=tralokinumab; BSL=baseline; WX=Week X; FEV1=forced expiratory flow in one second; ACQ-6=Asthma Control Questionnaire 6; AQLQ(S)=Asthma Quality of Life Questionnaire-Standardized Version. FIG. 17A (percent change from baseline in pre-bronchodilator FEV1 over time and DPP4>=Median), FIG. 17B (percent change from baseline in pre-bronchodilator FEV1 over time and DPP4<Median), FIG. 17C (change from baseline in mean ACQ-6 over time and DPP4>=Median), FIG. 17D (change from baseline in mean ACQ-6 over time and DPP4<Median), FIG. 17E (change from baseline in mean AQLQ(S) over time and DPP4>=Median), FIG. 17F (change from baseline in mean AQLQ(S) over time and DPP4<Median), FIG. 17G (percent change from baseline in pre-Bronchodilator FEV1 over time, baseline FEV1 reversibility >=12% and DPP4>=Median), FIG. 17H (change from baseline in mean ACQ-6 over time, baseline FEV1 reversibility >=12% and DPP4>=Median), and FIG. 17I (change from baseline in mean AQLQ(S) over time, baseline FEV1 reversibility >=12% and DPP4>=Median). An increase in the percent change from baseline in pre-bronchodilator FEV1 (17A), a decrease in the change from Baseline in Mean ACQ-6 (17C), and an increase in the change from baseline in mean AQLQ(S) (17E) compared to placebo at week 53 were observed in the tralokinumab 300 mg Q2W cohort for DPP4>=Median. (FIGS. 17A, 17C, 17E, respectively). These changes were lost in the DPP4 level <Median group (FIGS. 17B, 17D, 17F, respectively). Similarly, an increase in the percent change from baseline in pre-bronchodilator FEV1 (17G), a decrease in the change from Baseline in Mean ACQ-6 (17H), and an increase in the change from baseline in mean AQLQ(S) (17I) compared to placebo at week 53 were observed in the tralokinumab 300 mg Q2W cohort for the baseline FEV1 reversibility to a short-acting β2 agonist >=12%+DPP4>=Median group.

FIG. 18 is a continuous representation of asthma exacerbation rate reduction by DPP4 level showing an asthma exacerbation rate reduction (95% CI) for subjects with baseline DPP4>=cut-point treated with CAT-354 Q2W compared to placebo. The median DPP4 value used in the analysis and the effect of changing the median DPP4 value (baseline DPP4 cut-point) on asthma exacerbation rate reduction are shown. Q2W=every 2 weeks; CAT-354=tralokinumab; CI=confidence interval.

FIG. 19 is a continuous representation of the mean percent change from baseline in pre-BD FEV1 at week 53 (95% CI) for subjects with baseline DPP4>=cut-point treated with CAT-354 Q2W compared to placebo. The median DPP4 value used in the analysis and the effect of changing the median DPP4 value (baseline DPP4 cut-point) on mean percent change from baseline in pre-BD FEV1 are shown. Q2W=every 2 weeks; CAT-354=tralokinumab; CI=confidence interval; pre-BD=pre-bronchodilator; FEV1=FEV1=forced expiratory flow in one second.

FIG. 20 is a continuous representation of mean change from baseline in mean ACQ-6 score at week 53 (95% CI) for subjects with baseline DPP4>=cut-point treated with CAT-354 Q2W compared to placebo. The median DPP4 value used in the analysis and the effect of changing the median DPP4 value (baseline DPP4 cut-point) on the mean change from baseline in ACQ-6 are shown. Q2W=every 2 weeks; CAT-354=tralokinumab; CI=confidence interval; ACQ-6=Asthma Control Questionnaire 6.

FIG. 21 shows the relative distribution of subjects classified as DPP4-high (serum DPP4≧median); DPP-low (serum DPP4 below median); periostin-high (serum periostin ≧median); and periostin-low (serum periostin below median) irrespective of treatment group. In each quadrant, the upper number corresponds to the number of subjects while the lower number corresponds to the % of total subjects (e.g. DPP4-high, periostin-High=125 patients or 27.84% of the study subjects). There is a partial overlap between the DPP4-high and periostin-high groups.

FIG. 22 shows the relative distribution of subjects classified as DPP4-high (serum DPP4≧median); DPP-low (serum DPP4 below median); Th2-high (IgE >100 IU/mL and blood eosinophils ≧0.14×109/L); and Th2-low (subjects not classified as Th-2 high) irrespective of treatment group. In each quadrant, the upper number corresponds to the number of subjects while the lower number corresponds to the % of total subjects (e.g. DPP4-high, Th2-High=114 patients or 27.67% of the study subjects). There is a partial overlap between the DPP4-high and Th2-high groups.

FIG. 23 shows the relative distribution of subjects classified as DPP4-high (serum DPP4≧median); DPP-low (serum DPP4 below median); EOS-high (blood eosinophil count ≧300 cells/μL); and Eos-low (blood eosinophil count <300 cells/μL) irrespective of treatment group. In each quadrant, the upper number corresponds to the number of subjects while the lower number corresponds to the % of total subjects (e.g. DPP4-high, Eos-High=84 patients or 19.86% of the study subjects). There is a partial overlap between the DPP4-high and Eos-high groups.

FIG. 24 shows that mean and median serum DPP4 levels for subjects with (positive) or without (negative) chronic OCS use. The mean and median serum DPP4 levels are reduced in patients chronically treated with OCS. OCS=oral corticosteroid; N=number of subjects.

FIG. 25 shows AERR, Exacerbation rates, mean percent change from baseline FEV1, mean change from baseline for ACQ-6 and the mean change from baseline for AQLQ for CAT-354 compared to placebo in subjects not chronically treated with OCS with baseline FEV1 reversibility to a short-acting β2 agonist (≧12% or <12%) and high (≧median) or low (<median) serum DPP4. OCS=oral corticosteroid; CAT-354=tralokinumab; BL=baseline; FEV1=forced expiratory flow in one second; ACQ-6=Asthma Control Questionnaire 6; AQLQ=Asthma Quality of Life Questionnaire; pbo=placebo; N=number of subjects; CI=confidence interval.

FIG. 26 shows AERR, Exacerbation rates, mean percent change from baseline FEV1, mean change from baseline for ACQ-6 and the mean change from baseline for AQLQ for CAT-354 compared to placebo in subjects not chronically treated with OCS with baseline FEV1 reversibility to a short-acting β2 agonist (≧12% or <12%) and high (≧median) or low (<median) serum periostin. OCS=oral corticosteroid; CAT-354=tralokinumab; BL=baseline; FEV1=forced expiratory flow in one second; ACQ-6=Asthma Control Questionnaire 6; AQLQ=Asthma Quality of Life Questionnaire; pbo=placebo; N=number of subjects; CI=confidence interval.

FIG. 27 shows periostin (POSTN) mRNA expression intensity levels in normal skin and atopic dermatitis (AD) skin measured using two probes, probe 1555778 (Panel A) and probe 210809 (Panel B), respectively, using whole genome microarray (Affymetrix). Total RNA was isolated from either normal skin (31 donors) or skin from subjects diagnosed with atopic dermatitis (4 donors). Biotin-labeled amplified cRNA was generated from total RNA and fragmented for hybridization on Affymetrix Human Genome U133 Plus 2.0 GeneChip® arrays. Median expression intensity levels in each population are indicated with black bars. POSTN mRNA expression is elevated in AD skin samples compared to normal skin.

FIG. 28 shows DPP4 mRNA expression intensity levels in normal skin and atopic dermatitis (AD) skin measured using two probes, probe 203717 (Panel A) and probe 203716 (Panel B), respectively, using whole genome microarray (Affymetrix). Total RNA was isolated from either normal skin (31 donors) or skin from subjects diagnosed with atopic dermatitis (4 donors). Biotin-labeled amplified cRNA was generated from total RNA and fragmented for hybridization on Affymetrix Human Genome U133 Plus 2.0 GeneChip® arrays. Median expression intensity levels in each population are indicated with black bars. DPP4 mRNA expression is elevated in AD skin samples compared to normal skin.

FIG. 29 shows representative computed tomography (CT) images of lungs from a patient at visit 4 (Panel A) and at visit 30 (Panel B) showing various bronchial airways.

FIG. 30 shows relative changes in lumen area (LA) in patients treated with placebo or tralokinumab from baseline (visit 4) and visit 30 as measured using VIDA APOLLO® software of 3D computed tomography imaging scans of the lungs. Relative changes in LA corresponding to RB1 bronchial airway (Panel A), segmental airways (Panel B), and subsegmental airways (Panel C) are presented. A significant increase in lumen area of subsegmental airways was observed in patients treated with tralokinumab compared to placebo (p value=0.021). Tralo=tralokinumab; p=p value.

FIG. 31 shows relative changes in bronchial wall area percentage (WA %) in patients treated with placebo or tralokinumab (Tralo) from baseline (visit 4) and visit 30 as measured using VIDA APOLLO® software of 3D computed tomography imaging scans of the lungs. Relative changes in WA % corresponding to RB1 bronchial airway (Panel A), segmental airways (Panel B), and subsegmental airways (Panel C) are presented. A significant decrease in wall area percentage (WA %) of subsegmental airways was observed in patients treated with tralokinumab compared to placebo (p value=0.0049). Tralo=tralokinumab; p=p value.

FIG. 32 shows relative changes in airway resistance in patients treated with placebo or tralokinumab (Tralo) from baseline (visit 4) and visit 30 as measured using VIDA APOLLO® software of 3D computed tomography imaging scans of the lungs. See Example 6 for more details. Relative changes in airway resistance corresponding to RB1 bronchial airway (Panel A), segmental airways (Panel B), and subsegmental airways (Panel C) are presented. The dashed rectangle indicates the airway resistance data set that was reanalyzed in FIG. 33A according to a baseline WA % threshold value (median WA %). A significant decrease in airway resistance of subsegmental airways was observed in patients treated with tralokinumab compared to placebo (p value=0.0081). Tralo=tralokinumab; p=p value.

FIG. 33 shows relative changes from baseline (visit 4) and visit 30 in airway resistance of subsegmental airways (Panel A) and pre-bronchodilator FEV1 (Panel B) in patients treated with tralokinumab. Patients having a wall percentage (WA %) of subsegmental airways higher than 68% at baseline had significant reductions in airway resistance (p value=0.0037) and significant improvements in FEV1 response (p value=0.045) compared to patients having less than a 68% wall percentage (WA %) at baseline. Tralo=tralokinumab; p=p value.

FIG. 34 shows IL-13 specific up-regulation of CCL-26 (Panel A), DPP4 (Panel B), Periostin POSTN-745 (Panel C), and Periostin POST-815(Panel D) transcripts from highly differentiated bronchial epithelial cells grown at air liquid interfaces (EPIAIRWAY™ model). Samples corresponded to 3 normal donors, designated Normal-25, Normal-21 and Normal-30, respectively (bars 1-3 of each set, left to right), and 3 COPD donors, designated COPD-15, COPD-12 and COPD-18, respectively (bars 4-6 of each set, left to right).

FIG. 35 shows periostin (Panel A) and DPP4 (Panel B) expression levels in atopic dermatitis patients suffering from moderate (Mod) or severed (Sev) atopic dermatitis. Data is expressed as geometric mean (number next to central dot) with 95% confidence intervals (positive and negative bars). Each individual patient observation is shown as a separate dot. Horizontal lines and associated numbers represent the upper and lower confidence intervals around the moderate patient point estimate.

FIG. 36 shows serum levels of periostin in healthy controls (n=20 subjects), stable COPD (n=101 subjects) and acute exacerbations of COPD (AECOPD; n=61 subjects) measured by immunoassay. Serum periostin levels are significantly elevated in patients suffering from COPD (both stable and acute exacerbations of COPD) compared to healthy controls. P=p value; NS=no significant difference.

FIG. 37 shows serum levels of DPP4 in healthy controls (n=20 subjects), stable COPD (n=104 subjects) and acute exacerbations of COPD (AECOPD; n=72 subjects) measured by immunoassay. Serum DPP4 levels are significantly elevated in patients suffering from COPD (both stable and acute exacerbations of COPD) compared to healthy controls. P=p value; NS=no significant difference.

### DETAILED DESCRIPTION

The disclosure relates to the use of DPP4 (SEQ ID: 5, membrane bound form protein sequence; SEQ ID NO: 6, soluble form protein sequence; SEQ ID NO: 7, DPP4 gene cDNA sequence) as a biomarker for IL-13 mediated disease or disorders, e.g., asthma, IPF, COPD (e.g., stable COPD or acute exacerbations of COPD), or atopic dermatitis. Accordingly, the disclosure provides methods for diagnosing and treating a subject as having an IL-13-mediated disease or disorder, comprising administering an IL-13 antagonist, for example, an anti-IL-13 antibody, to the patient if the DPP4 level in a sample taken from the patient is above a predetermined DPP4 threshold level, or if the DPP4 level is elevated relative to the DPP4 level in one or more control samples. In particular, the presence of levels of the DPP4 biomarker above or below a predetermined DPP4 threshold level can be used, e.g., (i) to determine whether a patient suffering an IL-13-mediated disease or disorder is eligible or non-eligible for a specific treatment with an IL-13 antagonist (e.g., an antibody such as tralokinumab), (ii) to determine whether a specific treatment of an IL-13-mediated disease or disorder with an IL-13 antagonist should commence, be suspended, or be modified, (iii) to diagnose whether an IL-13-mediated disease or disorder is treatable or not treatable with a specific IL-13 antagonist, (iv) to prognosticate the outcome of treatment of an IL-13-mediated disease or disorder with a specific IL-13 antagonist, etc.

In some aspects, the presence of DPP4 levels above or below a predetermined DPP4 threshold level in samples (e.g., blood serum or skin) obtained from a patient suffering from an IL-13-mediated pulmonary disease or disorder (e.g., asthma, IPF or COPD) or an IL-13-mediated chronic inflammatory skin disease or disorder (e.g., atopic dermatitis) can be used, e.g., (i) to determine whether the patient is eligible or non-eligible for treatment with a specific therapeutic agent, (ii) to determine whether a specific treatment should commence, be suspended, or be modified, (iii) to diagnose whether the disease or disorder is treatable or not treatable with a specific therapeutic agent, (iv) to prognosticate the outcome of treatment of the disease or disorder (e.g., asthma, IPF, COPD or atopic dermatitis) with a specific therapeutic agent, etc.

In some aspects, the presence of DPP4 levels above or below a predetermined DPP4 threshold level in samples (e.g., blood serum or skin) obtained from a patient suffering from an IL-13-mediated disease or disorder (e.g., asthma, IPF or COPD) or an IL-13-mediated chronic inflammatory skin disease or disorder (e.g., atopic dermatitis) in combination with one or more of: (i) high periostin (≧median serum periostin or about 23 ng/mL, (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12%, (v) wall area % (WA %) of subsegmental airways above about 68% as measured via CT scan of the lungs, or (vi) combinations thereof, can be used, e.g., (i) to determine whether a patient suffering an IL-13-mediated disease or disorder is eligible or non-eligible for a specific treatment with an IL-13 antagonist (e.g., an antibody such as tralokinumab or lebrikizumab), (ii) to determine whether a specific treatment should commence, be suspended, or be modified, (iii) to diagnose whether the disease or disorder is treatable or not treatable with a specific therapeutic agent, (iv) to prognosticate the outcome of treatment of the disease or disorder (e.g., asthma, IPF, COPD or atopic dermatitis) with a specific therapeutic agent, etc.

In order that the present disclosure can be more readily understood, certain terms are first defined. Additional definitions are set forth throughout the detailed description.

### I. DEFINITIONS

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

The term “about” as used in connection with a numerical value throughout the specification and the claims denotes an interval of accuracy, familiar and acceptable to a person skilled in the art. In general, such interval of accuracy is ±15%.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “antibody” (or a fragment, variant, or derivative thereof) refers to at least the minimal portion of an antibody which is capable of binding to antigen, e.g., at least the variable domain of a heavy chain (VH) and the variable domain of a light chain (VL) in the context of a typical antibody produced by a B cell. Basic antibody structures in vertebrate systems are relatively well understood. See, e.g., Harlow et al., Antibodies: A Laboratory Manual, (Cold Spring Harbor Laboratory Press, 2nd ed. 1988).

Antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that an antibody or fragment, variant, or derivative thereof binds to an epitope via its antigen-binding domain, and that the binding entails some complementarity between the antigen binding domain and the epitope. According to this definition, an antibody is said to “specifically bind” to an epitope when it binds to that epitope via its antigen-binding domain more readily than it would bind to a random, unrelated epitope.

An antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

Antibodies or antigen-binding fragments, variants, or derivatives thereof disclosed herein can be described or specified in terms of the epitope(s) or portion(s) of an antigen, e.g., a target polysaccharide that they recognize or specifically bind. For example, the portion of human DPP4 that specifically interacts with the antigen-binding domain of an anti-DPP4 antibody is an “epitope.”

As used herein, the term “IL-13-mediated disease or disorder” refers to any pathology caused by (alone or in association with other mediators), exacerbated by, associated with, or prolonged by abnormal levels of IL-13 in the subject having the disorder. Non-limiting examples of IL-13-mediated diseases or disorders include asthma, idiopathic pulmonary fibrosis (IPF), chronic obstructive pulmonary disease (COPD), ulcerative colitis (UC), allergic rhinitis, chronic rhinosinusitis, and atopic dermatitis.

As used herein, the term “pulmonary disease or disorder” refers to any pathology affecting at least in part the lungs or respiratory system. Non-limiting examples include asthma, IPF, COPD, allergic rhinitis, or chronic rhinosinusitis. In certain aspects, the pulmonary disease or disorder is IL-13-mediated.

As used herein, the term “chronic inflammatory skin disease or disorder” refers to any pathology affecting at least in part the skin. Non-limiting examples include atopic dermatitis, skin fibrosis, allergic contact dermatitis, eczema or psoriasis. In certain aspects, the chronic inflammatory skin disease or disorder is IL-13-mediated.

The term “asthma” refers to diseases that present as reversible airflow obstruction and/or bronchial hyper-responsiveness that may or may not be associated with underlying inflammation. Examples of asthma include allergic asthma, atopic asthma, corticosteroid naive asthma, chronic asthma, corticosteroid resistant asthma, corticosteroid refractory asthma, asthma due to smoking, asthma uncontrolled on corticosteroids and other asthmas as mentioned, e.g., in the Expert Panel Report 3: Guidelines for the Diagnosis and Management of Asthma, National Asthma Education and Prevention Program (2007) (“NAEPP Guidelines”), incorporated herein by reference in its entirety.

The term “COPD” as used herein refers to chronic obstructive pulmonary disease.

The term “COPD” includes two main conditions: emphysema and chronic obstructive bronchitis. Thus, in the broadest sense, the term COPD as used herein refers to COPD itself and also its subconditions chronic bronchitis and emphysema. The Global Initiative for Chronic Obstructive Lung Disease (GOLD) has classified 4 different stages of COPD. GOLD classification for COPD Stage 0: At Risk for COPD. Symptoms of chronic cough and sputum production may be present, but patients have normal spirometry readings. Stage I: Mild COPD. Characterized by FEV₁>=80%, FEV₁/FVC <70%. Patients may have or not have chronic cough and increased sputum production. Stage II: Moderate COPD. Characterized by a worsening of airflow (30%>=FEV₁>80%). Patients with Stage II disease often are symptomatic, seek medical attention, and have shortness of breath with exertion. Stage II has 2 subcategories: IIA and IIB. IIA patients have a FEV₁ between 50% and 80%; stage IIB patient have a FEV₁ between 30% and 50%. Patients with FEV₁ below 50% are especially prone to acute exacerbations of disease. Stage III: Severe COPD. Characterized by an FEV₁ below 30%. Patients are also included in stage III if they have respiratory failure or right heart failure. The quality of life is severely affected in these patients. Acute exacerbations in this patient population often require hospitalization and are frequently life threatening.

As used herein, “early stage” COPD is intended to mean GOLD Stage 0 or a precursor condition thereto.

In some aspects, COPD is stable COPD. In other aspects, COPD refers to a COPD exacerbation. As used herein, the term “exacerbation” refers to a worsening of symptoms of COPD, relative to a patient's baseline condition. In certain embodiments, a COPD exacerbation may be defined as an event in the natural course of the disease characterized by a change in the patient's baseline lung function, dyspnea, cough, and/or sputum that is beyond normal day-to-day variations, is acute in onset and may warrant a change in medication in a patient with underlying COPD. In certain embodiments, exacerbation of COPD may be an abrupt increase in symptoms of shortness of breath and/or wheezing, and/or increase in production of purulent sputum.

The term “Idiopathic Pulmonary Fibrosis” (IPF) refers to a disease characterized by progressive scarring, or fibrosis, of the lungs. It is a specific type of interstitial lung disease in which the alveoli gradually become replaced by fibrotic tissue. With IPF, progressive scarring causes the normally thin and pliable tissue to thicken and become stiff, making it more difficult for the lungs to expand, preventing oxygen from readily getting into the bloodstream. See, e.g., Am. J. Respir. Crit. Care Med. 2000. 161:646-664.

As used herein, the term “atopic dermatitis” refers to a chronic inflammatory, relapsing, non-contagious and itchy skin disorder that is often associated with other atopic disorders such as allergic rhinitis and asthma (Bieber, New England Journal of Medicine, 2008, 358: 1483-1494). The term “atopic dermatitis” is equivalent to “neurodermatitis”, “atopic eczema” or “endogenous eczema”. Particular forms of atopic dermatitis, which get their names from the place where they occur or from their appearance or from the stress factors which provoke them, are, according to the present disclosure also comprised by the term “atopic dermatitis”. These include, but are not limited to, eczema flexurarum, eczema mulluscatum, eczema verrucatum, eczema vaccinatum, eczema dyskoides, dyshydrotic eczema, microbial eczema, nummular eczema, seborrhobic eczema and other forms of eczema; perioral dermatitis and periorbital dermatitis. As used herein, the term atopic dermatitis also comprises the frequently occurring bacterial secondary infections such as those due to e.g. Staphylococcus aureus infections, pyodermas such as impetigo contagiosa and its derivatives as well as the follicularis barbae or viral secondary infections. IL-13 is involved in the pathogenesis of the disease and is an important in vivo inducer. See, e.g., Oh et al., J. Immunol. 186:7232-42 (2011); Tazawa et al., Arch. Dermatol. Res. 295:459-464 (2004); Metwally et al. Egypt J. Immunol. 11:171-7 (2004).

As used herein the terms “treat,” “treatment,” or “treatment of” (e.g., in the phrase “treating a patient having an IL-13-mediated disease or disorder”) refers to reducing the potential for an IL-13-mediated disease or disorder, reducing the occurrence of the IL-13-mediated disease or disorder, and/or a reduction in the severity of the IL-13-mediated disease or disorder, preferably, to an extent that the subject no longer suffers discomfort and/or altered function due to it (for example, a relative reduction in asthma exacerbations when compared to untreated patients). For example, treating can refer to the ability of a therapy when administered to a subject, to prevent an IL-13-mediated disease or disorder from occurring and/or to cure or to alleviate IL-13-mediated disease symptoms, signs, or causes. Treating also refers to mitigating or decreasing at least one clinical symptom and/or inhibition or delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

The present disclosure provides methods and systems providing therapeutic benefit in the treatment of an IL-13-mediated disease or disorder. A therapeutic benefit is not necessarily a cure for a particular IL-13-mediated disease or disorder, but rather encompasses a result which most typically includes alleviation of the IL-13-mediated disease or disorder or increased survival, elimination of the IL-13-mediated disease or disorder, reduction of a symptom associate with the IL-13-mediated disease or disorder, prevention or alleviation of a secondary disease, disorder or condition resulting from the occurrence of a primary IL-13-mediated disease or disorder, and/or prevention of the IL-13-mediated disease or disorder.

The terms “subject” or “patient” as used herein refer to any subject, particularly a mammalian subject, for whom diagnosis, prognosis, or therapy of an IL-13-mediated disease or disorder is desired. As used herein, the terms “subject” or “patient” include any human or nonhuman animal. The term “nonhuman animal” includes all vertebrates, e.g., mammals and non-mammals, such as nonhuman primates, sheep, dogs, cats, horses, cows, bears, chickens, amphibians, reptiles, etc. As used herein, phrases such as “a patient having an IL-13-mediated disease or disorder” includes subjects, such as mammalian subjects, that would benefit from the administration of a therapy, imaging or other diagnostic procedure, and/or preventive treatment for that IL-13-mediated disease or disorder. In some aspects of the present disclosure, a subject is a naïve subject. A naïve subject is a subject that has not been administered a therapy, for example a therapeutic agent. In some aspects, a naïve subject has not been treated with a therapeutic agent prior to being diagnosed as having an IL-13-mediated disease or disorder, for example, asthma, IFP, COPD, UC, or atopic dermatitis. In another aspect, a subject has received therapy and/or one or more doses of a therapeutic agent (e.g., a therapeutic agent capable of modulating an inflammatory response associated with an IL-13-mediated disease or disorder, a pulmonary disease or disorder, an inflammatory bowel disease or disorder or a chronic inflammatory skin disease or disorder) prior to being diagnosed as having an IL-13-mediated disease or disorder. In one aspect, the therapeutic agent is a small molecule drug. In a specific aspect, the agent is a corticosteroid. In another aspect, the agent can be a leukotriene modifier such as montelukast, zafirlukast or zileuton. In a further aspect, the therapeutic agent can be a methylxanthine (e.g., theophylline) or a cromone (e.g., sodium cromolyn and nedocromil). In another aspect, the therapeutic agent can be a long-acting beta-2 agonist such as salmeterol, fomoterol, or indacaterol. In a further aspect, the agent can be methotrexate or cyclosporin.

In certain aspects, the therapeutic agent can be an agent used for preventing, treating, managing, or ameliorating asthma or COPD. Non-limiting examples of therapies for asthma or COPD include anti-cholinergics (e.g., ipratropium bromide and oxitropium bromide), beta-2 antagonists (e.g., albuterol (PROVENTIL® or VENTOLIN), bitolterol (TOMALATE®), fenoterol, formoterol, isoetharine, metaproterenol, pibuterol (MAXAIR®), salbutamol, salbutamol terbutaline, and salmeterol, terbutlaine (BRETHAIRE®)), corticosteroids (e.g., prednisone, beclomethasone dipropionate (VANCERIL® or BECLOVENT®), triamcinolone acetonide (AZMACORF®), flunisolide (AEROBID®), and fluticasone propionate (FLOVENT®)), leukotriene antagonists (e.g., montelukast, zafirlukast, and zileuton), theophylline (THEO-DUR®, UNIDUR® tablets, and SLO-BID® Gyrocaps), and salmeterol (SEREVENT®), cromolyn, and nedorchromil (INTAL® and TILADE®)), IgE antagonists, IL-4 antagonists (including antibodies), IL-5 antagonists (including antibodies), PDE4 inhibitors, NF-Kappa-B inhibitors, IL-13 antagonists (including antibodies), CpG, CD23 antagonists, selectin antagonist (e.g., TBC 1269), mast cell protease inhibitors (e.g., tryptase kinase inhibitors (e.g., GW-45, GW-58, and genisteine), phosphatidylinositide-3′ (PI3)-kinase inhibitors (e.g., calphostin C), and other kinase inhibitors (e.g., staurosporine), C2a receptor antagonists (including antibodies), and supportive respiratory therapy, such as supplemental and mechanical ventilation.

In some aspects, a subject has received at least one therapeutically effective dose of oral or inhaled corticosteroids. In some aspects, a subject has received multiple therapeutically effective doses of oral or inhaled corticosteroids. In some aspects, a subject is a chronic oral corticosteroid (OCS) user.

In certain aspects the subject has received a long-acting beta2-adrenergic agonist, e.g., salmeterol xinafoate. In some aspects the subject has received a synthetic glucocorticoid, e.g., fluticasone propionate. In certain aspects the subject has received a combination of salmeterol xinafoate and fluticasone propionate (ADVAIR®). In certain aspects the subject has received a beta2-adrenergic bronchodilator, e.g., albuterol sulfate.

In one aspect, the therapeutic agent used according to methods disclosed herein is an antibody, e.g., an anti-IL-13 antibody or an antigen-binding fragment thereof. Accordingly, in some aspects, a subject has received or is a candidate to receive at least one therapeutically effective dose of an antibody (e.g., an anti-IL-13 antibody or an antigen-binding fragment thereof) capable of neutralizing IL-13-mediated pathology. In some aspects, the anti-IL-13 antibody is tralokinumab (SEQ ID NOS: 3 and 4) or an antigen-binding fragment thereof. See U.S. Pat. No. 7,829,090, herein incorporated by reference in its entirety. Other anti-IL-13 monoclonal antibodies that can be used include those described in U.S. Pat. Appl. Publ. No. 2012-0052060, published Mar. 1, 2012. Other IL-13 antagonists include, without limitation: (a) an anti-human-IL-13 antibody, for example, Lebrikizumab (MILR1444A/RG3637, Roche/Genentech) (SEQ ID NOS: 1 and 2) or an antigen-binding fragment thereof, ABT-308 (Abbott), GSK679586 (GlaxoSmithKline) or QAX576 (Novartis); (b) an anti-human-IL-13Ral antibody, for example, Merck MK6105; (c) an IL-13-toxin conjugate such as IL-13-PE38QQR (NeoPharm, Inc.); (d) an IL-4 mutein AEROVANT™ (Aerovance, Inc.); (e) an anti-IL-4Rα antibody such as dupilumab/REGN668 (Regeneron); (f) a double-stranded oligonucleotide directed against IL-4Rα such as AIR645 (Isis); or (g) an IL-4/IL-13 bispecific antibody such as GSK2434735 (Glaxo SmithKline). In some aspects, a subject can be administered at least one therapeutically effective dose of an anti-IL-13 antibody or an antigen-binding fragment thereof disclosed herein if the subject's DPP4 level is above a predetermined DPP4 threshold level, or if the DPP4 level is elevated relative to the DPP4 level in one or more control samples. In other aspects, a subject can be deemed eligible to receive at least one therapeutically effective dose of an anti-IL-13 antibody or an antigen-binding fragment thereof disclosed herein if the subject's DPP4 level is above a predetermined DPP4 threshold level, or if the DPP4 level is elevated relative to the DPP4 level in one or more control samples.

As used herein, the term “IL-13 antagonist” refers to any agent that can affect the expression, activity, or half-life of IL-13 either in vitro or in vivo, or symptoms, pathology, or sequelae caused by or exacerbated by IL-13 in a subject with an IL-13-mediated disease or disorder, e.g., asthma. An IL-13 antagonist can be any “therapeutic agent” as defined herein, which either directly or indirectly can inhibit, lessen, or neutralize IL-13 activity, inhibit or reduce IL-13 expression, reduce IL-13 half-life, or can prevent exacerbation of symptoms due to IL-13. In certain aspects, an IL-13 antagonist is an anti-IL-13 monoclonal antibody, e.g., tralokinumab, or other anti-IL-13 monoclonal antibodies described, e.g., in U.S. Pat. Appl. Publ. No. 2012-0052060, published Mar. 1, 2012.

The term “therapy” as used herein includes any means for curing, mitigating, or preventing an IL-13-mediated disease or disorder, including, for example, therapeutic agents, instrumentation, supportive measures, and surgical or rehabilitative procedures. In this respect, the term therapy encompasses any protocol, method and/or therapeutic or diagnostic that can be used in prevention, management, treatment, and/or amelioration of an IL-13-mediated disease or disorder.

The term “therapeutic agent” as used herein refers to any therapeutically active substance that is administered to a subject having an IL-13-mediated disease or disorder to produce a desired, usually beneficial, effect. The term therapeutic agent includes, e.g., classical low molecular weight therapeutic agents commonly referred to as small molecule drugs and biologics including but not limited to: antibodies or active fragments thereof, peptides, lipids, protein drugs, protein conjugate drugs, enzymes, oligonucleotides, ribozymes, genetic material, prions, virus, bacteria, and eukaryotic cells. A therapeutic agent can also be a pro-drug, which metabolizes into the desired therapeutically active substance when administered to a subject. In some aspects, the therapeutic agent is a prophylactic agent. In addition, a therapeutic agent can be pharmaceutically formulated. A therapeutic agent can also be a radioactive isotope or agent activated by some other form of energy such as light or ultrasonic energy, or by other circulating molecules that can be systemically administered.

A “therapeutically effective” amount as used herein is an amount of therapeutic agent that provides some improvement or benefit to a subject having an IL-13-mediated disease or disorder, e.g., an IL-13-mediated pulmonary disease or disorder such as asthma, IPF or COPD; or a chronic inflammatory skin disease or disorder such as atopic dermatitis. Thus, a “therapeutically effective” amount is an amount that provides some alleviation, mitigation, and/or decrease in at least one clinical symptom of the IL-13-mediated disease or disorder, e.g., an IL-13-mediated pulmonary disease or disorder such as asthma, IPF, or COPD; or a chronic inflammatory skin disease or disorder such as atopic dermatitis. Clinical symptoms associated with the IL-13-mediated disease or disorders, e.g., IL-13-mediated pulmonary disease or disorders such as asthma, IPF or COPD; or a chronic inflammatory skin disease or disorder such as or atopic dermatitis that can be treated by the methods and systems of the disclosure are well known to those skilled in the art. Further, those skilled in the art will appreciate that the therapeutic effects need not be complete or curative, as long as some benefit is provided to the subject. In some aspects, the term “therapeutically effective” refers to an amount of a therapeutic agent therapeutic agent that is capable of reducing IL-13 activity in a patient in need thereof.

As used herein, a “sufficient amount” or “an amount sufficient to” achieve a particular result in a patient having an IL-13-mediated disease or disorder refers to an amount of a therapeutic agent (e.g., an antibody such as tralokinumab) that is effective to produce a desired effect, which is optionally a therapeutic effect (i.e., by administration of a therapeutically effective amount). In some aspects, such particular result is a reduction in IL-13 activity in a patient in need thereof.

The term “sample” as used herein includes any biological fluid or tissue, such as whole blood, serum, muscle, saliva, or skin obtained from a subject. Samples include any biological fluid or tissue, such as whole blood, serum, muscle, saliva, urine, synovial fluid, bone marrow, cerebrospinal fluid, nasal secretions, sputum, amniotic fluid, bronchoalveolar lavage fluid, lung tissue, peripheral blood mononuclear cells, total white blood cells, lymph node cells, spleen cells, tonsil cells, or skin. In some specific aspects, that sample is blood or a fraction thereof, muscle, skin, or a combination thereof. Samples can be obtained by any means known in the art. In some aspects, a sample is a computed tomography (CT) scan of a patient's organ or tissue including, but not limited to the lungs. In some aspects, a sample can be derived by taking biological samples from a number of subjects and pooling them or pooling an aliquot of each subjects' biological sample. The pooled sample can be treated as a sample from a single subject. The term sample also includes experimentally separated fractions of all of the preceding. For example, a blood sample can be fractionated into serum or into fractions containing particular types of cells. In some aspects, a sample can be a combination of samples from an individual, such as a combination of a tissue and fluid sample.

In order to apply the methods and systems of the disclosure, samples from a patient can be obtained before or after the administration of a therapy to treat an IL-13-mediated disease or disorder. In some cases, successive samples can be obtained from the patient after therapy has commenced or after therapy has ceased. Samples can, for example, be requested by a healthcare provider (e.g., a doctor) or healthcare benefits provider, obtained and/or processed by the same or a different healthcare provider (e.g., a nurse, a hospital) or a clinical laboratory, and after processing, the results can be forwarded to the original healthcare provider or yet another healthcare provider, healthcare benefits provider or the patient. Similarly, the measuring/determination of one or more scores, comparisons between scores, evaluation of the scores and treatment decisions can be performed by one or more healthcare providers, healthcare benefits providers, and/or clinical laboratories.

As used herein, the term “healthcare provider” refers to individuals or institutions that directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy to treat an IL-13-mediated disease or disorder. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy (e.g., a therapeutic agent that treats an IL-13-mediated disease or disorder such as asthma, IPF, COPD, ulcerative colitis, or atopic dermatitis), commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition a healthcare benefits provides can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

As used herein, the term “Computed Tomography” or “CT” refers to an imaging method using tomographic images (virtual ‘slices’) of specific areas of a scanned organ, tissue or object. Digital geometry processing is used to generate a three-dimensional (3D) image of the inside of an object or organ from a series of two-dimensional (2D) radiographic images taken around a single axis of rotation.

As used herein, the term “Computed Tomography scan” or “CT scan” refers to the production of tomographic images obtained using any method suitable including, but not limited to, x-rays, multidetector computed tomography (MDCT), high-resolution computed tomography (HRCT), positron emission tomography (PET), positron emission tomography computed tomography (PET-CT) single-photon emission computed tomography (SPECT), magnetic resonance imaging (MRI), computed axial tomography (CAT scan), computer-assisted tomography, xenon ventilation computed tomography, and hyperpolarized gas lung MRI ventilation imaging.

### II. DPP4 AS A BIOMARKER

The term “DPP4” as used herein refers to the dipeptidyl peptidase IV protein (EC 3.4.14.5; Uniprot: P27487) encoded by the DPP4 gene. DPP4 is also known as DPP-IV, adenosine deaminase complexing protein 2, or CD26 (cluster of differentiation 26). DPP4 is related to attractin, FAP, DPP8 and DPP9. DPP4 is a highly conserved multifunctional type II transmembrane glycoprotein, which is present both in circulation (plasma) and on the surface of several cell types, including epithelial, endothelial and lymphoid cells. DPP4 is part of the serine protease family that is involved in T-cell co-stimulation, chemokine biology, type II diabetes, and tumor biology (Zhong et al., Atherosclerosis 2013; 226:305-314). The endogenous substrates of DPP4 include a wide variety of proline-containing peptides such as growth factors, chemokines, neuropeptides and vasoactive peptides (Gorrell, M., Clin. Sci. 108, 277-292, 2005; McIntosh, C. H. S., et al. Int. J. Biochem. Cell Biol. 38, 860-872, 2006). A role for DPP4 in inflammatory respiratory diseases like asthma is suggested by Giovannini-Chami (Giovannini-Chami et al., European Respiratory Journal. 2012 May; 39(5):1197-205), who found elevated DPP4 transcripts (and other Th2 signature genes) in the nasal epithelia of children with dust mite allergic rhinitis, associated with uncontrolled asthma. The term DPP4 also includes fragments, variants (e.g., the K1R, V7I, S437I, T557I, D663E variants known in the arts), and derivatives thereof (e.g., glycosylated or aglycosilated protein forms of the DPP4 protein, or otherwise chemically modified forms of the protein). In some aspects, the term DPP4 refers to the DPP4 gene, which includes genomic DNA, cDNA, mRNA, and fragments thereof. In some aspects, the term DPP4 also refers to oligonucleotides capable of specifically hybridizing to the DPP4 gene under stringent conditions. In some aspects, the oligonucleotides comprise nucleobases different from A, T, C, G, or U, for example, universal bases.

The term “level”, e.g., as in “DPP4 level” refers to a measurement that is made using any analytical method for detecting presence or expression of DPP4 (protein expression or gene expression) in a biological sample and that indicates the presence, absence, absolute amount or concentration, relative amount or concentration, titer, expression level, ratio of measured levels, or the like, of, for, or corresponding to DPP4 in the biological sample. The exact nature of the “value” or “level” depends on the specific designs and components of the particular analytical method employed to detect DPP4 (e.g., immunoassays, mass spectrometry methods, in vivo molecular imaging, gene expression profiling, aptamer-based assays, etc.). See, e.g., U.S. 2010/00221752.

As used herein with reference to DPP4, the terms “elevated DPP4,” “high DPP4,” “elevated DPP4 level,” or “high DPP4 level” refer to a level in a biological sample (e.g., blood serum) that is higher than a normal level or range. The normal level or range for DPP4 is defined in accordance with standard practice. Thus, the level measured in a particular biological sample can be compared with level or range of levels determined in similar normal samples. In this context, a normal sample would be a sample obtained from an individual with no detectable IL-13-mediated disease symptoms. The level of DPP4 is said to be elevated wherein the DPP4 is present in the test sample at a higher level or range than in a normal sample.

The methods disclosed herein can be carried out using any sample that may contain soluble DPP4, as well as samples containing the membrane bound form of DPP4, its intracellular, transmembrane, or extracellular moieties, or any peptide fraction thereof. Convenient samples include, for example, blood, blood cells, serum, plasma, urine, etc. In some aspects, the sample can be pretreated as necessary by dilution in an appropriate buffer solution or concentrated. Any of a number of standard aqueous buffer solutions and/or protease inhibitor, employing any of a variety of buffers, such as phosphate, Tris, or the like, at physiological pH, can be used.

DPP4 levels (either expressed protein levels, or nucleic acid levels such as mRNA levels) can be detected and quantified by any of a number of methods well known to those of skill in the art. These methods include analytic biochemical methods such as electrophoresis, capillary electrophoresis, high performance liquid chromatography (HPLC), thin layer chromatography (TLC), hyperdiffusion chromatography, mass spectroscopy and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

In one aspect, DPP4 can be detected and/or quantified in an electrophoretic polypeptide separation (e.g., a 1- or 2-dimensional electrophoresis). Means of detecting polypeptides using electrophoretic techniques are well known to those skilled in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.). A variation of this aspect utilizes a Western blot (immunoblot) analysis to detect and quantify the presence of DPP4 in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the sample with antibodies that specifically bind the analyte. Antibodies that specifically bind to the analyte may be directly labeled or alternatively may be detected subsequently using labeled antibodies (e.g., labeled sheep anti-mouse antibodies) that specifically bind to a domain of the primary antibody.

In some aspects, the sample and/or DPP4 is transformed in some manner in the course of the detection and/or quantitation assay. For example, the sample can be fractionated such that DPP4 is separated from at least one other sample component. DPP4 can be recovered in a liquid fraction or can be detected while embedded in a separation medium, such as a gel. For mass spectroscopy, DPP4 is volatilized for detection.

In a specific aspect, DPP4 is detected and/or quantified in the biological sample using an immunoassay. For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991). In some aspects, the immunoassay can use one or more anti-DPP4 antibodies or antigen binding fragments thereof which recognize human DDP4.

In certain aspects, the immunoassay comprises a sandwich immunoassay, e.g., an enzyme-linked immunosorbent assay (ELISA) or a sandwich electrochemiluminescent (ECL) assay, in which a first anti-DPP4 “capture” antibody or antigen-binding fragment thereof is attached to a solid support, antigen from a sample or standard is allowed to bind to the capture antibody, and then a second anti-DPP4 “detection” antibody or antigen binding fragment thereof is added and detected either by an enzymatic reaction, an ECL reaction, radioactivity, or other detection method.

In certain aspects, the immunoassay comprises the following steps: First, the capture antibody or fragment thereof is allowed to bind to a solid support, e.g., a multi-well plate or other assay device known to those of ordinary skill in the art. The capture antibody is allowed to attach for a period of time, e.g., overnight, and then unbound antibody is removed. The plate can then be washed to remove any unbound capture antibody. The plate can then be treated with a blocking solution to allow non-specific protein to bind to any unbound regions of the solid support.

Typical blocking solutions include an unrelated protein, e.g., nonfat dry milk or serum albumin. The plate can then again be washed to remove any unbound blocking solution. Next, a sample suspected of containing DPP4 is added to the plate. Samples are typically serially diluted and plated in duplicate or triplicate. Controls, including standard amounts of DPP4 or a suitable fragment thereof and various negative controls are also included. The antigen is allowed to bind to the capture antibody for a period of time, e.g., one hour at room temperature. Following incubation, the plate can then be washed to remove any unbound antigen.

Next, a detection antibody is added. The detection antibody is typically an anti-DPP4 antibody that binds to a different DPP4 epitope than the capture antibody. The detection antibody can be labeled or unlabeled. Where the detection antibody is unlabeled, an addition step of addition a labeled secondary antibody will be required, as is well known by those of ordinary skill in the art. The detection antibody can be directly labeled with an enzyme, e.g., horseradish peroxidase or alkaline phosphatase, or can be labeled with a tag that will allow an enzyme to bind. For example the detection antibody can be conjugated to biotin, and the enzyme attached in a subsequent step by allowing enzyme-conjugated streptavidin to bind to the biotin tag.

Alternatively the detection antibody can be conjugated to a chemiluminescent, fluorescent, or ECL tag. An example of the latter is a ruthenium chelate. Following incubation, the plate can then be washed to remove any unbound detection antibody.

Detection of the detection antibody can be accomplished by methods that vary based on the type of detection antibody that is used. If the detection antibody is tagged with biotin, then enzyme-conjugated streptavidin is added, unbound streptavidin is washed away, and a substrate is added which provides a colorimetric reaction that can be read, e.g., on a spectrophotometer. If the detection antibody is conjugated to a ruthenium chelate, the plate is subjected to electrical current, and light emission is measured.

In certain aspects, the method directly measures DPP4 levels in a patient sample, where absolute levels are calculated by plotting the immunoassay results on a standard curve using, e.g., purified full length or a DPP4 fragment. The detected signal from the detection antibody can then be quantitated based on the various standards and controls included on the plate. By plotting the results on a standard curve, the absolute levels of DPP4 in the test samples can be calculated, e.g., in ng DPP4/mL or ng DPP4/mg protein.

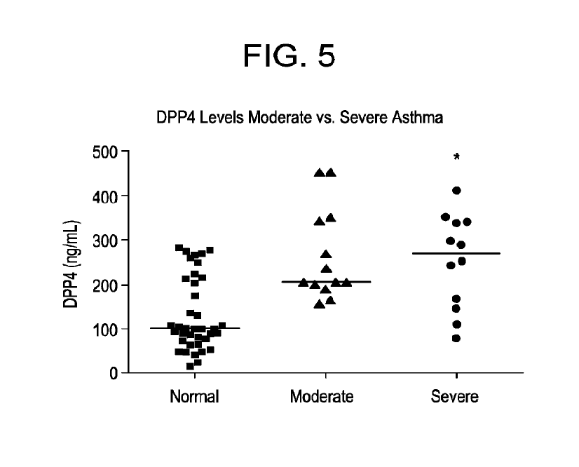

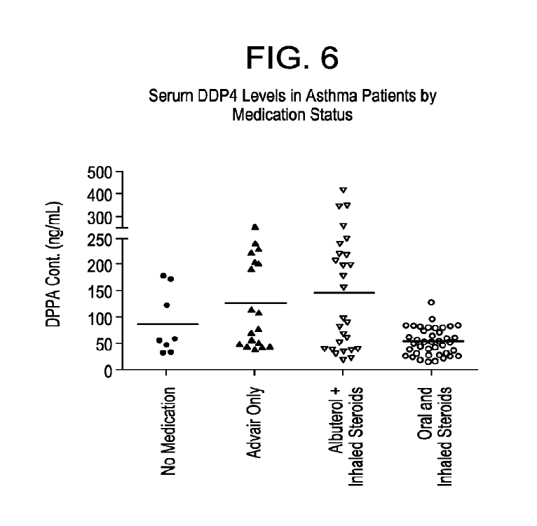

Based on comparison to known control samples, a “DPP4 threshold level” can be determined, and test samples that fall above that DPP4 threshold level (e.g., a DPP4 protein expression and/or gene expression threshold level) can indicate that the patient from whom the sample of taken may benefit from treatment with an IL-13 antagonist, for example, an anti-IL-13 antibody such as tralokinumab. DPP4 threshold levels (e.g., protein expression levels or gene expression levels) must be predetermined, and must be matched as to the type of sample (e.g., serum, lung tissue, skin), the type of disease (e.g., asthma, IPF, COPD, UC, or atopic dermatitis), and in some instances, the assay used. In some aspects, the predetermined DPP4 threshold level in a serum sample can be a DPP4 median or DPP4 mean level as depicted in FIG. 5-6, 18-20 or 24. In some aspects, the predetermined DPP4 threshold level in a serum sample can be at least about 100 ng DPP4/mL serum to about 1000 ng DPP4/mL serum, e.g., at least about 100 ng DPP4/mL serum, at least about 150 ng DPP4/mL serum, at least about 200 ng DPP4/mL serum, at least about 250 ng DPP4/mL serum, about 300 ng DPP4/mL serum, at least about 350 ng DPP4/mL serum, at least about 400 ng DPP4/mL serum, at least about 450 ng DPP4/mL serum, at least about 500 ng DPP4/mL serum, at least about 550 ng DPP4/mL serum, at least about 600 ng DPP4/mL serum, at least about 650 ng DPP4/mL serum, at least about 700 ng DPP4/mL serum, at least about 750 ng DPP4/mL serum, at least about 800 ng DPP4/mL serum, at least about 850 ng DPP4/mL serum, or at least about 900 ng DPP4/mL serum. In some aspects, the predetermined DPP4 threshold level is at least about 300 ng DPP4/mL, at least about 310 ng DPP4/mL, at least about 320 ng DPP4/mL, at least about 330 ng DPP4/mL, at least about 340 ng DPP4/mL, at least about 350 ng DPP4/mL, at least about 360 ng DPP4/mL, at least about 370 ng DPP4/mL, at least about 380 ng DPP4/mL, at least about 390 ng DPP4/mL, at least 400 ng DPP4/mL, at least about 410 ng DPP4/mL, at least about 420 ng DPP4/mL, at least about 430 ng DPP4/mL, at least about 440 ng DPP4/mL, at least about 450 ng DPP4/mL, at least about 460 ng DPP4/mL, at least about 470 ng DPP4/mL, at least about 480 ng DPP4/mL, at least about 490 ng DPP4/mL, at least 500 ng DPP4/mL, at least about 510 ng DPP4/mL, at least about 520 ng DPP4/mL, at least about 530 ng DPP4/mL, at least about 540 ng DPP4/mL, at least about 550 ng DPP4/mL, at least about 560 ng DPP4/mL, at least about 570 ng DPP4/mL, at least about 580 ng DPP4/mL, at least about 590 ng DPP4/mL, or at least 600 ng DPP4/mL.

In some aspects, the predetermined DPP4 threshold level in a serum sample can be at least about 200 ng DPP4/mL serum to about 500 ng DPP4/mL serum. In some aspects, the predetermined DPP4 threshold level in a serum sample can be at least about 300 ng DPP4/mL serum to about 400 ng DPP4/mL serum. In some aspects, the predetermined DPP4 threshold level in a serum sample can be at least about 315 ng DPP4/mL serum to about 380 ng DPP4/mL serum. In some aspects, DPP4 levels in serum are measured using ELISA. In some specific aspects, the ELISA is a QUANTIKINE® assay.

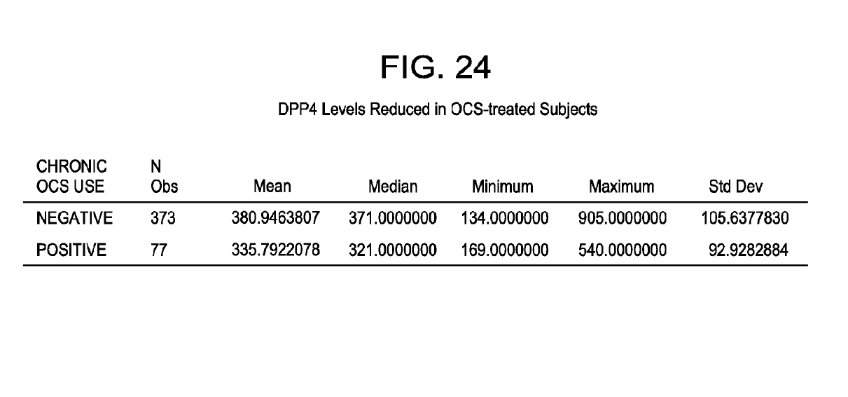

DPP4 levels quantified obtained using a QUANTIKINE® DPP4 assay (Example 2) and serum samples from a population chronic oral corticosteroid users indicated that the median value was 371 ng/mL, with a minimum value of 134 ng/mL, and a maximum value of 905 ng/mL. See FIG. 24. Accordingly, in some aspects, the predetermined DPP4 threshold is such median value, i.e., about 371 ng DPP4/mL of serum.

DPP4 levels quantified obtained using a QUANTIKINE® DPP4 assay (Example 2) and serum samples from a population of non-users or chronic oral corticosteroid (OCS) users indicated that the median value was 321 ng/mL, with a minimum value of 169 ng/mL, and a maximum value of 540 ng/mL. See FIG. 24. Accordingly, in some aspects, the predetermined DPP4 threshold is such median value, i.e., about 321 ng DPP4/mL of serum.

DPP4 levels quantified obtained using a QUANTIKINE® DPP4 assay (Example 2) and serum samples from 437 subjects enrolled in a clinical study (see Example 3) indicated that the median value was 364 ng/mL, with a minimum value of 134 ng/mL, and a maximum value of 905 ng/mL. Accordingly, in some aspects, the predetermined DPP4 threshold is such median value, i.e., about 364 ng DPP4/mL of serum. In the placebo group, the median value was 343 ng DPP4/mL of serum. See Table 5. Thus, in some aspects, the predetermined DPP4 threshold is about 343 ng DPP4/mL of serum. In the group treated with tralokinumab every two weeks (Q2W) the median value was 372 ng DPP4/mL of serum. See Table 5. Accordingly, in some aspects the predetermined DPP4 threshold is about 372 ng DPP4/mL of serum. In the group treated with tralokinumab every 4 weeks (Q4W) the median value was 375 DPP4 ng/mL of serum. See Table 5. Thus, in some aspects, the predetermined DPP4 threshold is about 375 DPP4 ng/mL of serum.

As indicated in the QUANTIKINE® DPP4 assay manufacturer's manual, normal DPP4 levels in serum, EDTA plasma, or heparin plasma using a QUANTIKINE® human DPP4 immunoassay are 197-615 ng/mL, 187-604 ng/mL, and 159-588 ng/mL respectively. For urine, normal DPP4 levels are 2.26-13.3 ng/mL. For saliva, normal DPP4 levels measured are 13.0-69.9 ng/mL.

The DPP4 threshold level (e.g., a protein expression level or a gene expression level) can vary based on the nature of the assay, e.g., the capture and detection antibodies used, the source, purity, and composition of the DPP4 standard, and the like. In one aspect, instead of using an arbitrary threshold level to determine whether a patient can benefit from treatment with an IL-13 antagonist (e.g., an anti-IL-13 antibody such as tralokinumab), the patient's DPP4 levels can be compared to one or more control DPP4 levels. According to this aspect, the test sample (e.g., a sample from a patient suffering from an IL-13-mediated disease or disorder) is compared to one or more control samples (e.g., samples taken from normal healthy individuals, earlier samples taken from the same patient, samples taken from patients with a non-IL-13-mediated subset of the patient's disease, e.g., asthma, COPD, IPF, UC, or atopic dermatitis, a pre-determined standard amount of isolated DPP4, or a combination thereof).

The results can be expressed as a ratio with the control samples to determine a percent increase or a percent decrease in the patient's DPP4 levels (e.g., a protein expression level or a gene expression level) compared to the control DPP4 levels. According to this aspect, the control sample can be a matched pair with the patient sample, e.g., one or more of whole blood if the patient sample is whole blood, serum if the patient sample is serum, plasma if the patient sample is plasma, saliva if the patient sample is saliva, urine if the patient sample is urine, sputum if the patient sample is sputum, bronchoalveolar lavage fluid if the patient sample is bronchoalveolar lavage fluid, lung tissue if the patient sample is lung tissue, or skin if the patient sample is skin.

A DPP4 level (e.g., a protein expression level or a gene expression level) is considered to be increased if it is at least about 10%, at least 20%, at least about 30%, at least about 40%, at least about 50%, at least about 60%, at least about 70%, at least about 80%, at least about 90%, or at least about 100% higher than the control DPP4 level. A DPP4 is considered to be decreased if it is at least about 10%, at least 20%, at least about 30%, at least about 40%, at least about 50%, at least about 60%, at least about 70%, at least about 80%, at least about 90%, or at least about 100% lower than the control DPP4 level.

Immunoassays for detecting DPP4 can be either competitive or noncompetitive. Noncompetitive immunoassays are assays in which the amount of captured analyte is directly measured. In competitive assays, the amount of analyte in the sample is measured indirectly by measuring the amount of an added (exogenous) labeled analyte displaced (or competed away) from a capture agent by the analyte present in the sample. In one competitive assay, a known amount of, in this case, labeled DPP4 is added to the sample, and the sample is then contacted with a capture agent. The amount of labeled DPP4 bound to the antibody is inversely proportional to the concentration of DPP4 present in the sample.

DPP4 detection assays can be scored (as positive or negative or quantity of analyte) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of analyte concentration.

Once determined, a DPP4 level (e.g., a protein expression level or a gene expression level) can be recorded in a patient medical record. In some aspects, the methods disclosed herein include making a diagnosis, often a differential diagnosis, based at least in part on the DPP4 level.

As used herein, the term “differential diagnosis” refers to the determination of which of two or more diseases with similar symptoms is likely responsible for a subject's symptom(s), based on an analysis of the clinical data. The term can also refer to the determination of whether a patient is susceptible to treatment with an IL-13-antagonist depending on whether the measured DPP4 level (e.g., a protein expression level or a gene expression level) in a sample from the patient sample is above a predetermined DPP4 threshold level, or is elevated relative to the DPP4 level in one or more control samples.

In particular aspects, the methods disclosed herein include informing the subject of a result of the DPP4 assay and/or of a diagnosis based at least in part on the DPP4 level. The patient can be informed verbally, in writing, and/or electronically.

This diagnosis can also be recorded in a patient medical record. For example, in various aspects, the diagnostic of an IL-13-mediated disease or disorder treatable with a specific IL-13 antagonist is recorded in a medical record. The term “medical record” or “patient medical record” refers to an account of a patient's examination and/or treatment that typically includes one or more of the following: the patient's medical history and complaints, the physician's physical findings, the results of diagnostic tests and procedures, and patient medications and therapeutic procedures. A medical record is typically made by one or more physicians and/or physicians' assistants and it is a written, transcribed or otherwise recorded record and/or history of various illnesses or injuries requiring medical care, and/or inoculations, and/or allergies, and/or treatments, and/or prognosis, and/or frequently health information about parents, siblings, and/or occupation. The record may be reviewed by a physician in diagnosing the condition.

The medical record can be in paper form and/or can be maintained in a computer-readable medium. The medical record can be maintained by a laboratory, physician's office, a hospital, a healthcare maintenance organization, an insurance company, and/or a personal medical record website. In some aspects, a diagnosis, based at least in part on the DPP4 level, is recorded on or in a medical alert article such as a card, a worn article, and/or a radiofrequency identification (RFID) tag. As used herein, the term “worn article” refers to any article that can be worn on a subject's body, including, but not limited to, a tag, bracelet, necklace, arm band, or head band.

The methods disclosed herein also include prescribing, initiating, and/or altering prophylaxis and/or therapy, e.g., for an IL-13 mediated disease or disorder (e.g., asthma, IPF, COPD or atopic dermatitis). In certain aspects, the methods can entail ordering and/or performing one or more additional assays. For example, if the DPP4 level (e.g., a protein expression level or a gene expression level) is determined to be within a normal range (i.e., not elevated), the DPP4 assay may be repeated to rule out a false negative result, and/or one or more additional DPP4 assays may be performed to monitor the subject's status. If the DPP4 level (e.g., a protein expression level or a gene expression level) is determined to be elevated, it may be desirable repeat the DPP4 assay to rule out a false positive result. In certain aspects, it will be desirable to assay another indicator of, e.g., IL-13 mediated disease (e.g., asthma, IPF, COPD or atopic dermatitis), to confirm a diagnosis.

A person skilled in the art would understand that DPP4 levels (e.g., a protein expression level or a gene expression level) can be used according to the methods disclosed herein, including but not limited to treatment, diagnostic, and monitoring methods, as (i) positive selectors, i.e., a specific action would be taken (e.g., treating a patient having an IL-13-mediated disease or disorder with an IL-13 antagonist) if the DPP4 level (e.g., a protein expression level or a gene expression level) in a sample taken from the patient is above a predetermined DPP4 threshold level, or is elevated relative to the DPP4 level in one or more control samples; or (ii) negative selectors, i.e., a specific action would be taken (e.g., treating a patient having an IL-13-mediated disease or disorder with an IL-13 antagonist) if the DPP4 level in a sample taken from the patient is below a predetermined DPP4 threshold level, or is lower relative to the DPP4 level in one or more control samples; or (iii) both positive and negative selectors, for example, a specific treatment could cease (e.g., oral corticosteroid treatment) and a different treatment could commence (e.g., treatment with an anti-IL-13 antibody) if the DPP4 level in a sample taken from the patient is above/below a predetermined DPP4 threshold level, or is higher/lower relative to the DPP4 level in one or more control samples

### III. METHODS OF DIAGNOSIS AND TREATMENT

This disclosure provides a method of treating a patient having an IL-13-mediated disease or disorder, or a patient with a pulmonary disease or disorder, inflammatory bowel disease or disorder, or chronic inflammatory skin disease or disorder of unknown etiology which might be IL-13-mediated, comprising administering an IL-13 antagonist to the patient if the DPP4 level in a sample (e.g., a protein expression level or a gene expression level) taken from the patient is above a predetermined DPP4 threshold level, or is elevated relative to the DPP4 level in one or more control samples. In one aspect, the patient's DPP4 level is measured in an immunoassay employing one or more anti-DPP4 antibodies or antigen binding fragments thereof which recognize human DPP4, or antigen-binding fragments, variants or derivatives thereof.

This disclosure also provides methods, assays, and kits to facilitate a determination by a healthcare provider, a healthcare benefits provider, or a clinical laboratory to as to whether a patient will benefit from treatment with an IL-13 antagonist, e.g., an ant-IL-13 antibody or antigen-binding fragment thereof, e.g., tralokinumab, or a fragment, variant, or derivative thereof, an antibody or fragment thereof that binds to the same IL-13 epitope as tralokinumab, or an antibody or fragment thereof that competitively inhibits binding of tralokinumab to IL-13. The methods assays and kits provided herein will also facilitate a determination by a healthcare provider, a healthcare benefits provider, or a clinical laboratory to as to whether a patient will benefit from treatment with any other IL-13 antagonist IL-13 disclosed herein, or known to those of ordinary skill in the art.

The present disclosure provides a method of treating a patient having an interleukin-13 (IL-13)-mediated disease or disorder (e.g., asthma, IPF, COPD or atopic dermatitis), comprising administering an IL-13 antagonist to the patient if the level of DPP4 (e.g., a protein expression level or a gene expression level) in a sample taken from the patient is above a predetermined DPP4 threshold level, or is above the DPP4 level in one or more control samples. In some aspects, the sample is obtained from the patient and is submitted for measurement of the level of DPP4 in the sample, for example, to a clinical laboratory.

Also provided is a method of treating a patient having an IL-13-mediated disease or disorder comprising (a) submitting a sample taken from the patient for measurement of the DPP4 level in the sample, wherein the patient's DPP4 level is measured, for example, in an immunoassay employing one or more anti-DPP4 antibodies or antigen binding fragments thereof which recognize human DPP4; and, (b) administering an IL-13 antagonist to the patient if the patient's DPP4 level in the sample is above a predetermined DPP4 threshold level, or is above the DPP4 level in one or more control samples.

The disclosure also provides a method of treating a patient having an IL-13-mediated disease or disorder comprising (a) measuring the DPP4 level in a sample obtained from a patient having an IL-13-mediated disease or disorder, wherein the patient's DPP4 level in the sample is measured, for example, in an immunoassay employing one or more anti-DPP4 antibodies or antigen binding fragments thereof which recognize human DPP4; (b) determining whether the patient's DPP4 level in the sample is above a predetermined DPP4 threshold level, or is above the DPP4 level in one or more control samples; and, (c) advising a healthcare provider to administer an IL-13 antagonist to the patient if the patient's DPP4 level is above a predetermined DPP4 threshold level, or is above the DPP4 level in one or more control samples.

In some aspects, the patient's DPP4 level is measured in an immunoassay employing one or more anti-DPP4 antibodies or antigen binding fragments thereof which recognize human DPP4. In other aspects, the patients DPP4 level (e.g., DNA or RNA level) is measured in an assay employing one or more oligonucleotide probes capable of specifically measuring the expression of the DPP4 gene.

In certain aspects, the DPP4 detection assay (e.g., an immunoassay) is performed on a sample obtained from the patient, by the healthcare professional treating the patient (e.g., using an immunoassay as described herein, formulated as a “point of care” diagnostic kit). In some aspects, a sample is obtained from the patient and is submitted, e.g., to a clinical laboratory, for measurement of the DPP4 level in the sample according to the healthcare professional's instructions (e.g., using an immunoassay as described herein). In certain aspects, the clinical laboratory performing the assay will advise the healthcare provide as to whether the patient can benefit from treatment with an IL-13 antagonist based on whether the patient's DPP4 level is above a predetermined DPP4 threshold value or is elevated relative to one or more control samples.

In certain aspects, this disclosure provides a method of treating a patient having an IL-13-mediated disease or disorder over a period of time, comprising: measuring a first DPP4 level (e.g., protein expression level or gene expression level) in a first sample taken from the patient, or submitting a first sample taken from the patient for measurement of a first DPP4 level in the sample, wherein the patient's DPP4 level is, for example, measured in an immunoassay employing one or more anti-DPP4 antibodies or antigen binding fragments thereof which recognize human DPP4, and administering an IL-13 antagonist to the patient if the patient's DPP4 level in the first sample is above a predetermined DPP4 threshold level, or is elevated relative to the DPP4 level in one or more control samples. The test can be performed by a healthcare provider or a clinical laboratory as noted above.

According to this aspect, the method can further comprise: measuring a second DPP4 level (e.g., protein expression level or gene expression level) in a second sample taken from the patient, or submitting a second sample taken from the patient for measurement of a second DPP4 level in the sample, wherein the patient's DPP4 level is again measured, for example, in an immunoassay employing one or more anti-DPP4 antibodies or antigen binding fragments thereof which recognize human DPP4; comparing the first and second DPP4 levels in the patient, and altering the dose, e.g., increasing or maintaining the amount or frequency of the IL-13 antagonist administered to the patient, or even discontinuing IL-13 antagonist therapy if the patient's DPP4 level in the second sample is higher than the DPP4 level in the first sample, or maintaining or reducing the amount or frequency of the IL-13 antagonist administered to the patient if the patient's DPP4 level in the second sample is lower than or about the same as the DPP4 level in the first sample.

In certain aspects of all method of treatment aspects provided herein, a “loading” dose of an IL-13 antagonist is administered to achieve a desired therapeutic level in the patient. If the loading dose does not affect the patient's DPP4 levels (e.g., protein expression levels or gene expression levels) significantly or the patient's DPP4 levels rise, a decision could be made to discontinue treatment—e.g., to use a non-IL-13 antagonist therapy. If the loading dose results in steady or reduced DPP4 levels in the patient a decision could be made to reduce the dose size or frequency to a “maintenance” dose. It is important to note that the methods provided here are guidelines for a healthcare provider to administer treatment, and the ultimate treatment decision will be based on the healthcare provider's sound judgment.

In certain aspects, results of an immunoassay as provided herein can be submitted to a healthcare benefits provider for determination of whether the patient's insurance will cover treatment with an IL-13 antagonist.

In certain aspects this disclosure provides a method of treating a patient having an IL-13-mediated disease or disorder comprising: measuring, e.g., in a clinical laboratory, the DPP4 level (e.g., protein expression level or gene expression level) in a first sample obtained from a patient having an IL-13-mediated disease or disorder, e.g., a sample provided by a healthcare provider, wherein the patient's DPP4 level in the first sample is, for example, measured in an immunoassay employing one or more anti-DPP4 antibodies or antigen binding fragments thereof which recognize human DPP4, determining whether the patient's DPP4 level in the first sample is above a predetermined DPP4 threshold level, or is elevated relative to the DPP4 level in one or more control samples; and advising a healthcare provider to administer an IL-13 antagonist to the patient if the patient's DPP4 level is above a predetermined DPP4 threshold level, or is elevated relative to the DPP4 level in one or more control samples.

In certain aspects, this method can further comprise: measuring the DPP4 level (e.g., protein expression level or gene expression level) in a second sample obtained from the patient, e.g., a sample provided by a healthcare provider, wherein the patient's DPP4 level is again measured, for example, in an immunoassay employing one or more anti-DPP4 antibodies or antigen binding fragments thereof which recognize human DPP4; determining whether the patient's DPP4 level in the second sample is higher than, about the same as, or lower than the DPP4 level measured in the first sample; and advising a healthcare provider to adjust the IL-13 antagonist therapy if indicated, e.g., to increase or maintain the amount or frequency of the IL-13 antagonist administered to the patient, or discontinuing IL-13 antagonist therapy, if the patient's DPP4 level in the second sample is higher than the DPP4 level in the first sample, or to maintain or reduce the amount or frequency of the IL-13 antagonist administered to the patient if the patient's DPP4 level in the second sample is lower than or about the same as the DPP4 level in the first sample.

In some aspects, a sample is obtained from the patient and is submitted, e.g., to a clinical laboratory, for measurement of the DPP4 level (e.g., protein expression level or gene expression level) in the sample, e.g., using an immunoassay. In certain aspects, the clinical laboratory performing the assay will advise the healthcare provide as to whether the patient can benefit from treatment with an IL-13 antagonist based on whether the patient's DPP4 level (e.g., protein expression level or gene expression level) is above a predetermined DPP4 threshold value or is elevated relative to one or more control samples.

Similarly, this disclosure provides a method of monitoring the therapeutic efficacy of an IL-13 antagonist therapeutic regimen in a patient having an IL-13-mediated disease or disorder comprising: measuring, or instructing a clinical laboratory to measure the DPP4 level (e.g., protein expression level or gene expression level) in a first sample obtained from a patient having an IL-13-mediated disease or disorder, wherein the patient's DPP4 level is measured, for example, in an immunoassay employing one or more anti-DPP4 antibodies or antigen binding fragments thereof which recognize human DPP4; administering, or advising a healthcare professional to administer an IL-13 antagonist to a patient if the patient's DPP4 level in the first sample is above a predetermined DPP4 threshold level, or is elevated relative to the DPP4 level in one or more control samples; measuring the DPP4 level in a second sample obtained from the patient, wherein the patient's DPP4 level is again measured, for example, in an immunoassay employing one or more anti-DPP4 antibodies or antigen binding fragments thereof which recognize human DPP4, and determining, or obtaining results indicating whether the patient's DPP4 level in the second sample is higher than, about the same as, or lower than the DPP4 level measured in the first sample; wherein the IL-13 antagonist therapeutic regimen is effective if the patient's DPP4 level in the second sample is lower than or about the same as the DPP4 level in the first sample.

In certain aspects, a patient is diagnosed with a pulmonary disease or disorder, and in the course of diagnosis a determination can be made as whether to treat the patient with an IL-13 antagonist. Accordingly, in certain aspects this disclosure provides a method of treating a patient diagnosed with a pulmonary disease or disorder, comprising administering an IL-13 antagonist to the patient if the DPP4 level (e.g., protein expression level or gene expression level) in a sample taken from the patient is above a predetermined DPP4 threshold level, or is above the DPP4 level in one or more control samples; wherein the patient's DPP4 level is measured, for example, in an immunoassay employing one or more anti-DPP4 antibodies or antigen binding fragments thereof which recognize human DPP4.

In certain aspects this disclosure provides a method of treating a patient diagnosed with a pulmonary disease or disorder (e.g., asthma, IPF or COPD) or a chronic inflammatory skin disease or disorder (e.g., or atopic dermatitis) comprising:

(a) submitting a sample taken from the patient for measurement of the DPP4 level (e.g., protein expression level or gene expression level) in the sample, wherein the patient's DPP4 level is, for example, measured in an immunoassay employing one or more anti-DPP4 antibodies or antigen binding fragments thereof which recognize human DPP4; and,
(b) administering an IL-13 antagonist to a patient if the patient's DPP4 level in the sample is above a predetermined DPP4 threshold level, or is above the DPP4 level in one or more control samples.

In certain aspects this disclosure also provides a method of determining whether to treat a patient diagnosed with a pulmonary disease or disorder (e.g., asthma, IPF or COPD) or a chronic inflammatory skin disease or disorder (e.g., or atopic dermatitis) with an IL-13 antagonist therapeutic regimen comprising:

(a) measuring, or instructing a clinical laboratory to measure the DPP4 level (e.g., protein expression level or gene expression level) in a sample obtained from a patient diagnosed with a pulmonary disease or disorder (e.g., asthma, IPF or COPD) or a chronic inflammatory skin disease or disorder (e.g., or atopic dermatitis), wherein the patient's DPP4 level is measured, for example, in an immunoassay employing one or more anti-DPP4 antibodies or antigen binding fragments thereof which recognize human DPP4; and,
(b) treating, or instructing a healthcare provider to treat the patient with an IL-13 antagonist therapeutic regimen if the patient's DPP4 level in the sample is above a predetermined DPP4 threshold level, or is above the DPP4 level in one or more control samples.

In certain aspects, this disclosure provides a method of treating a patient diagnosed with a pulmonary disease or disorder (e.g., asthma, IPF or COPD) or a chronic inflammatory skin disease or disorder (e.g., or atopic dermatitis) comprising: submitting a first sample taken from the patient for measurement of a first DPP4 level (e.g., protein expression level or gene expression level) in the sample, wherein the patient's DPP4 level is measured, for example, in an immunoassay employing one or more anti-DPP4 antibodies or antigen binding fragments thereof which recognize human DPP4; administering an IL-13 antagonist to a patient if the patient's DPP4 level in the first sample is above a predetermined DPP4 threshold level, or is elevated relative to the DPP4 level in one or more control samples. The DPP4 levels can be measured by a healthcare professional or by a clinical laboratory that obtains a patient sample from a healthcare professional, and is instructed to measure the DPP4 in the sample by the healthcare professional.

In certain aspects the method of treatment provided above can further comprise submitting a second sample taken from the patient for measurement of a second DPP4 level (e.g., protein expression level or gene expression level) in the sample, wherein the patient's DPP4 level is again measured, for example, in an immunoassay employing one or more anti-DPP4 antibodies or antigen binding fragments thereof which recognize human DPP4; increasing or maintaining the amount or frequency of the IL-13 antagonist administered to the patient, or even discontinuing IL-13 antagonist therapy if the patient's DPP4 level in the second sample is higher than the DPP4 level in the first sample, or maintaining or reducing the amount or frequency of the IL-13 antagonist administered to the patient if the patient's DPP4 level in the second sample is lower than or about the same as the DPP4 level in the first sample. It is important to note that the methods provided here are guidelines for a healthcare provider to administer treatment, and the ultimate treatment decision will be based on the healthcare provider's sound judgment.

In certain aspects, this disclosure provides a method of determining whether to treat a patient diagnosed with a pulmonary disease or disorder (e.g., asthma, IPF or COPD); or a chronic inflammatory skin disease or disorder (e.g., or atopic dermatitis) with an IL-13 antagonist therapeutic regimen comprising measuring, or instructing a clinical laboratory to measure the DPP4 level (e.g., protein expression level or gene expression level) in a first sample obtained from a patient diagnosed with a pulmonary disease or disorder (e.g., asthma, IPF or COPD); or a chronic inflammatory skin disease or disorder (e.g., or atopic dermatitis), wherein the patient's DPP4 level is measured, for example, in an immunoassay employing one or more anti-DPP4 antibodies or antigen binding fragments thereof which recognize human DPP4; and treating, or instructing a healthcare provider to treat the patient with an IL-13 antagonist therapeutic regimen if the patient's DPP4 level in the first sample is above a predetermined DPP4 threshold level, or is elevated relative to the DPP4 level in one or more control samples. In certain aspects, the results of the DPP4 level measuring assay (e.g., an immunoassay) can be submitted to a healthcare benefits provider to determine whether the patient's insurance will cover treatment with an IL-13 antagonist.

In some aspects, the methods disclosed herein can be used to diagnose COPD. In other aspects, the methods disclosed herein can be used to prevent progression of COPD in a subject from one stage to a subsequent stage in the COPD GOLD classification. In another aspect, the methods disclosed herein allow monitoring the progression of COPD disease from one stage to a subsequent stage in the GOLD classification. The COPD markers disclosed herein are also suited to discriminate between humans suffering from COPD stage I/II and COPD stage III/IV as defined above. The discrimination between these COPD stages is important to determine the appropriate therapy. In another aspect, the methods disclosed herein allow monitoring the progression of CPOD from one stage to a subsequent stage in the GOLD classification. In another aspect, the methods disclosed herein allows monitoring the progress of a COPD therapy. In yet another aspect, the methods disclosed herein can be used to prevent or ameliorate the progression of COPD in a subject from one stage to a subsequent stage in the COPD GOLD classification. In some aspects, the methods disclosed herein can be used to prevent, treat, or ameliorate COPD exacerbations.

In certain aspects, the patient has been treated or is being treated with one or more additional medications, either before, during, or after administration of an IL-13 antagonist. Various other medications useful for treating, e.g., asthma, IPF, COPD, UC and atopic dermatitis are described elsewhere herein. In certain aspects the patient has been treated, continues to be treated, or will be treated with one or more additional medications comprising, e.g., a steroid, a bronchodilator, or a combination thereof. In certain aspects, the steroid is a corticosteroid. In some aspects, the corticosteroid is an oral corticosteroid. In some aspects, the steroid is fluticasone or budesonide. In some aspects, the bronchodilator is salbutamol or salmeterol. In certain aspects, the additional medication comprises at least one steroid, wherein the steroid is fluticasone or budesonide, and at least one bronchodilator, wherein the bronchodilator is salbutamol or salmeterol. In certain aspects, the one or more additional medications are administered by inhalation, by oral administration, by injection, or a combination thereof. In some aspect inhalation administration is conducted using a metered dose inhaler (MDI) or a dry powder inhaler (DPI).

In some aspects, the steroid is administered at a high dose. The term high dose when application to an inhaled corticosteroid (ICS) can refer, for example, to a total daily dose of at least 500 μg of ICS (e.g., fluticasone) DPI or at least 440 μg ICS MDI. In some aspects, the high ICS total daily dose is at least about 300 μg, at least about 350 μg, at least about 400 μg, at least about 450 μg, at least about 500 μg, at least about 550 μg, at least about 600 μg, at least about 650 μg, at least about 700 μg, at least about 750 μg, at least about 800 μg, at least about 850 μg, at least about 900 μg, at least about 950 μg, or at least 1000 μg of ICS (e.g., fluticasone) DPI. In some aspects, the high ICS total daily dose is at least about 300 μg, at least about 350 μg, at least about 400 μg, at least about 450 μg, at least about 500 μg, at least about 550 μg, at least about 600 μg, at least about 650 μg, at least about 700 μg, at least about 750 μg, at least about 800 μg, at least about 850 μg, at least about 900 μg, at least about 950 μg, or at least 1000 μg of ICS (e.g., fluticasone) MPI.

The term “high dose” when application to an inhaled corticosteroid (ICS) (e.g., fluticasone) in combination treatments (e.g., with a bronchodilator such as salmeterol) can refer, for example, to about 230 μg fluticasone and about 21 μg salmeterol as MDI at a dose of 2 inhalations twice per day, or to about 500 μg fluticasone and about 50 μg salmeterol as single dose DPI. Concentrations of corticosteroids considered to be high-dose alone as well as in combination with other therapeutic agents are well known in the art.

In certain aspects, the IL-13 antagonist comprises one or more of an anti-IL-13 antibody or antigen-binding fragment thereof e.g., tralokinumab, an IL-13 mutein, e.g., IL-13E13K (Kioi M, et al., Cell Immunol. 2004 229:41-51), an IL-4 mutein, e.g., Pitrakinra (AER-001, BAY-16-9996) (Antoniu S A., Curr Opin Investig Drugs. 2010 11:1286-94), an anti-IL-13Rα1 antibody or antigen-binding fragment thereof, or an anti-IL-4Rα antibody or antigen-binding fragment thereof. In certain aspects, the IL-13 antagonist is an anti-IL13 antibody, or antigen-binding fragment thereof. In certain aspects, the anti-IL-13 antibody or fragment thereof binds to the same IL-13 epitope as tralokinumab or competitively inhibits binding of tralokinumab to IL-13, or both. In certain aspects the antibody comprises tralokinumab or an antigen-binding fragment thereof. In other aspects, the antibody or fragment thereof consists of tralokinumab or an antigen-binding fragment thereof.

In some aspects, the anti-IL-13 antibody or fragment thereof binds to the same IL-13 epitope as lebrikizumab or competitively inhibits binding of lebrikizumab to IL-13, or both. In some aspects, the anti-IL-13 antibody or fragment thereof comprises lebrikizumab or an antigen-binding fragment thereof. In some aspects, the anti-IL-13 antibody or fragment thereof consists of lebrikizumab or an antigen-binding fragment thereof.

In some aspects, the samples used in the methods disclosed herein are taken from a patient and comprise one or more of whole blood, serum, plasma, saliva, sputum, bronchoalveolar lavage fluid, urine, lung epithelial cells, skin, or nasal polyps. In particular aspects, the sample taken from the patient is blood serum.

In some aspects, the airway dimensions at baseline (i.e. prior to administration of an IL-13 antagonist), for example, Wall Area % as determined using a CT scan of the lungs of subsegmental airways (WA %) can be used to predict treatment response (for example, improvements in airway resistant and/or FEV₁) in patients treated or candidates for treatment with an IL-13 antagonist (for example an anti-IL-13 antibody such as tralokinumab or lebrikizumab). The term “wall area” as used herein refers to the cross-sectional area of a bronchial tube wall (e.g. segmental and subsegmental bronchi in the upper lobes). Wall area percentage (WA %) is calculated as follows: 100*wall area/(wall area+lumen area). Tools to measure wall area and wall area percentage are well known in the art. See, e.g., Gupta et al., J Allergy Clin Immunol. 133(3): 729-738 (2014); Gupta et al., Thorax. 65(9):775-81 (2010). In some aspects, airway dimensions are measured from Computed Tomography (CT) imaging data of the lungs. Such imaging data can be processed, for example, using commercially available software such as VIDA Apollo (e.g., the Volumetric Information Display and Analysis (VIDA) Pulmonary Workstation, VIDA Diagnostics, Coralville, Iowa). In some aspects, WA % of subsegmental airways from CT scan data of the lungs can be used to determine, for example, whether to treat, to modify the treatment, or to monitor the treatment of a patient suffering from an IL-13-mediated disease, e.g., asthma, COPD, emphysema, IPF, UC, or atopic dermatitis. In some aspects, WA % of subsegmental airways from CT scan data can be used alone or in combination with other biomarkers (e.g., periostin, DPP4, and/or clinical characteristics such as FEV₁ reversibility) to identify a patient population suffering from and IL-13-mediated disease, e.g., asthma, COPD, emphysema, IPF, UC, or atopic dermatitis, that is responsive to anti-IL-13 therapeutic agents (e.g., tralokinumab or lebrikizumab). Accordingly, the methods provided in the present disclosure comprise evaluating WA % measured from CT scan imaging data of the lungs of a patient, and determining whether WA % of subsegmental airways is above or below a predetermined WA % threshold level, or it is above or below the WA % in one or more control CT scans, wherein patients suffering from an IL-13-mediated disease (e.g., asthma, COPD, emphysema, IPF, UC, or atopic dermatitis) having a WA % value of subsegmental airways above a predetermined WA % threshold level or above the WA % in one or more control CT scans are treated with an IL-13 antagonist (for example an anti-IL-13 antibody such as tralokinumab or lebrikizumab). WA % can be used in the methods disclosed herein independently or in combination with periostin levels, DPP4 levels and/or clinical characteristics such as FEV₁ reversibility.

In some aspects, the predetermined WA % threshold level useful in the methods disclosed herein is about 40%, about 45%, about 50%, about 55%, about 60%, about 65%, about 70%, about 75%, about 80%, about 85%, or about 90%. In some aspects, the predetermined WA % threshold level useful in the methods disclosed herein is between about 60% and about 80%. In other aspects, the predetermined WA % threshold level useful in the methods disclosed herein is between about 65% and about 75%. In other aspects, the predetermined WA % threshold level useful in the methods disclosed herein is about 60%. In other aspects, the predetermined WA % threshold level useful in the methods disclosed herein is about 68%. In specific aspects, the predetermined WA % threshold level useful in the methods disclosed herein is 68%. In some aspects, the predetermined threshold WA % level useful in the methods disclosed herein is the mean WA % value of a population of patients. In other aspects, the predetermined threshold WA % level useful in the methods disclosed herein is the median WA % value of a population of patients. In some aspects, the patients have been treated with an IL-13 antagonist (e.g., tralokinumab or lebrikizumab). In some aspects, the patients have not been treated with an IL-13 antagonist (e.g., they have been treated with a non-IL-13 antagonist therapeutic agent, or not treated with any therapeutic agent).

In some aspects, WA % is measured using 3D airway analysis of CT scan data of lung scans using segmental bronchi. In other aspects, WA % is measured using 3D airway analysis of CT scan data of lung scans using subsegmental bronchi. In some aspects, the segmental or subsegmental bronchi are from the upper lobes. In some aspects, the segmental or subsegmental bronchi are from the entire lung. In some aspects, the segmented airways are right apical (RB1), right anterior (RB2), right posterior (RB3), left apicoposterior (LB1+2), LB3 (left anterior), or combinations thereof. In some aspects, the subsegmental airways are RB1a, RB1b, RB2a, RB2b, RB3a, RB3b, LB1, LB1a, LB1b, LB2, LB2a, LB2b, LB3a, LB3b, or combinations thereof. See Naidich, et al, Imaging of the Airways—Functional and Radiologic Correlations, 2005. In some aspects, airway parameters are calculated for each airway segment separately, and then averaged over segmental and/or subsegmental airways in each subject.

In some aspects, patients with WA % above the specified threshold (e.g., WA % at least 60% at subsegmental level) display a statistically significant improvement in airway resistance. In some aspects, patients with WA % above the specified threshold (e.g., WA % at least 60% at subsegmental level) display a statistically significant improvement in pre-bronchodilator FEV1. In some aspects, WA % can be combined with other biomarkers obtained using 3D airway analysis of CT scan data for example lumen area (LA), wall area (WA), wall thickness area (WT), airway resistance, or combinations thereof.

In some aspects, in addition to the determination of the level of DPP4 (e.g., protein expression level or gene expression level), the method of the present disclosure can further comprise determining, submitting a sample taken from the patient for determination, or instructing a clinical laboratory to determine:

(i) the level of the patient's IgE levels,
(ii) the patient's eosinophil count,
(iii) the patient's Fraction of Exhaled Nitric Oxide (FE_{NO}),
(iv) the patient's Eosinophil/Lymphocyte and Eosinophil/Neutrophil (ELEN) index (see WO2012158954, which is herein incorporated by reference in its entirety),
(v) the patient's EOS index (see WO2012158954, which is herein incorporated by reference in its entirety),
(vi) the patient's wall area percentage (WA %) of subsegmental airways from CT scan data of the lungs, or
(vii) a combination of two or more thereof.

Accordingly, in certain aspects described above, the patient having an IL-13-mediated disease or disorder has been diagnosed with a pulmonary disease or disorder an inflammatory bowel disease or disorder or a chronic inflammatory skin disease or disorder, which, in a subset of differential diagnoses, can be IL-13-mediated. See, e.g., U.S. Pat. Appl. Publication 2012-0328606 incorporated herein by reference in its entirety. In certain aspects, the disease or disorder suspected of having IL-13-mediated pathology is asthma, idiopathic pulmonary fibrosis (IPF), chronic obstructive pulmonary disease (COPD), ulcerative colitis (UC), or atopic dermatitis.

In some aspects, in addition to the determination of the level of DPP4 (e.g., protein expression level or gene expression level), the methods disclosed herein can comprise determining, submitting a sample taken from the patient for determination, or instructing a clinical laboratory to determine the expression level or activity of isoforms 1, 2, 3, or 4 of human periostin, or combinations thereof. The use periostin as a biomarker for IL-13-mediated diseases has been disclosed, e.g., in Jia, et al., J Allergy Clin. Immunol 2012 130:647-654; Takayama, et al., J Allergy Clin Immunol 2006 118:98-104; and PCT Publ. No. WO 2012/083132, each herein incorporated by reference in their entirety.

The term “periostin” as used herein refers to the osteoblast specific factor protein (Uniprot: Q15063) encoded by the POSTN gene. Periostin is also known as osteoblast-specific factor 2 (OSF-2). Periostin functions as a ligand for alpha-V/beta-3 and alpha-V/beta-5 integrins to support adhesion and migration of epithelial cells. Periostin is a gla domain vitamin K dependent factor.

The term periostin also includes fragments, variants (e.g., isoforms produced by alternative splicing), and derivatives thereof (e.g., glycosylated or aglycosilated protein forms of the protein, or otherwise chemically modified forms of the protein). Seven isoforms produced by alternative splicing are known in the art: Isoform 1 (Uniprot: Q15063-1), also known as OSF-20S, which is 836 amino acids long; Isoform 2 (Uniprot: Q15063-2), also known as OSF-2p1, which is 779 amino acids long; Isoform 3 (Uniprot: Q15063-3), which is 781 amino acids long; Isoform 4 (Uniprot: Q15063-4), which is 751 amino acids long; Isoform 5 (Uniprot: Q15063-5), which is 809 amino acids long; Isoform 6 (Uniprot: Q15063-6), which is 749 amino acids long; and Isoform 7 (Uniprot: Q15063-7), which is 721 amino acids long. Known periostin variants include those with any of the following sequence differences with respect to the canonical Isoform-1 sequence: I290F, D421V, T339I, or V814M.

In some aspects, the term periostin refers to the periostin gene, which includes genomic DNA, cDNA, mRNA, and fragments thereof. In some aspects, the term periostin also refers to oligonucleotides capable of specifically hybridizing to the periostin gene under stringent conditions. In some aspects, the oligonucleotides comprise nucleobases different from A, T, C, G, or U, for example, universal bases. See Takeshita et al. Biochem J. 294:271-278 (1993); Sasaki et al. Cancer 92:843-848 (2001); Blanchard et al. Mucosal Immunol. 1:289-296 (2008); Blanchard & Rothenberg, Immunol. Allergy Clin. North. Am. 29:141-148 (2009); Sidhu et al., Proc. Natl. Acad. Sci. USA 107:14170-14175 (2010); Kanemitsu et al., J. Allergy Clin. Immunol. β2:305-12 (2013), which are herein incorporated by reference in their entireties.

In other aspects, in addition to the determination of the level of DPP4 (e.g., protein expression level or gene expression level) and/or periostin (e.g., protein expression level or gene expression level), the methods disclosed herein can comprise determining, submitting a sample taken from the patient for determination, or instructing a clinical laboratory to determine a patient's blood eosinophil cell count, the level of the patient's IgE levels, pre- or post-bronchodilator FEV1 reversibility, the wall area percentage (WA %) of subsegmental airways from CT scan data of the lungs, or combinations thereof.

In other aspects, in addition to the determination of the level of DPP4 (e.g., protein expression level or gene expression level), the methods disclosed herein can comprise determining, submitting a sample taken from the patient for determination, or instructing a clinical laboratory to determine the expression level or activity of sCTLA-3 (soluble CTLA-3; also known as Cytotoxic T-Lymphocyte-Associated serine Esterase 3, granzyme A, or granzyme 1; Uniprot: P12544), sCD28 (soluble CD28; also known as cluster of differentiation 28 or Tp44; Uniprot: P10747), CCL5 (chemokine C-C motif ligand 5; also known as RANTES; Uniprot: P13501), CCL11 (C-C motif chemokine 11; also known as eosinophil chemotactic protein or eotaxin-1; Uniprot: P51671), CCL22 (C-C motif chemokine 22; Uniprot: 000626), or combinations thereof. These biomarkers have been disclosed in IL-13 mediated disease, e.g., in Lun et al., J. Clin. Immunol. 2007 27:430-437.

In some aspects, in addition to the determination of the level of DPP4, the methods disclosed herein can further comprising determining, submitting a sample taken from the patient for determination, or instructing a clinical laboratory to determine the expression level or activity of CCL26, FZD5, DOK1, CST2, ZNF436, C20orf100, NAGS, CST1, CDH13, HRH1, TMEM132B, NTRK1, SLCO2A1, IgE, FETUB, KRT31IKRT34, C6orf138, ATP5J, TUBAL3, JAM2, NOVA2, NOS2A, HS3ST4, GRM8, IL1R2, CTDSPL, CEP72, LOC199800, LYPD1, DISP1, NKX1-2, C4orf38, LOXL4, PRKD1, PAM124B, GPR44, HIGD1B, CLCA1, SEPT11, CYYR1, CD36, ALOX15, AADAC, ACTA1, ODC1, DKFZp434F142, ACHE, CSF3, LOC100132552, C12orf27, ZNF331, GK5, DUSP1IDUSP4, LRWD1, PGLYRP4, GUSBL2, CLGN, NR1I2, EST, LRRC37B, SAA4, SLC12A3, TMEM45A, FLJ37464, MUC5B, CXCL6, GLRB, DKFp686K01114, FOLR1, TSPAN6, AKR1C1, KIAA0232, PTP4A1, PCYT2, RHOV, PROS1, C11orf63, TCTN1, PIP5K1B, OSBPL6, NSUM7, GJB7, IRS2, or combinations thereof. These genes are part of the Th-2 signature as disclosed in Choi et al, J. Immunol. 186(3):1861-9 (2011) and WO2009124090, both of which are herein incorporated by reference in their entireties.

In some aspects, in addition to the determination of the level of DPP4 (e.g., protein expression level or gene expression level), the methods disclosed herein can further comprising determining, submitting a sample taken from the patient for determination, or instructing a clinical laboratory to determine the expression level or activity of POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C20RF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1 (SEQ ID NO:36), ALOX15 (SEQ ID NO:37), and combinations thereof.

Examples of POSTN (periostin) include a polypeptide comprising SEQ ID NO: 8 and other POSTN native sequence polypeptides, such as naturally occurring variants and native sequence polypeptides encoded by a nucleic acid sequence that can hybridize under stringent conditions to SEQ ID NOs:38 and/or 39. Also included are nucleic acids encoding such POSTN and fragments thereof, and their complementary sequences.

Examples of CST1 (cystatin-SN; Uniprot: P01037) include a polypeptide comprising SEQ ID NO:9 and other CST1 native sequence polypeptides, such as naturally occurring variants and native sequence polypeptides encoded by a nucleic acid sequence that can hybridize under stringent conditions to SEQ ID NO:40. Also included are nucleic acids encoding such CST1 and fragments thereof, and their complementary sequences.

Examples of CCL26 (chemokine (C-C motif) ligand 26; Uniprot: Q9Y258) include a polypeptide comprising SEQ ID NO:10 and other CCL26 native sequence polypeptides, such as naturally occurring variants and native sequence polypeptides encoded by a nucleic acid sequence that can hybridize under stringent conditions to SEQ ID NO:41. Also included are nucleic acids encoding such CCL26 and fragments thereof, and their complementary sequences.

Examples of CLCA1 (calcium-activated chloride channel regulator 1; Uniprot: A8K7I4) include a polypeptide comprising SEQ ID NO:11 and other CLCA1 native sequence polypeptides, such as naturally occurring variants and native sequence polypeptides encoded by a nucleic acid sequence that can hybridize under stringent conditions to SEQ ID NO:42. Also included are nucleic acids encoding such CLCA1 and fragments thereof, and their complementary sequences.

Examples of CST2 (cystatin-SA; Uniprot: P09228) include a polypeptide comprising SEQ ID NO:12 and other CST native sequence polypeptides, such as naturally occurring variants and native sequence polypeptides encoded by a nucleic acid sequence that can hybridize under stringent conditions to SEQ ID NO:43. Also included are nucleic acids encoding such CST2 and fragments thereof, and their complementary sequences.

Examples of PRR4 (proline-rich protein 4; Uniprot: Q16378) include a polypeptide comprising SEQ ID NO:13 and other PRR4 native sequence polypeptides, such as naturally occurring variants and native sequence polypeptides encoded by a nucleic acid sequence that can hybridize under stringent conditions to SEQ ID NO:44. Also included are nucleic acids encoding such PRR4 and fragments thereof, and their complementary sequences.

Examples of SERPINB2 (plasminogen activator inhibitor-2 (placental PAI), also known as HsT1201, PAI, PAI-2, PAI2 or PLANH2; Uniprot: P05120) include a polypeptide comprising SEQ ID NO:14 and other SERPINB2 native sequence polypeptides, such as naturally occurring variants and native sequence polypeptides encoded by a nucleic acid sequence that can hybridize under stringent conditions to SEQ ID NO:45. Also included are nucleic acids encoding such SERPINB2 and fragments thereof, and their complementary sequences.

Examples of CEACAM5 (carcinoembryonic antigen-related cell adhesion molecule 5 (CEACAM5) also known as CD66e (cluster of differentiation 66); Uniprot: P06731) include a polypeptide comprising SEQ ID NO:15 and other CEACAM5 native sequence polypeptides, such as naturally occurring variants and native sequence polypeptides encoded by a nucleic acid sequence that can hybridize under stringent conditions to SEQ ID NO:46. Also included are nucleic acids encoding such CEACAM5 and fragments thereof, and their complementary sequences.

Examples of iNOS (inducible NOS, known as iNOS or NOS2) include a polypeptide comprising SEQ ID NO:16 and other iNOS native sequence polypeptides, such as naturally occurring variants and native sequence polypeptides encoded by a nucleic acid sequence that can hybridize under stringent conditions to SEQ ID NO:47. Also included are nucleic acids encoding such iNOS and fragments thereof, and their complementary sequences.

Examples of SERPINB4 (serpin peptidase inhibitor, clade B (ovalbumin), member 4, also known as LEUPIN, PI11, SCCA-2, SCCA1, or SCCA2; Uniprot: P48594) include a polypeptide comprising SEQ ID NO:17 and other SERPINB4 native sequence polypeptides, such as naturally occurring variants and native sequence polypeptides encoded by a nucleic acid sequence that can hybridize under stringent conditions to SEQ ID NOs:48 and/or 49. Also included are nucleic acids encoding such SERPINB4 and fragments thereof, and their complementary sequences.

Examples of CST4 (cystatin S; Uniprot: P01036) include a polypeptide comprising SEQ ID NO:18 and other CST4 native sequence polypeptides, such as naturally occurring variants and native sequence polypeptides encoded by a nucleic acid sequence that can hybridize under stringent conditions to SEQ ID NO:50. Also included are nucleic acids encoding such CST4 and fragments thereof, and their complementary sequences.

Examples of PRB4 (basic salivary proline-rich protein 4; Uniprot: P10163) include a polypeptide comprising SEQ ID NO:19 and other PRB4 native sequence polypeptides, such as naturally occurring variants and native sequence polypeptides encoded by a nucleic acid sequence that can hybridize under stringent conditions to SEQ ID NO:51. Also included are nucleic acids encoding such PRB4 and fragments thereof, and their complementary sequences.

Examples of TPSD1 (tryptase Delta 11, also known as delta-tryptase, mast cell MMCP-7 like protein, or HmMCP-3-like tryptase III; Uniprot: Q9BZJ3) include a polypeptide comprising SEQ ID NO:20 and other TPSD1 native sequence polypeptides, such as naturally occurring variants and native sequence polypeptides encoded by a nucleic acid sequence that can hybridize under stringent conditions to a sequence selected from the group consisting of SEQ ID NO:52-58. Also included are nucleic acids encoding such TPSD1 and fragments thereof, and their complementary sequences.

Examples of TPSG1 (tryptase gamma 1, also known as TMT, tryptase gamma I, tryptase gamma II, serine protease 31, lung tryptase, mast cell protease II, mast cell tryptase, or skin tryptase; Uniprot: Q9NRR2) include a polypeptide comprising SEQ ID NO:21 and other TPSG1 native sequence polypeptides, such as naturally occurring variants and native sequence polypeptides encoded by a nucleic acid sequence that can hybridize under stringent conditions a sequence selected from the group consisting of SEQ ID NO:59-62. Also included are nucleic acids encoding such TPSG1 and fragments thereof, and their complementary sequences.

Examples of MFSD2 (major facilitator superfamily domain containing 2A protein; Uniprot: Q8NA29) include a polypeptide comprising SEQ ID NO:22 and other MFSD2 native sequence polypeptides, such as naturally occurring variants and native sequence polypeptides encoded by a nucleic acid sequence that can hybridize under stringent conditions to SEQ ID NO:63. Also included are nucleic acids encoding such MFSD2 and fragments thereof, and their complementary sequences.

Examples of CPA3 (carboxypeptidase A3, also known as mast cell carboxypeptidase A, tissue carboxypeptidase A, or MC-CPA2; Uniprot: P15088) include a polypeptide comprising SEQ ID NO:23 and other CPA3 native sequence polypeptides, such as naturally occurring variants and native sequence polypeptides encoded by a nucleic acid sequence that can hybridize under stringent conditions to SEQ ID NO:64. Also included are nucleic acids encoding such CPA3 and fragments thereof, and their complementary sequences.

Examples of GPR105 (G-Protein coupled receptor 105, also known as G protein coupled receptor for UDP-Glucose, P2Y purinoceptor 14, BPR105, UDP-Glucose receptor, purinergic receptor P2Y G-Protein coupled 14, or P2RY14; Uniprot: Q15391) include a polypeptide comprising SEQ ID NO:24 and other GPR105 native sequence polypeptides, such as naturally occurring variants and native sequence polypeptides encoded by a nucleic acid sequence that can hybridize under stringent conditions to SEQ ID NO:65. Also included are nucleic acids encoding such GPR105 and fragments thereof, and their complementary sequences.

Examples of CDH26 (cadherin 26, also known as VR20; Uniprot: Q8IXH8) include a polypeptide comprising SEQ ID NO:25 and other CDH26 native sequence polypeptides, such as naturally occurring variants and native sequence polypeptides encoded by a nucleic acid sequence that can hybridize under stringent conditions to SEQ ID NO:66. Also included are nucleic acids encoding such CDH26 and fragments thereof, and their complementary sequences.

Examples of GSN (gelsolin, also known as brevin, ADF, AGEL, or Actin-Depolymerizing Factor 2; Uniprot: P06396) include a polypeptide comprising SEQ ID NO:26 and other GSN native sequence polypeptides, such as naturally occurring variants and native sequence polypeptides encoded by a nucleic acid sequence that can hybridize under stringent conditions to SEQ ID NO: 67. Also included are nucleic acids encoding such GSN and fragments thereof, and their complementary sequences.

Examples of C2ORF32 (cannabinoid receptor interacting protein 11, also known as CRIP-1; Uniprot: Q96F85) include a polypeptide comprising SEQ ID NO:27 and other C2ORF32 native sequence polypeptides, such as naturally occurring variants and native sequence polypeptides encoded by a nucleic acid sequence that can hybridize under stringent conditions to SEQ ID NO:68. Also included are nucleic acids encoding such C2ORF32 and fragments thereof, and their complementary sequences.

Examples of TRACH2000196 (transmembrane protein 711 or TMEM71; Uniprot: Q6P5X7) include a polypeptide comprising SEQ ID NO:28 and other TRACH2000196 (TMEM71) native sequence polypeptides, such as naturally occurring variants and native sequence polypeptides encoded by a nucleic acid sequence that can hybridize under stringent conditions to SEQ ID NO: 69. Also included are nucleic acids encoding such TRACH2000196 and fragments thereof, and their complementary sequences.

Examples of DNAJC12 (DnaJ (Hsp40) homolog, subfamily C, member 121, also known as JDP1 or J Domain protein 1; Uniprot: Q9UKB3) include a polypeptide comprising SEQ ID NO:29 and other DNAJC12 native sequence polypeptides, such as naturally occurring variants and native sequence polypeptides encoded by a nucleic acid sequence that can hybridize under stringent conditions to SEQ ID NO: 70. Also included are nucleic acids encoding such DNAJC12 and fragments thereof, and their complementary sequences.

Examples of RGS 13 (regulator of G-protein signaling 13; Uniprot: 014921) include a polypeptide comprising SEQ ID NO:30 and other RGS 13 native sequence polypeptides, such as naturally occurring variants and native sequence polypeptides encoded by a nucleic acid sequence that can hybridize under stringent conditions to SEQ ID NO: 71. Also included are nucleic acids encoding such RGS 13 and fragments thereof, and their complementary sequences.

Examples of SLC18A2 (vesicular monoamine transporter 2 (VMAT2) also known as solute carrier family 18 member 2 (SLC18A2); Uniprot: Q05940) include a polypeptide comprising SEQ ID NO:31 and other SLC18 A2 native sequence polypeptides, such as naturally occurring variants and native sequence polypeptides encoded by a nucleic acid sequence that can hybridize under stringent conditions to SEQ ID NO: 72. Also included are nucleic acids encoding such SLC18A2 and fragments thereof, and their complementary sequences.

Examples of SERPINB10 (serpin peptidase inhibitor, clade B (ovalbumin), member 10; Uniprot: P48595) include a polypeptide comprising SEQ ID NO:32 and other SERPINB10 native sequence polypeptides, such as naturally occurring variants and native sequence polypeptides encoded by a nucleic acid sequence that can hybridize under stringent conditions to SEQ ID NO: 73. Also included are nucleic acids encoding such SERPINB10 and fragments thereof, and their complementary sequences.

Examples of SH3RF2 (SH3 ring finger 2 protein) include a polypeptide comprising SEQ ID NO:33 and other SH3RF2 native sequence polypeptides, such as naturally occurring variants and native sequence polypeptides encoded by a nucleic acid sequence that can hybridize under stringent conditions to SEQ ID NO: 74. Also included are nucleic acids encoding such SH3RF2 and fragments thereof, and their complementary sequences.

Examples of FCER1B (high affinity immunoglobulin epsilon receptor subunit beta or MS4A2; Uniprot: Q01362) include a polypeptide comprising SEQ ID NO:34 and other FCER1B native sequence polypeptides, such as naturally occurring variants and native sequence polypeptides encoded by a nucleic acid sequence that can hybridize under stringent conditions to SEQ ID NO:75. Also included are nucleic acids encoding such FCER1B and fragments thereof, and their complementary sequences.

Examples of RUNX2 (runt-related transcription factor 2; also known as core-binding factor subunit alpha-1 or CBF-alpha-1; Uniprot: Q13950) include a polypeptide comprising SEQ ID NO:35 and other RUNX2 native sequence polypeptides, such as naturally occurring variants and native sequence polypeptides encoded by a nucleic acid sequence that can hybridize under stringent conditions to SEQ ID NO: 76. Also included are nucleic acids encoding such RUNX2 and fragments thereof, and their complementary sequences.

Examples of PTGS1 (cyclooxygenase-1, COX-1, also known as prostaglandin G/H synthase 1, prostaglandin-endoperoxide synthase 1 or prostaglandin H2 synthase 1; Uniprot: P23219) include a polypeptide comprising SEQ ID NO:36 and other PTGS1 native sequence polypeptides, such as naturally occurring variants and native sequence polypeptides encoded by a nucleic acid sequence that can hybridize under stringent conditions to SEQ ID NO: 77. Also included are nucleic acids encoding such PTGS1 and fragments thereof, and their complementary sequences.

Examples of ALOX15 (arachidonate 15-lipoxygenase; Uniprot: P16050) include a polypeptide comprising SEQ ID NO:37 and other ALOX 15 native sequence polypeptides, such as naturally occurring variants and native sequence polypeptides encoded by a nucleic acid sequence that can hybridize under stringent conditions to SEQ ID NO:78. Also included are nucleic acids encoding such ALOX15 and fragments thereof, and their complementary sequences.

In some aspects, the IL-13 antagonist is administered at a fixed dose. In some specific aspects, the IL-antagonist is tralokinumab and the fixed dose is about 300 mg/dose. In some aspects, the IL-13 antagonist, e.g., an anti-IL-13 antibody such as tralokinumab, is administered in two or more doses. In some aspects, the IL-13 antagonist, e.g., an anti-IL-13 antibody such as tralokinumab, is administered weekly, biweekly or monthly. In some aspects, the IL-13 antagonist, e.g., an anti-IL-13 antibody such as tralokinumab, is administered biweekly. In some aspects, the IL-13 antagonist, e.g., an anti-IL-13 antibody such as tralokinumab, is administered intravenously, intramuscularly, subcutaneously, or a combination thereof.

In some aspects, the one or more control samples are obtained from normal healthy individuals; patients with a non-IL-13-mediated subset of asthma; asthma patients naïve for corticosteroid treatment; asthma patients treated with corticosteroids; a pre-determined standard amount of isolated DPP4 (e.g., protein expression level or gene expression level); or a combination thereof.

In some aspects, the administration of the IL-13 antagonist, e.g., an anti-IL-13 antibody such as tralokinumab, to the patient results in:

(a) AER (Acute Exacerbation Rate) reduction;
(b) FEV₁ (Forced Expiratory Volume in one second) increase;
(c) improved ACQ-6 (Asthma Control Questionnaire, 6-item version) results;
(d) improved AQLQ (Asthma Quality of Life Questionnaire) results; or,
(e) a combination thereof.

In some aspects the AER reduction after administration of an IL-13 antagonist (e.g., biweekly administration of a 300 mg/dose fixed dose of tralokinumab) is at least about 5%, at least about 10%, at least about 15%, at least about 20%, at least about 25%, at least about 30%, at least about 35%, at least about 40%, at least about 45%, at least about 50%, at least about 55%, or at least 60% compared to the AER observed in a population of patients treated with a placebo. In some specific aspects the AER reduction after administration of an IL-13 antagonist (e.g., biweekly administration of a 300 mg/dose fixed dose of tralokinumab) is about 28% compared to the mean AER observed in a population of patients treated with a placebo.

In some aspects the FEV₁ increase after administration of an IL-13 antagonist (e.g., biweekly administration of a 300 mg/dose fixed dose of tralokinumab) is at least about 3%, at least about 5%, at least about 7%, at least about 9%, at least about 11%, at least about 13%, at least about 15%, least about 17%, or at least about 19% compared to the FEV₁ observed in a population of patients treated with a placebo. In some aspects the FEV₁ increase after administration of an IL-13 antagonist (e.g., biweekly administration of a 300 mg/dose fixed dose of tralokinumab) is about 10% compared to the mean FEV₁ observed in a population of patients treated with a placebo.

In some aspects the ACQ-6 change after administration of an IL-13 antagonist (e.g., biweekly administration of a 300 mg/dose fixed dose of tralokinumab) is about −0.5 compared to the mean ACQ-6 observed in a population of patients treated with a placebo. In some aspects the AQLQ change after administration of an IL-13 antagonist (e.g., biweekly administration of a 300 mg/dose fixed dose of tralokinumab) is about −0.5 compared to the mean AQLQ observed in a population of patients treated with a placebo.

In certain aspects this disclosure provides a method of identifying a patient as a candidate for treatment with an IL-13 antagonist (e.g., anti-IL13 antibody including tralokinumab, or an antigen-binding fragment thereof, or lebrikizumab, or an antigen-binding fragment thereof) comprising measuring the level of DPP4 (dipeptidyl peptidase-4) in a sample taken from the patient, wherein a level of DPP4 above a predetermined DPP4 threshold level, or above the DPP4 level in one or more control samples identifies the patient as a candidate for treatment with the IL-13 antagonist.

In some aspects, the methods of identifying a patient as a candidate for treatment with an IL-13 antagonist (e.g., anti-IL13 antibody including tralokinumab, or an antigen-binding fragment thereof, or lebrikizumab, or an antigen-binding fragment thereof) further comprise measuring one or more of periostin, eosinophil cell count, IgE and FEV1 reversibility, wherein a level of DPP4 above a predetermined DPP4 threshold level, or above the DPP4 level in one or more control samples and one or more of the following: (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×109/L), (iv) FEV1 reversibility to a short-acting β2 agonist ≧12%, or (v) patient's wall area percentage (WA %) of subsegmental airways from a CT scan of the lungs above about 68% identifies the patient as a candidate for treatment with the IL-13 antagonist.

In certain aspects, the patient identified as a candidate for treatment with the IL-13 antagonist (e.g., anti-IL13 antibody including tralokinumab, or an antigen-binding fragment thereof, or lebrikizumab, or an antigen-binding fragment thereof) has asthma, IPF, COPD, chronic rhinosinusitis, allergic rhinitis, or atopic dermatitis. In certain aspects, the patient identified as a candidate for treatment with the IL-13 antagonist has allergic asthma, atopic asthma, corticosteroid naive asthma, chronic asthma, corticosteroid resistant asthma, corticosteroid refractory asthma, asthma due to smoking, or asthma uncontrolled on corticosteroids.

In some aspects, the predetermined DPP4 threshold level (e.g., protein expression level or gene expression level) in a serum sample used to identify the patient as a candidate for treatment with an IL-13 antagonist (e.g., anti-IL13 antibody including tralokinumab, or an antigen-binding fragment thereof, or lebrikizumab, or an antigen-binding fragment thereof) is at least about 250 ng/ml, at least about 350 ng/mL, at least about 365 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA (e.g. a QUANTIKINE® assay).

In certain aspects the one or more control samples used to identify the patient as a candidate for treatment with a IL-13 antagonist are obtained from normal healthy individuals; patients with a non-IL-13-mediated subset of asthma; asthma patients naïve for corticosteroid treatment; asthma patients treated with corticosteroids; a pre-determined standard amount of isolated DPP4; or a combination thereof; and can comprise one or more of whole blood, serum, plasma, saliva, sputum, bronchoalveolar lavage fluid, lung epithelial cells, urine, or a combination thereof.

In certain aspects this disclosure provides a method of diagnosing an IL-13 mediated disease or disorder in a patient comprising measuring the level of DPP4 (dipeptidyl peptidase-4) in a sample taken from the patient, wherein the patient is diagnosed with the IL-13 mediated disease or disorder if the level of DPP4 is above a predetermined DPP4 threshold level, or above the DPP4 level in one or more control samples.

In addition this disclosure further provides methods of diagnosing an IL-13 mediated disease or disorder in a patient comprising measuring the level of DPP4 (dipeptidyl peptidase-4) in a sample taken from the patient, and one or more of periostin, eosinophil cell count, IgE, FEV1 reversibility or wall area percentage (WA %) of subsegmental airways from a CT scan of the lungs, wherein the patient is diagnosed with the IL-13 mediated disease or disorder if the level of DPP4 is above a predetermined DPP4 threshold level, or above the DPP4 level in one or more control samples and the patient has one or more of the following: (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×109/L), (iv) FEV1 reversibility to a short-acting β2 agonist ≧12%, or (v) patient's wall area percentage (WA %) of subsegmental airways from a CT scan of the lungs is above about 68%.

In certain aspects, the IL-13 mediated disease or disorder diagnosed using the methods disclosed herein is asthma, IPF, COPD, chronic rhinosinusitis, allergic rhinitis, allergic asthma, atopic asthma, corticosteroid naive asthma, chronic asthma, corticosteroid resistant asthma, corticosteroid refractory asthma, asthma due to smoking, asthma uncontrolled on corticosteroids, or atopic dermatitis.

In some aspects, the predetermined DPP4 threshold level in a sample used to diagnose the patient with an IL-13 mediated disease or disorder in a patient is at least about 250 ng/ml, at least about 350 ng/mL, at least about 365 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA (e.g. a QUANTIKINE® assay).

In certain aspects the one or more control samples used to diagnose the patient as having an IL-13 mediated disease or disorder are obtained from normal healthy individuals; patients with a non-IL-13-mediated subset of asthma; asthma patients naïve for corticosteroid treatment; asthma patients treated with corticosteroids; a pre-determined standard amount of isolated DPP4; or a combination thereof; and can comprise one or more of whole blood, serum, plasma, saliva, sputum, bronchoalveolar lavage fluid, lung epithelial cells, urine, skin or a combination thereof.

### IV. DPP4 Detection Assays and Kits

This disclosure also provides kits for detecting DPP4 (e.g., protein expression level or gene expression level), for example, through an immunoassay method. Such kits can comprise containers, each with one or more of the various reagents (e.g., in concentrated form) utilized in the method, including, for example, one or more anti-DPP4 antibodies. One or more anti-DPP4 antibodies, e.g., capture antibodies, can be provided already attached to a solid support. One or more antibodies, e.g., detection antibodies, can be provided already conjugated to a detectable label, e.g., biotin or a ruthenium chelate. The kit can also provide reagents for coupling a detectable label to an antibody (as well as the label itself), buffers, and/or reagents and instrumentation to support the practice of the assays provided herein. In certain aspects, a labeled secondary antibody can be provided that binds to the detection antibody. A kit provided according to this disclosure can further comprise suitable containers, plates, and any other reagents or materials necessary to practice the assays provided herein.

In some aspects, a kit comprises one or more nucleic acid probes (e.g., oligonucleotides comprising naturally occurring and/or chemically modified nucleotide units) capable of hybridizing a subsequence of the DPP4 gene sequence (SEQ ID NO: 7) under high stringency conditions. In some aspects, one or more nucleic acid probes (e.g., oligonucleotides comprising naturally occurring and/or chemically modified nucleotide units) capable of hybridizing a subsequence of the DPP4 gene sequence (SEQ ID NO: 7) under high stringency conditions are attached to a microarray chip.

A kit provided according to this disclosure can also comprise brochures or instructions describing the process. For DPP4 detection immunoassays, and in particular sandwich immunoassays, e.g., an ELISA assay or an ECL assay, the sandwich immunoassay process comprises a first anti-DPP4 “capture” antibody or antigen-binding fragment thereof attached to a solid support, and a second anti-DPP4 “detection” antibody or antigen binding fragment thereof. The immunoassay can be performed by methods provided herein or methods well known and understood by those of ordinary skill in the art. In one aspect, the immunoassay comprises attaching a capture antibody or fragment thereof to a solid support; applying the test sample or a control sample, allowing DPP4, if present in the sample, to bind to the capture antibody or fragment thereof; applying the detection antibody or fragment thereof, which can bind to DPP4 already bound to the capture antibody or fragment thereof; and measuring the amount of detection antibody or fragment thereof bound to DPP4. In certain aspects, the assay can further include washing steps, blocking steps and incubation steps.

Test kits can include instructions for carrying out one or more DPP4 detection assays, e.g., immunoassays or nucleic acid detection assays. Instructions included in the kits can be affixed to packaging material or can be included as a package insert. While the instructions are typically written or printed materials they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### V. COMPUTER METHODS AND SOFTWARE

The methods disclosed herein can comprise collecting or otherwise obtaining a biological sample and performing an analytical method to detect and measure DPP4 levels (e.g., protein expression levels or gene expression levels) alone or in combination with other biomarkers (e.g., a panel a genes used to derive a gene signature, such as a Th-2 signature). Biomarkers that can be combined with DPP4 include isoforms 1, 2, 3, or 4 of human periostin, sCTLA-3, sCD28, CCL5, CCL11, CCL22, CCL26, FZD5, DOK1, CST2, ZNF436, C20orf100, NAGS, CST1, CDH13, HRH1, TMEM132B, NTRK1, SLCO2A1, IgE, FETUB, KRT31IKRT34, C6orf138, ATP5J, TUBAL3, JAM2, NOVA2, NOS2A, HS3ST4, GRM8, IL1R2, CTDSPL, CEP72, LOC199800, LYPD1, DISP1, NKX1-2, C4orf38, LOXL4, PRKD1, PAM124B, GPR44, HIGD1B, CLCA1, SEPT11, CYYR1, CD36, ALOX15, AADAC, ACTA1, ODC1, DKFZp434F142, ACHE, CSF3, LOC100132552, C12orf27, ZNF331, GK5, DUSP1IDUSP4, LRWD1, PGLYRP4, GUSBL2, CLGN, NR1I2, EST, LRRC37B, SAA4, SLC12A3, TMEM45A, F1137464, MUC5B, CXCL6, GLRB, DKFp686K01114, FOLR1, TSPAN6, AKR1C1, KIAA0232, PTP4A1, PCYT2, RHOV, PROS1, C11orf63, TCTN1, PIP5K1B, OSBPL6, NSUM7, GJB7, IRS2, or combinations thereof. See Lun et al., J. Clin. Immunol. 27:430-437 (2007), Choi et al, J. Immunol. 186(3):1861-9 (2011), and WO2009124090A1, which are herein incorporated by reference in their entireties. Standard names, aliases, etc. of proteins and genes designated by identifiers used throughout this application (e.g., PIP5K1B) can be identified, for example, via Genecards (www.genecards.org) or Uniprot (www.uniprot.org).

Biomarkers that can be combined with DPP4 include POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C2ORF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1(SEQ ID NO:36), ALOX15 (SEQ ID NO:37), and combinations thereof.

DPP4 levels (e.g., protein expression levels or gene expression levels) or normalized scores derived from measured DPP4 levels can be used alone (e.g., for treatment, diagnostic, prognostic, or monitoring purposes), or in combination with levels or normalized scores derived from other biomarkers (e.g., a panel a genes used to derive a gene signature, such as a Th-2 signature). These scores can also be combined with scores corresponding, for example, to (i) the level of the patient's IgE levels, (ii) the patient's eosinophil count, (iii) the patient's Fraction of Exhaled Nitric Oxide (FE_{N})), (iv) the patient's Eosinophil/Lymphocyte and Eosinophil/Neutrophil (ELEN) index, (v) the patient's EOS index, (vi) the patient's IgE levels, (vii), pre- or post-bronchodilator FEV1, FVC measurements or reversibility, (viii) wall area percentage (WA %) of subsegmental airways from CT scan of the lungs, or (ix) a combination of two or more thereof, to yield a diagnostic score. In this approach, the diagnostic score may be a single number determined from the sum of all the marker calculations that is compared to a preset DPP4 threshold value that is an indication of the presence or absence of disease. Or the diagnostic score may be a series of bars that each represent a biomarker value and the pattern of the responses may be compared to a pre-set pattern for determination of the presence or absence of disease.

At least some aspects of the methods described herein, due to the complexity of the calculations involved, a method comprising the use of DPP4 as a biomarker can be implemented with the use of a computer. In some aspects, the computer system comprises hardware elements that are electrically coupled via bus, including a processor, input device, output device, storage device, computer-readable storage media reader, communications system, processing acceleration (e.g., DSP or special-purpose processors), and memory. The computer-readable storage media reader can be further coupled to computer-readable storage media, the combination comprehensively representing remote, local, fixed and/or removable storage devices plus storage media, memory, etc. for temporarily and/or more permanently containing computer-readable information, which can include storage device, memory and/or any other such accessible system resource.

A single architecture might be utilized to implement one or more servers that can be further configured in accordance with currently desirable protocols, protocol variations, extensions, etc. However, it will be apparent to those skilled in the art that embodiments may well be utilized in accordance with more specific application requirements. Customized hardware might also be utilized and/or particular elements might be implemented in hardware, software or both. Further, while connection to other computing devices such as network input/output devices (not shown) may be employed, it is to be understood that wired, wireless, modem, and/or other connection or connections to other computing devices might also be utilized.

In one aspect, the system further comprises one or more devices for providing input data to the one or more processors. The system further comprises a memory for storing a data set of ranked data elements. In another aspect, the device for providing input data comprises a detector for detecting the characteristic of the data element, e.g., such as a fluorescent plate reader, mass spectrometer, or gene chip reader.

The system additionally may comprise a database management system. User requests or queries can be formatted in an appropriate language understood by the database management system that processes the query to extract the relevant information from the database of training sets. The system may be connectable to a network to which a network server and one or more clients are connected. The network may be a local area network (LAN) or a wide area network (WAN), as is known in the art. Preferably, the server includes the hardware necessary for running computer program products (e.g., software) to access database data for processing user requests. The system can be in communication with an input device for providing data regarding data elements to the system (e.g., expression values). In one aspect, the input device can include a gene expression profiling system including, e.g., a mass spectrometer, gene chip or array reader, and the like.

Some aspects described herein can be implemented so as to include a computer program product. A computer program product may include a computer readable medium having computer readable program code embodied in the medium for causing an application program to execute on a computer with a database. As used herein, a “computer program product” refers to an organized set of instructions in the form of natural or programming language statements that are contained on a physical media of any nature (e.g., written, electronic, magnetic, optical or otherwise) and that may be used with a computer or other automated data processing system. Such programming language statements, when executed by a computer or data processing system, cause the computer or data processing system to act in accordance with the particular content of the statements.

Computer program products include without limitation: programs in source and object code and/or test or data libraries embedded in a computer readable medium. Furthermore, the computer program product that enables a computer system or data processing equipment device to act in pre-selected ways may be provided in a number of forms, including, but not limited to, original source code, assembly code, object code, machine language, encrypted or compressed versions of the foregoing and any and all equivalents.

In one aspect, a computer program product is provided to implemented the treatment, diagnostic, prognostic, or monitoring methods disclosed herein, for example, to determine whether to administer an IL-13 antagonist (e.g., an anti-IL-13 antibody such as tralokinumab) to a patient in need thereof if the level of DPP4 in a sample taken from the patient is above a predetermined DPP4 threshold level.

The computer program product includes a computer readable medium embodying program code executable by a processor of a computing device or system, the program code comprising:

(a) code that retrieves data attributed to a biological sample from a subject, wherein the data comprises DPP4 level values (or data otherwise derived from these level values) alone or combination with values corresponding to other biomarkers in the biological sample (e.g., a panel a genes used to derive a gene signature, such as a Th-2 signature or periostin). These values can also be combined with values corresponding, for example, to (i) the level of the patient's IgE levels, (ii) the patient's eosinophil count, (iii) the patient's Fraction of Exhaled Nitric Oxide (FE_{NO}), (iv) the patient's Eosinophil/Lymphocyte and Eosinophil/Neutrophil (ELEN) index, (v) the patient's EOS index, (vi) wall area percentage (WA %) of subsegmental airways from CT scan data of the lungs, (vii) the patient's IgE levels, (viii), pre- or post-bronchodilator FEV1, FVC measurements or reversibility, or (ix) a combination of two or more thereof; and,
(b) code that executes a classification method that indicates, e.g., whether to administer an IL-13 antagonist to a patient in need thereof.

While various aspects have been described as methods or apparatuses, it should be understood that aspects can be implemented through code coupled with a computer, e.g., code resident on a computer or accessible by the computer. For example, software and databases could be utilized to implement many of the methods discussed above. Thus, in addition to aspects accomplished by hardware, it is also noted that these aspects can be accomplished through the use of an article of manufacture comprised of a computer usable medium having a computer readable program code embodied therein, which causes the enablement of the functions disclosed in this description. Therefore, it is desired that aspects also be considered protected by this patent in their program code means as well.

Furthermore, some aspects can be code stored in a computer-readable memory of virtually any kind including, without limitation, RAM, ROM, magnetic media, optical media, or magneto-optical media. Even more generally, some aspects could be implemented in software, or in hardware, or any combination thereof including, but not limited to, software running on a general purpose processor, microcode, PLAs, or ASICs.

It is also envisioned that some aspects could be accomplished as computer signals embodied in a carrier wave, as well as signals (e.g., electrical and optical) propagated through a transmission medium. Thus, the various types of information discussed above could be formatted in a structure, such as a data structure, and transmitted as an electrical signal through a transmission medium or stored on a computer readable medium.

### V. EMBODIMENTS

Embodiments are designated according to an “En” schema, where E means “embodiment” and n is the embodiment ordinal number.

### E1

A method of treating a patient having an interleukin-13 (IL-13)-mediated disease or disorder, comprising administering an IL-13 antagonist to the patient if the level of DPP4 (dipeptidyl peptidase-4) in a sample taken from the patient is above a predetermined DPP4 threshold level, or is above the DPP4 level in one or more control samples.

### E2

A method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12%, (v) wall area percentage (WA %) of subsegmental airways from a CT scan of the lungs ≧68%, or (vi) combinations thereof.

### E3

A method of treating a patient having an interleukin-13 (IL-13)-mediated disease or disorder, comprising administering an IL-13 antagonist to the patient if (a) the level of DPP4 in a sample taken from the patient is above a predetermined DPP4 threshold level, or is above the DPP4 level in one or more control samples, and (b) the patient presents a level of at least one additional biomarker in a sample taken from the patient which is above a predetermined biomarker threshold level, or is above the biomarker level in one or more control samples, wherein said additional biomarker is selection from the group consisting of POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C20RF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1 (SEQ ID NO:36), ALOX15 (SEQ ID NO:37), and combinations thereof.

### E4

The method according to embodiments E1 to E3, wherein a sample is obtained from the patient and is submitted for measurement of the level of DPP4 in the sample.

### E5

The method according to embodiments E1 to E4, wherein the patient's DPP4 level is measured in an immunoassay.

### E6

The method according to embodiment E5, wherein the immunoassay employs one or more anti-DPP4 antibodies or antigen binding fragments thereof which recognize human DDP4.

### E7

A method of treating a patient having an IL-13-mediated disease or disorder comprising (a) submitting a sample taken from the patient for measurement of the DPP4 level in the sample, wherein the patient's DPP4 level is measured with one or more anti-DPP4 antibodies or antigen binding fragments thereof which recognize human DPP4; and, (b) administering an IL-13 antagonist to the patient if the patient's DPP4 level in the sample is above a predetermined DPP4 threshold level, or is above the DPP4 level in one or more control samples.

### E8

A method of treating a patient having an IL-13-mediated disease or disorder comprising (a) measuring the DPP4 level in a sample obtained from a patient having an IL-13-mediated disease or disorder, wherein the patient's DPP4 level in the sample is measured with one or more anti-DPP4 antibodies or antigen binding fragments thereof which recognize human DPP4; (b) determining whether the patient's DPP4 level in the sample is above a predetermined DPP4 threshold level, or is above the DPP4 level in one or more control samples; and, (c) advising a healthcare provider to administer an IL-13 antagonist to the patient if the patient's DPP4 level is above a predetermined DPP4 threshold level, or is above the DPP4 level in one or more control samples.

### E9

The method according to any one of embodiments E1 to E8, wherein the IL-13-mediated disease or disorder is a pulmonary disease or disorder, an inflammatory bowel disease or disorder, or a chronic inflammatory skin disease or disorder.

### E10

The method according to embodiment E9, wherein the pulmonary disease or disorder is asthma or allergic rhinitis.

### E11

A method of treating a patient diagnosed with a pulmonary disease or disorder or a chronic inflammatory skin disease or disorder comprising administering an IL-13 antagonist to the patient if the DPP4 level in a sample taken from the patient is above a predetermined DPP4 threshold level, or is above the DPP4 level in one or more control samples.

### E12

The method according to embodiment E11, wherein the patient's DPP4 level is measured in an immunoassay employing one or more anti-DPP4 antibodies or antigen binding fragments thereof which recognize human DPP4.

### E13

A method of treating a patient diagnosed with a pulmonary disease or disorder or a chronic inflammatory skin disease or disorder comprising (a) submitting a sample taken from the patient for measurement of the DPP4 level in the sample, wherein the patient's DPP4 level is measured with one or more anti-DPP4 antibodies or antigen binding fragments thereof which recognize human DPP4; and (b) administering an IL-13 antagonist to a patient if the patient's DPP4 level in the sample is above a predetermined DPP4 threshold level, or is above the DPP4 level in one or more control samples.

### E14

A method of determining whether to treat a patient diagnosed with a pulmonary disease or disorder or a chronic inflammatory skin disease or disorder with an IL-13 antagonist therapeutic regimen comprising (a) measuring, or instructing a clinical laboratory to measure the DPP4 level in a sample obtained from a patient diagnosed with a pulmonary disease or disorder or a chronic inflammatory skin disease or disorder, wherein the patient's DPP4 level is measured with one or more anti-DPP4 antibodies or antigen binding fragments thereof which recognize human DPP4; and (b) treating, or instructing a healthcare provider to treat, the patient with an IL-13 antagonist therapeutic regimen if the patient's DPP4 level in the sample is above a predetermined DPP4 threshold level, or is above the DPP4 level in one or more control samples.

### E15

A method of selecting a patient diagnosed with a pulmonary disease or disorder or a chronic inflammatory skin disease or disorder as a candidate for treatment with an IL-13 antagonist therapeutic regimen comprising (a) measuring, or instructing a clinical laboratory to measure the DPP4 level in a sample obtained from a patient diagnosed with a pulmonary disease or disorder or a chronic inflammatory skin disease or disorder, wherein the patient's DPP4 level is measured with one or more anti-DPP4 antibodies or antigen binding fragments thereof which recognize human DPP4; and (b) treating, or instructing a healthcare provider to treat the patient with an IL-13 antagonist therapeutic regimen if the patient's DPP4 level in the sample is above a predetermined DPP4 threshold level, or is above the DPP4 level in one or more control samples.

### E16

The method according to any one of embodiments E12 to E15, wherein the pulmonary disease or disorder is asthma, IPF, COPD, chronic rhinosinusitis, or allergic rhinitis or wherein the chronic inflammatory skin disease or disorder is atopic dermatitis, allergic contact dermatitis, eczema or psoriasis.

### E17

The method according to embodiment E16, wherein the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids.

### E18

The method according to any one of embodiments E1 to E17, wherein the IL-13 antagonist comprises one or more of an anti-IL-13 antibody or antigen-binding fragment thereof, an IL-13 mutein, and IL-4 mutein, an anti-IL-13Rα1 antibody or antigen-binding fragment thereof, or an anti-IL-4Rα antibody or antigen-binding fragment thereof.

### E19

The method according to any one of embodiments E1 to E18, wherein the patient has been treated with one or more additional medications, either before, during, or after administration of an IL-13 antagonist.

### E20

The method according to embodiment E19, wherein the one or more additional medications comprises a steroid.

### E21

The method according to embodiment E19 or embodiment E20, wherein the one or more additional medications further comprises a bronchodilator.

### E22

The method according to embodiment E20 or embodiment E21, wherein the steroid is fluticasone or budesonide.

### E23

The method according to embodiment E21 or embodiment E22, wherein the bronchodilator is salbutamol or salmeterol.

### E24

The method according to any one of embodiments E19 to E23, wherein the one or more additional medications are administered by inhalation, by oral administration, by injection, or by a combination thereof.

### E25

The method according to embodiment E24, wherein inhalation administration is conducted using a metered dose inhaler (MDI) or a dry powder inhaler (DPI).

### E26

The method according to embodiments E20 to E25, wherein the steroid is administered at a high dose.

### E27

The method according to any one of embodiments E1 to E26, wherein the IL-13 antagonist is an anti-IL13 antibody, or antigen-binding fragment thereof.

### E28

The method according to embodiment E27, wherein the antibody or fragment thereof binds to the same IL-13 epitope as tralokinumab or competitively inhibits binding of tralokinumab to IL-13, or both.

### E29

The method according to embodiment E27 or embodiment E28, wherein the antibody or fragment thereof comprises tralokinumab or an antigen-binding fragment thereof.

### E30

The method according to any one of embodiments E27 to E29, wherein the antibody or fragment thereof consists of tralokinumab or an antigen-binding fragment thereof.

### E31

The method according to embodiment E27, wherein the antibody or fragment thereof binds to the same IL-13 epitope as lebrikizumab or competitively inhibits binding of lebrikizumab to IL-13, or both.

### E32

The method according to embodiment E27 or embodiment E31, wherein the antibody or fragment thereof comprises lebrikizumab or an antigen-binding fragment thereof.

### E33

The method according to embodiment E27, embodiment E31, or embodiment E32, wherein the antibody or fragment thereof consists of lebrikizumab or an antigen-binding fragment thereof.

### E34

The method according to any one of embodiments E1 to E33, wherein the sample taken from the patient comprises one or more of whole blood, serum, plasma, saliva, sputum, bronchoalveolar lavage fluid, lung epithelial cells, urine, skin, or nasal polyps.

### E35

The method according to embodiment E34, wherein the sample taken from the patient is blood serum.

### E36

The method according to any one of embodiments E1 to E35, further comprising determining, submitting a sample taken from the patient for determination, or instructing a clinical laboratory to determine (i) the level of the patient's IgE levels, (ii) the patient's eosinophil count, (iii) the patient's Fraction of Exhaled Nitric Oxide (FENO), (iv) the patient's Eosinophil/Lymphocyte and Eosinophil/Neutrophil (ELEN) index, (v) the patient's EOS index, (vi) the patients wall area percentage (WA %) of subsegmental airways from a CT scan of the lungs, or (vii) a combination of two or more thereof.

### E37

The method according to any one of embodiments E1 to E36, further comprising determining, submitting a sample taken from the patient for determination, or instructing a clinical laboratory to determine the expression level or activity of isoforms 1, 2, 3, or 4 of human periostin, a patient's blood eosinophil cell count, the level of the patient's IgE levels, pre- or post-bronchodilator FEV1 reversibility, wall area percentage (WA %) of subsegmental airways from a CT scan of the lungs, or combinations thereof.

### E38

The method according to any one of embodiments E1 to E37, further comprising determining, submitting a sample taken from the patient for determination, or instructing a clinical laboratory to determine the expression level or activity of sCTLA-3, sCD28, CCL5, CCL11, CCL22, or combinations thereof.

### E39

The method according to any one of embodiments E1 to E38, further comprising determining, submitting a sample taken from the patient for determination, or instructing a clinical laboratory to determine the expression level or activity of POSTN (SEQ ID NO:8), CST1 (SEQ ID NO:9), CCL26 (SEQ ID NO:10), CLCA1 (SEQ ID NO:11), CST2 (SEQ ID NO:12), PRR4 (SEQ ID NO:13), SERPINB2 (SEQ ID NO:14), CEACAM5 (SEQ ID NO:15), iNOS (SEQ ID NO:16), SERPINB4 (SEQ ID NO:17), CST4 (SEQ ID NO:18), PRB4 (SEQ ID NO:19), TPSD1 (SEQ ID NO:20), TPSG1 (SEQ ID NO: 21), MFSD2 (SEQ ID NO:22), CPA3 (SEQ ID NO:23), GPR105 (SEQ ID NO:24), CDH26 (SEQ ID NO:25), GSN (SEQ ID NO:26), C20RF32 (SEQ ID NO:27), TRACH2000196 (TMEM71) (SEQ ID NO:28), DNAJC12 (SEQ ID NO:29), RGS13 (SEQ ID NO: 30), SLC18A2 (SEQ ID NO: 31), SERPINB10 (SEQ ID NO:32), SH3RF2 (SEQ ID NO:33), FCER1B (SEQ ID NO:34), RUNX2 (SEQ ID NO:35), PTGS1 (SEQ ID NO:36), ALOX15 (SEQ ID NO:37), and combinations thereof.

### E40

The method according to any one of embodiments E1 to E39, wherein the IL-13 antagonist is administered at a fixed dose.

### E41

The method according to embodiment E30, wherein tralokinumab is administered at a fixed dose of about 300 mg/dose.

### E42

The method according to any one of embodiments E1 to E41, wherein the IL-13 antagonist is administered in two or more doses.

### E43

The method according to any one of embodiments E1 to E42, wherein the IL-13 antagonist is administered week, biweekly or monthly.

### E44

The method according to any one of embodiments E1 to E43, wherein the IL-13 antagonist is administered biweekly.

### E45

The method according to any one of embodiments E1 to E44, wherein the IL-13 antagonist is administered intravenously, intramuscularly, subcutaneously, or a combination thereof.

### E46

The method according to any one of embodiments E1 to E45, wherein the predetermined DPP4 threshold level is at least about 250 ng/ml, at least about 350 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA.

### E47

The method according to embodiment E46, wherein the ELISA is a QUANTIKINE® assay.

### E48

The method according to embodiment E46 or embodiment E47, wherein the predetermined DPP4 threshold level is about 365 ng/mL.

### E49

The method according to any one embodiments E1 to E48, wherein the one or more control samples are obtained from normal healthy individuals; patients with a non-IL-13-mediated subset of asthma; asthma patients naïve for corticosteroid treatment; asthma patients treated with corticosteroids; a pre-determined standard amount of isolated DPP4; or a combination thereof.

### E50

The method according to embodiment E49, wherein the one or more control samples comprise one or more of whole blood, serum, plasma, saliva, sputum, bronchoalveolar lavage fluid, lung epithelial cells, urine, skin, or a combination thereof.

### E51

The method according to any one of embodiments E1 to E50, wherein administration of the IL-13 antagonist results in

(a) AER (Acute Exacerbation Rate) reduction;
(b) FEV1 (Forced Expiratory Volume in one second) increase;
(c) improved ACQ-6 (Asthma Control Questionnaire, 6-item version) results;
(d) improved AQLQ (Asthma Quality of Life Questionnaire) results; or,
(e) a combination thereof.

### E52

The method according to embodiment E51, wherein the AER reduction is at least 5%, at least 10%, at least 15%, at least 20%, at least 25%, at least 30%, at least 35%, at least 40%, or at least 45% compared to the AER observed in a population of patients treated with a placebo.

### E53

The method according to embodiment E51 or embodiment E52, wherein the mean AER reduction is about 28% compared to the mean AER observed in a population of patients treated with a placebo.

### E54

The method according to embodiment E51, wherein the FEV1 increase is at least 3%, at least 5%, at least 7%, at least 9%, at least 11%, at least 13%, at least 15%, least 17%, or at least 19% compared to the FEV1 observed in a population of patients treated with a placebo.

### E55

The method according to embodiment E51 or embodiment E54, wherein the mean FEV1 increase is about 10% compared to the mean FEV1 observed in a population of patients treated with a placebo.

### E56

The method according to any one embodiments E3 to E55, comprising administering the IL-13 antagonist to the patient if (a) the level of DPP4 in a sample taken from the patient is above a predetermined DPP4 threshold level, or is above the DPP4 level in one or more control samples, and (b) the patient presents (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12%, (v) wall area percentage (WA %) of subsegmental airways from CT scan of the lungs ≧68%, or (vi) combinations thereof.

### E57

The method according to any one embodiments E7 to E56, wherein DPP4 is measured in an immunoassay.

### E58

A method of identifying a patient as a candidate for treatment with an IL-13 antagonist comprising measuring the level of DPP4 (dipeptidyl peptidase-4) in a sample taken from the patient, wherein a level of DPP4 above a predetermined DPP4 threshold level, or above the DPP4 level in one or more control samples identifies the patient as a candidate for treatment with the IL-13 antagonist.

### E59

The method according to embodiment E58 further comprising measuring one or more of periostin, eosinophil cell count, IgE and FEV1 reversibility, wherein a level of DPP4 above a predetermined DPP4 threshold level, or above the DPP4 level in one or more control samples and one or more of the following: (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) wall area percentage (WA %) of subsegmental airways from a CT scan of the lungs ≧68%, or (v) FEV1 reversibility to a short-acting β2 agonist ≧12%, identifies the patient as a candidate for treatment with the IL-13 antagonist.

### E60

The method according to any one of embodiments E58 or E59, wherein the patient has asthma, IPF, COPD, chronic rhinosinusitis, allergic rhinitis, or atopic dermatitis.

### E61

The method according to embodiment E60, wherein the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids.

### E62

The method according to any one of embodiments E58 to E61, wherein the IL-13 antagonist is an anti-IL13 antibody, or antigen-binding fragment thereof.

### E63

The method according to embodiment E62, wherein the anti-IL13 antibody, or antigen-binding fragment thereof comprises tralokinumab, or an antigen-binding fragment thereof, or lebrikizumab, or an antigen-binding fragment thereof.

### E64

The method according to any one of embodiments E58 to E63, wherein the predetermined DPP4 threshold level is at least about 250 ng/ml, at least about 350 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA.

### E65

The method according to embodiment E64, wherein the ELISA is a QUANTIKINE® assay.

### E66

The method according to embodiment E64 or embodiment E65, wherein the predetermined DPP4 threshold level is about 365 ng/mL.

### E67

The method according to any one embodiments E58 to E66, wherein the one or more control samples are obtained from normal healthy individuals; patients with a non-IL-13-mediated subset of asthma; asthma patients naïve for corticosteroid treatment; asthma patients treated with corticosteroids; untreated atopic dermatitis patients; treated atopic dermatitis patients; a pre-determined standard amount of isolated DPP4; or a combination thereof.

### E68

The method according to embodiment E67, wherein the one or more control samples comprise one or more of whole blood, serum, plasma, saliva, sputum, bronchoalveolar lavage fluid, lung epithelial cells, urine, skin, or a combination thereof.

### E69

A method of diagnosing an IL-13 mediated disease or disorder in a patient comprising measuring the level of DPP4 (dipeptidyl peptidase-4) in a sample taken from the patient, wherein the patient is diagnosed with the IL-13 mediated disease or disorder if the level of DPP4 is above a predetermined DPP4 threshold level, or above the DPP4 level in one or more control samples.

### E70

The method according to embodiment E69 further comprising measuring one or more of periostin, eosinophil cell count, IgE and FEV1 reversibility, wherein the patient is diagnosed with the IL-13 mediated disease or disorder if the level of DPP4 is above a predetermined DPP4 threshold level, or above the DPP4 level in one or more control samples and the patient has one or more of the following: (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) wall area percentage (WA %) of subsegmental airways from a CT scan of the lungs ≧68%, or (v) FEV1 reversibility to a short-acting β2 agonist ≧12%.

### E71

The method according to any one of embodiments E69 or E70, wherein the IL-13 mediated disease or disorder is asthma, IPF, COPD, chronic rhinosinusitis, allergic rhinitis, or atopic dermatitis.

### E72

The method according to embodiment E71, wherein the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids.

### E73

The method according to any one of embodiments E69 to E72, wherein the predetermined DPP4 threshold level is at least about 250 ng/ml, at least about 350 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA.

### E74

The method according to embodiment E73, wherein the ELISA is a QUANTIKINE® assay.

### E75

The method according to embodiment E73 or embodiment E74, wherein the predetermined DPP4 threshold level is about 365 ng/mL.

### E76

The method according to any one embodiments E69 to E75, wherein the one or more control samples are obtained from normal healthy individuals; patients with a non-IL-13-mediated subset of asthma; asthma patients naïve for corticosteroid treatment; asthma patients treated with corticosteroids; a pre-determined standard amount of isolated DPP4; or a combination thereof.

### E77

The method according to embodiment E76, wherein the one or more control samples comprise one or more of whole blood, serum, plasma, saliva, sputum, bronchoalveolar lavage fluid, lung epithelial cells, urine, skin, or a combination thereof.

### E78

The method according to any one of embodiments E1 to E77, wherein the method further comprises (a) determining the wall area percentage (WA %) from a Computed Tomography (CT) scan taken from the patient's lungs; (b) submitting a CT scan taken from the patient's lung for determination of WA % from the CT scan; or, (c) instructing a clinical laboratory to determine WA % from the CT scan.

### E79

A method of treating a patient having an interleukin-13 (IL-13)-mediated disease or disorder, comprising administering an IL-13 antagonist to the patient if wall area percentage (WA %) measured from a CT scan of the patient's lung is above a predetermined WA % threshold level, or is above a WA % threshold level calculated from one or more control CT scans.

### E80

A method of treating a patient having an IL-13-mediated disease or disorder comprising (a) submitting a CT scan of the patient's lungs for measurement of a wall area percentage (WA %) from the CT scan; and, (b) administering an IL-13 antagonist to the patient if the patient's WA % from the CT scan is above a predetermined WA % threshold level, or is above a WA % threshold level calculated from one or more control CT scans.

### E81

A method of treating a patient having an IL-13-mediated disease or disorder comprising (a) measuring wall area percentage (WA %) from a CT scan obtained from a patient's lungs having an IL-13-mediated disease or disorder; (b) determining whether the patient's WA % is above a predetermined WA % threshold level, or is above a WA % threshold level calculated from one or more control CT scans; and, (c) advising a healthcare provider to administer an IL-13 antagonist to the patient if the patient's WA % is above a predetermined WA % threshold level, or is above a WA % threshold level calculated from one or more control CT scans.

### E82

The method according to any one of embodiments E79 to E81, wherein the IL-13-mediated disease or disorder is a pulmonary disease or disorder, or an inflammatory bowel disease or disorder, or a chronic inflammatory skin disease or disorder.

### E83

The method according to embodiment E82, wherein the pulmonary disease or disorder is asthma, IPF, COPD, emphysema, chronic rhinosinusitis, or allergic rhinitis; or wherein the chronic inflammatory skin disease or disorder is atopic dermatitis, allergic contact dermatitis, eczema or psoriasis.

### E84

The method according to embodiment E83, wherein the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids.

### E85

The method according to any one of embodiments E79 to E84, wherein the IL-13 antagonist is an anti-IL13 antibody, or antigen-binding fragment thereof.

### E86

The method according to embodiment E85, wherein the antibody or fragment thereof comprises tralokinumab or an antigen-binding fragment thereof or lebrikizumab or an antigen-binding fragment thereof.

### E87

The method according to any one of embodiments E79 to E86, wherein the predetermined wall area percentage (WA %) threshold level is at least about 50%, at least about 55%, at least about 60%, at least about 65%, at least about 70%, at least about 75%, or at least about 80%, as measured using 3D analysis of a CT scan of the subsegmental bronchi in the upper lobes.

### E88

The method according to any one of embodiments E79 to E86, wherein the predetermined wall area percentage (WA %) threshold level is about 68% as measured using 3D analysis of a CT scan of the subsegmental bronchi in the upper lobes.

All patents and publications referred to herein are expressly incorporated by reference in their entireties.

Aspects of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### EXAMPLES

### Example 1

### Identification of Peripheral Markers of IL-13 Activation in the Lung

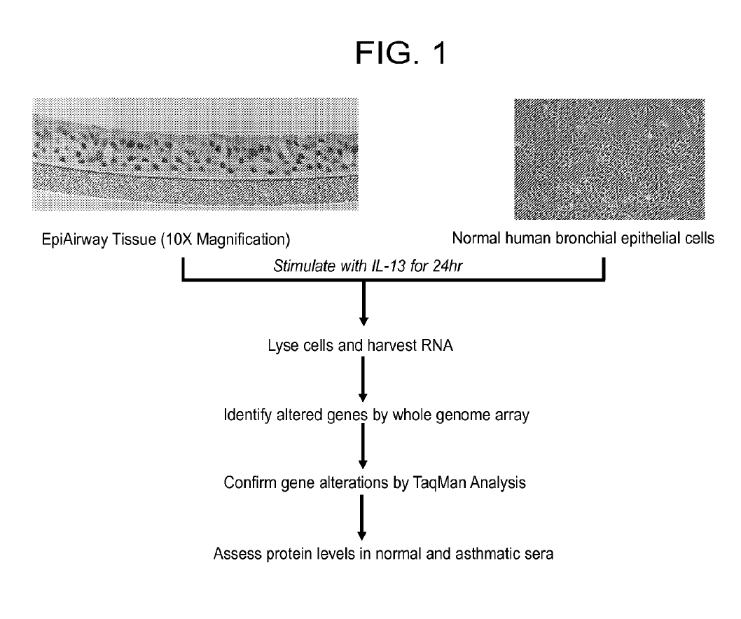

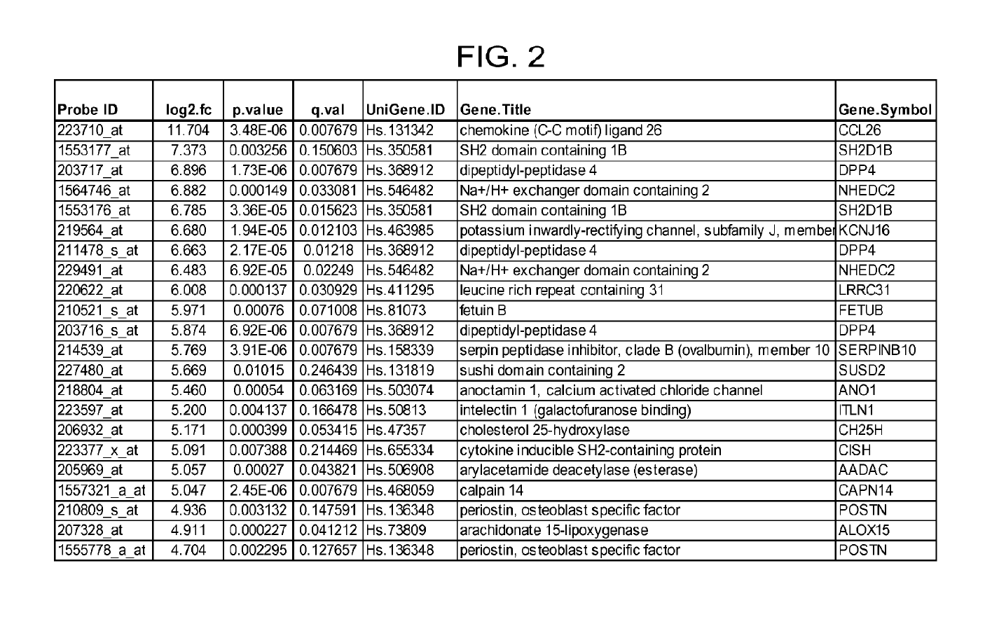

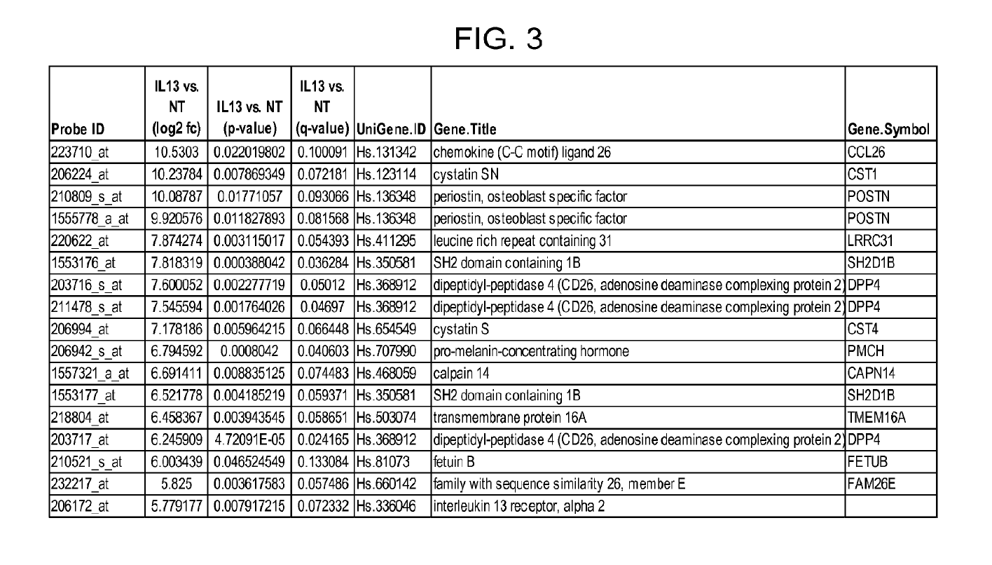

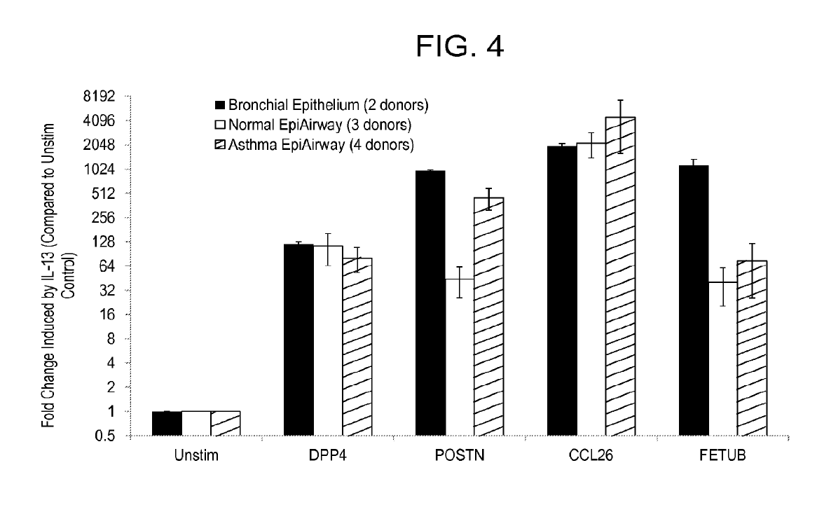

To identify genes that are regulated by IL-13 in the lung, human lung epithelial cells, and an air-liquid interface model of normal human bronchial epithelial cells were stimulated with IL-13 and the resulting transcriptional alterations were analyzed by whole genome array (WGA) and TAQMAN® PCR (see FIG. 1). Results from these stimulations revealed a number of potential markers for IL-13 pathway activation. WGA results for the EpiAirway Model and normal human bronchial epithelial cells are shown in FIG. 2 and FIG. 3, respectively. The results obtained via WGA were confirmed using TAQMAN® qPCR as shown in FIG. 4. As expected, periostin was found to be considerably up-regulated in response to IL-13. In addition to periostin up-regulation, other genes altered by IL-13 were identified, namely DPP4, CCL26, FETUB, and CST1. Accordingly, selecting one or more of these genes (or their respective expressed proteins) as biomarkers could be useful in selecting patients likely to be responsive to IL-13 antagonist therapy, for example treatment with an anti-IL-13 antibody such as tralokinumab.

One such up-regulated gene was dipeptidyl peptidase-4 (DPP4)/CD26, a highly conserved type II transmembrane glycoprotein that also exists as a shed or secreted form. DPP4 has been found in the circulation in several disease settings and DPP4 inhibitors are currently used in the treatment of type II diabetes. Additionally, DPP4 is expressed on multiple cell types in the lung and has previously been shown to be up-regulated in plasma from allergic asthmatic patients, independently of inhaled corticosteroid treatment (Lun et al. Increased expression of plasma and CD4+ T lymphocyte co-stimulatory molecule CD26 in adult patients with allergic asthma. J Clin Immunol 2007; 27:430-437).

Levels of DPP4 were determined in serum samples from asthma patients using a commercially available test (R&D Systems QUANTIKINE® Human DPPIV ELISA). A statistically significant increase in DPP4 levels in severe asthma patients compared to normal donors (p <0.05) was found, with a trend toward increasing DPP4 levels in asthma patients with moderate disease. FIG. 5. Conversely, DPP4 protein levels were lower in serum from asthma patients taking oral and inhaled steroids compared to patients taking no medication, ADVAIR® only, or Albuterol and inhaled steroids (FIG. 6). The findings that IL-13 regulates DPP4, that serum DPP4 is increased in asthma patients with moderate or severe disease, and that oral and inhaled steroids lower serum DPP4 levels suggested that DPP4: (1) could be used as a peripheral marker of IL-13 pathway activation in asthmatic lungs; (2) could be informative in electing potential therapies for asthma patients, and (3) could be useful in selecting patients responsive to therapy using an IL-13 antagonist, for example, an anti-IL-13 antibody such as tralokinumab.

### Example 2

### Method for DPP4 Quantification in Human Serum

DPP4 was determined from human serum samples using a modified human DPP4/CD26 QUANTIKINE® ELISA kit (R&D Systems; Cat. No. DC260). The QUANTIKINE® immunoassay can be used for quantitative determination of human DPP4 concentrations in cell culture supernatants, serum, plasma, saliva, and urine. The immunoassay contains NS0-expressed recombinant human DPP4, and antibodies raised against the recombinant factor. The assay employed the quantitative sandwich enzyme immunoassay technique. A monoclonal antibody specific for DPP4 was pre-coated onto a microplate. Standards and samples were pipetted into the wells, and any DPP4 present was bound by the immobilized antibody. After washing away any unbound substances, an enzyme-linked polyclonal antibody specific for DPP4 was added to the wells. Following a wash to remove any unbound antibody-enzyme reagent, a substrate solution was added to the wells and color developed in proportion to the amount of DPP4 bound in the initial step. The color development was stopped and the intensity of the color is measured.

DPPIV Reagents:

(1) DPPIV Microplate, R&D Part 892951: 96 well polysterene microplate (12 strips of 8 wells) coated with a rat monoclonal antibody against DPP4.
(2) DPPIV Conjugate, R&D Part 892952: 21 mL of polyclonal antibody against DPP4 conjugated to horseradish peroxidase with preservatives.
(3) DPPIV Standard, R&D Part 892953: 200 ng of recombinant human DPP4 in a buffer with preservatives, lyophilized.
(4) Assay Diluent RD1-57, R&D Part 895207: 11 mL of a buffered protein base with blue dye and preservatives.
(5) Calibrator Diluent RD5-33, R&D Part 895813: 3 vials (21 mL/vial) of a buffered protein base with preservatives, for serum/plasma samples (and alternative calibrator diluent RD5K, R&D Part 895119, consisting of 21 mL of an animal serum with preservatives can be used for cell culture supernatant, saliva, and urine samples).
(6) Color Reagent A, R&D Part 895000: 12.5 mL of stabilized hydrogen peroxide.
(7) Color Reagent B, R&D Part 895001: 12.5 mL of stabilized chromogen (tetramethylbenzidine).
(8) Stop Solution, R&D Part 895032: 6 mL of 2 N sulfuric acid.
(9) Plate Covers: 4 adhesive strips.

Sample collection and storage: sample were collected and stored by the following methods.

(1) Cell culture supernatants: Particulates were removed by centrifugation and assayed immediately, or samples were aliquoted and stored at a temperate of −20° C. or lower. Repeated freeze-thaw cycles were avoided.
(2) Serum: A serum separator tube (SST) was used. Samples were allowed to clot for 30 minutes before centrifugation for 15 minutes at 1000×g. Serum was removed and assayed immediately or samples were aliquoted and stored at a temperature of −20° C. or lower. Repeated freeze-thaw cycles were avoided.
(3) Plasma: Plasma was collected using heparin or EDTA as anticoagulant. Plasma was centrifuged for 15 minutes at 1000×g within 30 minutes of collection. Plasma samples were assayed immediately or aliquoted and stored at a temperature of −20° C. or lower. Repeated freeze-thaw cycles were avoided.
(4) Saliva: Saliva was collected using a collection device such as SALIVETTE® or equivalent. Saliva samples were assayed immediately or aliquoted and stored at a temperature of −20° C. or lower. Repeated freeze-thaw cycles were avoided.
(5) Urine: The first urine of the day (mid-stream) was aseptically collected and voided directly into a sterile container. The samples was centrifuged to remove particulate matter and assayed immediately, or aliquoted and stored at a temperature of −20° C. or lower. Repeated freeze-thaw cycles were avoided.

DPP4 Assay Procedure:

All reagents and samples were brought to room temperature before use. All samples, standards, and controls were assayed in duplicate.

(1) All reagents, standard dilutions, and samples were prepared as indicated in the standard Quantikine® manufacturer's protocol.
(2) Samples were tested at 1:50 MRD (minimal required dilution). All the samples were tested at 2% serum matrix. Samples were thawed at room temperature and diluted with Calibrator Diluent RD5-33.

TABLE 1
Standard dilution scheme

| | | | | Source | Calibrator | |
|---|---|---|---|---|---|---|
| | Target | | | Solution | Diluent | |
| Solution | Concentration | | Source | Vol | RD5-33 | Dilution |
| ID | (ng/mL) | | Solution | (μL) | Vol (μL) | Factor |
| STD1 | 20 | ng/mL | RS Pre-Dil | 40 | 360 | 10 |
| STD2 | 10 | ng/mL | STD1 | 200 | 200 | 2 |
| STD3 | 5 | ng/mL | STD2 | 200 | 200 | 2 |
| STD4 | 2.5 | ng/mL | STD3 | 200 | 200 | 2 |
| STD5 | 1.25 | ng/mL | STD4 | 200 | 200 | 2 |
| STD6 | 0.62 | ng/mL | STD5 | 200 | 200 | 2 |
| STD7 | 0.31 | ng/mL | STD6 | 200 | 200 | 2 |
| Blank | 0.01 | ng/Ml | N/A | N/A | 200 | N/A |

TABLE 2
QC dilution scheme

| | | | Source | Calibrator | |
|---|---|---|---|---|---|
| | Target | | Solution | Diluent | |
| Solution | Concentration | Source | Vol | RD5-33 | Dilution |
| ID | (ng/mL) | Solution | (μL) | Vol (μL) | Factor |
| Pre- | 200 | QCS | N/A | N/A | N/A |
| Dilution A | | 200 ng/mL | | | |
| Pre- | 15 | Pre-Dilution | 20 | 246 | 13.3 |
| Dilution B | | A | | | |
| QC1 | 10 | Pre-Dilution | 20 | 380 | 20 |
| (High QC) | | A | | | |
| QC2 | 5 | Pre-Dilution | 70 | 140 | 3 |
| (Medium | | B | | | |
| QC) | | | | | |
| QC3 | 1.3 | Pre-Dilution | 20 | 210 | 11.5 |
| (Low QC) | | B | | | |

(3) 100 μL of Assay Diluent RD1-57 was added to each well.
(4) 50 μL of Standard, control, or sample were added to each well. Samples were covered with a plate sealer, and incubated at room temperature for 2 hours.
(5) Each well was aspirated and washed, repeating the process 3 times for a total of 4 washes.
(6) 200 μL of DPP4 Conjugate was added to each well. Samples were covered with a new plate sealer, and incubated at room temperature for 2 hours.
(7) Well contents were aspirated and wells were washed 4 times.
(8) 200 μL Substrate Solution was added to each well. Samples were incubated at room temperature for 30 minutes, protected from light.
(9) 50 μL of Stop Solution was added to each well. Optical density was measured for each well at 450 nm within 30 minutes.

### Example 3

### A Phase 2b, Randomized, Double-Blind Study to Evaluate the Efficacy and Safety of SC Tralokinumab in Adults with Uncontrolled, Severe Asthma

TABLE 3
List of Abbreviations

| | |
|---|---|
| ACQ-6 | Asthma Control Questionnaire (6-item version) |
| AER | asthma exacerbation rate |
| AHR | airway hyperresponsiveness |
| AQLQ(S) | Standardized Asthma Quality of Life Questionnaire |
| AQLQ(s) + 12 | Standardized Asthma Quality of Life Questionnaire for |
| | 12 years and older |
| BASE | baseline FEV₁ |
| CI | confidence interval |
| CPAP | continuous positive airway pressure |
| DPI | dry powder inhaler |
| ePRO | electronic patient reported outcome |
| FEV₁ | forced expiratory volume in one second |
| HRQoL | health-related quality of life |
| IC₅₀ | half-maximal inhibitory concentration |
| ICS | inhaled corticosteroids |
| IgE | immunoglobulin E |
| IL-13 | interleukin-13 |
| LABA | long-acting β2 agonist |
| MCID | minimal clinical important change |
| MDI | metered dose inhaler |
| MRD | minimum required dilution |
| OCS | oral corticosteroid(s) |
| PEF | peak expiratory flow |
| PEFR | peak expiratory flow rate |
| Q2W | every 2 weeks |
| Q2/4W | every 2 weeks for 12 weeks followed by every 4 weeks |
| Q4W | every 4 weeks |
| SABA | short-acting β2 agonist |
| SC | Subcutaneous |
| SD | standard deviation |
| t_{1/2} | half-life |
| TEAE | treatment-emergent adverse event |
| TESAE | treatment-emergent serious adverse event |
| Th2 | T helper type 2 |

### I. Study Objectives & Design

Study CD-RI-CAT-354-1049 was a Phase 2b, randomized, double-blind, placebo-controlled, parallel-arm, multi-center study to evaluate the efficacy and safety of two SC treatment regimens of tralokinumab in adults with uncontrolled, severe asthma requiring high-dose ICS and LABA with or without additional controller medications (high-dose ICS defined as a total daily dose >500 μg fluticasone DPI or >440 μg metered dose inhaler (MDI; Global Strategy for Asthma Management and Prevention, Global Initiative for Asthma (GINA) 2012. Available from www.ginasthma.org; National Heart, Lung, and Blood Institute National Asthma Education and Prevention Program Expert Panel Report 3: Guidelines for the Diagnosis and Management of Asthma Full Report 2007.). A 5-week screening/run-in period (Week −5 to −1 [Day −1]) preceded randomisation. Starting at Week −4 (Day −28), patients received a fixed-dose combination product of fluticasone/salmeterol, either as an MDI (230 μg/21 μg) at a dose of 2 inhalations twice per day or as a DPI (500 μg/50 μg) at a dose of one inhalation twice per day. If the patient was also taking additional asthma controller medications (including leukotriene modifiers, theophylline, cromones, a secondary ICS, or oral prednisolone ≦20 mg/day or equivalent OCS), then these medications were to be continued at a stable dose during the screening/run-in and treatment period.

Key inclusion criteria:

(i) Documented physician-diagnosed asthma for at least 12 months prior to the screening/run-in period and either:

(a) Proof of post-bronchodilator reversibility of FEV₁≧12% and ≧200 mL to a SABA documented within 36 months prior to Visit 1; OR

(b) Proof of a positive response to a methacholine (20% fall in FEV₁ [PC₂₀]≦8 mg/mL), histamine or mannitol challenge documented within 36 months prior to Visit 1; OR

(c) A post-bronchodilator increase in FEV₁≧12% and ≧200 mL at Visit 1 or 2. (A maximum of 400 μg salbutamol administered for the reversibility assessment.)

(ii) An asthma controller regimen consistent with that described at Step 4 or 5 of the GINA guidelines (GINA, 2009) for at least 6 of the 12 months prior to the screening/run-in period and must have used physician prescribed high-dose ICS in combination with LABA for at least 30 days prior to the screening/run-in period
(iii) A history of at least 2 but no more than 6 documented asthma exacerbation events within the 12 months prior to the screening/run-in period
(iv) At least one of the following; a morning prebronchodilator FEV1 value of between 40% and 80% predicted or an ACQ-6 score for the preceding week of ≧1.5 at both screening and randomisation visits.

At least 390 patients aged between 18-75 years of age were planned to be randomised in a 1:1 ratio to one of 2 cohorts (Cohort 1 or Cohort 2). Within each cohort, patients were randomised in a 2:1 ratio to receive tralokinumab (300 mg) or placebo as follows:

Cohort 1: Tralokinumab 300 mg (n=130) or Placebo (n=65) as 2 SC injections Q2W for 50 weeks for a total of 26 doses.
Cohort 2: Tralokinumab 300 mg (n=130) or Placebo (n=65) as 2 SC injections Q2W for 12 weeks followed by Q4W for 38 weeks for a total of 16 doses.

Patients were stratified at screening by the number of asthma exacerbations in the past 12 months (2 versus >2 but ≦6 exacerbations) and by chronic OCS use (presence versus absence).

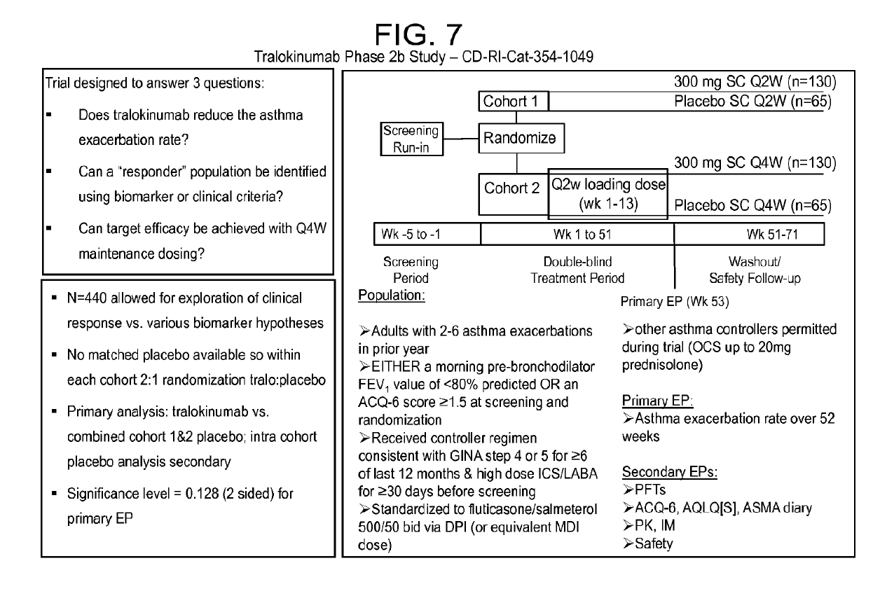

The primary objective of this study was to evaluate the effect of two SC treatment regimens of tralokinumab (300 mg Q2W and 300 mg Q2/4W) on AER over 52 weeks. Secondary objectives were to evaluate the safety and tolerability of tralokinumab, the effect of tralokinumab on pulmonary function, patient reported outcomes (including ACQ-6 score and HRQoL using AQLQ[S], and asthma symptoms using the asthma daily diary). The design of the trial and key primary and secondary endpoints is summarized in FIG. 7.

Asthma exacerbation was defined as a progressive increase of asthma symptoms (cough, wheeze, chest tightness, and/or shortness of breath) that did not resolve after the initiation of rescue medications and remained troublesome for the patient resulting in either:

1. Use of systemic corticosteroids (tablets, suspension, or injection) or increase of a stable systemic maintenance dose for a duration of at least 3 consecutive days OR
2. Patient initiation of systemic corticosteroids for a duration of at least 3 consecutive days.

The trial was powered to detect a 40% reduction in annual AER for each tralokinumab treatment group compared to combined placebo from Cohorts 1 and 2 assuming an annual exacerbation rate in placebo group of 1.2 with 80% power and a significance level of 0.15. The sample size was adequate for prespecified subanalysis to explore the relationship between the clinical response to tralokinumab and the presence of peripheral blood biomarkers associated with upregulation of IL-13 in the asthmatic lung including serum periostin, peripheral eosinophil count, and Th2 status (Th2 high defined as IgE >100 IU/mL and blood eosinophils ≧0.14×109/L; Woodruff et al., Am. J. Respir. Crit. Care Med. 2009; 180:388-395).

A total of 452 patients were randomised from 15 countries (Argentina, Canada, Chile, Czech Republic, France, Germany, Japan, Mexico, Philippines, Poland, Russia, South Korea, Spain, UK and US). All the efficacy and safety data collected through Week 53 have been analysed.

### II. Disease Evaluation and Methods

### Pre- and Post-Bronchodilator FEV1 and FVC Measurements Including Reversibility Calculations

Pre- and post-bronchodilator FEV1 and FVC were performed at each spirometry assessment. The reversibility assessment was performed as follows:

(1) Prebronchodilator FEV1 measurement was assessed using spirometry. Spirometry was performed on equipment provided by a central vendor according to American Thoracic Society (ATS)/European Respiratory Society (ERS) guidelines (Miller et al., Eur. Respir. J. 2005 26:153-61). The following values were captured: pre- and postbronchodilator FEV1, FEV6, FVC, and IC. Spirometry testing was performed in the morning between 6:00 AM and 11:00 AM. On treatment days, spirometry testing was performed before administration of investigational product. All morning spirometry testing was completed between 6:00 AM and 11:00 AM and within ±1 hour of the time the screening spirometry was completed. For example, if the screening spirometry was at 8:00 AM, then all spirometry testing at subsequent visits had to be completed between 7:00 AM and 9:00 AM. Subjects were required to refrain from strenuous exercise for 30 minutes prior to spirometry testing.
(2) After a gentle and incomplete expiration, a dose of 100 μg of salbutamol (or equivalent short acting bronchodilator) was inhaled in one breath to total lung capacity from a spacer device.
(3) Breath was then held for 5-10 seconds before the subject exhaled. Four separate doses of 100 μg of salbutamol were delivered at 30 second intervals.
(4) Subjects waited for 15-30 minutes (30 minutes if a short-acting anticholinergic agent was used).
(5) Postbronchodilator FEV1 measurement was assessed and the 2 best efforts that meet the ATS/ERS acceptability and reproducibility criteria were recorded. The maximum FEV1 of the 2 best efforts was used for the analysis. Each subject used the same dose and type of short acting bronchodilator throughout the study. Total doses of less than 400 μg of salbutamol or equivalent were used for the reversibility assessment.
Reversibility was calculated as follows:

% Reversibility=(post-bronchodilator FEV1−pre-bronchodilator FEV1)×100/pre-bronchodilator FEV1

### ACQ-6: Asthma Control Questionnaire

The ACQ is a patient-reported questionnaire assessing asthma symptoms (night-time waking, symptoms on waking, activity limitation, shortness of breath, wheezing) and daily rescue bronchodilator use and FEV1 (Juniper et al., Eur. Respir. J. 1999 14:902-7). The ACQ-6 is a shortened version of the ACQ that assesses asthma symptoms (night-time waking, symptoms on waking, activity limitation, shortness of breath, wheezing, and short-acting β2 agonist use) omitting the FEV1 measurement from the original ACQ score. The ACQ-6 was completed during Visit 1 (Week −5). Subjects were provided with the ePRO device at Visit 2 and completed the ACQ-6 at home weekly between Visits 2 and 4, and every 4 weeks thereafter through Visit 33 (Week 75). Subjects were asked to recall how their asthma had been during the previous week by responding to one bronchodilator use question and 5 symptom questions. Questions were weighted equally and scored from 0 (totally controlled) to 6 (severely uncontrolled). The mean ACQ score was the mean of the responses. Mean scores of ≦0.75 indicated well-controlled asthma, scores between 0.75 and ≦1.5 indicated partly-controlled asthma, and a score >1.5 indicated uncontrolled asthma (Juniper et al., Respir. Med. 2006 100:616-21). Individual changes of at least 0.5 were considered to be clinically meaningful (Juniper et al., Respir. Med. 2005 99:553-8).

### AQLQ: Asthma Quality of Life Questionnaire (Standardized Version)

The AQLQ(S) is a 32-item questionnaire that measures the HRQoL experienced by asthma patients (Juniper et al., Chest. 1999 May; 115(5):1265-70) and was completed at the Week 1 visit, and then every 4 weeks at home through the Week 75 visit using an ePRO device. The questionnaire comprised 4 separate domains (symptoms, activity limitations, emotional function, and environmental stimuli). Subjects were asked to recall their experiences during the previous 2 weeks and to score each of the 32 questions on a 7-point scale ranging from 7 (no impairment) to 1 (severe impairment). The overall score was calculated as the mean response to all questions. The 4 individual domain scores (symptoms, activity limitations, emotional function, and environmental stimuli) were the means of the responses to the questions in each of the domains. Individual improvements in both the overall score and individual domain scores of 0.5 were identified as a minimally important change, with score changes of >1.5 identified as large meaningful changes (Juniper et al., J. Clin. Epidemiol. 1994; 47: 81-7).

### Asthma Exacerbations

For the purpose of this study, an asthma exacerbation occurring after Visit 1 was defined as a progressive increase of asthma symptoms (cough, wheeze, chest tightness, and/or shortness of breath) that did not resolve after the initiation of rescue medications and remained troublesome for the subject resulting in either (1) use of systemic corticosteroids (tablets, suspension or injection) or increase of a stable systemic maintenance dose for a duration of at least 3 consecutive days; or (2) initiation of systemic corticosteroids for a duration of at least 3 consecutive days.

An asthma exacerbation event was considered resolved 7 days after the last dose of OCS was administered (10 days after administration of an injectable corticosteroid). Courses of corticosteroids initiated after this time period were considered a separate new asthma exacerbation.

Asthma exacerbation severity was classified as follows:

(a) Moderate: Worsening symptoms requiring systemic corticosteroids for at least 3 consecutive days; and,
(b) Severe: Worsening symptoms requiring systemic corticosteroids and requiring urgent care evaluation and/or hospital admission.

### III. Endpoints

### Effect of Tralokinumab on Pulmonary Function

The effect of tralokinumab on pulmonary function was measured by pre- and post-bronchodilator FEV1, FEV6, FVC, and IC at clinic visits (morning); and PEF and FEV1 measured at home. Change from baseline in the mean values and percent change from baseline at various time points were summarized using descriptive statistics. Two-sample t-test were used to compare the changes from baseline and percent changes from baseline in the subject's pulmonary function between the individual tralokinumab treatment group and combined placebo.

### Effect of Tralokinumab on Patient Reported Outcomes

The change from baseline in the mean ACQ-6 score was analyzed. The proportion of subjects achieving ACQ-6≦0.75, ACQ-6≦1.5, and a reduction from baseline in the mean ACQ-6 score ≧0.5 during the study was compared between the individual tralokinumab treatment group and the combined placebo using the Fisher's exact test. A stratified log-rank test was conducted to compare the time to first asthma control, defined as a reduction from baseline in the mean ACQ-6 score ≧0.5 was first observed. Health-related quality of life was evaluated using the AQLQ(S) and EQ-5D. The overall and 4 domain scores from the AQLQ(S) responses along with their respective changes from baseline were summarized using descriptive statistics. Additionally, the proportion of AQLQ(S) responders was reported; subjects with >0.5 improvement and subjects with >1.5 improvement from baseline in AQLQ(S) scores at each visit were reported separately.

The EQ-5D questionnaire assessed 5 dimensions: mobility, self-care, usual activities, pain/discomfort, and anxiety/depression. Each dimension had 3 response options (no problem, some or moderate problems, and unable or extreme problems) that reflect increasing levels of difficulty. The questionnaire also included a visual analog scale, where the patients were asked to rate their current health on a scale of 0-100, with 0 being the worst imaginable health state. The responses from each dimension and the visual analog scale were summarized by treatment group and visits. The shift tables were provided for each dimension. The change from baseline in visual analog scale was summarized with descriptive statistics by visit.

### IV. RESULTS

### Efficacy

The primary efficacy analysis was based on the Intent-to-Treat (ITT) population (n=452). The ITT population included all patients who were randomised into the study. Treatment arm was assigned according to the initial randomisation, regardless of whether patients received any investigational product or received an investigational product different from that to which they were randomised.

### Demography and Background Disease Characteristics

Baseline demographic characteristics were generally similar between the placebo and tralokinumab groups (TABLE 4). The mean age of the patient population was 50.3 years (range 18-75 years). The majority of patients were not Hispanic or Latino, approximately half of patients were White, and a third were Asian. Approximately two thirds of the population was female.

TABLE 4
Summary of Baseline Demographic
Characteristics (ITT Population)

| | | 300 mg | 300 mg |
|---|---|---|---|
| | | Tralokinumab | Tralokinumab |
| | Placebo | Q2W | Q2/4W |
| Characteristic | (n = 151) | (n = 150) | (n = 151) |

Age (years)

| | | | | | | |
|---|---|---|---|---|---|---|
| Mean (SD) | 50.3 | (12.9) | 49.7 | (12.2) | 50.5 | (11.8) |

| | | | |
|---|---|---|---|
| Range | 18-75 | 19-75 | 18-74 |
| (min-max) | | | |

Gender

| | | | | | | |
|---|---|---|---|---|---|---|
| Male | 54 | (35.8%) | 50 | (33.3%) | 51 | (33.8%) |
| Female | 97 | (64.2%) | 100 | (66.7%) | 100 | (66.2%) |

Ethnicity

| | | | | | | |
|---|---|---|---|---|---|---|
| Hispanic or | 30 | (19.9%) | 38 | (25.3%) | 35 | (23.2%) |
| Latino | | | | | | |
| Not Hispanic | 121 | (80.1%) | 112 | (74.7%) | 116 | (76.8%) |
| or Latino | | | | | | |

Race

| | | | | | | |
|---|---|---|---|---|---|---|
| American | 10 | (6.6%) | 9 | (6.0%) | 8 | (5.3%) |
| Indian or | | | | | | |
| Alaskan | | | | | | |
| Native | | | | | | |
| Asian | 52 | (34.4%) | 53 | (35.3%) | 53 | (35.1%) |
| Black or | 4 | (2.6%) | 4 | (2.7%) | 3 | (2.0%) |
| African | | | | | | |
| American | | | | | | |
| White | 84 | (55.6%) | 83 | (55.3%) | 86 | (57.0%) |
| Other | 1 | (0.7%) | 1 | (0.7%) | 1 | (0.7%) |

BMI (kg/m²)

| | | | | | | |
|---|---|---|---|---|---|---|
| Mean (SD) | 27.944 | (5.154) | 26.512 | (4.314) | 27.411 | (5.047) |

| | | | |
|---|---|---|---|
| Range | 18.90-40.00 | 16.00-38.54 | 17.27-39.90 |
| (min-max) | | | |

BMI = body mass index;
ITT = intent-to-treat;
Q2W = every 2 weeks;
Q2/4W = 2 injections Q2W for 12 weeks followed by Q4W for 38 weeks;
SD = standard deviation

Baseline disease characteristics were generally similar between the placebo and tralokinumab groups (TABLE 5), including serum periostin levels, blood eosinophil counts, and Th2 status. Between 16% and 18% of patients were reported to receive chronic OCS. DPP4 was measured as described in Example 2.

Mean FEV1% reversibility ranged between 10.0% and 12.7%, with approximately a third of patients showed FEV1 reversibility ≧12% at baseline. The mean ACQ-6 scores and % predicted pre-bronchodilator FEV1 reflect a patient population with asthma that was not well controlled at baseline.

TABLE 5
Summary of Baseline Disease Characteristics (ITT Population

| | | | 300 mg | 300 mg |
|---|---|---|---|---|
| | | | Tralokinumab | Tralokinumab |
| | | Placebo | Q2W | Q2/4W |
| Parameter | Measure | (n = 151) | (n = 150) | (n = 151) |

Markers of Asthma Control

| Pre-BD FEV₁ (L) | N | 149 | | 146 | | 149 | |
|---|---|---|---|---|---|---|---|
| | Mean (SD) | 1.924 | (0.599) | 1.922 | (0.682) | 1.939 | (0.706) |
| Pre-BD FEV₁ % | N | 149 | | 146 | | 149 | |
| Predicted | Mean (SD) | 68.1 | (16.2) | 68.3 | (19.6) | 69.3 | (18.6) |
| FEV₁ | N | 148 | | 144 | | 146 | |
| Reversibility (%) | Mean (SD) | 12.734 | (16.994) | 10.763 | (14.404) | 10.060 | (13.183) |
| | Median | 9.042 | | 7.902 | | 7.312 | |
| Proportion of | ≧12% | 57 | (38.5%) | 43 | (28.7%) | 49 | (33.6%) |
| patients with | <12% | 91 | (61.5%) | 101 | (70.1%) | 97 | (66.4%) |
| FEV₁ | Missing | 3 | | 6 | | 5 | |
| Reversibility | | | | | | | |
| Mean ACQ-6 ^{a} | N | 146 | | 146 | | 147 | |
| | Mean (SD) | 2.54 | (0.88) | 2.59 | (1.07) | 2.52 | (0.96) |
| Overall | N | 131 | | 130 | | 127 | |
| AQLQ(S) | Mean (SD) | 4.01 | (1.03) | 3.96 | (1.05) | 4.02 | (1.00) |
| Asthma Daily | N | 151 | | 147 | | 145 | |
| Diary Symptom | Mean (SD) | 1.60 | (0.71) | 1.49 | (0.77) | 1.56 | (0.69) |
| Score | | | | | | | |

Population Descriptors

| Chronic OCS | Negative | 124 | (82.1%) | 124 | (82.7%) | 127 | (84.1%) |
|---|---|---|---|---|---|---|---|
| Use | Positive | 27 | (17.9%) | 26 | (17.3%) | 24 | (15.9%) |
| Atopic Asthma | Non-atopic | 50 | (34.2%) | 42 | (28.6%) | 55 | (37.7%) |
| Status | Atopic | 96 | (65.8%) | 105 | (71.4%) | 91 | (62.3%) |
| | Missing | 5 | | 3 | | 5 | |
| Periostin | N | 147 | | 150 | | 149 | |
| | Mean (SD) | 23.959 | (9.137) | 25.531 | (10.656) | 25.480 | (10.037) |
| | Median | 22.040 | | 23.600 | | 23.250 | |
| Blood Eosinophil | N | 142 | | 141 | | 142 | |
| Count (10³/UL) | Mean (SD) | 0.370 | (0.578) | 0.383 | (0.388) | 0.344 | (0.430) |
| Th2 Status ^{b} | Low | 73 | (51.4%) | 61 | (45.5%) | 70 | (51.1%) |
| | High | 69 | (48.6%) | 73 | (54.5%) | 67 | (48.9%) |

| | | | | |
|---|---|---|---|---|
| | Missing | 9 | 16 | 14 |
| DPP4 (ng/mL) | Median | 343.0 | 372.0 | 375.0 |

ACQ-6 = Asthma Control Questionnaire 6;
AQLQ(S) = Asthma Quality of Life Questionnaire - Standardised Version;
FEV₁ = forced expiratory volume in 1 second;
ITT = intent-to-treat;
OCS = oral corticosteroid;
Pre-BD = pre-bronchodilator;
Q2W = every 2 weeks;
Q2/4W = 2 injections Q2W for 12 weeks followed by Q4W for 38 weeks;
SD = standard deviation;
Th2 = T helper type 2
^{a} Mean ACQ-6 score: ≦0.75 = well-controlled; scores >0.75 and <1.5 = partly controlled; scores ≧1.5 = uncontrolled
^{b} Th2-high: IgE >100 IU/mL and blood eosinophils ≧0.14 × 10⁹/L (Woodruff et. al. Am J Respir Crit Care Med. 180: 388-395 (2009)).

Approximately a third of patients had childhood asthma with a median age of adult onset asthma 35-37 years (TABLE 6). Allergies were reported as an asthma trigger for over half of the patients. Over a third of patients reported 3-6 asthma exacerbations in the prior year (patients were required to have between 2-6 exacerbations in the prior year at study entry) and approximately 17% to 21% of patients reported an asthma-related hospital admission.

TABLE 6
Summary of Asthma History (ITT Population)

| | | | 300 mg | 300 mg |
|---|---|---|---|---|
| | | | Tralokinumab | Tralokinumab |
| | | Placebo | Q2W | Q2/4W |
| Parameter | Measure | (n = 151) | (n = 150) | (n = 151) |
| Childhood Asthma | Yes | 53 (35.1%) | 50 (33.3%) | 49 (32.5%) |
| Age of Adult Asthma | Median | 35.00 | 36.00 | 37.00 |
| Onset (years) | Range | 18.0-73.0 | 18.0-65.0 | 18.0-73.0 |
| Triggers: Allergies | Yes | 87 (57.6%) | 95 (63.3%) | 91 (60.3%) |
| Symptoms: Night-time | >2/week | 59 (39.1%) | 77 (51.3%) | 73 (48.3%) |
| Awakening (Last 3 | | | | |
| Months) | | | | |
| Symptoms: SABA Use | 7 days/week | 63 (41.7%) | 67 (44.7%) | 72 (47.7%) |
| (Last 3 Months) | | | | |
| Symptoms: Duration on | Median | 12.00 | 12.00 | 12.00 |
| High Dose ICS/LABA | | | | |
| (Months) | | | | |
| Exacerbation in the last | >2-6 | 35.8% | 35.3% | 37.1% |
| 12 months ^{a} | | | | |
| Hospital Admissions: | Yes | 17.9% | 20.7% | 16.6% |
| Last 12 Months | | | | |
| Smoking History: Have | Yes | 38 (25.2%) | 46 (30.7%) | 38 (25.2%) |
| You Ever Smoked | | | | |
| Comorbidity: Obesity | Current | 36 (23.8%) | 21 (14.0%) | 26 (17.2%) |

ICS = inhaled corticosteroids;
ITT = intent-to-treat;
LABA = long-acting beta agonist;
Q2W = every 2 weeks;
Q2/4W = 2 injections Q2W for 12 weeks followed by Q4W for 38 weeks;
SABA = short-acting beta agonist
^{a} Patients were required to have between 2-6 exacerbations in the prior year at study entry

### Analyses of the Primary and Key Secondary Efficacy Endpoints: ITT Population

### Summary of Annual Asthma Exacerbation Rate at Week 53

In the ITT population, the annual AERs at Week 53 were similar between the placebo and tralokinumab groups (TABLE 7).

TABLE 7
Summary of Annual Asthma Exacerbation
Rate at Week 53 (ITT Population)

| | | 300 mg | 300 mg |
|---|---|---|---|
| | | Tralokinumab | Tralokinumab |
| | Placebo | Q2W | Q2/4W |
| Parameter | (n = 151) | (n = 150) | (n = 151) |
| AER Rate ^{a} | 0.90 | 0.90 | 0.97 |
| 95% CI of Rate ^{a} | 0.75, 1.08 | 0.75, 1.07 | 0.81, 1.14 |
| Rate Ratio (RR) ^{b} | — | 0.93 | 1.02 |
| 95% CI of RR ^{b} | — | 0.67, 1.30 | 0.71, 1.46 |
| P-value ^{c} | — | 0.669 | 0.909 |

AER = asthma exacerbation rate;
CI = confidence interval;
ITT = intent-to-treat;
Q2W = every 2 weeks;
Q2/4W = 2 injections Q2W for 12 weeks followed by Q4W for 38 weeks;
RR = rate ratio
^{a} Rate = Total number of asthma exacerbations in each group/Total person-year follow-up in each group; and 95% CI rate is based on the exact 95% Poisson CI
^{b} Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence) and geographical region as the covariates
^{c} P-value from the Poisson regression based on pairwise comparison against placebo

### Key Secondary Efficacy Endpoints: Effect of Tralokinumab on FEV1

A key secondary efficacy endpoint of the study was to evaluate the effect of tralokinumab on pulmonary function as assessed by changes from baseline in spirometry variables, in particular pre- and post-bronchodilator FEV1. Statistically significant mean increases from baseline were observed for both pre- and post-bronchodilator FEV1 at Week 53 for the tralokinumab 300 mg Q2W group compared with placebo (p <0.004; TABLE 8). No statistically significant mean changes from baseline were observed for either pre- or post-bronchodilator FEV1 at Week 53 for the tralokinumab 300 mg Q2/4W group compared with placebo.

TABLE 8
Summary of Change from Baseline in
FEV1 at Week 53 (ITT Population)

| | | | |
|---|---|---|---|
| | | 300 mg | 300 mg |
| | | Tralokinumab | Tralokinumab |
| | Placebo | Q2W | Q2/4W |
| Parameter | (n = 151) | (n = 150) | (n = 151) |

Pre-BD FEV₁ Change from Baseline (%)

| | | | |
|---|---|---|---|
| N | 125 | 129 | 121 |
| Mean Estimate | 1.55 | 8.65 | 3.12 |
| Difference vs | — | 7.10 | 1.57 |
| Placebo | | | |
| 95% CI of | — | 2.35, 11.84 | −3.22, 6.35 |
| Difference | | | |
| P-value ^{c} | — | 0.003 | 0.521 |

Post-BD FEV₁ Change from Baseline (%)

| | | | |
|---|---|---|---|
| N | 125 | 125 | 119 |
| Mean Estimate | −1.84 | 5.75 | 0.99 |
| Difference vs | — | 7.60 | 2.83 |
| Placebo | | | |
| 95% CI of | — | 3.87, 11.32 | −0.92, 6.58 |
| Difference | | | |
| P-value ^{c} | — | <0.001 | 0.139 |

Pre-BD FEV₁ Change from Baseline (L)

| | | | |
|---|---|---|---|
| N | 125 | 129 | 121 |
| Mean Estimate | 0.02 | 0.13 | 0.04 |
| Difference vs | — | 0.12 | 0.03 |
| Placebo | | | |
| 95% CI of | — | 0.04, 0.20 | −0.05, 0.11 |
| Difference | | | |
| P-value ^{c} | — | 0.004 | 0.495 |

Post-BD FEV₁ Change from Baseline (L)

| | | | |
|---|---|---|---|
| N | 125 | 125 | 119 |
| Mean Estimate | −0.05 | 0.09 | 0.01 |
| Difference vs | — | 0.14 | 0.06 |
| Placebo | | | |
| 95% CI of | — | 0.08, 0.21 | −0.01, 0.13 |
| Difference | | | |
| P-value | — | <0.001 | 0.073 |

CI = confidence interval;
FEV₁ = forced expiratory volume in 1 second;
ITT = intent-to-treat;
Post-BD = Post-bronchodilator;
Pre-BD = Pre-bronchodilator;
Q2W = every 2 weeks;
Q2/4W = 2 injections Q2W for 12 weeks followed by Q4W for 38 weeks
Change from baseline = current visit value − baseline value
Percent change from baseline = (current visit value − baseline value)/baseline value × 100
P-values are from a mixed effects repeated measure model comparing treatment effect between tralokinumab and placebo within each cohort at Week 53

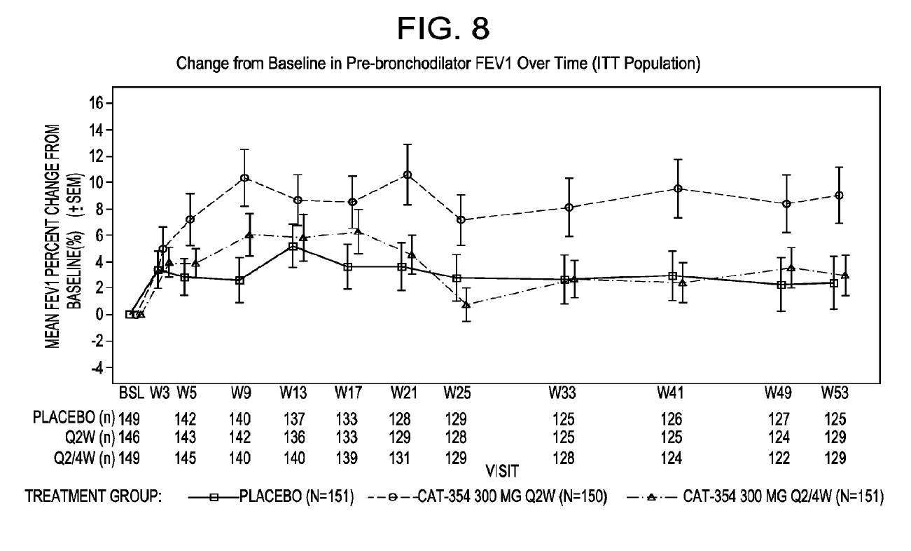

Relative increases in FEV1 were observed in both treatment groups during the first 17 weeks of the study compared to placebo (FIG. 8). These increases were maintained through to Week 53 in the tralokinumab 300 mg Q2W group but were lost in the tralokinumab 300 mg Q2/4W group suggesting that Q4W maintenance dosing is inadequate to maintain improvements in airflow obstruction.

### Key Secondary Efficacy Endpoints: Effect of Tralokinumab on Patient Reported Outcomes ACQ-6 and AQLQ(S)

The ACQ-6 is a shortened version of the ACQ that assesses asthma symptoms (night-time waking, symptoms on waking, activity limitation, shortness of breath, wheezing, and SABA use) omitting the FEV1 measurement from the original ACQ score. Questions were weighted equally and scored from 0 (totally controlled) to 6 (severely uncontrolled). The mean ACQ score is the mean of the responses. An ACQ score of ≧1.5 has a positive predictive value of 0.88 that the patient has inadequately controlled asthma (Juniper et al, 2006). Mean scores of ≦0.75 indicate well-controlled asthma, scores between 0.75 and <1.5 indicate partly-controlled asthma, and a score ≧1.5 indicates uncontrolled asthma (Juniper et al., Respir Med. 2006 100:616-21). Individual changes of at least 0.5 are considered to be clinically meaningful (Juniper et al., Respir. Med. 2005 99:553-8.).

In this study, the ACQ-6 was completed at home using an ePRO device Q4W during the treatment period. The changes from baseline to Week 53 were evaluated. No statistically significant differences in ACQ-6 scores were found when comparing the placebo and tralokinumab groups at Week 53 (TABLE 9).

The AQLQ(S) is a 32-item questionnaire that measures the HRQoL experienced by asthma patients (Juniper et al., Chest. 1999 115:1265-70). The questionnaire comprised 4 separate domains (symptoms, activity limitations, emotional function, and environmental stimuli). Patients were asked to recall their experiences during the previous 2 weeks and to score each of the 32 questions on a 7-point scale ranging from 7 (no impairment) to 1 (severe impairment). The overall score was calculated as the mean response to all questions. Individual improvement in the overall score of 0.5 has been identified as a minimally important change, with score changes of >1.5 identified as large meaningful changes (Juniper et al., J. Clin. Epidemiol. 1994 47:81-7).

In this study, the AQLQ(S) was completed at home using an ePRO device Q4W during the treatment period. The changes from baseline to Week 53 were evaluated. No statistically significant differences in AQLQ(S) scores were found when comparing the placebo and tralokinumab groups at Week 53 (TABLE 9).

TABLE 9
Summary of Change from Baseline in Mean ACQ-6
and AQLQ(S) at Week 53 (ITT Population)

| | | | |
|---|---|---|---|
| | | 300 mg | 300 mg |
| | | Tralokinumab | Tralokinumab |
| | Placebo | Q2W | Q2/4W |
| Parameter | (n = 151) | (n = 150) | (n = 151) |

Mean ACQ-6: Change from Baseline

| | | | |
|---|---|---|---|
| N | 118 | 115 | 112 |
| Mean Estimate | −0.66 | −0.84 | −0.78 |
| Difference vs | — | −0.18 | −0.12 |
| Placebo | | | |
| 95% CI of | — | −0.43, 0.06 | −0.36, 0.12 |
| Difference | | | |
| P-value | — | 0.137 | 0.335 |

AQLQ(S) Overall Score: Change from Baseline

| | | | |
|---|---|---|---|
| N | 107 | 109 | 101 |
| Mean Estimate | 0.70 | 0.91 | 0.89 |
| Difference vs | — | 0.21 | 0.19 |
| Placebo | | | |
| 95% CI of | — | −0.05, 0.46 | −0.07, 0.45 |
| Difference | | | |
| P-value | — | 0.114 | 0.159 |

ACQ-6 = Asthma Control Questionnaire 6;
AQLQ(S) = Asthma Quality of Life Questionnaire-Standardised Version;
CI = confidence interval;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Key Secondary Efficacy Endpoints: Effect of Tralokinumab on the Asthma Daily Diary

The asthma daily diary is a 13-item questionnaire. The asthma daily diary was assessed each morning from Visit 2 (Week −4) through the Week 75 visit using an ePRO device. Patients were asked to recall their experience with daytime and night-time symptom frequency and severity, activity avoidance and limitation, asthma-related anxiety, and fatigue, as well as rescue medication use. The overall symptom score was calculated as the average of daytime severity score, daytime frequency score, and night-time severity score, and ranges from zero (no symptom) to 4 (worst possible symptom).

The changes from baseline to Week 53 were evaluated. No statistically significant differences in asthma daily diary overall asthma symptom scores were found when comparing the placebo and tralokinumab groups at Week 53 (TABLE 10).

TABLE 10
Summary of Change from Baseline in Asthma
Daily Diary at Week 53 (ITT Population)

| | | | |
|---|---|---|---|
| | | 300 mg | 300 mg |
| | | Tralokinumab | Tralokinumab |
| | Placebo | Q2W | Q2/4W |
| Parameter | (n = 151) | (n = 150) | (n = 151) |

Overall Asthma Symptom Score

| | | | |
|---|---|---|---|
| N | 112 | 108 | 108 |
| Mean | −0.30 | −0.36 | −0.39 |
| Estimate | | | |
| Difference | — | −0.06 | −0.09 |
| vs Placebo | | | |
| 95% CI of | — | −0.21, 0.10 | −0.25, 0.06 |
| Difference | | | |
| P-value | — | 0.454 | 0.234 |

CI = confidence interval;
ITT—intent to treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Subgroup Analyses of the Key Primary and Secondary Efficacy Endpoints

A number of pre-specified subgroup analyses were defined to explore the relationship between the clinical response to tralokinumab and baseline clinical criteria. Subgroups defined by baseline FEV1% reversibility (≧12% vs <12%), and OCS use (presence vs absence) are presented. In addition, subgroup analysis by prior exacerbation in the past 12 months at baseline (2 vs >2≦6) was performed, no relationship with response to tralokinumab was observed. To explore the relationship between the clinical response to tralokinumab and peripheral blood biomarkers associated with upregulation of IL-13, subgroups defined by baseline serum periostin (≧median vs <median), peripheral eosinophil count (≧300 cells/4 vs <300 cells/μL), and Th2 status (Th2 high defined vs Th2 low) are presented.

### Subgroup Analysis: Baseline FEV1 Reversibility

Subgroup analysis was undertaken based on the degree of FEV1 reversibility to SABA following administration of bronchodilator at the randomisation visit. A high reversible subgroup (≧12% change in FEV1 from baseline after SABA) and low reversible subgroup (<12% change) were defined.

Analysis at Week 53 showed a reduction in the annual AER (34% [95% CI: −32, 67%]) in the tralokinumab 300 mg Q2W cohort compared with placebo in the high reversible group. No reduction in annual AER was observed in the low reversible group (TABLE 11).

Within the tralokinumab 300 mg Q2/4W cohort, the reduction in annual AER in the high reversible subgroup (24% [95% CI: −54, 63%]) was also numerically greater than in the low reversible group (2% [95% CI: −60, 40%]).

TABLE 11
Summary of Annual Asthma Exacerbation Rate at Week 53
By FEV1 Reversibility at Baseline (ITT Population)

| | | | | | | | |
|---|---|---|---|---|---|---|---|
| FEV₁ | | | | | | | |
| Reversibility | | | | 95% CI | | 95% CI | P- |
| Cut-point | Treatment Group | N | Rate | of Rate | RR | of RR | value |

AER

| | | | | | | | |
|---|---|---|---|---|---|---|---|
| ≧12% | Placebo | 57 | 0.88 | 0.65, 1.18 | — | — | — |
| | Tralokinumab 300 mg Q2W | 43 | 0.68 | 0.45, 0.99 | 0.66 | 0.33, 1.32 | 0.245 |
| | Tralokinumab 300 mg Q2/4W | 49 | 1.08 | 0.80, 1.42 | 0.76 | 0.37, 1.54 | 0.438 |
| <12% | Placebo | 91 | 0.93 | 0.73, 1.16 | — | — | — |
| | Tralokinumab 300 mg Q2W | 101 | 0.98 | 0.79, 1.20 | 1.00 | 0.65, 1.55 | 0.993 |
| | Tralokinumab 300 mg Q2/4W | 97 | 0.90 | 0.71, 1.12 | 0.98 | 0.60, 1.60 | 0.931 |

CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks;
RR = rate ratio
Rate = Total number of asthma exacerbations in each group/Total person-year follow-up in each group; and 95% CI rate is based on the exact 95% Poisson CI
Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence), and geographical region as the covariates
P-value from the Poisson regression based on pairwise comparison against placebo

Analysis of Week 53 showed an increase from baseline in FEV1 in the tralokinumab 300 mg Q2W compared with placebo in both the high (11.07% [95% CI: 0.99, 21.14) and low reversible (7.48% [2.55, 12.40]) subgroups; this increase was numerically higher in the high reversible subgroup. Clinically relevant changes in FEV1 were not observed in the tralokinumab 300 mg Q2/4W cohort in either high or low reversible subgroup (TABLE 12).

TABLE 12
Summary of Change from Baseline in Key Secondary Efficacy Endpoints
at Week 53 By FEV1 Reversibility at Baseline (ITT Population)

| | | | | | | |
|---|---|---|---|---|---|---|
| FEV₁ | | | | | | |
| Reversibility | | | Mean | Difference | | |
| Cut-point | Treatment Group | N | Estimate | vs Placebo | 95% CI | P-value |

Change from Pre-bronchodilator Baseline FEV₁ (%)

| | | | | | | |
|---|---|---|---|---|---|---|
| ≧12% | Placebo | 49 | 8.21 | — | — | — |
| | Tralokinumab 300 mg Q2W | 35 | 19.28 | 11.07 | 0.99, 21.14 | 0.031 |
| | Tralokinumab 300 mg Q2/4W | 40 | 8.84 | 0.63 | −9.30, 10.56 | 0.901 |
| <12% | Placebo | 76 | −3.58 | — | — | — |
| | Tralokinumab 300 mg Q2W | 92 | 3.90 | 7.48 | 2.55, 12.40 | 0.003 |
| | Tralokinumab 300 mg Q2/4W | 80 | −1.13 | 2.45 | −2.51, 7.42 | 0.332 |

Change from Pre-bronchodilator Baseline FEV₁ (L)

| | | | | | | |
|---|---|---|---|---|---|---|
| ≧12% | Placebo | 49 | 0.12 | — | — | — |
| | Tralokinumab 300 mg Q2W | 35 | 0.30 | 0.18 | 0.02, 0.34 | 0.031 |
| | Tralokinumab 300 mg Q2/4W | 40 | 0.15 | 0.03 | −0.13, 0.19 | 0.709 |
| <12% | Placebo | 76 | −0.07 | — | — | — |
| | Tralokinumab300 mg Q2W | 92 | 0.06 | 0.13 | 0.05, 0.22 | 0.002 |
| | Tralokinumab 300 mg Q2/4W | 80 | −0.03 | 0.04 | −0.05, 0.12 | 0.386 |

ACQ-6

| | | | | | | |
|---|---|---|---|---|---|---|
| ≧12% | Placebo | 43 | −0.33 | — | — | — |
| | Tralokinumab 300 mg Q2W | 33 | −0.77 | −0.44 | −0.89, 0.01 | 0.055 |
| | Tralokinumab 300 mg Q2/4W | 35 | −0.71 | −0.37 | −0.82, 0.08 | 0.104 |
| <12% | Placebo | 73 | −0.86 | — | — | — |
| | Tralokinumab 300 mg Q2W | 78 | −0.92 | 0.06 | −0.37, 0.24 | 0.685 |
| | Tralokinumab 300 mg Q2/4W | 75 | −0.83 | 0.03 | −0.27, 0.33 | 0.846 |

AQLQ(S) Overall Score

| | | | | | | |
|---|---|---|---|---|---|---|
| ≧12% | Placebo | 39 | 0.56 | — | — | — |
| | Tralokinumab 300 mg Q2W | 29 | 1.15 | 0.59 | 0.10, 1.09 | 0.020 |
| | Tralokinumab 300 mg Q2/4W | 32 | 0.85 | 0.29 | −0.18, 0.77 | 0.226 |
| <12% | Placebo | 66 | 0.81 | — | — | — |
| | Tralokinumab 300 mg Q2W | 76 | 0.81 | 0.00 | −0.32, 0.33 | 0.983 |
| | Tralokinumab 300 mg Q2/4W | 67 | 0.89 | 0.08 | −0.24, 0.41 | 0.610 |

Asthma Daily Diary Overall Symptom Score

| | | | | | | |
|---|---|---|---|---|---|---|
| ≧12% | Placebo | 40 | −0.19 | — | — | — |
| | Tralokinumab 300 mg Q2W | 30 | −0.42 | −0.23 | −0.51, −0.04 | 0.094 |
| | Tralokinumab 300 mg Q2/4W | 34 | −0.32 | −0.13 | −0.40, 0.15 | 0.358 |
| <12% | Placebo | 70 | −0.34 | — | — | — |
| | Tralokinumab 300 mg Q2W | 74 | −0.36 | −0.02 | −0.22, 0.19 | 0.859 |
| | Tralokinumab 300 mg Q2/4W | 72 | −0.43 | −0.08 | −0.29, 0.12 | 0.407 |

ACQ-6 = Asthma Control Questionnaire 6;
AQLQ(S) = Asthma Quality of Life Questionnaire-Standardised Version;
CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

In patients receiving tralokinumab 300 mg Q2W, the mean change in ACQ-6 score compared to placebo was numerically larger in the high reversible subgroup (−0.44 [95% CI: −0.89, 0.01]), and approximated the minimal clinical important change (MCID; −0.5) compared to the low reversible subgroup (0.06 [95% CI: −0.37, 0.24]). The same relationship was observed between the subgroups within the tralokinumab 300 mg Q2/4W cohort but the MCID was not reached in the high reversible subgroup (TABLE 12).

In patients receiving tralokinumab 300 mg Q2W, improvement in mean change in AQLQ(S) Overall score compared to placebo was observed in the high reversible subgroup (0.59 [95% CI: 0.10, 1.09]) and reached the MCID of 0.5. No improvement in AQLQ(S) was observed in the low reversible subgroup. The same relationship was observed between the subgroups within the tralokinumab 300 mg Q2/4W cohort, but the MCID was not reached in the high reversible subgroup (TABLE 12).

In patients receiving tralokinumab 300 mg Q2W a numerically greater reduction from baseline at Week 53 in the overall asthma daily diary symptom score compared to placebo was observed in the high reversible subgroup (−0.23 [95% CI: −0.51, −0.04]) compared with the low reversible subgroup (−0.02 [95% CI: −0.22, 0.19]). The same relationship was observed between the subgroups within the tralokinumab 300 mg Q2/4W cohort (TABLE 12).

### Subgroup Analysis: OCS Use

Analysis at Week 53 showed that in patients without chronic OCS use there was a reduction in the annual AER compared to placebo in both the tralokinumab 300 mg Q2W (21% [95% CI: −17, 47%]) and the Q2/4W cohorts (13% [95% CI: −34, 43%]). No reduction in annual AER was observed for the tralokinumab 300 mg Q2W and Q2/4W cohorts compared with placebo in patients with chronic OCS use (TABLE 13).

TABLE 13
Summary of Annual Asthma Exacerbation Rate at Week 53
By Chronic Oral Corticosteroid Use (ITT Population)

| | | | | | | | |
|---|---|---|---|---|---|---|---|
| Chronic OCS | | | | 95% CI | | 95% CI | P- |
| Use | Treatment Group | N | Rate | of Rate | RR | of RR | value |

AER

| | | | | | | | |
|---|---|---|---|---|---|---|---|
| With chronic | Placebo | 27 | 1.37 | 0.93, 1.94 | — | — | — |
| OCS use | Tralokinumab 300 mg Q2W | 26 | 1.99 | 1.47, 2.64 | 1.18 | 0.58, 2.41 | 0.647 |
| | Tralokinumab 300 mg Q2/4W | 24 | 2.20 | 1.62, 2.91 | 1.29 | 0.61, 2.74 | 0.506 |
| Without chronic | Placebo | 124 | 0.81 | 0.66, 1.00 | — | — | — |
| OCS use | Tralokinumab 300 mg Q2W | 124 | 0.68 | 0.54, 0.84 | 0.79 | 0.53, 1.17 | 0.240 |
| | Tralokinumab 300 mg Q2/4W | 127 | 0.74 | 0.59, 0.91 | 0.87 | 0.57, 1.34 | 0.525 |

CI = confidence interval;
ITT = intent-to-treat;
OCS = oral corticosteroid use;
Q2W = every 2 weeks;
Q2/4W = 2 injections Q2W for 12 weeks followed by Q4W for 38 weeks;
RR = rate ratio
Rate = Total number of asthma exacerbations in each group/Total person-year follow-up in each group; and 95% CI rate is based on the exact 95% Poisson CI
Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence), and geographical region as the covariates
P-value from the Poisson regression based on pairwise comparison against placebo

Analysis at Week 53 showed a numerically greater increase from baseline in pre-bronchodilator FEV1 in the tralokinumab 300 mg Q2W cohort compared with placebo in patients without chronic OCS use (7.33% [95% CI: 2.52, 12.14]) compared with patients with chronic OCS use (0.87% [95% CI: −14.70, 16.44]; TABLE 14). Clinically relevant changes in FEV1 were not observed in the tralokinumab 300 mg Q2/4W cohort in either the with or without chronic OCS use subgroups.

Clinically important changes from placebo for ACQ-6, AQLQ(S), and asthma daily diary symptom scores were not observed in the with or without chronic OCS use subgroups for either tralokinumab cohort.

TABLE 14
Summary of Change from Baseline in Key Secondary Efficacy Endpoints
at Week 53 By Chronic Oral Corticosteroid Use (ITT Population)

| | | | | | | |
|---|---|---|---|---|---|---|
| Chronic OCS | | | Mean | Difference | | |
| Use | Treatment Group | N | Estimate | vs Placebo | 95% CI | P-value |

Change from Pre-bronchodilator Baseline FEV₁ (%)

| | | | | | | |
|---|---|---|---|---|---|---|
| With chronic | Placebo | 20 | 4.63 | — | — | — |
| OCS use | Tralokinumab 300 mg Q2W | 21 | 5.51 | 0.87 | −14.70, 16.44 | 0.912 |
| | Tralokinumab 300 mg Q2/4W | 16 | 3.17 | −1.46 | −17.76, 14.84 | 0.860 |
| Without chronic | Placebo | 105 | 3.66 | — | — | — |
| OCS use | Tralokinumab 300 mg Q2W | 108 | 10.99 | 7.33 | 2.52, 12.14 | 0.003 |
| | Tralokinumab 300 mg Q2/4W | 105 | 4.58 | 0.92 | −3.89, 5.73 | 0.708 |

Change from Pre-bronchodilator Baseline FEV₁ (L)

| | | | | | | |
|---|---|---|---|---|---|---|
| With chronic | Placebo | 20 | 0.07 | — | — | — |
| OCS use | Tralokinumab 300 mg Q2W | 21 | 0.06 | −0.01 | −0.26, 0.24 | 0.932 |
| | Tralokinumab 300 mg Q2/4W | 16 | −0.03 | −0.10 | −0.36, 0.16 | 0.464 |
| Without chronic | Placebo | 105 | 0.04 | — | — | — |
| OCS use | Tralokinumab300 mg Q2W | 108 | 0.16 | 0.13 | 0.04, 0.21 | 0.003 |
| | Tralokinumab 300 mg Q2/4W | 105 | 0.06 | 0.03 | −0.06, 0.11 | 0.530 |

ACQ-6

| | | | | | | |
|---|---|---|---|---|---|---|
| With chronic | Placebo | 20 | −0.79 | — | — | — |
| OCS use | Tralokinumab 300 mg Q2W | 17 | −1.16 | −0.37 | −0.97, 0.23 | 0.226 |
| | Tralokinumab 300 mg Q2/4W | 18 | −0.63 | 0.15 | −0.44, 0.75 | 0.613 |
| Without chronic | Placebo | 98 | −0.83 | — | — | — |
| OCS use | Tralokinumab 300 mg Q2W | 98 | −1.00 | −0.17 | −0.43, 0.09 | 0.208 |
| | Tralokinumab 300 mg Q2/4W | 94 | −1.01 | −0.18 | −0.44, 0.09 | 0.186 |

AQLQ(S) Overall Score

| | | | | | | |
|---|---|---|---|---|---|---|
| With chronic | Placebo | 17 | 0.54 | — | — | — |
| OCS use | Tralokinumab 300 mg Q2W | 16 | 0.82 | 0.28 | −0.37, 0.93 | 0.398 |
| | Tralokinumab 300 mg Q2/4W | 16 | 0.22 | −0.32 | −0.95, 0.32 | 0.327 |
| Without chronic | Placebo | 90 | 0.79 | — | — | — |
| OCS use | Tralokinumab 300 mg Q2W | 93 | 1.02 | 0.23 | −0.05, 0.52 | 0.109 |
| | Tralokinumab 300 mg Q2/4W | 85 | 1.09 | 0.30 | 0.02, 0.59 | 0.039 |

Asthma Daily Diary Overall Symptom Score

| | | | | | | |
|---|---|---|---|---|---|---|
| With chronic | Placebo | 20 | −0.36 | — | — | — |
| OCS use | Tralokinumab 300 mg Q2W | 17 | −0.42 | −0.06 | −0.42, 0.30 | 0.749 |
| | Tralokinumab 300 mg Q2/4W | 18 | −0.12 | 0.24 | −0.12, 0.60 | 0.196 |
| Without chronic | Placebo | 92 | −0.39 | — | — | — |
| OCS use | Tralokinumab 300 mg Q2W | 91 | −0.46 | −0.07 | −0.24, 0.11 | 0.456 |
| | Tralokinumab 300 mg Q2/4W | 90 | −0.55 | −0.15 | −0.32, 0.02 | 0.084 |

ACQ-6 = Asthma Control Questionnaire 6;
AQLQ(S) = Asthma Quality of Life Questionnaire-Standardised Version;
CI = confidence interval;
FEV₁ = forced expiratory volume in 1 second;
OCS = oral corticosteroids;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Subgroup Analysis: Baseline Serum Periostin Level

Subgroup analysis at Week 53 by serum periostin level at baseline showed that reductions in the annual AER were observed in the tralokinumab 300 mg Q2W cohort compared with placebo in the high periostin group (≧median serum periostin level at baseline; 25% [95% CI: −19, 53%]). No reduction in AER was observed in the low periostin group (<median serum periostin level at baseline; TABLE 15).

TABLE 15
Summary of Asthma Exacerbation Rate Through Week 53
By Serum Periostin Level at Baseline (ITT Population)

| | | | | | | | |
|---|---|---|---|---|---|---|---|
| Baseline Serum | | | | | | | |
| Periostin | | | | 95% CI | | 95% CI | P- |
| Cut-point | Treatment Group | N | Rate | of Rate | RR | of RR | value |

AER

| | | | | | | | |
|---|---|---|---|---|---|---|---|
| ≧Median | Placebo | 67 | 1.13 | 0.88, 1.43 | — | — | — |
| | Tralokinumab 300 mg Q2W | 80 | 0.84 | 0.65, 1.08 | 0.75 | 0.47, 1.19 | 0.219 |
| | Tralokinumab 300 mg Q2/4W | 78 | 1.29 | 1.04, 1.57 | 0.97 | 0.58, 1.64 | 0.914 |
| <Median | Placebo | 83 | 0.74 | 0.56, 0.96 | — | — | — |
| | Tralokinumab 300 mg Q2W | 70 | 0.97 | 0.75, 1.23 | 1.08 | 0.68, 1.73 | 0.742 |
| | Tralokinumab 300 mg Q2/4W | 72 | 0.62 | 0.44, 0.84 | 0.89 | 0.56, 1.46 | 0.645 |

CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks;
RR = rate ratio
Rate = Total number of asthma exacerbations in each group/Total person-year follow-up in each group; and 95% CI rate is based on the exact 95% Poisson CI
Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence) and geographical region as the covariates
P-value from the Poisson regression based on pairwise comparison against placebo

Improvements from baseline in FEV1 compared to placebo was observed in both the high periostin (6.75% [95% CI-0.31, 13.82]) and the low periostin subgroups (7.06% [95% CI: 0.51, 13.60]; TABLE 16).

Clinically relevant changes in FEV1 were not observed in the tralokinumab 300 mg Q2/4W cohort in either periostin subgroup. Clinically important changes from placebo for ACQ-6, AQLQ(S), and asthma daily diary symptom scores were not observed in the high or low periostin subgroups for either tralokinumab cohort.

TABLE 16
Summary of Change from Baseline in Key Secondary Efficacy Endpoints
at Week 53 By Serum Periostin Level at Baseline (ITT Population)

| | | | | | | |
|---|---|---|---|---|---|---|
| Baseline Serum | | | | | | |
| Periostin | | | Mean | Difference | | P- |
| Cut-point | Treatment Group | N | Estimate | vs Placebo | 95% CI | value |

Change from Pre-bronchodilator Baseline FEV₁ (%)

| | | | | | | |
|---|---|---|---|---|---|---|
| ≧Median | Placebo | 54 | 4.68 | — | — | — |
| | Tralokinumab 300 mg Q2W | 69 | 11.43 | 6.75 | −0.31, 13.82 | 0.061 |
| | Tralokinumab 300 mg Q2/4W | 63 | 5.31 | 0.64 | −6.55, 7.83 | 0.862 |
| <Median | Placebo | 70 | −1.27 | — | — | — |
| | Tralokinumab 300 mg Q2W | 60 | 5.79 | 7.06 | 0.51, 13.60 | 0.035 |
| | Tralokinumab 300 mg Q2/4W | 57 | −0.76 | 0.51 | −6.06, 7.07 | 0.879 |

Change from Pre-bronchodilator Baseline FEV₁ (L)

| | | | | | | |
|---|---|---|---|---|---|---|
| ≧Median | Placebo | 54 | 0.07 | — | — | — |
| | Tralokinumab 300 mg Q2W | 69 | 0.17 | 0.10 | −0.01, 0.21 | 0.062 |
| | Tralokinumab 300 mg Q2/4W | 63 | 0.09 | 0.02 | −0.09, 0.13 | 0.762 |
| <Median | Placebo | 70 | −0.03 | — | — | — |
| | Tralokinumab 300 mg Q2W | 60 | 0.10 | 0.13 | 0.01, 0.25 | 0.0.29 |
| | Tralokinumab 300 mg Q2/4W | 57 | −0.02 | 0.01 | −0.11, 0.13 | 0.846 |

ACQ-6

| | | | | | | |
|---|---|---|---|---|---|---|
| ≧Median | Placebo | 51 | −0.71 | — | — | — |
| | Tralokinumab 300 mg Q2W | 64 | −0.94 | −0.23 | −0.56, 0.09 | 0.163 |
| | Tralokinumab 300 mg Q2/4W | 59 | −0.65 | 0.06 | −0.27, 0.39 | 0.711 |
| <Median | Placebo | 66 | −0.66 | — | — | — |
| | Tralokinumab 300 mg Q2W | 51 | −0.68 | −0.02 | −0.38, 0.34 | 0.919 |
| | Tralokinumab 300 mg Q2/4W | 53 | −0.94 | −0.28 | −0.64, 0.08 | 0.125 |

AQLQ(S) Overall Score

| | | | | | | |
|---|---|---|---|---|---|---|
| ≧Median | Placebo | 46 | 0.65 | — | — | — |
| | Tralokinumab 300 mg Q2W | 62 | 0.87 | 0.22 | −0.15, 0.59 | 0.245 |
| | Tralokinumab 300 mg Q2/4W | 55 | 0.81 | 0.16 | −0.22, 0.53 | 0.412 |
| <Median | Placebo | 60 | 0.74 | — | — | — |
| | Tralokinumab 300 mg Q2W | 47 | 0.95 | 0.21 | −0.16, 0.58 | 0.272 |
| | Tralokinumab 300 mg Q2/4W | 46 | 0.99 | 0.25 | −0.13, 0.62 | 0.193 |

Asthma Daily Diary Overall Symptom Score

| | | | | | | |
|---|---|---|---|---|---|---|
| ≧Median | Placebo | 48 | −0.29 | — | — | — |
| | Tralokinumab 300 mg Q2W | 56 | −0.35 | −0.06 | −0.29, 0.16 | 0.595 |
| | Tralokinumab 300 mg Q2/4W | 58 | −0.34 | −0.06 | −0.28, 0.17 | 0.619 |
| <Median | Placebo | 64 | −0.33 | — | — | — |
| | Tralokinumab 300 mg Q2W | 52 | −0.37 | −0.04 | −0.26, 0.17 | 0.685 |
| | Tralokinumab 300 mg Q2/4W | 50 | −0.43 | −0.10 | −0.32, 0.12 | 0.368 |

### Subgroup Analysis: Baseline Peripheral Blood Eosinophil Count

Subgroup analysis at Week 53 by blood eosinophil count at baseline showed a reduction in the AER in the tralokinumab 300 mg Q2W cohort compared with placebo in the high eosinophil group (blood eosinophil count ≧300 cells/4 at baseline; 22% [95% CI: −31, 54%]). No reduction in the annual AER in the 300 mg Q2W cohort was observed for the low eosinophil group (blood eosinophil count <300 cells/μL). See TABLE 17.

Conversely, no reduction in annual AER was observed in the high eosinophil subgroup for the tralokinumab 300 mg Q2/4W cohort; whereas, in the low eosinophil subgroup there was a reduction in the annual AER (23%).

TABLE 17
Summary of Asthma Exacerbation Rate Through Week 53 By Peripheral
Blood Eosinophil Count at Baseline (ITT Population)

| | | | | | | | |
|---|---|---|---|---|---|---|---|
| Blood Eosinophil | | | | 95% CI | | 95% CI | P- |
| Count Cut-point | Treatment Group | N | Rate | of Rate | RR | of RR | value |

AER

| | | | | | | | |
|---|---|---|---|---|---|---|---|
| ≧300 | Placebo | 53 | 1.02 | 0.76, 1.35 | — | — | — |
| cells/μL | Tralokinumab 300 mg Q2W | 59 | 0.96 | 0.72, 1.26 | 0.78 | 0.46, 1.31 | 0.350 |
| | Tralokinumab 300 mg Q2/4W | 50 | 1.56 | 1.23, 1.97 | 1.26 | 0.67, 2.35 | 0.476 |
| <300 | Placebo | 89 | 0.89 | 0.70, 1.12 | — | — | — |
| cells/μL | Tralokinumab 300 mg Q2W | 82 | 0.83 | 0.64, 1.06 | 1.06 | 0.67, 1.68 | 0.794 |
| | Tralokinumab 300 mg Q2/4W | 92 | 0.63 | 0.47, 0.83 | 0.77 | 0.48, 1.22 | 0.258 |

CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks;
RR = rate ratio
Rate = Total number of asthma exacerbations in each group/Total person-year follow-up in each group; and 95% CI rate is based on the exact 95% Poisson CI
Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence) and geographical region as the covariates
P-value from the Poisson regression based on pairwise comparison against placebo

In the high eosinophil subgroup the percentage increase from baseline in FEV1 compared to placebo was numerically higher in patients receiving tralokinumab 300 mg Q2W (13.49% [95% CI: 4.99, 22.00]) compared to the low eosinophil subgroup (4.38% [95% CI: −1.51, 10.273]); TABLE 18). Clinically relevant changes in FEV1 were not observed in the tralokinumab 300 mg Q2/4W cohort in the high eosinophil subgroup.

The mean change in ACQ-6 score compared to placebo was larger in the high eosinophil subgroup in patients receiving tralokinumab 300 mg Q2W (−0.49 [95% CI: −0.88, −0.09]) compared to the low eosinophil subgroup (−0.02 [95% CI: −0.34, 0.290]) and approximated the MCID of −0.50. This difference between subgroups was not apparent in the tralokinumab 300 mg Q2/4W cohort.

Clinically important changes from placebo for AQLQ(S) scores were not observed for either tralokinumab treatment cohort in the high eosinophil subgroup.

Numerically greater reductions in asthma daily diary symptom score compared with placebo were observed for both the 300 mg tralokinumab Q2W and Q2/4W cohorts in the high eosinophil subgroup (−0.21 [95% CI: −0.47, 0.04] and −0.19 [95% CI: −0.45, 0.07], respectively) compared with the low eosinophil subgroup (0.03 [95% CI: −0.17, 0.23] and −0.12 [95% CI: −0.32, 0.07]).

TABLE 18
Summary of Change from Baseline in Key Efficacy Endpoints at Week 53.
By Peripheral Blood Eosinophil Count at Baseline (ITT Population)

| | | | | | | |
|---|---|---|---|---|---|---|
| Blood Eosinophil | | | Mean | Difference | | P- |
| Count Cut-point | Treatment Group | N | Estimate | vs Placebo | 95% CI | value |

Change from Pre-bronchodilator Baseline FEV₁ (%)

| | | | | | | |
|---|---|---|---|---|---|---|
| ≧300 | Placebo | 44 | 1.78 | — | — | — |
| cells/μL | Tralokinumab 300 mg Q2W | 50 | 15.27 | 13.49 | 4.99, 22.00 | 0.002 |
| | Tralokinumab 300 mg Q2/4W | 39 | 6.79 | 5.01 | −3.80, 13.82 | 0.264 |
| <300 | Placebo | 75 | 1.67 | — | — | — |
| cells/μL | Tralokinumab 300 mg Q2W | 72 | 6.05 | 4.38 | −1.51, 10.27 | 0.145 |
| | Tralokinumab 300 mg Q2/4W | 73 | 2.15 | 0.48 | −5.32, 6.28 | 0.871 |

Change from Pre-bronchodilator Baseline FEV₁ (L)

| | | | | | | |
|---|---|---|---|---|---|---|
| ≧300 | Placebo | 44 | 0.01 | — | — | — |
| cells/μL | Tralokinumab 300 mg Q2W | 50 | 0.24 | 0.23 | 0.09, 0.37 | 0.001 |
| | Tralokinumab 300 mg Q2/4W | 39 | 0.11 | 0.10 | −0.04, 0.24 | 0.170 |
| <300 | Placebo | 75 | 0.03 | — | — | — |
| cells/μL | Tralokinumab 300 mg Q2W | 72 | 0.10 | 0.07 | −0.03, 0.17 | 0.177 |
| | Tralokinumab 300 mg Q2/4W | 73 | 0.03 | 0.00 | −0.10, 0.10 | 0.992 |

ACQ-6

| | | | | | | |
|---|---|---|---|---|---|---|
| ≧300 | Placebo | 42 | −0.71 | — | — | — |
| cells/μL | Tralokinumab 300 mg Q2W | 44 | −1.20 | −0.49 | −0.88, −0.09 | 0.016 |
| | Tralokinumab 300 mg Q2/4W | 37 | −0.92 | −0.21 | −0.62, 0.20 | 0.322 |
| <300 | Placebo | 71 | −0.64 | — | — | — |
| cells/μL | Tralokinumab 300 mg Q2W | 65 | −0.66 | −0.02 | −0.34, 0.29 | 0.888 |
| | Tralokinumab 300 mg Q2/4W | 68 | −0.86 | −0.22 | −0.53, 0.09 | 0.164 |

AQLQ(S) Overall Score

| | | | | | | |
|---|---|---|---|---|---|---|
| ≧300 | Placebo | 38 | 0.72 | — | — | — |
| cells/μL | Tralokinumab 300 mg Q2W | 40 | 0.91 | 0.19 | −0.24, 0.62 | 0.376 |
| | Tralokinumab 300 mg Q2/4W | 35 | 0.78 | 0.07 | −0.38, 0.51 | 0.765 |
| <300 | Placebo | 64 | 0.67 | — | — | — |
| cells/μL | Tralokinumab 300 mg Q2W | 63 | 0.92 | 0.24 | −0.08, 0.57 | 0.146 |
| | Tralokinumab 300 mg Q2/4W | 59 | 1.04 | 0.37 | 0.04, 0.69 | 0.028 |

Asthma Daily Diary Overall Symptom Score

| | | | | | | |
|---|---|---|---|---|---|---|
| ≧300 | Placebo | 41 | −0.26 | — | — | — |
| cells/μL | Tralokinumab 300 mg Q2W | 42 | −0.47 | −0.21 | −0.47, 0.04 | 0.097 |
| | Tralokinumab 300 mg Q2/4W | 37 | −0.45 | −0.19 | −0.45, 0.07 | 0.152 |
| <300 | Placebo | 67 | −0.33 | — | — | — |
| cells/μL | Tralokinumab 300 mg Q2W | 61 | −0.30 | 0.03 | −0.17, 0.23 | 0.777 |
| | Tralokinumab 300 mg Q2/4W | 64 | −0.45 | −0.12 | −0.32, 0.07 | 0.220 |

ACQ-6 = Asthma Control Questionnaire 6;
AQLQ(S) = Asthma Quality of Life Questionnaire-Standardised Version;
CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Subgroup Analysis: Baseline Th2 Status

Subgroup analysis at Week 53 by Th2 status at baseline showed a reduction in the annual AER in the tralokinumab 300 Q2W cohort compared with placebo in the high Th2 group at baseline (23% [95% CI: −25, 52%). No reduction in annual AER was observed in the low Th2 group (TABLE 19).

No increase in treatment effect for annual AER in the high or low Th2 subgroups was observed in the tralokinumab 300 mg Q2/4W cohort.

TABLE 19
Summary of Annual Asthma Exacerbation Rate at Week
53 By Th2 Status at Baseline (ITT Population)

| | | | | | | | |
|---|---|---|---|---|---|---|---|
| | | | | 95% CI | | 95% CI | P- |
| Th2 Status | Treatment Group | N | Rate ^{a} | of Rate ^{a} | RR ^{b} | of RR ^{b} | value ^{c} |

AER

| | | | | | | | |
|---|---|---|---|---|---|---|---|
| Th2 High | Placebo | 69 | 0.98 | 0.75, 1.25 | — | — | — |
| | Tralokinumab300 mg Q2W | 73 | 0.90 | 0.69, 1.16 | 0.77 | 0.48, 1.25 | 0.298 |
| | Tralokinumab 300 mg Q2/4W | 67 | 1.09 | 0.85, 1.39 | 0.97 | 0.56, 1.67 | 0.900 |
| Th2 Low | Placebo | 73 | 0.90 | 0.69, 1.16 | — | — | — |
| | Tralokinumab 300 mg Q2W | 61 | 0.88 | 0.66, 1.16 | 1.11 | 0.67, 1.84 | 0.685 |
| | Tralokinumab 300 mg Q2/4W | 70 | 0.86 | 0.65, 1.12 | 1.06 | 0.66, 1.70 | 0.820 |

CI = confidence interval;
FEV₁ = forced expiratory volume in one second;
ITT = intent-to-treat;
Q2W = every 2 weeks;
Q2/4W = 2 injections Q2W for 12 weeks followed by Q4W for 38 weeks;
RR = rate ratio
^{a} Rate = Total number of asthma exacerbations in each group/Total person-year follow-up in each group; and 95% CI rate is based on the exact 95% Poisson CI
^{b} Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence) and geographical region as the covariates
^{c} P-value from the Poisson regression based on pairwise comparison against placebo

Improvement in the percentage increase from baseline in FEV1 compared to placebo was observed patients receiving tralokinumab 300 mg Q2W in both the high Th2 subgroup (8.64% [95% CI: 1.57, 15.716]) and the low Th2 subgroup (3.42% [95% CI: −2.61, 9.44]). Clinically relevant changes in FEV1 were not observed in the tralokinumab 300 mg Q2/4W cohort in the high or low Th2 subgroup (TABLE 20).

Clinically important changes from placebo for ACQ-6, AQLQ(S), and asthma daily diary symptom scores were not observed in the high or low Th2 subgroups for either tralokinumab treatment cohort (TABLE 20).

TABLE 20
Summary of Change from Baseline in Key Secondary Efficacy Endpoints
at Week 53 By Th2 Status at Baseline (ITT Population)

| | | | | | | |
|---|---|---|---|---|---|---|
| | | | Mean | Difference | | P- |
| Th2 Status | Treatment Group | N | Estimate | vs Placebo | 95% CI | value |

Change from Pre-bronchodilator Baseline FEV₁ (%)

| | | | | | | |
|---|---|---|---|---|---|---|
| Th2 High | Placebo | 62 | 4.13 | — | — | — |
| | Tralokinumab 300 mg Q2W | 61 | 12.77 | 8.64 | 1.57, 15.71 | 0.017 |
| | Tralokinumab 300 mg Q2/4W | 56 | 7.55 | 3.42 | −3.71, 10.56 | 0.346 |
| Th2 Low | Placebo | 57 | −1.57 | — | — | — |
| | Tralokinumab 300 mg Q2W | 52 | 1.85 | 3.42 | −2.61, 9.44 | 0.266 |
| | Tralokinumab 300 mg Q2/4W | 53 | −2.82 | −1.25 | −7.14, 4.64 | 0.677 |

Change from Pre-bronchodilator Baseline FEV₁ (L)

| | | | | | | |
|---|---|---|---|---|---|---|
| Th2 High | Placebo | 62 | 0.05 | — | — | — |
| | Tralokinumab 300 mg Q2W | 61 | 0.21 | 0.16 | 0.04, 0.27 | 0.008 |
| | Tralokinumab 300 mg Q2/4W | 56 | 0.13 | 0.08 | −0.04, 0.20 | 0.188 |
| Th2 Low | Placebo | 57 | −0.03 | — | — | — |
| | Tralokinumab 300 mg Q2W | 55 | 0.01 | 0.04 | −0.07, 0.15 | 0.439 |
| | Tralokinumab 300 mg Q2/4W | 53 | −0.06 | −0.03 | −0.14, 0.07 | 0.522 |

ACQ-6

| | | | | | | |
|---|---|---|---|---|---|---|
| Th2 High | Placebo | 58 | −0.73 | — | — | — |
| | Tralokinumab 300 mg Q2W | 56 | −0.99 | −0.25 | −0.61, 0.10 | 0.154 |
| | Tralokinumab 300 mg Q2/4W | 55 | −0.98 | −0.25 | −0.60, 0.11 | 0.170 |
| Th2 Low | Placebo | 55 | −0.55 | — | — | — |
| | Tralokinumab 300 mg Q2W | 47 | −0.66 | −0.11 | −0.47, 0.25 | 0.537 |
| | Tralokinumab 300 mg Q2/4W | 48 | −0.66 | −0.11 | −0.46, 0.24 | 0.538 |

AQLQ(S) Overall Score

| | | | | | | |
|---|---|---|---|---|---|---|
| Th2 High | Placebo | 53 | 0.61 | — | — | — |
| | Tralokinumab 300 mg Q2W | 52 | 0.93 | 0.31 | −0.06, 0.69 | 0.102 |
| | Tralokinumab 300 mg Q2/4W | 50 | 0.92 | 0.30 | −0.07, 0.68 | 0.115 |
| Th2 Low | Placebo | 49 | 0.73 | — | — | — |
| | Tralokinumab 300 mg Q2W | 45 | 0.74 | 0.01 | −0.36, 0.38 | 0.973 |
| | Tralokinumab 300 mg Q2/4W | 42 | 0.91 | 0.18 | −0.18, 0.55 | 0.329 |

Asthma Daily Diary Overall Symptom Score

| | | | | | | |
|---|---|---|---|---|---|---|
| Th2 High | Placebo | 55 | −0.31 | — | — | — |
| | Tralokinumab 300 mg Q2W | 54 | −0.44 | −0.13 | −0.37, 0.10 | 0.261 |
| | Tralokinumab 300 mg Q2/4W | 52 | −0.41 | −0.11 | −0.34, 0.13 | 0.366 |
| Th2 Low | Placebo | 53 | −0.29 | — | — | — |
| | Tralokinumab 300 mg Q2W | 43 | −0.20 | 0.08 | −0.14, 0.31 | 0.468 |
| | Tralokinumab 300 mg Q2/4W | 47 | −0.40 | −0.11 | −0.33, 0.10 | 0.309 |

ACQ-6 = Asthma Control Questionnaire 6;
AQLQ(S) = Asthma Quality of Life Questionnaire-Standardised Version;
CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Post-Hoc Subgroup Analyses

In the tralokinumab 300 mg Q2W cohort, the presence of FEV1 reversibility to short acting bronchodilator was identified as an important patient characteristic indicating the potential for clinically important benefit on the annual AER, FEV1, ACQ-6, AQLQ(S), and asthma daily diary symptom score. In addition, in the subgroups postulated to be associated with upregulated IL-13 (high periostin, high eosinophils, high Th2) the reductions in annual AER were numerically greater than in the corresponding ‘low’ subgroups.

In order to further explore the clinical response to tralokinumab and identify a group of patients with the optimal response to tralokinumab, further subgroup analysis explored the combination of high vs low FEV1 reversibility based on the 12% cut-point and peripheral blood biomarkers.

### Post-Hoc Subgroup Analyses: Baseline FEV1 Reversibility and Serum Periostin Level

Within the high reversible group, analysis by serum periostin level at baseline showed that reductions in the annual AER were numerically greater in the tralokinumab 300 mg Q2W cohort compared with placebo in the high periostin group (54% [95% CI: −65, 87%) compared to the low periostin group (4% [95% CI: −140, 61%]; TABLE 21).

TABLE 21
Summary of Annual Asthma Exacerbation Rate at Week 53 By FEV1
Reversibility and Serum Periostin Level (ITT Population)

| | | | | | | | | |
|---|---|---|---|---|---|---|---|---|
| | Baseline | | | | | | | |
| Baseline | Serum | | | | | | | |
| FEV₁ | Periostin | | | | 95% CI | | 95% CI | P- |
| Reversibility | Level | Treatment Group | N | Rate ^{a} | of Rate ^{a} | RR ^{b} | of RR ^{b} | value ^{c} |

AER

| | | | | | | | | |
|---|---|---|---|---|---|---|---|---|
| ≧12% | ≧median | Placebo | 26 | 1.17 | 0.77, 1.71 | — | — | — |
| | | Tralokinumab | 22 | 0.64 | 0.34, 1.10 | 0.46 | 0.13, 1.65 | 0.236 |
| | | 300 mg Q2W | | | | | | |
| | | Tralokinumab | 28 | 1.43 | 1.02, 1.96 | 0.82 | 0.28, 2.37 | 0.713 |
| | | 300 mg Q2/4W | | | | | | |
| | <median | Placebo | 31 | 0.65 | 0.39, 1.02 | — | — | — |
| | | Tralokinumab | 21 | 0.72 | 0.39, 1.21 | 0.96 | 0.39, 2.40 | 0.932 |
| | | 300 mg Q2W | | | | | | |
| | | Tralokinumab | 21 | 0.57 | 0.28, 1.02 | 0.59 | 0.18, 1.92 | 0.383 |
| | | 300 mg Q2/4W | | | | | | |
| <12% | ≧median | Placebo | 39 | 1.13 | 0.81, 1.54 | — | — | — |
| | | Tralokinumab | 57 | 0.91 | 0.67, 1.20 | 0.81 | 0.46, 1.43 | 0.468 |
| | | 300 mg Q2W | | | | | | |
| | | Tralokinumab | 48 | 1.15 | 0.86, 1.50 | 0.86 | 0.41, 1.80 | 0.694 |
| | | 300 mg Q2/4W | | | | | | |
| | <median | Placebo | 51 | 0.79 | 0.56, 1.09 | — | — | — |
| | | Tralokinumab | 44 | 1.07 | 0.79, 1.43 | 1.01 | 0.55, 1.88 | 0.964 |
| | | 300 mg Q2W | | | | | | |
| | | Tralokinumab | 48 | 0.65 | 0.43, 0.94 | 0.81 | 0.43, 1.50 | 0.501 |
| | | 300 mg Q2/4W | | | | | | |

CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
RR = rate ratio
^{a} Rate = Total number of asthma exacerbations in each group/Total person-year follow-up in each group; and 95% CI rate is based on the exact 95% Poisson CI
^{b} Rate ratio and 95% CI for the rate ratio were estimated from the Poisson regression (Pearson correction) with treatment group, age, gender, number of exacerbations in past year (2 vs >2 but ≦6), atopic asthma status (atopic/non-atopic), chronic OCS use (presence versus absence) and geographical region as the covariates
^{c} P-value from the Poisson regression based on pairwise comparison against placebo

In addition, in the high periostin subgroup the percentage increase from baseline in FEV1 compared to placebo was numerically higher (13.85% [95% CI: −0.18, 27.87]) compared to the low periostin subgroup (7.62% [95% CI: −7.60, 22.84]; TABLE 22).

TABLE 22
Subgroup Analysis: Change from Baseline in Pre-bronchodilator at Week
53 By FEV1 Reversibility and Serum Periostin Level (ITT Population)

| | | | | | | | |
|---|---|---|---|---|---|---|---|
| | Baseline | | | | | | |
| Baseline | Serum | | | | | | |
| FEV₁ | Periostin | | | Mean | Difference | | |
| Reversibility | Level | Treatment Group | N | Estimate | vs Placebo | 95% CI | P-value |

Change from Pre-bronchodilator Baseline FEV₁ (%)

| | | | | | | | |
|---|---|---|---|---|---|---|---|
| ≧12% | ≧median | Placebo | 21 | 8.34 | — | — | — |
| | | Tralokinumab | 18 | 22.18 | 13.85 | −0.18, 27.87 | 0.053 |
| | | 300 mg Q2W | | | | | |
| | | Tralokinumab | 23 | 3.01 | −5.33 | −19.09, 8.43 | 0.446 |
| | | 300 mg Q2/4W | | | | | |
| | <median | Placebo | 28 | 8.24 | — | — | — |
| | | Tralokinumab | 17 | 15.86 | 7.62 | −7.60, 22.84 | 0.323 |
| | | 300 mg Q2W | | | | | |
| | | Tralokinumab | 17 | 12.53 | 4.29 | −10.99, 19.56 | 0.579 |
| | | 300 mg Q2/4W | | | | | |
| <12% | ≧median | Placebo | 33 | 1.08 | — | — | — |
| | | Tralokinumab | 51 | 6.06 | 4.98 | −2.89, 12.85 | 0.214 |
| | | 300 mg Q2W | | | | | |
| | | Tralokinumab | 40 | 2.54 | 1.46 | −6.59, 9.51 | 0.721 |
| | | 300 mg Q2/4W | | | | | |
| | <median | Placebo | 42 | −6.23 | — | — | — |
| | | Tralokinumab | 41 | 2.21 | 8.44 | 2.31, 14.57 | 0.007 |
| | | 300 mg Q2W | | | | | |
| | | Tralokinumab | 39 | −5.86 | 0.37 | −5.78, 6.52 | 0.906 |
| | | 300 mg Q2/4W | | | | | |

CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

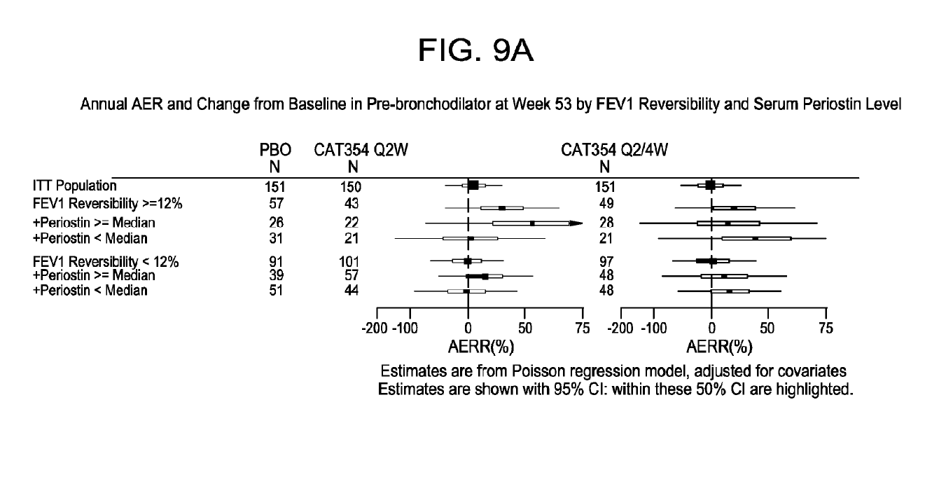

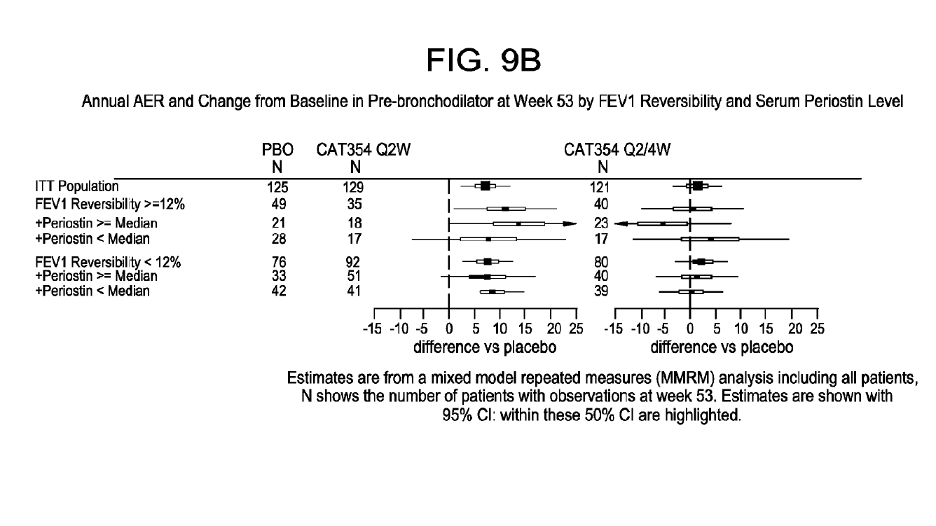

In patients in the tralokinumab 300 mg Q2W cohort with FEV1 reversibility ≧12% at baseline, improvements in AER and pre-bronchodilator FEV1 were observed; this treatment effect was enhanced in those patients that also had periostin >median. Within tralokinumab 300 mg Q2/4W cohort effect sizes on these endpoints were generally lower and the effect of periostin >median at baseline was not observed (TABLE 22 and FIG. 9).

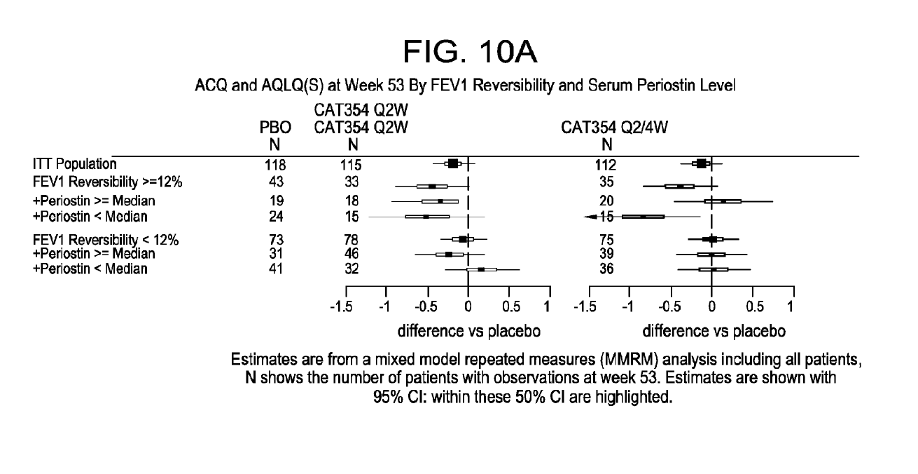

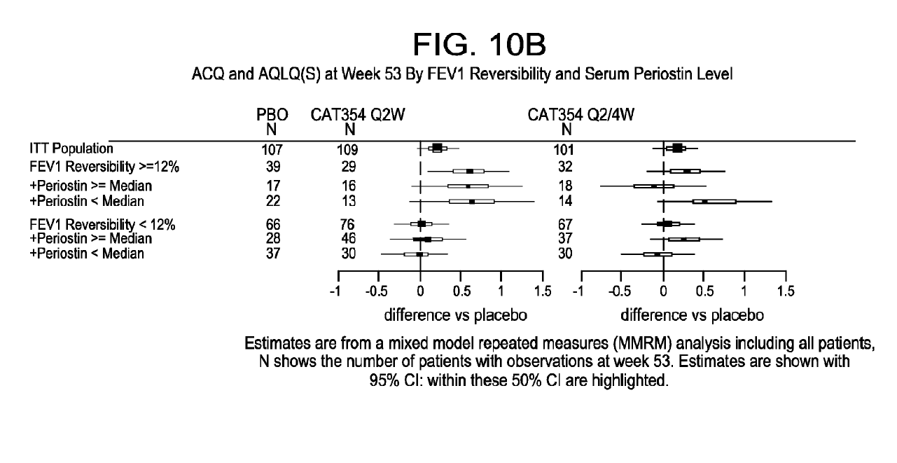

On the endpoints of ACQ-6, AQLQ(S), and asthma daily diary for both the tralokinumab 300 mg Q2W and Q2/4W cohorts, the treatment effect observed in those patients that had FEV1 reversibility >12% at baseline was not further enhanced in those that also had periostin >median (TABLE 23 and FIG. 10).

Note that for the tralokinumab 300 mg Q2W cohort, changes in ACQ-6 and AQLQ(S) reached or approximated the MCID for patients that had FEV1 reversibility ≧12% at baseline (TABLE 23); this was not observed for the Q2/4W cohort.

TABLE 23
Subgroup Analysis - Change from Baseline in Patient Reported Outcomes at
Week 53 By FEV1 Reversibility and Serum Periostin Level (ITT Population)

| | | | | | | | |
|---|---|---|---|---|---|---|---|
| | Baseline | | | | | | |
| Baseline | Serum | | | | | | |
| FEV₁ | Periostin | | | Mean | Difference | | |
| Reversibility | Level | Treatment Group | N | Estimate | vs Placebo | 95% CI | P-value |

Change from Pre-bronchodilator Baseline FEV₁ (%)
ACQ-6

| | | | | | | | |
|---|---|---|---|---|---|---|---|
| ≧12% | ≧median | Placebo | 19 | −0.59 | — | — | — |
| | | Tralokinumab | 18 | −0.92 | −0.34 | −0.93, 0.25 | 0.260 |
| | | 300 mg Q2W | | | | | |
| | | Tralokinumab | 20 | −0.44 | 0.15 | −0.45, 0.75 | 0.626 |
| | | 300 mg Q2/4W | | | | | |
| | <median | Placebo | 24 | −0.15 | — | — | — |
| | | Tralokinumab | 15 | −0.65 | −0.50 | −1.21, 0.21 | 0.164 |
| | | 300 mg Q2W | | | | | |
| | | Tralokinumab | 15 | −0.98 | −0.83 | −1.52, −0.14 | 0.019 |
| | | 300 mg Q2/4W | | | | | |
| <12% | ≧median | Placebo | 31 | −0.73 | — | — | — |
| | | Tralokinumab | 46 | −0.95 | −0.23 | −0.65, 0.19 | 0.291 |
| | | 300 mg Q2W | | | | | |
| | | Tralokinumab | 39 | −0.72 | 0.00 | −0.42, 0.43 | 0.986 |
| | | 300 mg Q2/4W | | | | | |
| | <median | Placebo | 41 | −0.96 | — | — | — |
| | | Tralokinumab | 32 | −0.79 | 0.17 | −0.28, 0.63 | 0.460 |
| | | 300 mg Q2W | | | | | |
| | | Tralokinumab | 36 | −0.92 | 0.04 | −0.40, 0.48 | 0.857 |
| | | 300 mg Q2/4W | | | | | |

AQLQ(S) Overall Score

| | | | | | | | |
|---|---|---|---|---|---|---|---|
| ≧12% | ≧median | Placebo | 17 | 0.57 | — | — | — |
| | | Tralokinumab | 16 | 1.15 | 0.58 | −0.11, 1.26 | 0.097 |
| | | 300 mg Q2W | | | | | |
| | | Tralokinumab | 18 | 0.47 | −0.10 | −0.76, 0.56 | 0.771 |
| | | 300 mg Q2/4W | | | | | |
| | <median | Placebo | 22 | 0.62 | — | — | — |
| | | Tralokinumab | 13 | 1.26 | 0.63 | −0.13, 1.39 | 0.103 |
| | | 300 mg Q2W | | | | | |
| | | Tralokinumab | 14 | 1.28 | 0.66 | −0.05, 1.36 | 0.067 |
| | | 300 mg Q2/4W | | | | | |
| <12% | ≧median | Placebo | 28 | 0.64 | — | — | — |
| | | Tralokinumab | 46 | 0.73 | 0.09 | −0.38, 0.55 | 0.713 |
| | | 300 mg Q2W | | | | | |
| | | Tralokinumab | 37 | 0.92 | 0.27 | −0.21, 0.75 | 0.262 |
| | | 300 mg Q2/4W | | | | | |
| | <median | Placebo | 37 | 0.94 | — | — | — |
| | | Tralokinumab | 30 | 0.90 | −0.04 | −0.50, 0.43 | 0.876 |
| | | 300 mg Q2W | | | | | |
| | | Tralokinumab | 30 | 0.89 | −0.05 | −0.50, 0.40 | 0.836 |
| | | 300 mg Q2/4W | | | | | |

Asthma Daily Diary Overall Symptom Score

| | | | | | | | |
|---|---|---|---|---|---|---|---|
| ≧12% | ≧median | Placebo | 16 | −0.27 | — | — | — |
| | | Tralokinumab | 17 | −0.47 | −0.20 | −0.59, 0.19 | 0.305 |
| | | 300 mg Q2W | | | | | |
| | | Tralokinumab | 20 | −0.25 | 0.02 | −0.36, 0.41 | 0.899 |
| | | 300 mg Q2/4W | | | | | |
| | <median | Placebo | 24 | −0.08 | — | — | — |
| | | Tralokinumab | 13 | −0.37 | −0.29 | −0.70, 0.12 | 0.160 |
| | | 300 mg Q2W | | | | | |
| | | Tralokinumab | 14 | −0.31 | −0.23 | −0.65, 0.20 | 0.293 |
| | | 300 mg Q2/4W | | | | | |
| <12% | ≧median | Placebo | 31 | −0.28 | — | — | — |
| | | Tralokinumab | 39 | −0.30 | −0.02 | −0.33, 0.28 | 0.874 |
| | | 300 mg Q2W | | | | | |
| | | Tralokinumab | 38 | −0.37 | −0.09 | −0.39, 0.22 | 0.580 |
| | | 300 mg Q2/4W | | | | | |
| | <median | Placebo | 39 | −0.45 | — | — | — |
| | | Tralokinumab | 35 | −0.44 | 0.00 | −0.27, 0.28 | 0.992 |
| | | 300 mg Q2W | | | | | |
| | | Tralokinumab | 34 | −0.46 | −0.02 | −0.29, 0.25 | 0.891 |
| | | 300 mg Q2/4W | | | | | |

ACQ-6 = Asthma Control Questionnaire 6;
AQLQ(S) = Asthma Quality of Life Questionnaire-Standardised Version;
CI = confidence interval;
FEV₁ = forced expiratory volume in 1 second;
ITT = intent-to-treat;
Q2W = every 2 weeks;
Q2/4W = 2 injections Q2W for 12 weeks followed by Q4W for 38 weeks
P-values are from a mixed effects repeated measure model comparing treatment effect between tralokinumab and placebo within each cohort at Week 53

### Summary of Efficacy

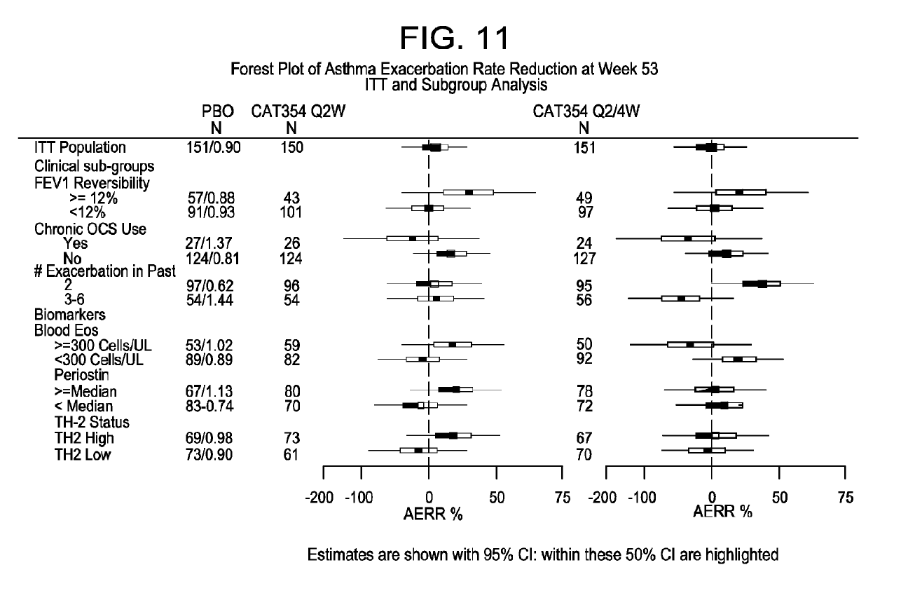

In the ITT population, a reduction in the primary endpoint, the annual AER, was not observed in either tralokinumab treatment cohort compared to placebo; however, trends towards reductions in AER in patients receiving tralokinumab were observed in a number of prespecified subgroups (FIG. 11). In particular, the presence of FEV1 reversibility to SABA ≧12% at baseline was identified as an important clinical characteristic with 34% (95% CI: −32, 67%) reduction in the annual AER observed in the high reversible subgroup in the tralokinumab 300 mg Q2W cohort and 24% [95% CI: −54, 63%] reduction in the Q2/4W cohort; reductions in AER were not observed in the low reversible subgroups in either cohort.

In the high periostin, high eosinophil, and high Th2 subgroups, reductions in annual AER of 25% (95% CI: −19, 53%), 22% (95% CI: −31, 54%), and 23% (95% CI: −25, 52%), respectively, were observed in the tralokinumab 300 mg Q2W cohort with no reductions in corresponding low subgroups. Reductions in annual AER were not observed in Q2/4W cohort in the high or low biomarker subgroups.

Reductions in AER were not observed in the subgroups receiving chronic OCS in either tralokinumab treatment cohort.

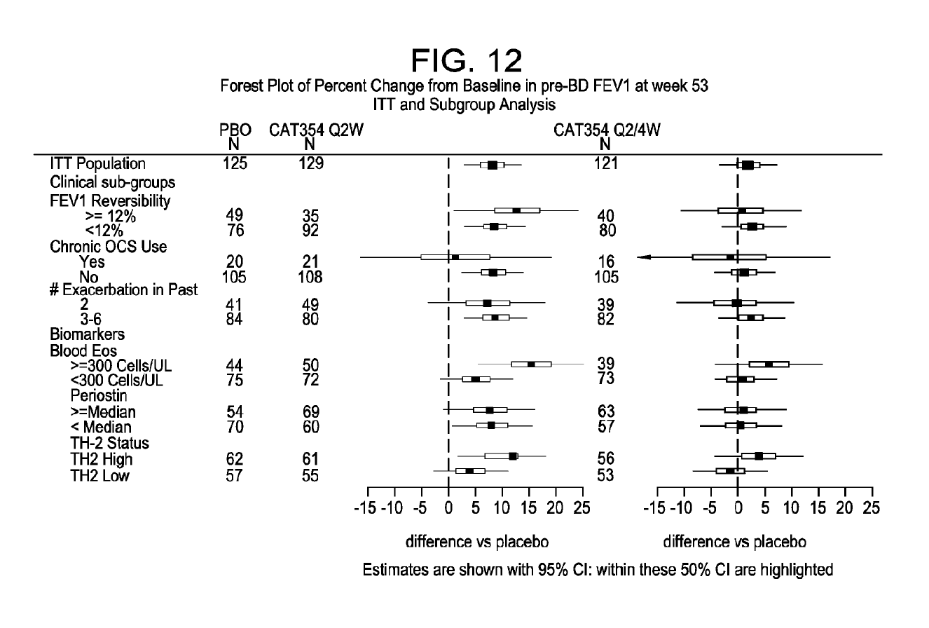

In the ITT population, a statistically significant increase from baseline in pre-bronchodilator FEV1 at Week 53 compared to placebo 7.10% (95% CI: 2.35, 11.84%) was observed in the tralokinumab 300 mg Q2W cohort. The effect size in the tralokinumab 300 mg Q2/4W cohort was lower 1.57% (95% CI: −3.22, 6.35%) indicating a dose-response relationship; this relationship was also observed across the prespecified subgroups with increases in pre-bronchodilator FEV1 at Week 53 consistently higher in the tralokinumab 300 mg Q2W cohort compared to the Q2/4W cohort (FIG. 12). Within the tralokinumab 300 mg Q2W cohort at Week 53, increases in pre-bronchodilator FEV1 compared to placebo were closely matched in both the high and low periostin subgroups and were numerically higher in the high reversible, high eosinophil and high Th2 subgroups than in the corresponding low subgroups.

No increase in pre-bronchodilator FEV1 was observed in the subgroups receiving chronic OCS in either tralokinumab treatment cohort.

In the ITT population, clinically important changes in ACQ-6, AQLQ(S), and asthma daily diary symptom score were not observed in either tralokinumab treatment cohort compared to placebo. In the high reversible subgroup, the mean changes compared to placebo for ACQ-6 and AQLQ scores approximated the MCID in the tralokinumab 300 mg Q2W cohort and were numerically higher than in the Q2/4W cohort; these improvements were not seen in the low reversible subgroup in either dose regimen. The largest reduction in the asthma daily diary symptom score was also observed in the high reversible subgroup in tralokinumab 300 mg Q2W cohort. In high periostin, high eosinophil and high Th2 subgroups clinically important changes in ACQ-6, AQLQ(S), and ASMA symptom score were not consistently observed in either tralokinumab cohort.

Three main conclusions were reached from the pre-specified ITT and subgroup analysis:

(i) In the ITT population and in the majority of subgroups tested, an increase in pre-bronchodilator FEV₁ was observed in the the tralokinumab 300 mg Q2W cohort. A dose response was evident with effects on this endpoint either smaller or absent in the Q2/4W cohort.
(ii) Subgroup analysis within the tralokinumab 300 mg Q2W cohort identified potential responder populations in which reductions in annual AER were observed. The key subgroups identified were:

(a) Patients with FEV₁ reversibility to SABA ≧12% at baseline reduction. In this subgroup, consistent improvements in key secondary endpoints were observed (increase in FEV₁ 11.07% (95% CI: 0.99, 21.14), reduction in mean ACQ-6-0.44 (95% CI: −0.89, 0.01), and increase in AQLQ(S) 0.59 (95% CI: 0.10, 1.09).

(b) Patients in those subgroups postulated to be associated with the presence of up-regulated airway IL-13 (high periostin, high eosinophils, high Th2) suggesting that blood biomarkers associated with up-regulation of IL-13 may be important in identifying patients that will derive most benefit.

(iii) Reductions in AER were not clearly replicated in the same key subgroups in the tralokinumab 300 mg Q2/4W cohorts.

Post hoc analysis explored the hypothesis that patients with FEV1 reversibility ≧12% and serum periostin ≧median at baseline had an enhanced treatment response to tralokinumab 300 mg Q2W. In this subset of patients the reduction in AER was 54% (95% CI: −65, 87%) and the percentage increase from baseline in pre-bronchodilator FEV1 compared to placebo 13.85% (95% CI: −0.18, 27.87), was numerically greater than in those subjects with FEV1 reversibility ≧12% and serum periostin <median (reduction in AER 4% [95% CI: −140, 61%] and increase in FEV1 7.62% [95% CI: −7.60, 22.84]).

In summary, the addition of tralokinumab to high dose ICS and other asthma controller therapies at a dose of 300 mg Q2W results in an increase in pre-bronchodilator FEV₁ in the ITT population and reduction in AER in biologically relevant subgroups. Clinically important improvements in these endpoints were observed in patients responsive to bronchodilator at baseline and these improvements were enhanced further in those patients that also had serum periostin ≧median at baseline. The maintenance dosing regimen of tralokinumab 300 mg Q4W was shown to be inadequate in this study.

### Summary of Safety

The assessment of the overall safety data available from the study has not identified medically important risks associated with tralokinumab at either the 300 mg Q2W or 300 mg Q2/4W regimen. The frequencies of TEAEs were the same between the placebo (84.8%) and tralokinumab 300 mg Q2/4W cohort (84.8%) and slightly higher in the tralokinumab 300 mg Q2W cohort (89.3%). The majority of patients had TEAEs that were mild or moderate in severity and not related to investigational product. The rate of injection site TEAEs was higher in subjects in the Q2W tralokinumab cohort (23.3%) compared to patients receiving placebo Q2W (9.2%) but similar to patients receiving either tralokinumab Q2/4W (20.5%) or placebo Q2/4W (18.7%) and the majority of events were mild to moderate in severity and few patients discontinued investigational product as a result. The frequencies of TESAEs were similar between the placebo (13.9%) and tralokinumab 300 mg Q2W cohort (12.0%) and slightly higher in the tralokinumab 300 mg Q2/4W cohort (16.6%), with few patients having TESAEs related to investigational product. As expected in a study of this duration in a population of patients with severe asthma, there were a number of asthma exacerbations reported as TESAEs but the frequencies of these events were balanced between placebo (4.0%), tralokinumab 300 mg Q2W (6.0%), and tralokinumab 300 mg Q2/4W cohort (6.6%) with only 2 events in the Q2/4W cohort considered related to the product.

### Periostin

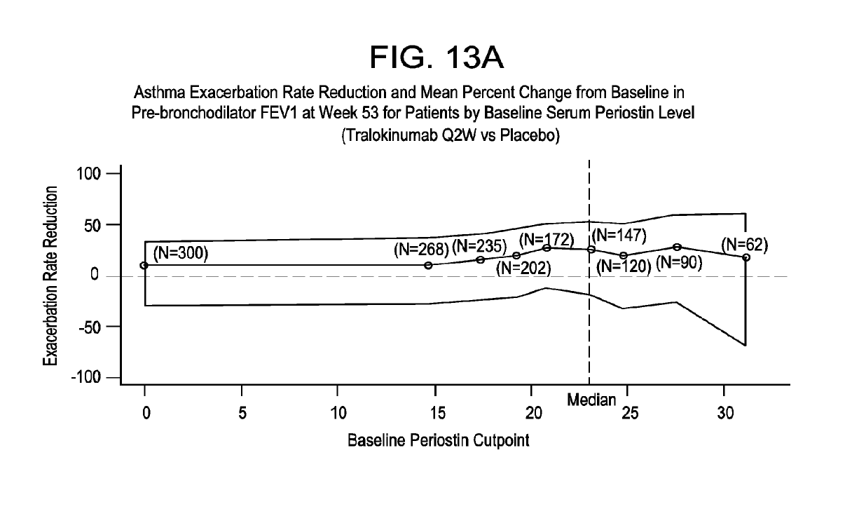

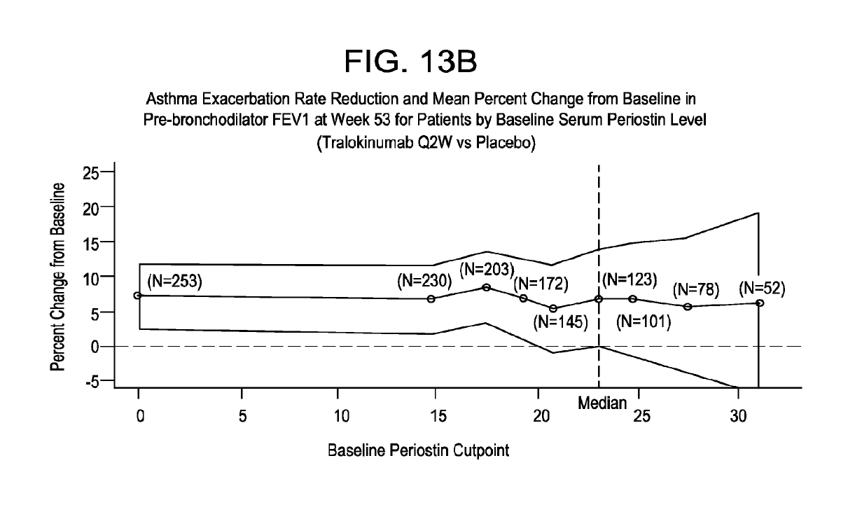

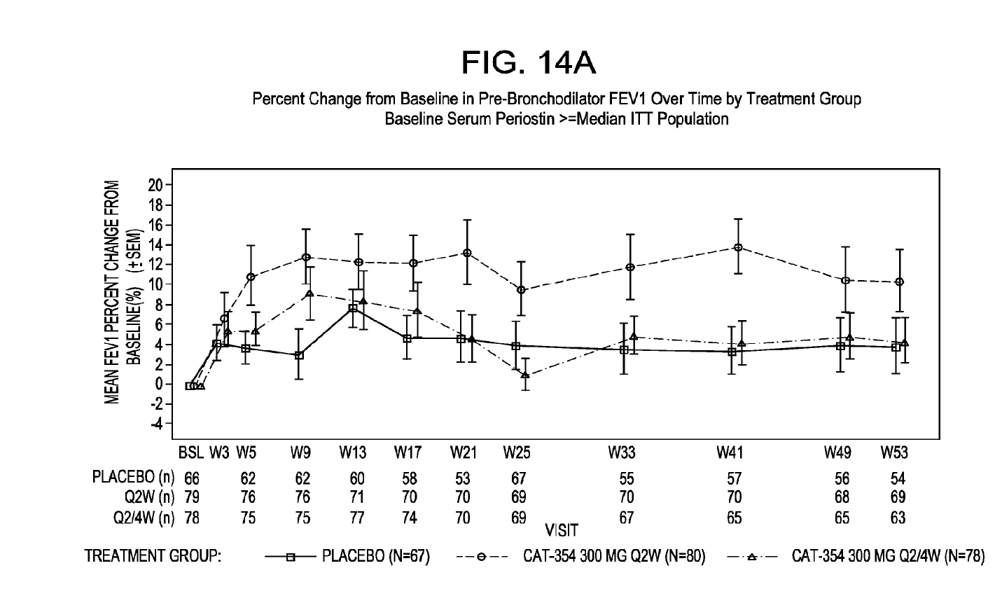

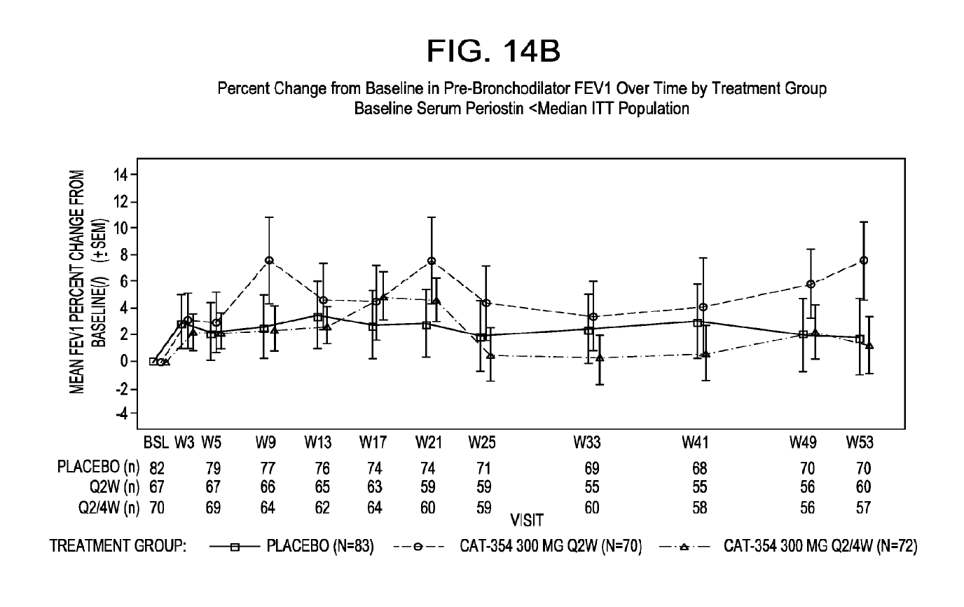

Periostin levels were measured in baseline serum samples, i.e., prior to tralokinumab treatment, that were collected from patients randomised in the Phase 2b study. Key study endpoints including AER reduction, FEV₁, and ACQ-6 stratified by the median serum periostin level to determine if patients with baseline serum periostin levels at or above the median derive greater benefit from tralokinumab compared with those below the median. An increase in FEV₁ (6.75% for patients with baseline serum periostin at or above median vs 8.65% for patients regardless of serum periostin level) and greater AER reduction (25% for patients with serum periostin at or above median vs 7% for patients regardless of serum periostin level) with tralokinumab 300 mg Q2W were observed at Week 53. FIG. 14A and FIG. 14B. The median periostin level used in the study to define high periostin was a baseline serum periostin of ≧23 ng/mL (i.e., high periostin) as measured by the ARCHITECT platform from Abbott Diagnostics. For a continuous representation of AER reduction by periostin level and percent change from baseline in pre-bronchodilator FEV₁ by serum periostin level, see FIG. 13.

In post-hoc analysis, reversible patients (post-bronchodilator reversibility of FEV₁≧12%) with baseline serum periostin levels ≧median had a greater increase in FEV₁ (13.85% for reversible patients with serum periostin at or above median vs 11.07% for reversible patients regardless of serum periostin level) and greater AER reduction (54% for reversible patients with serum periostin at or above median vs 34% for reversible patients regardless of serum periostin level; TABLES 21 and 22) were observed.

Serum periostin levels were substantially reduced by tralokinumab soon after the first dose and remained low for the duration of the study. Greater reduction in serum periostin levels was observed in those patients whose serum periostin levels at baseline were above the median compared to those below the median. These results provided further support for the hypothesis that serum periostin is a surrogate marker for the IL-13 pathway.

### Example 4

### DPP4 as a Peripheral Asthma Biomarker

To identify other potential novel peripheral biomarkers of IL-13 in asthmatics beyond periostin, experiments were conducted to identify a panel of genes upregulated in IL-13 stimulated cultures of bronchial cells from normal human subjects. FIGS. 1-4. Within this IL-13-induced panel, normal and asthmatic serum samples were then interrogated to identify proteins with different levels in the serum of asthmatics and with plausible asthma biology. FIGS. 4-5. Elevated levels of dipeptidyl peptidase 4 (DPP4 [CD 26]) were observed in asthma serum samples compared to normal serum, similar to findings of Lun (Lun et al., J Clin Immunol. 2007; 430-37), who showed DPP4 elevations in plasma from asthma patients compared to control serum that correlated with other Th2 cytokines. In addition, they found increased membrane DPP4 expression on asthmatic CD4+ T cells.

In addition, it was observed that serum DPP4 levels were reduced in subjects taking oral and inhaled steroids (FIG. 6) and in subjects chronically treated with oral corticosteroids (see FIG. 24).

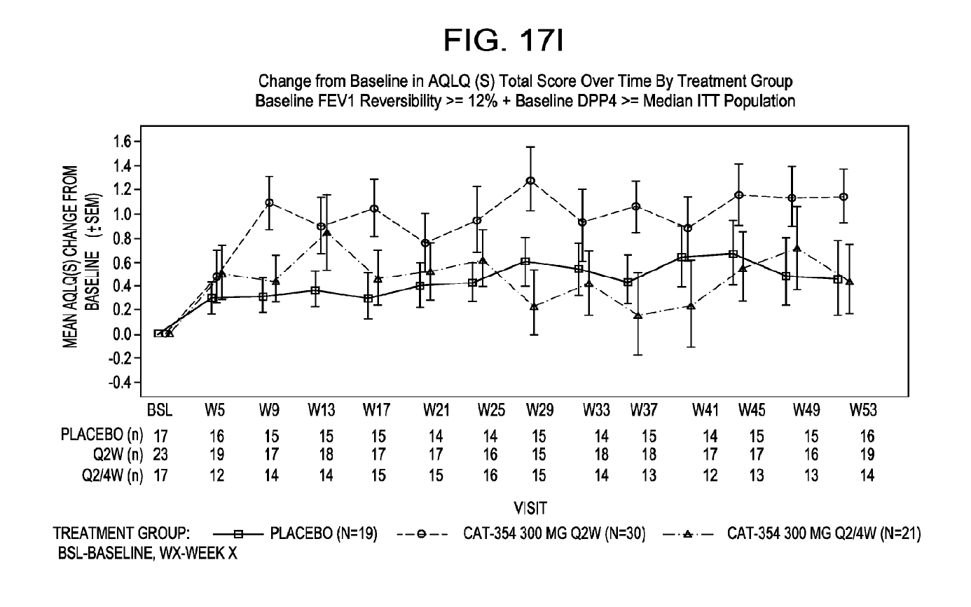

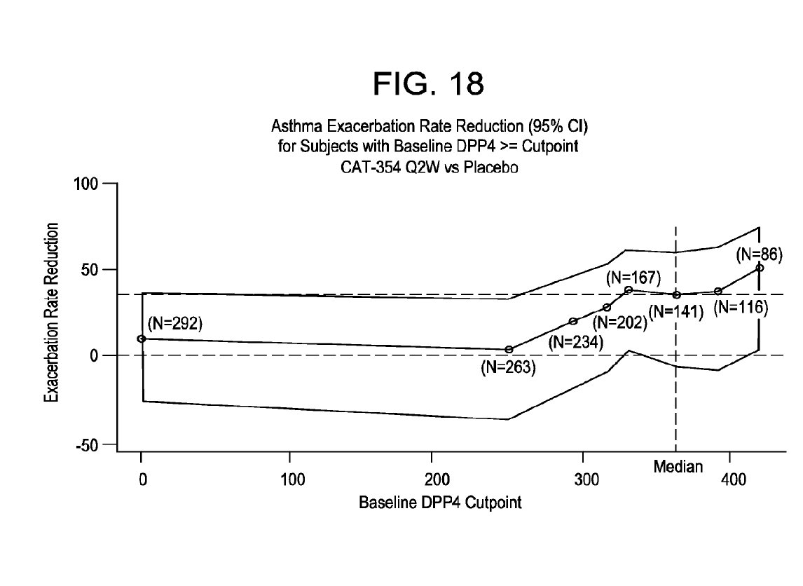

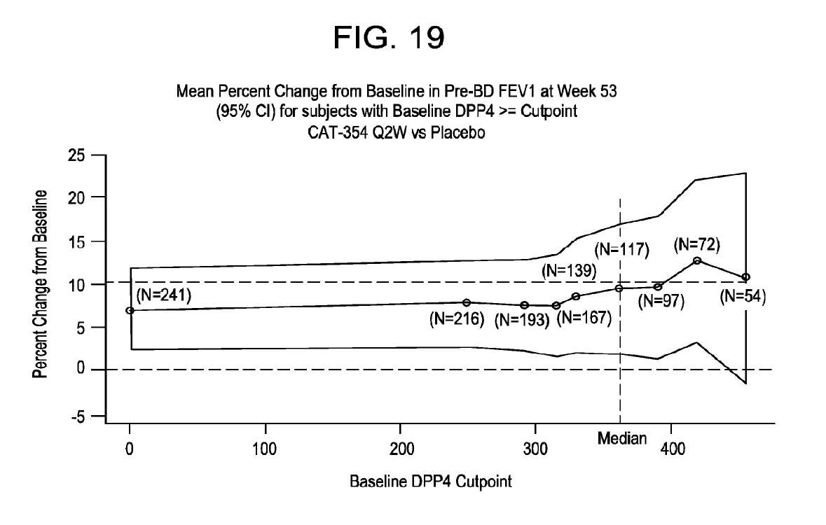

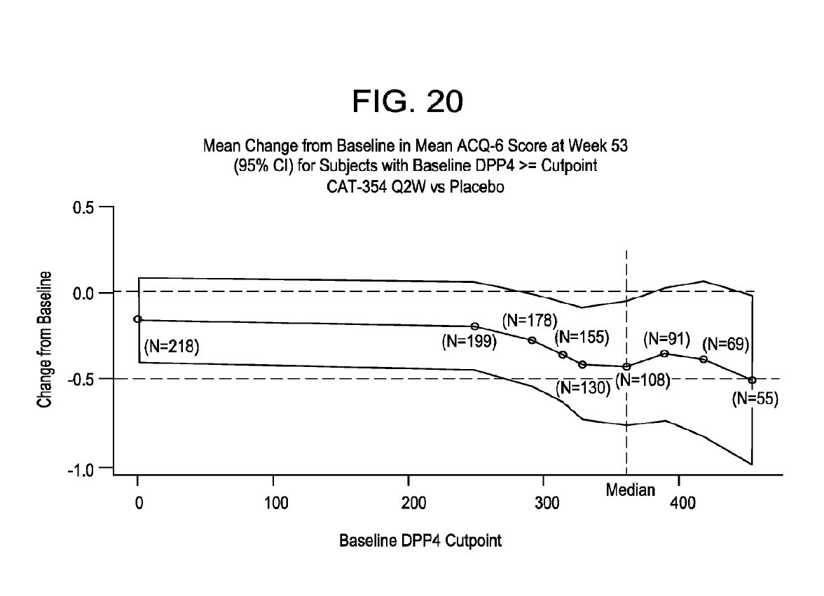

As a preliminary post-hoc analysis, we evaluated primary and secondary endpoints from the tralokinumab Phase 2B study (see Example 3) including AER reduction, FEV1, and ACQ-6 stratified by the median serum DPP4 level (FIGS. 18, 19, and 20) to determine if patients with baseline serum DPP4 levels at or above the median derive greater benefit from tralokinumab compared with those below the median. Serum DPP4 was measured as described in Example 2.

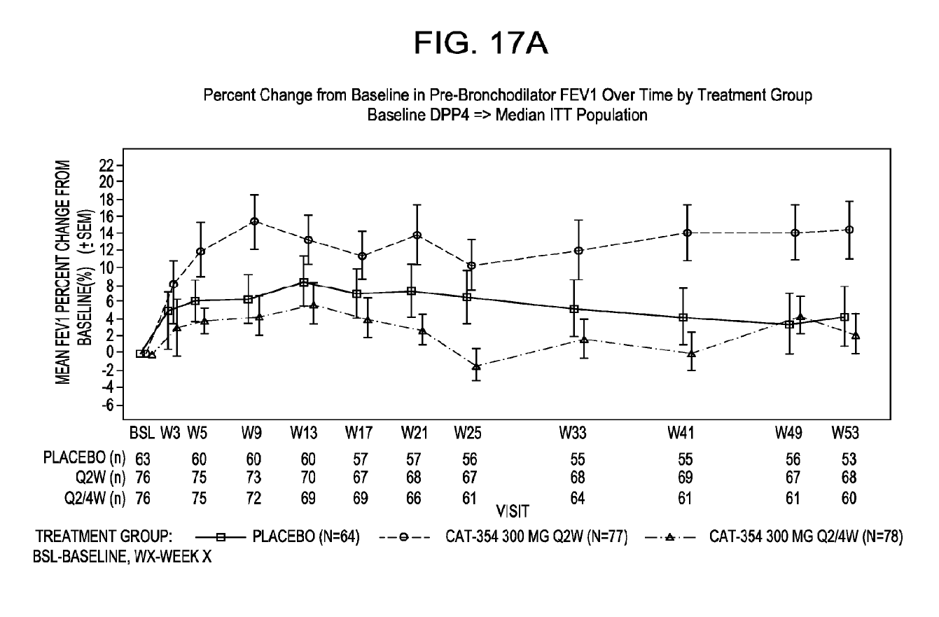

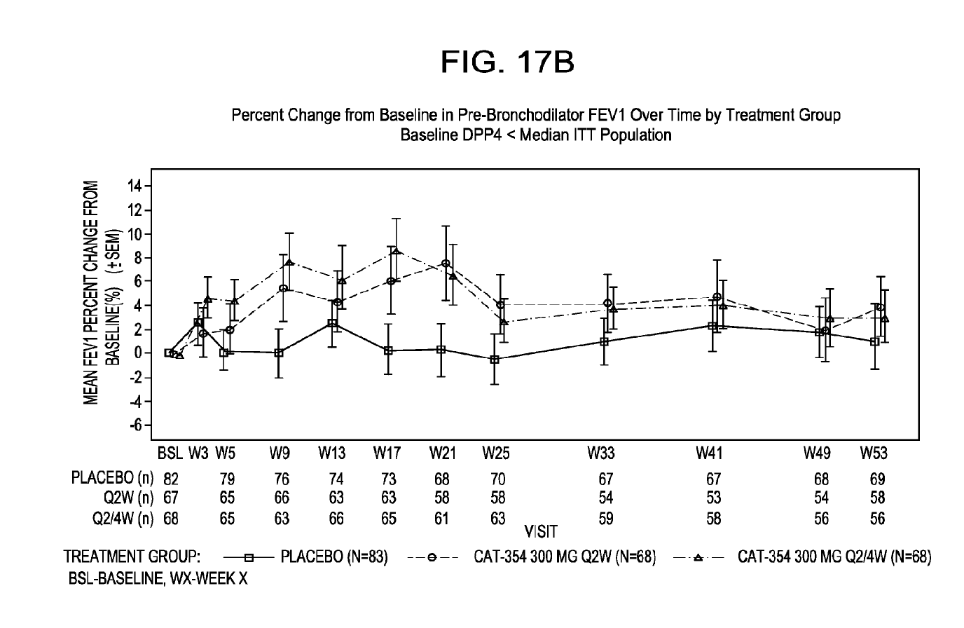

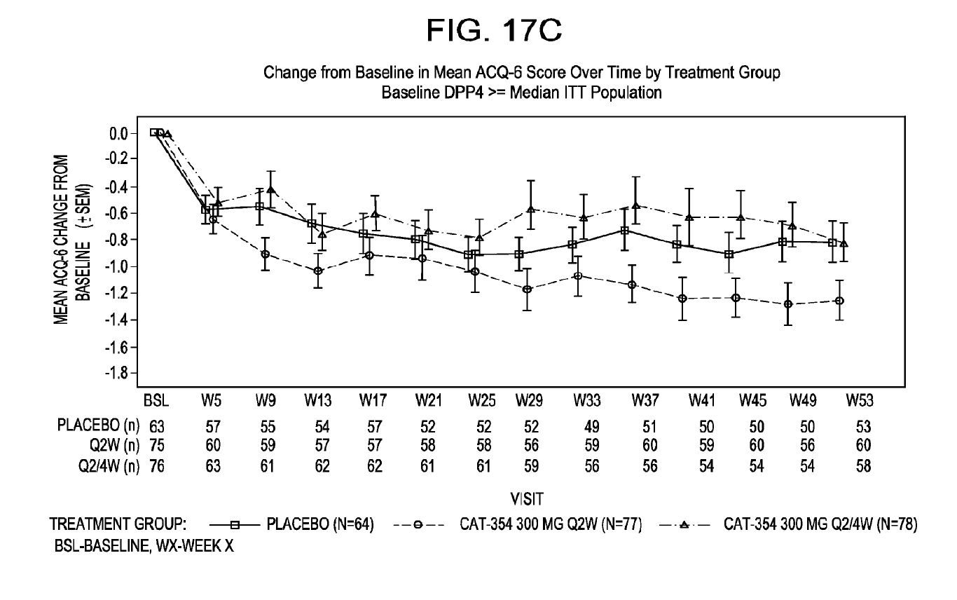

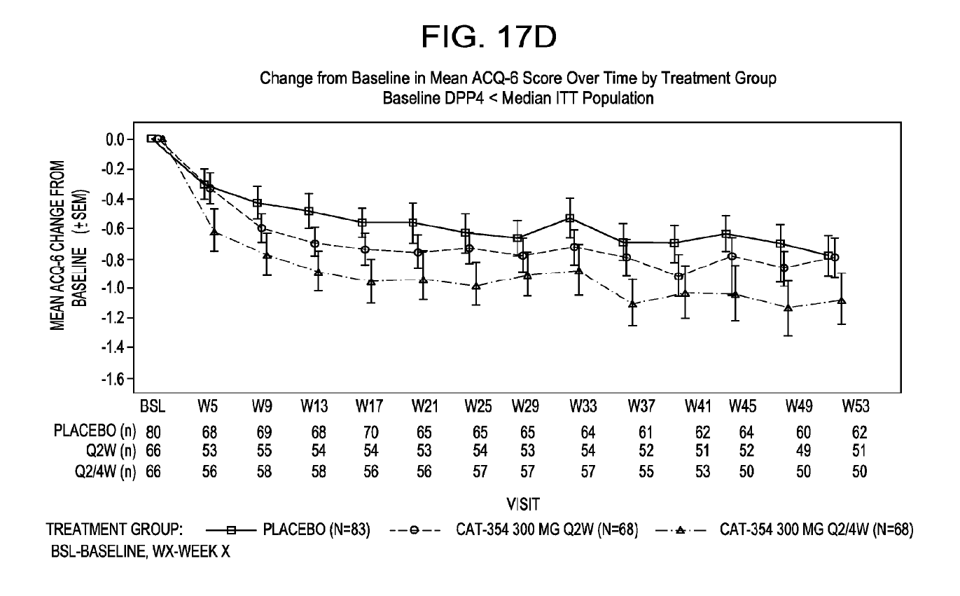

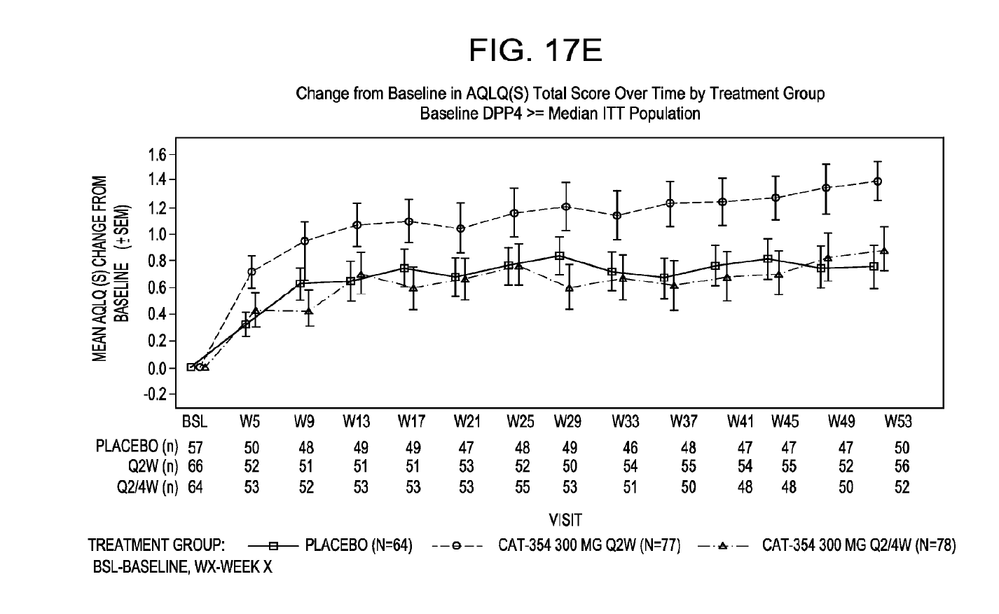

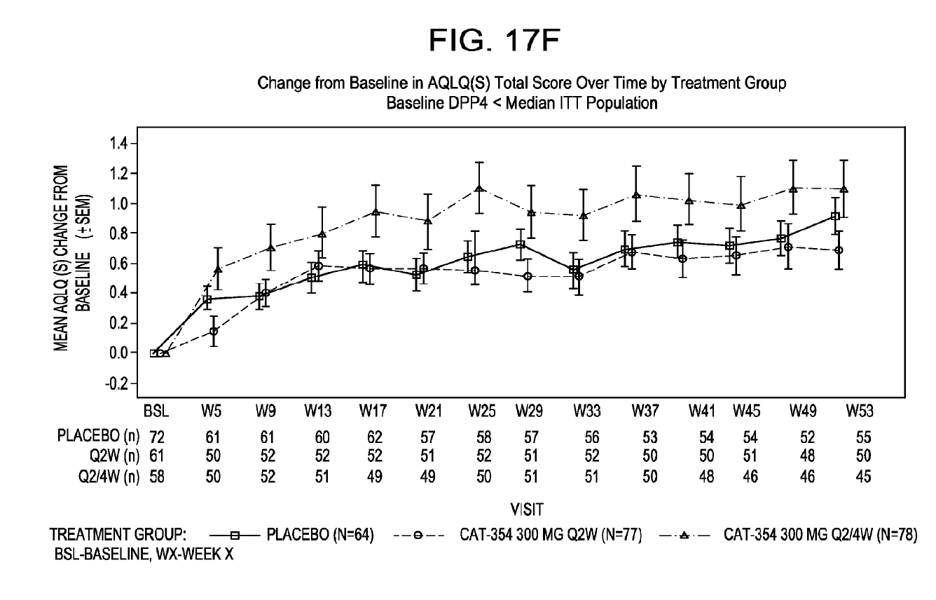

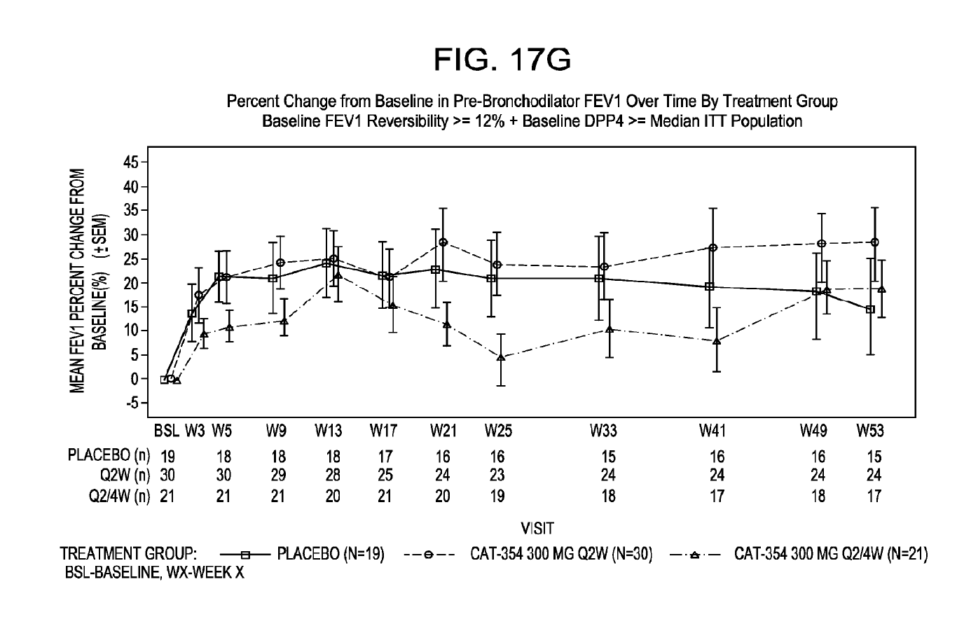

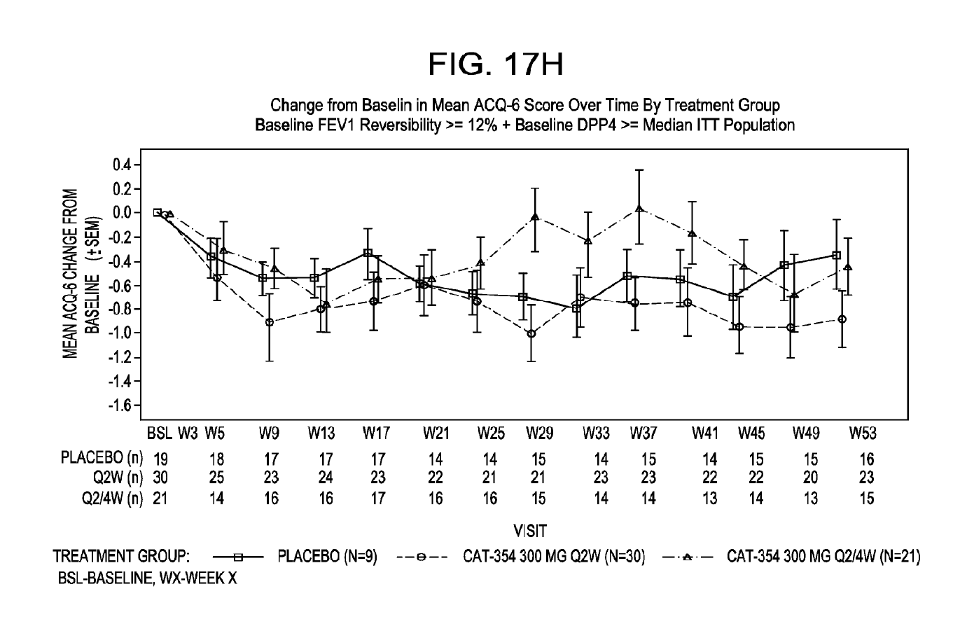

A statistically significant increase in percent change from baseline in pre-bronchodilator FEV1 (see FIG. 17A and FIG. 17B), change from baseline in mean ACQ-6 (see FIG. 17C and FIG. 17D), and change from baseline in mean AQLQ(S) (see FIG. 17E and FIG. 17F) in patients with serum DPP4 at or above median treated with tralokinumab (300 mg Q2W) were observed at Week 53 (TABLE 24). Additionally, unlike periostin, statistically significant changes in ACQ-6 and AQLQ were also observed for patients with serum DPP4 at or above median (TABLE 24). The evaluation of various DPP4 cut-points for ACQ-6, FEV1, and AER Reduction (FIGS. 18-20) supported use of the median value for analysis and showed that altering from the median would not result in significantly greater efficacy for these endpoints.

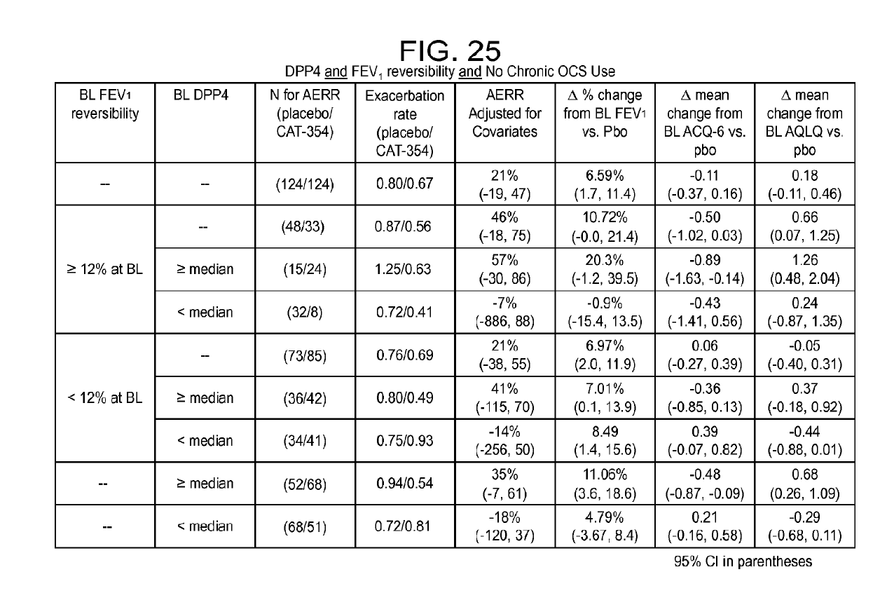

In post-hoc analysis, reversible patients (post-bronchodilator reversibility of FEV1 to a short-acting beta agonist ≧12%) with baseline serum DPP4 levels >=median saw increases in FEV1 (see FIG. 17G) and greater AER reduction. See also FIG. 17H, FIG. 17I, and FIG. 25.

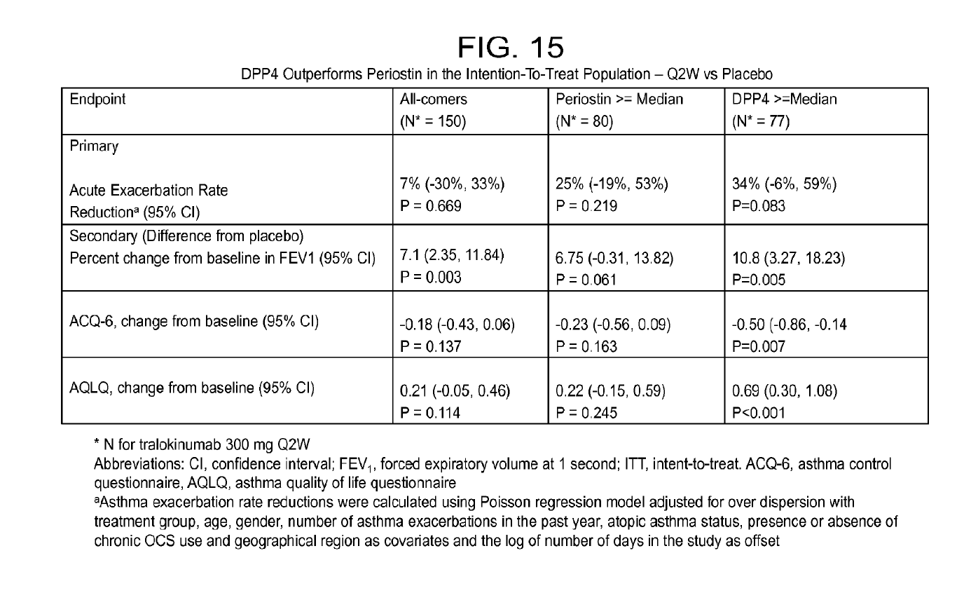

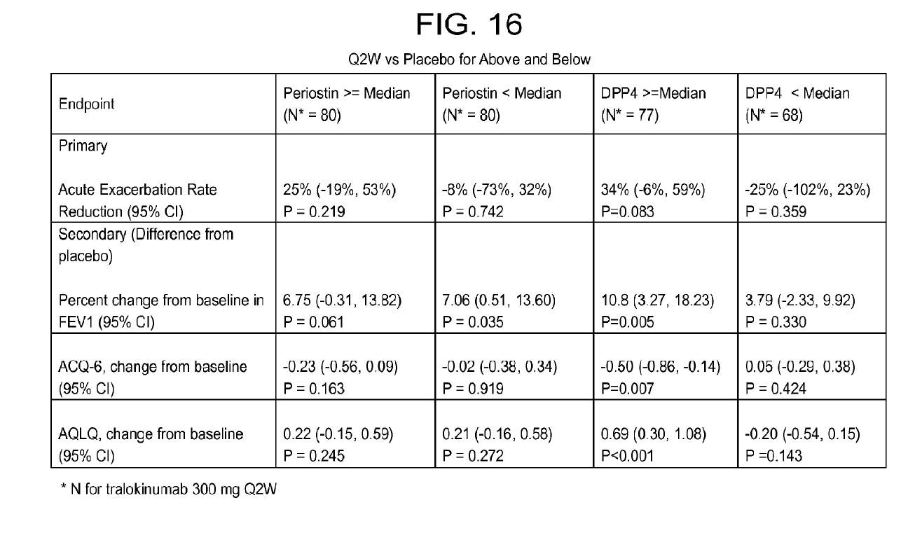

DPP4 outperformed periostin in a variety of endpoints including: acute exacerbation rate reduction, percent change from baseline in pre-bronchodilator FEV1, change from baseline in mean ACQ-6, and change from baseline in mean AQLQ(S). FIGS. 15-16. These findings strongly support the utility of serum DPP4 (e.g. baseline serum DPP4 levels >=median) for identifying patients more likely to benefit from treatment with an IL-13 antagonist (e.g. an anti-IL13 antibody such as tralokinumab or lebrikizumab). These results also strongly support the utility of serum DPP4 (e.g. baseline serum DPP4 levels >=median) to predict exacerbations, exacerbation rate, FEV1 response and asthma symptoms (e.g., night-time waking, symptoms on waking, activity limitation, shortness of breath, wheezing, and SABA use) in asthma patients.

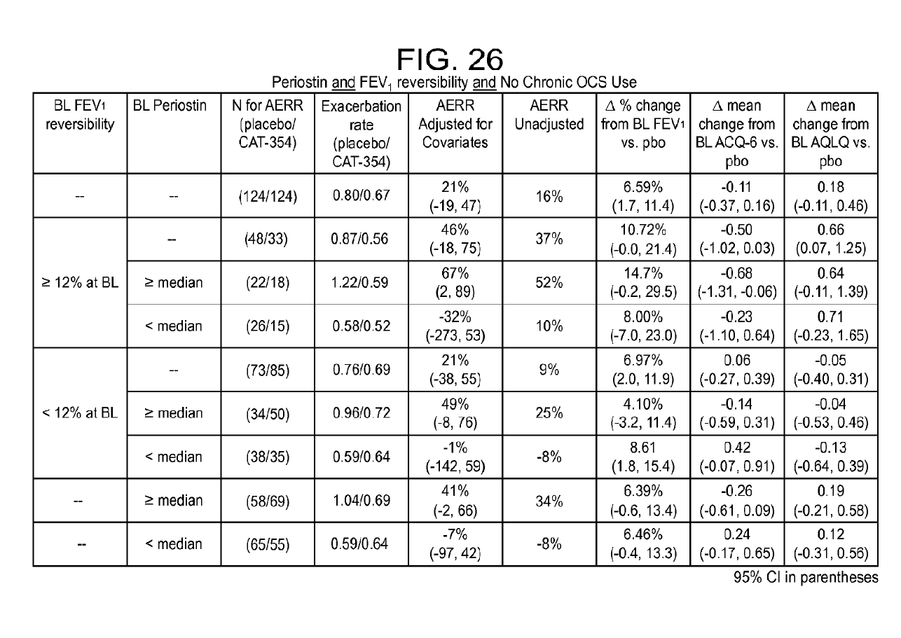

Observations of greater reductions in AER in subgroups defined by both biomarker and clinical characteristics led to exploration of the population of subjects reversible at baseline not receiving OCS (n=33). In this group, a reduction in AER and significant improvements in FEV1, ACQ-6, and AQLQ(S) vs placebo were observed (TABLE 25). Evidence of further improvements in efficacy was observed in those subjects with elevated periostin or DPP4 at baseline (TABLE 25). See also FIG. 25 and FIG. 26 to compare DPP4 and periostin in reversible patients (post bronchodilator reversibility of FEV1 to a short-acting beta agonist ≧12%) with baseline serum DPP4 or Periostin levels >=median and not on chronic oral corticoid steroid treatment.

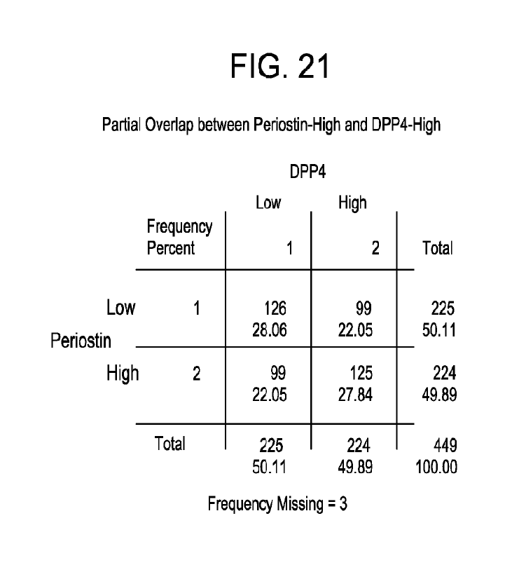

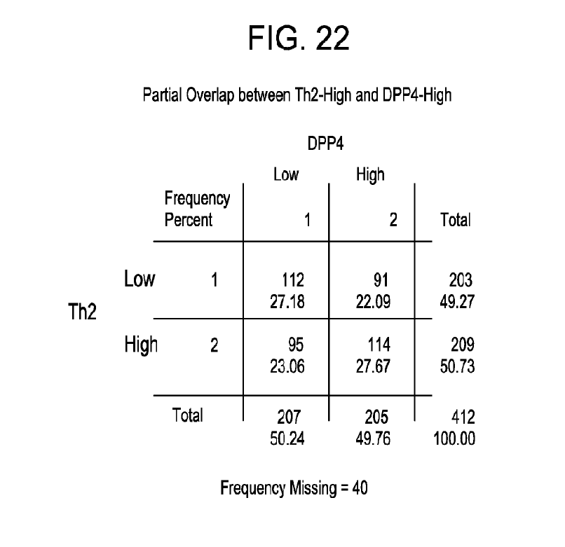

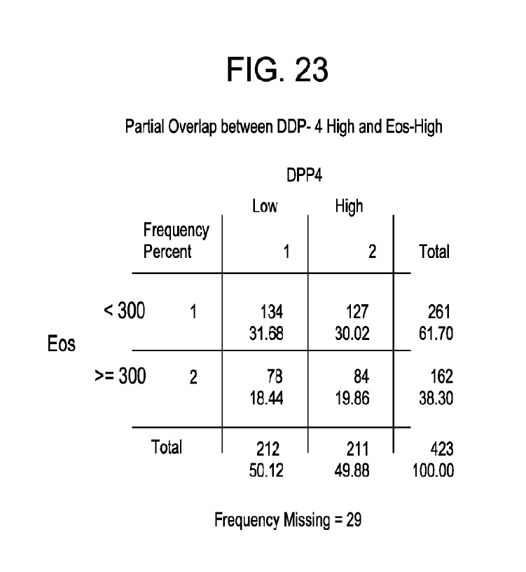

DPP4 can be combined with other markers/classifiers to identify patients more likely to benefit from treatment with an IL-13 antagonist (e.g. an anti-IL13 antibody such as tralokinumab or lebrikizumab), including, e.g., high periostin (Periostin-high), i.e., ≧median serum periostin or about 23 ng/mL; high eosinophil cell count (Eos-high), i.e., blood eosinophil count ≧300 cells/4; or high Th2 (th2-high), i.e., IgE >100 IU/mL and blood eosinophils ≧0.14×10⁹/L. The partial overlap between the DPP4-high (DPP4 levels >=median) group and other groups, namely, periostin-high (periostin levels >=median) (FIG. 21), Th2-high (FIG. 22), and EOS-high (Eosinophil count >=300) (FIG. 23) supports the notion that DPP4 can be used in combination with one or more of these biomarkers (e.g., Th2, periostin, and/or Eos) to identify patients more likely to benefit from treatment with an IL-13 antagonist (e.g. an anti-IL13 antibody such as tralokinumab or lebrikizumab).

TABLE 24
Summary of Primary and secondary efficacy endpoints for tralokinumab
300 mg Q2W ITT and subgroups (FEV1 reversibility, periostin and DPP4)

| | | | | |
|---|---|---|---|---|
| | | | | FEV₁ revers- |
| | | | | ibility ≧ 12% |
| | FEV₁ | | | (N = 43) & |
| | revers- | Periostin ≧ | DPP4 ≧ | Periostin ≧ |
| ITT | ibility ≧ 12% | Median | Median | Median |
| (N = 150) | (N = 43) | (N = 80) | (N = 77) | (N = 22) |

Primary endpoint

| | | | | | |
|---|---|---|---|---|---|
| Asthma | 7% | 34% | 25% | 34% | 54% |
| exacerbation | (−30%, 33%) | (−32%, 67%) | (−19%, 53%) | (−6%, 59%) | (−65%, 87%) |
| rate reduction^{a} | P = 0.669 | P = 0.245 | P = 0.219 | P = 0.083 | P = 0.236 |
| (95% CI) | | | | | |

Secondary endpoints (difference from placebo)

| | | | | | |
|---|---|---|---|---|---|
| Percent change | 7.1 | 11.1 | 6.8 | 10.8 | 13.8 |
| from baseline | (2.35, 11.84) | (0.99, 21.14) | (−0.31, 13.82) | (3.27, 18.23) | (−0.18, 27.87) |
| in FEV₁ | P = 0.003 | P = 0.031 | P = 0.061 | P = 0.005 | P = 0.053 |
| (95% CI) | | | | | |
| Change from | −0.18 | −0.44 | −0.23 | −0.50 | −0.34 |
| baseline in | (−0.43, 0.06) | (−0.89, 0.01) | (−0.56, 0.09) | (−0.86, −0.14) | (−0.93, 0.25) |
| ACQ-6 | P = 0.137 | P = 0.055 | P = 0.163 | P = 0.007 | P = 0.260 |
| (95% CI) | | | | | |
| Change from | 0.21 | 0.59 | 0.22 | 0.69 | 0.58 |
| baseline in | (−0.05, 0.46) | (0.10, 1.09) | (−0.15, 0.59) | (0.30, 1.08) | (−0.11, 1.26) |
| AQLQ | P = 0.114 | P = 0.020 | P = 0.245 | P < 0.001 | P = 0.097 |
| (95% CI) | | | | | |

Abbreviations:
CI, confidence interval;
FEV₁, forced expiratory volume at 1 second;
ITT, intent-to-treat.
ACQ-6, asthma control questionnaire,
AQLQ, asthma quality of life questionnaire
^{a}Asthma exacerbation rate reductions were calculated using Poisson regression model adjusted for over dispersion with treatment group, age, gender, number of asthma exacerbations in the past year, atopic asthma status, presence or absence of chronic OCS use and geographical region as covariates and the log of number of days in the study as offset
N is in each base the number of subjects in the 300 mg Q2W group

TABLE 25
AER reduction, FEV1, ACQ-6, and AQLQ(S) for tralokinumab
Q2W in subjects reversible at baseline and not receiving
chronic OCS vs placebo (post hoc exploratory analyses)

| | Tralokinumab 300 mg (Q2W) reversible without OCS use | | | | |
|---|---|---|---|---|---|
| Parameter vs | | Periostin-high | Periostin-low | DPP4-high | DPP4-low |
| placebo | (n = 33) | (n = 18) | (n = 15) | (n = 24) | (n = 8) |
| AER | 44 | 67 | −32 | 57 | −7 |
| reduction, % | (−22, 74) | (2, 89) | (−273, 53) | (−30, 86) | (−886, 88) |
| (95% CI) | 0.147 | 0.046 | 0.597 | 0.134 | 0.950 |
| P-value | | | | | |
| FEV1 % | 12 | 14.7 | 8.0 | 20.3 | −0.9 |
| change from | (1.5, 22.5) | (−0.2, 29.5) | (−7.0, 23.0) | (1.2, 39.5) | (−15.4, 13.5) |
| baseline | 0.025 | 0.054 | 0.294 | 0.038 | 0.897 |
| (95% CI) | | | | | |
| P-value | | | | | |
| ACQ-6 | −0.55 | −0.68 | −0.23 | −0.89 | −0.43 |
| change from | (−1.07, −0.04) | (−1.31, −0.06) | (−1.10, 0.64) | (−1.63, −0.14) | (−1.41, 0.56) |
| baseline | 0.036 | 0.033 | 0.596 | 0.020 | 0.390 |
| (95% CI) | | | | | |
| P-value | | | | | |
| AQLQ(S) | 0.70 | 0.64 | 0.71 | 1.26 | 0.24 |
| change from | (0.12, 1.28) | (−0.11, 1.39) | (−0.23, 1.65) | (0.48, 2.04) | (−0.87, 1.35) |
| baseline | 0.019 | 0.095 | 0.138 | 0.002 | 0.663 |
| (95% CI) | | | | | |
| P-value | | | | | |

In conclusion, this double-blind phase 2b study enrolled adults with severe asthma, post-bronchodilator forced expiratory volume in 1 second (FEV1) reversibility ≧12% and ≧200 mL within 3 years/at screening and ≧2 asthma exacerbations in the previous year. Subjects received fluticasone/salmeterol 500 μg/50 μg bid (or equivalent) and continued pre-study controller medications. Following 5-week run-in, subjects with FEV1 40-80% predicted or Asthma Control Questionnaire 6 (ACQ-6) score ≧1.5 were randomized to tralokinumab 300 mg/placebo (2:1) every 2 weeks (Q2W) or tralokinumab 300 mg/placebo (2:1) Q2W for 12 weeks followed by every 4 weeks (Q4W). The primary endpoint was asthma exacerbation rate (AER) over 52 weeks. Secondary endpoints included FEV1, ACQ-6, Asthma Quality of Life Questionnaire (AQLQ), and safety. The trial was powered to detect a 40% reduction in AER for each tralokinumab group (Q2W or Q4W) vs. combined placebo groups with 80% power and significance level 0.15. Subjects with baseline FEV1 reversibility ≧12% defined a “reversible” subgroup. Baseline levels of serum DPP4 and periostin, genes whose expression is highly induced by IL-13, were assessed as potential surrogate biomarkers with subgroups defined by median levels.

Analyses were based on intent-to-treat population (ITT, N=452). Baseline characteristics, mean (SD): age: 50.2 (12.3); ACQ-6: 2.55 (0.97); FEV1% predicted: 68.6 (18.1). AER at week 53 was similar in both tralokinumab groups vs. placebo. Trends towards AER reduction in Q2W were observed in reversible, periostin-high, and DPP4-high subgroups (TABLE 24). Reversible and periostin-high subgroup AER reductions were 54% (−65, 87%), and when excluding subjects receiving oral corticosteroid, 67% (2, 89%). At week 53, a statistically significant increase in pre-bronchodilator FEV1 was observed for Q2W and increases were evident in all subgroups (TABLE 24). ACQ-6 and AQLQ were significantly different from placebo in the reversible and DPP4-high subgroups for Q2W (TABLE 24). No significant differences vs. placebo were observed for secondary endpoints in Q4W group or subgroups. Frequencies of treatment emergent serious adverse events/adverse events were similar within the safety population (tralokinumab Q2W: 12.0/89.3%; Q4W: 16.6/84.8%; placebo: 13.9/84.8%).

### Example 5

### Identification of Peripheral Markers of IL-13 Activation in Atopic Dermatitis

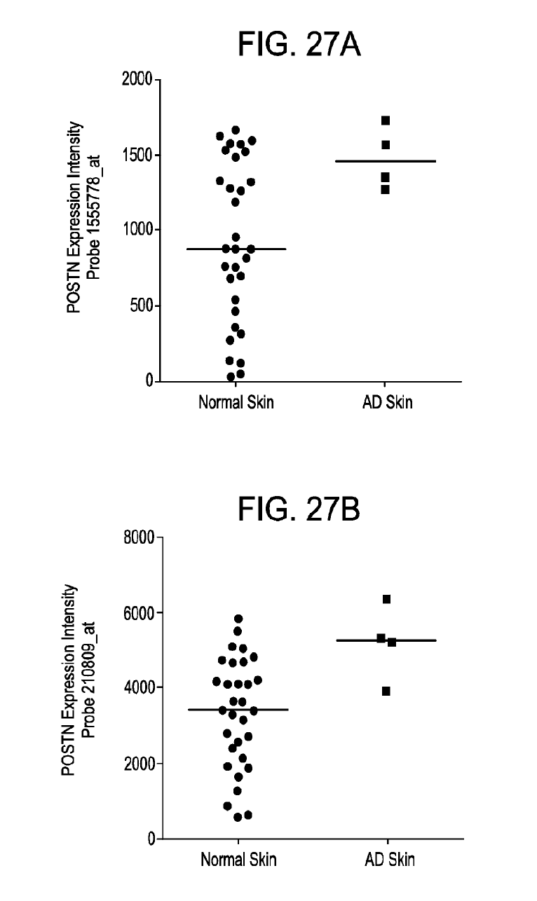

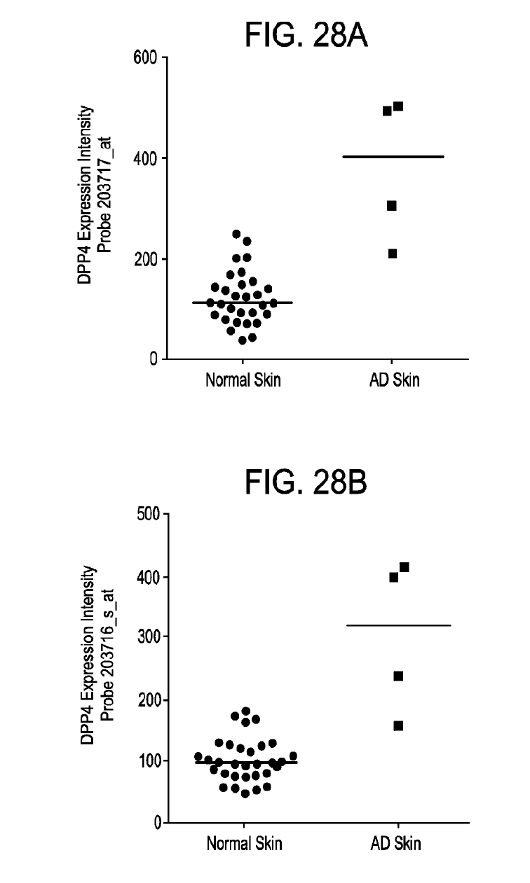

To examine whether periostin and/or DPP4 are also up-regulated in the human skin of patients suffering from atopic dermatitis, transcriptional alterations in four atopic dermatitis skin samples and 31 normal skin samples were analyzed using whole genome microarray. Briefly, biotin-labeled amplified cRNA was generated from total RNA using cDNA Synthesis and IVT Labeling kits and fragmented for hybridization on Affymetrix Human Genome U133 Plus 2.0 GeneChip® arrays. Data capture and quality assessments were performed with the GeneChip Operating Software tool. The R statistical analysis tool was used to calculate probe-level summaries (frma) from the array CEL files. Expression intensity data (linear) from whole genome array analysis showed that mRNA expression of periostin (FIG. 27) and DPP4 (FIG. 28) were elevated in atopic dermatitis skin compared to normal skin.

The finding that DPP4 and periostin expression levels are increased in the skin of atopic dermatitis patients indicates that DPP4 and/or periostin gene expression levels: (1) could be used as a peripheral markers of IL-13 pathway activation in atopic dermatitis patients; (2) could be informative in electing potential therapies for atopic dermatitis patients, and (3) could be useful in selecting patients responsive to therapy using an IL-13 antagonist, for example, an anti-IL-13 antibody such as tralokinumab or lebrikizumab.

The results obtained using skin samples from atopic dermatitis patients suggest that expression levels (e.g., gene expression and/or protein expression) of DPP4 and/or periostin in serum can also be used as biomarkers in atopic dermatitis. Protein levels of DPP4 and periostin were also elevated in serum of atopic dermatitis patients (See Example 8). Accordingly, these findings that serum and skin DPP4 and/or periostin protein levels are increased in atopic dermatitis suggest that DPP4 and/or periostin levels: (1) could be used as a peripheral marker of IL-13 pathway activation in atopic dermatitis patients; (2) could be informative in electing potential therapies for atopic dermatitis patients, and (3) could be useful in selecting patients responsive to therapy using an IL-13 antagonist, for example, an anti-IL-13 antibody such as tralokinumab or lebrikizumab.

### Example 6

### CAT-354-1049 Computed Tomography (CT) Image Data Analysis: 3D Airway Analysis

As discussed herein, there is a need to identify patients who would benefit from therapeutic intervention with an IL-13 antagonist and predict the outcome of the treatment with IL-13 antagonists such as anti-IL-13 antibodies. This is achieved using biochemical biomarkers such as DPP4, periostin and/or clinical characteristics such as FEV1 reversibility, or combinations thereof, as described above in Examples 3-5. Another approach is using Computed Tomography (CT) imaging data as high-performance CT scanners are available at most hospitals, and standardized image analysis can be obtained as a service. A change in airway dimensions, and in particular airway resistance that can be estimated from the subsegmental airway dimensions, should relate to the improvements in lung function, and be a more objective measure than standard lung function tests such as FEV1.

Other groups have studied the change in dimensions for large airways (RB1) as one of the indicators of treatment effect (see, e.g., Haldar, et al 2009). Here, we investigated the treatment effect following Tralokinumab administration as measured by changes in subsegmental airway dimensions from baseline, as quantifying the dimensions of more peripheral airways, such as the subsegmental bronchi, should have a greater potential to reflect efficacy since these airways are not as rigid as the larger airways, including RB1.

Computed tomography (CT) imaging data of lung scans obtained from patients enrolled in the CAT-354-1049 clinical trial (described in Example 3) were analyzed using VIDA APOLLO® software (version 1.2.001_Investigator; VIDA Diagnostics, Inc., Coralville, Iowa) using methods well known in the art. See, e.g., Gupta et al., J Allergy Clin Immunol. 133(3):729-738 (2014). In the clinical trial, CT scanning was used to determine the effects of tralokinumab administration on airway wall structural change. The image data was obtained using spiral/helical MSCT (multislice computed tomography) imaging. CT scanners from GE Healthcare, Philips and Siemens were used in the multicenter trial. Imaging and reconstruction parameters were standardized with tube voltage 120kVp, slice thickness <=1.0 mm, recon kernel Standard (GE), B (Philips) and B30f (Siemens), respectively. All images were acquired at full inspiration (TLC). In this study, only the upper part of the lung was imaged, allowing the analysis of the segmental and sub-segmental airways in the upper lobes. The VIDA APOLLO® software allowed the 3D analysis of parameters related to airway dimensions, in particular, bronchial tubes. As used herein, the term “bronchial tube” means a bronchus or any of its branches, including bronchia and bronchioles. The parameters measured for each bronchial tube (or segment) were average cross-sectional lumen area (LA), wall area (WA), wall area percentage (WA %), and wall thickness (WT). The term “lumen” refers to the inner open space or cavity of a bronchial tube. The term “wall area” refers to the cross-sectional area of a bronchial tube wall. Wall area percentage was calculated as follows: 100*wall area/(wall area+lumen area). Measurements were performed in all imaged segmental and subsegmental bronchi in the upper lobes. Segmental airways (up to five in each subject) were right apical (RB1), right anterior (RB2), right posterior (RB3); left apicoposterior (LB1+2), and left anterior (LB3). Subsegmental airways (up to 14 in each subject) were RB1a & b, RB2a & b, RB3a & b, LB1, LB1a* & b*, LB2, LB2a* & b*, LB3a & b, with the areas labeled with an asterisk being sub-subsegmental airways. LB1 and LB2 could alternatively be named LB1+2a and LB1+2b, respectively. The corresponding sub-subsegmental airways (labeled with an asterisk above) can alternatively be named LB1+2ai, LB1+2aii, LB1+2bi, LB1+2bi, respectively. See e.g. Naidich, et al, Imaging of the Airways—Functional and Radiologic Correlations, 2005.

The baseline measurements disclosed herein were consistent with the baseline measurements observed in other CT studies in asthma, including Gupta et al., J Allergy Clin Immunol. 133(3): 729-738 (2014).

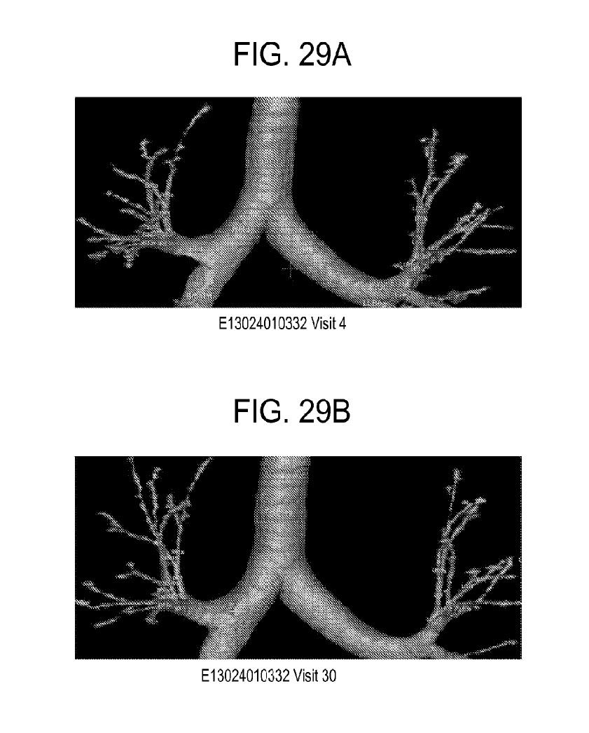

Changes in the airway parameters described above were calculated for each airway segment separately, and then averaged over segmental and subsegmental airways in each subject. Calculations of airway resistance and averages of cross-sectional airways were performed in Matlab (Matlab R2010a (MathWorks, Natick, Mass.)). Only relative changes between baseline (visit 4) (see FIG. 29, panel A) and follow up (visit 30) (see FIG. 29, panel B) were calculated. Group differences were calculated only between total tralokinumab (i.e. 300 mg Q2+300 mgQ2/4W cohorts) and total placebo.

The analysis dataset used all subjects that had CT scans at both baseline, i.e., visit 4, and follow-up, i.e., visit 30. The most severe CT protocol deviations were excluded (i.e., change in image slice thickness or reconstruction kernel between visits 4 and 30).

Airway Resistance was calculated assuming laminar airflow and airway segments that were substantially longer than their diameter. Thus, airway resistance was theoretically calculated as:

$$R = \frac{8 μ lV}{π {r}^{4}}$$

where r is the radius of the airway (μ=viscosity, l=length, V=flow rate). Since area is approximately r², relative change in resistance can consequently be estimated as:

$$\frac{{R}_{2} - {R}_{1}}{{R}_{1}} = \frac{1 \text{/} {LA}_{2}^{2} - 1 \text{/} {LA}_{1}^{2}}{1 \text{/} {LA}_{1}^{2}}$$

Relative change in airway resistance was calculated for each airway segment prior to averaging across several bronchi.

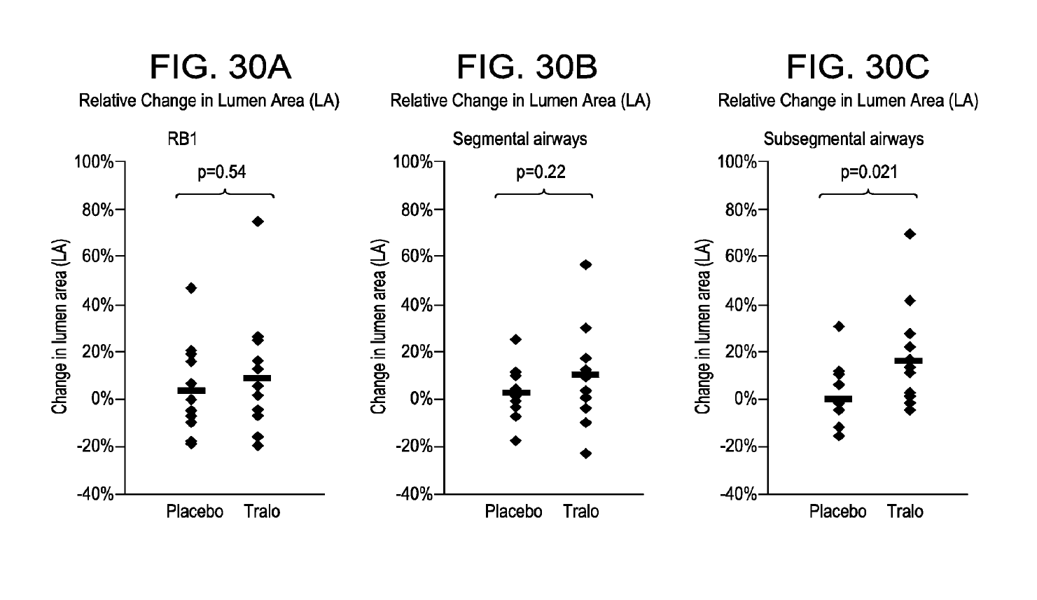

The relative changes in Luman Area (LA) from baseline to visit 30 are shown in TABLE 26 and FIG. 30.

TABLE 26
Relative change in Lumen Area (LA) from baseline to visit 30

| | | Total | | |
|---|---|---|---|---|
| | Total Placebo, | Tralokinumab, | Group | |
| | n = 12 | n = 14 | differ- | P |
| | Mean (S.D.) | Mean (S.D.) | ence | value |
| RB1 | +4.15% (19.06%) | +9.54% (24.58%) | +5.39% | 0.54 |
| Seg- | +3.25% (10.41%) | +11.16% (19.20%) | +7.90% | 0.22 |
| mental | | | | |
| Subseg- | +0.28% (13.24%) | +16.77% (19.52%) | +16.49% | 0.021 |
| mental | | | | |
| All | +1.12% (10.52%) | +15.34% (18.36%) | +14.22% | 0.026 |

Relative change in LA should not be affected by normalization with body surface area (BSA) at baseline, i.e.

$$\frac{{LA}_{2} \text{/} BSA - {LA}_{1} \text{/} BSA}{{LA}_{1} \text{/} BSA} = \frac{{LA}_{2} - {LA}_{1}}{{LA}_{1}}$$

The relative changes in Wall Area (WA) from baseline to visit 30 are shown in TABLE 27.

TABLE 27
Relative change in Wall Area (WA) from baseline to visit 30

| | | Total | | |
|---|---|---|---|---|
| | Total Placebo, | Tralokinumab, | Group | |
| | n = 12 | n = 14 | differ- | P |
| | Mean (S.D.) | Mean (S.D.) | ence | value |
| RB1 | +5.17% (20.06%) | +17.17% (32.69%) | +11.99% | 0.28 |
| Seg- | +8.59% (12.02%) | +12.18% (16.67%) | +3.59% | 0.54 |
| mental | | | | |
| Subseg- | +1.89% (10.04%) | +10.29% (14.40%) | +8.40% | 0.10 |
| mental | | | | |
| All | +4.02% (9.19%) | +11.19% (12.79%) | +7.17% | 0.12 |

Relative change in WA should not be affected by normalization with body surface area (BSA) at baseline, i.e.

$$\frac{{WA}_{2} \text{/} BSA - {WA}_{1} \text{/} BSA}{{WA}_{1} \text{/} BSA} = \frac{{WA}_{2} - {WA}_{1}}{{WA}_{1}}$$

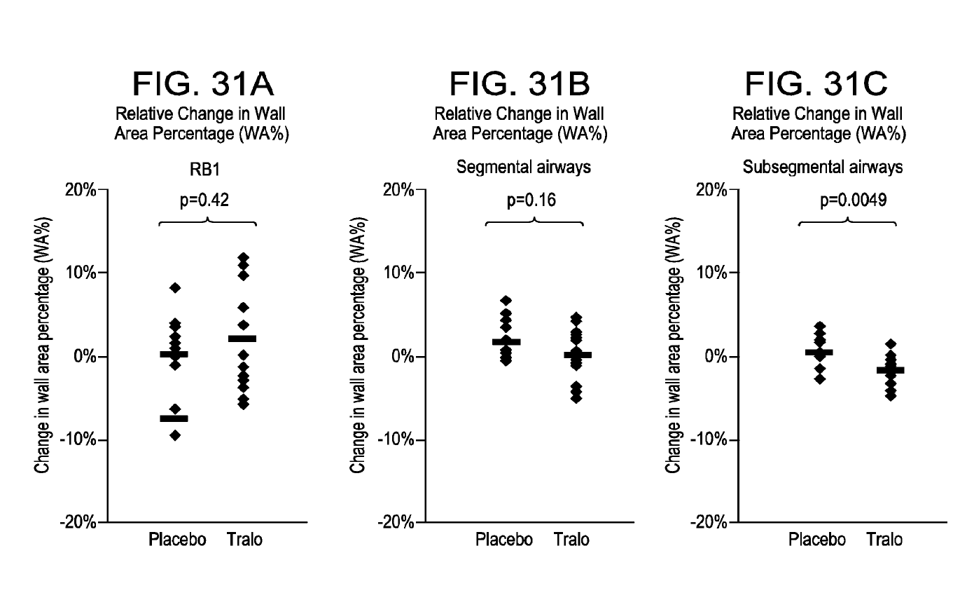

The relative changes in Wall Area Percentage (WA %) from baseline to visit 30 are shown in TABLE 28 and FIG. 31.

TABLE 28
Relative change in (Wall Area Percentage)
WA % from baseline to visit 30

| | | Total | | |
|---|---|---|---|---|
| | Total Placebo, | Tralokinumab, | Group | |
| | n = 12 | n = 14 | differ- | P |
| | Mean (S.D.) | Mean (S.D.) | ence | value |
| RB1 | +0.43% (4.67%) | +2.27% (6.34%) | +1.83% | 0.42 |
| Seg- | +1.96% (2.38%) | +0.39% (3.10%) | −1.57% | 0.17 |
| mental | | | | |
| Subseg- | +0.62% (1.82%) | −1.63% (1.87%) | −2.25% | 0.0049 |
| mental | | | | |
| All | +1.08% (1.48%) | −1.01% (1.82%) | −2.09% | 0.0040 |

The relative changes in Wall Thickness (WT) from baseline to visit 30 are shown in TABLE 29.

TABLE 29
Relative change in Wall Thickness (WT) from baseline to visit 30

| | | Total | | |
|---|---|---|---|---|
| | Total Placebo, | Tralokinumab, | Group | |
| | n = 12 | n = 14 | differ- | P |
| | Mean (S.D.) | Mean (S.D.) | ence | value |
| RB1 | +2.70% (11.29%) | +9.71% (18.27%) | +7.00% | 0.26 |
| Segmental | +6.32% (8.21%) | +5.38% (7.72%) | −0.94% | 0.77 |
| Subseg- | +1.99% (5.81%) | +3.41% (9.31%) | +1.41% | 0.65 |
| mental | | | | |
| All | +3.40% (5.58%) | +4.22% (7.00%) | +0.81% | 0.75 |

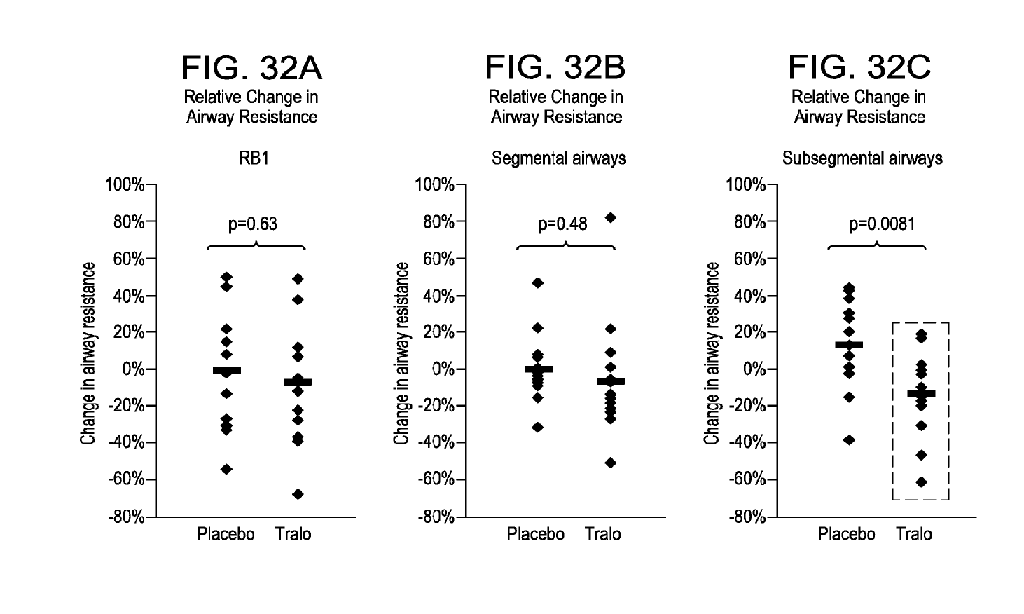

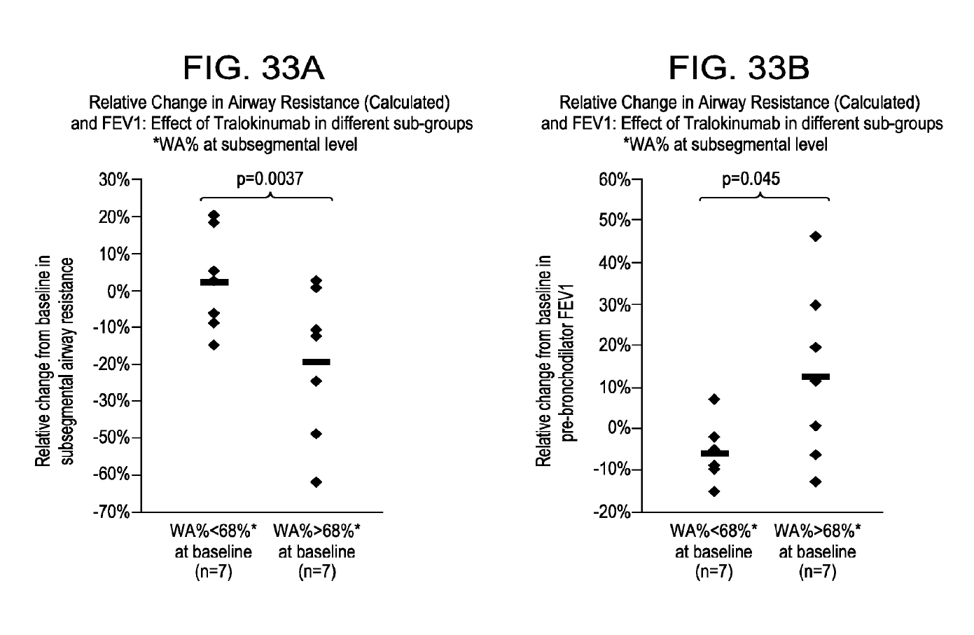

The relative changes in Airway Resistance from baseline to visit 30 are shown in TABLE 30 and FIG. 32. The dataset for tralokinumab (dashed box in FIG. 32) was split into two sub-groups according to WA % at baseline, for subsequent analysis of relative improvement in sub-segmental airway resistance and FEV1%. A median cut-off based on WA % was used, resulting in a cut-off level of 68% (see FIG. 33).

TABLE 30
Relative change in Airway Resistance from baseline to visit 30

| | | Total | | |
|---|---|---|---|---|
| | Total Placebo, | Tralokinumab, | Group | |
| | n = 12 | n = 14 | differ- | P |
| | Mean (S.D.) | Mean (S.D.) | ence | value |
| RB1 | −0.06% (31.89%) | −6.47% (35.06%) | −6.42% | 0.63 |
| Seg- | +1.51% (19.45%) | −5.80% (30.76%) | −7.31% | 0.48 |
| mental | | | | |
| Subseg- | +14.09% (24.87%) | −12.56% (22.16%) | −26.65% | 0.0081 |
| mental | | | | |
| All | +10.42% (20.20%) | −10.68% (22.37%) | −21.10% | 0.019 |

### Conclusions:

A number of conclusions can be made from these studies including:

(i) 3D measurements of WA % were more consistent with published data, and with less variability than 2D measurements;
(ii) 2D and 3D measurements in the right apical segmental bronchus (RB1) were relatively consistent for lumen area, but more variable for wall measurements;
(iii) Averaging relative change in each parameter over multiple airways in the 3D analysis reduced the variability;
(iv) Tralokinumab had a greater effect on LA and WA % in smaller (subsegmental) airways than in segmental bronchi;
(v) Lumen area, wall area percentage, airway resistance in subsegmental bronchi were significantly improved with tralokinumab compared to placebo (p=0.021, p <0.005 and p <0.01 respectively);
(vi) No significant treatment effects were seen in wall area and wall thickness;
(vii) The effect of Tralokinumab treatment on airway resistance was significantly higher (p=0.037) in the half of the tralokinumab group with the highest wall area percentage (WA % of subsegmental airways higher than the 68% median cut-off) at baseline compared to the half with the lowest wall area percentage (WA % of subsegmental airways lower than the 68% median cut-off). See FIG. 33. Indeed, patients with WA % above the specified threshold (e.g., WA % at least 68% at subsegmental level) display a statistically significant improvement in airway resistance and display a statistically significant improvement in pre-bronchodilator FEV1. See FIG. 33.

Taken together, these studies suggest that Wall Area % as determined using a CT scan of the lungs of subsegmental airways (WA %) can be used to predict treatment response (for example, improvements in airway resistance and/or FEV1) in patients treated or candidates for treatment with an IL-13 antagonist (for example an anti-IL-13 antibody such as tralokinumab or lebrikizumab). Moreover, these studies suggest that patients suffering from an IL-13-mediated disease (e.g., asthma, COPD, IPF, UC, or atopic dermatitis) having a WA % value above a predetermined WA % threshold level or above the WA % in one or more control samples (e.g., a WA % threshold level of about 68%, above about 60% or between 60%-80% of subsegmental airways) prior to treatment are good candidates for treatment with an IL-13 antagonist (for example an anti-IL-13 antibody such as tralokinumab or lebrikizumab). In addition, wall area percentage (WA %) can be combined with other measurements obtained using 3D airway analysis of CT scan data for example lumen area (LA), wall area (WA), wall thickness area (WT), airway resistance, or combinations thereof to identify populations of patients amenable for treatment with an IL-13 antagonist (for example an anti-IL-13 antibody such as tralokinumab or lebrikizumab).

Wall area percentage (WA %) at baseline describes how constricted/thickened the airways are and consequently the potential improvement that can be achieved with treatment with an IL-13 antagonist such as tralokinumab or lebrikizumab. Since WA % is computed as a ratio, it is automatically normalized with the airway dimensions for an individual patient, making this parameter a logical choice for baseline characterization.

In addition to the use in severe asthma, this method would be applicable in other pulmonary diseases, including but not limited to, COPD, emphysema, and IPF.

### Example 7

### Induction of Periostin and DPP4 Expression in Airway Epithelium from COPDSubjects

Airway inflammation within COPD is heterogeneous and modulated by a variety of inflammatory mediators including IL-17, IL-33 and IL-13. Differentiated normal and COPD—bronchial epithelial cells at (EPIAIRWAY™ tissue) air-liquid interfaces were procured from MATTEK (MA,TTEK Corporation, MA) and cultured for 24 hours at 37° C. in 5% CO₂-rich incubator. The tissues were then rinsed twice with PBS and cultured in medium devoid of serum or steroids for an additional 24 hours. Following this period the cells were stimulated with 25 ng/mL of IL-13, IL-17A, IL-17F, IL-17E, IL-13, TSLP or IL-33 (PeproTech, NJ) for an additional 24 h. Total RNA was then extracted using mirVana Isolation protocols (Life Technologies, MD), reverse transcribed by The SuperScript® III First-Strand Synthesis System (Life Technologies, MD) and quantified by TAQMAN® gene expression PCR assays (Life Technologies, MD).

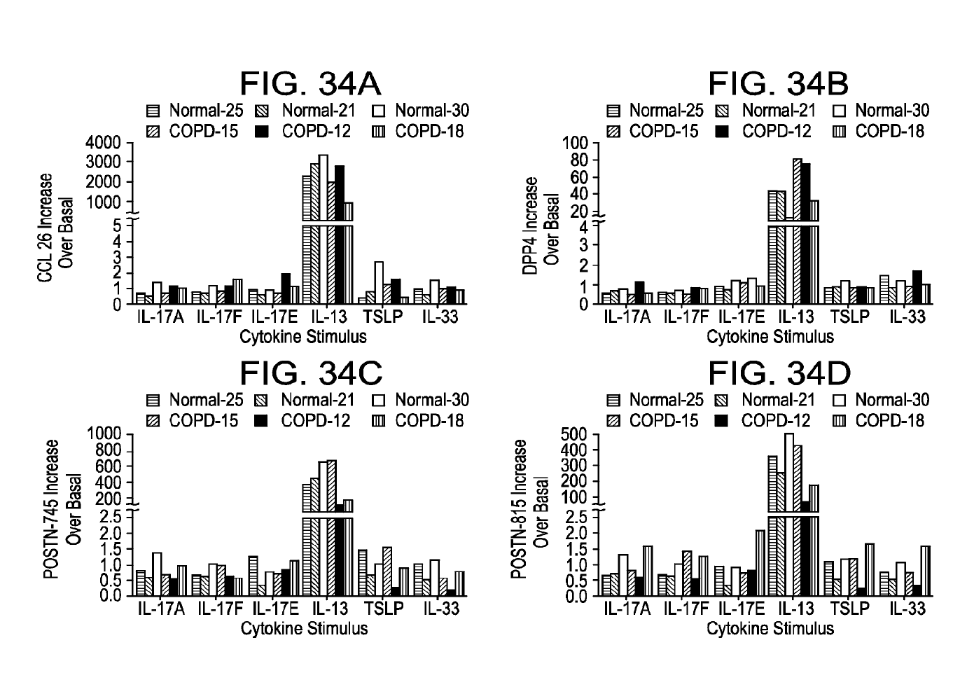

IL-13 specific up-regulation of CCL-26, DPP4, periostin POSTN-745, and periostin POST-815 was observed in transcripts obtained from highly differentiated bronchial epithelial cells from normal subjects and COPD subjects. The bronchial epithelial cells were grown at air liquid interfaces (EPIAIRWAY™ model). See FIG. 34. The data presented in FIG. 34 represent log 2 fold change (fc) in CCL-26, DPP4, periostin POSTN-745, and periostin POST-815 gene transcripts, relative to basal/untreated basal condition after stimulation with 25 ng/mL of IL-17A, IL-17F, IL-17E, IL-13, TSLP or IL-33 for 24 hours as indicated above. These findings indicate that periostin and DPP4 are specific markers for IL-13 mediated COPD. This experimental data, corresponding to differentiated airway epithelial cells, shows that IL-13 mediated inflammation can be distinguished from other phenotypes by specific expression of CCL-26, DPP4 and periostin. The data also shows that IL-13 (but not IL-17A/F/E, TSLP, or IL-33) significantly induce periostin and DPP4 expression in airway epithelium from COPD subjects. The preservation of these outcomes across normal and diseased epithelium, indicates that induced periostin and DPP4 can be used as biomarkers for identifying COPD patients affected by IL-13 mediated airway inflammation.

### Example 8

### Expression of Periostin and DPP4 in Atopic Dermatitis Patients

Subject Selection:

Atopic dermatitis patients provided serum samples and clinical characteristics with informed consent. Samples were accessed and selected anonymously corresponding to 100 patients with moderate atopic dermatitis and 100 patients with severe atopic dermatitis. In order to balance between the moderate and severe comparison groups, available patient samples were matched with respect to gender and age.

DPP4 Quantification:

DPP4 was quantified using the Human DPPIV/CD26 Quantikine ELISA Kit. The Reference Standard Stock Solution (RS) was the Human DPPIV Standard from the kit. The QC Sample Stock Solution (QCS) was Human DPPIV Standard from the kit. Quality Controls (QCs) were prepared the day prior to the start of an assay. At least 2 Reference Standard (DPPIV Standard, Part 892953) (RS) vials provided in the kits were reconstituted. Each RS was reconstituted with 1000 μL of diH2O as per product insert. The reconstituted concentration produced a stock solution of 200 ng/mL. Test Samples were prepared at 1:70 MRD. Samples were thawed at room temperature and diluted with Calibrator Diluent RD5-33. To prepare Reference Standard Stock Solution (RS), RS was reconstituted with 1000 μL of diH2O. The reconstituted concentration produced a stock solution of 200 ng/mL. Reference Standards were prepared starting with Reference Standard Stock (RS). Quality Control (QC) Samples were prepared starting with frozen QC Stock (QCS). 50 μL of prepared Reference Standards, QCs, and diluted Test Samples were pipetted into appropriate wells. The plate(s) were sealed and incubated for 2 hour ±15 minutes at room temperature with shaking at approximately 450 rpm on an orbital plate shaker. The plate(s) were washed four times using a plate washer. After the wash, any remaining Wash Buffer was removed by blotting against clean paper towels. 200 μL of DPPIV Conjugate were added to each well. The plate(s) were sealed and incubated for 2 hour ±15 minutes at room temperature with shaking at approximately 450 rpm on an orbital plate shaker. The plate(s) were washed again four times using a plate washer. Substrate Reagent was prepared by adding equal volume of Substrate A and B prior to adding it to the wells. 200 μL of Substrate Reagent were added to each well. The plate(s) was sealed and incubated for 30 minutes±5 min at room temperature with shaking at approximately 450 rpm on an orbital plate shaker. Samples were protect from light. 50 μL of Stop Solution were added, and absorbance in each well was measured at 450 nm using a spectrophotometric microplate reader. Wells were read within 30 minutes of adding the Stop Solution. Data was analyzed using a 4-PL non-linear fit (SoftMax ProGxP v5.2). Blank values were not subtracted from Standard Curve values when back-calculating to the concentrations. For acceptance of an assay, Standard Curve and Quality Control replicates on the assay plate had to pass the acceptance criteria of 100±30% recovery and ≦25% CV. Test sample replicates on the assay plate had to pass the acceptance criteria of ≦25% CV.

Periostin Quantification:

An MSD assay plate (MSD L15XA) (MSD, Gaithersburg, Md.) was coated with an anti-human Periostin antibody (MedImmune, clone#4B4.B11, as disclosed in U.S. Provisional Patent Application No. 61/936,967, herein incorporated by reference, and deposited with the at the American Type Culture Collection, Manassas, Va. (the ATCC) under Deposit No. PTA-120210 on Apr. 17, 2013). The Capture Antibody in 1×PBS (Lonza, Catalog #17-516Q or equivalent) to a final concentration of 2 μg/ml. 50 μl/well of 2 μg/ml Capture Antibody was added to each well, and the plate was covered with an adhesive microplate sealer. The plate was incubated overnight at 2° C. to 8° C. and subsequently washed with ELISA Wash Buffer.

The coated assay plate was washed three times with 1× ELISA Wash Buffer (0.05% Tween-20, 1×PBS) and blocked with 150 μl/well I-Block Buffer (IBB) (I-Block Buffer: 0.5% Tween-20, 1×PBS, 0.2% I-Block Buffer) (Tropix I-Block™, Applied Biosystems, Cat# T2015) for a minimum of one hour at room temperature (RT) with gentle shaking (for ≧60 minutes but no more than 4 hours).

Recombinant human Periostin (R&D Systems, Catalog #3548-F2) was used as a standard. Reference standards (RS), quality controls (QC) and negative control (NC) prepared in IBB, and serum test samples diluted to the Minimum Required Dilution of 1:10 in IBB, were added to the plate and incubated for approximately one hour at RT on a plate shaker with gentle shaking Unbound analyte was removed by washing the plate with ELISA Wash Buffer. To detect bound analyte, Ruthenylated-anti-human Periostin (Ru-7B5, conjugate antibody clone#7B5.C4, MedImmune, as disclosed in U.S. Provisional Patent Application No. 61/936,967, herein incorporated by reference, and deposited with the at the American Type Culture Collection, Manassas, Va. (the ATCC) under Deposit No. PTA-120211 on Apr. 17, 2013) was prepared to a final concentration of 2 μg/ml, and after washing each well with 200 μl/well of 1× ELISA Wash Buffer, 30 μl/well of the Detection Antibody was added to each wells and the plate was incubated for approximately one hour (60 minutes±10 minutes) on a plate shaker with gentle shaking at RT (protected from light exposure). Unbound detection antibody was removed by washing the plate with ELISA Wash Buffer.

Read Buffer (MSD) was prepared by the diluting “4× Read Buffer T” (4×, MSD, Cat # R92TC-1) stock to “1×” using distilled water. Read Buffer was added to the plate and the plate was read on an MSD Plate Reader. Raw data, in Electrochemiluminescence Units (ECLU), was transferred to the SoftMax Pro software (SoftMax® Pro v5.2 GxP) and to an Excel spreadsheet for further analysis. The reference standard curve for each assay was plotted using the 4-parameter logistic weighted (1/ŷ2) curve fit method. Periostin concentrations were interpolated for each QC level, NC and for Serum Test Samples from the fitted curve.

Results and Conclusions:

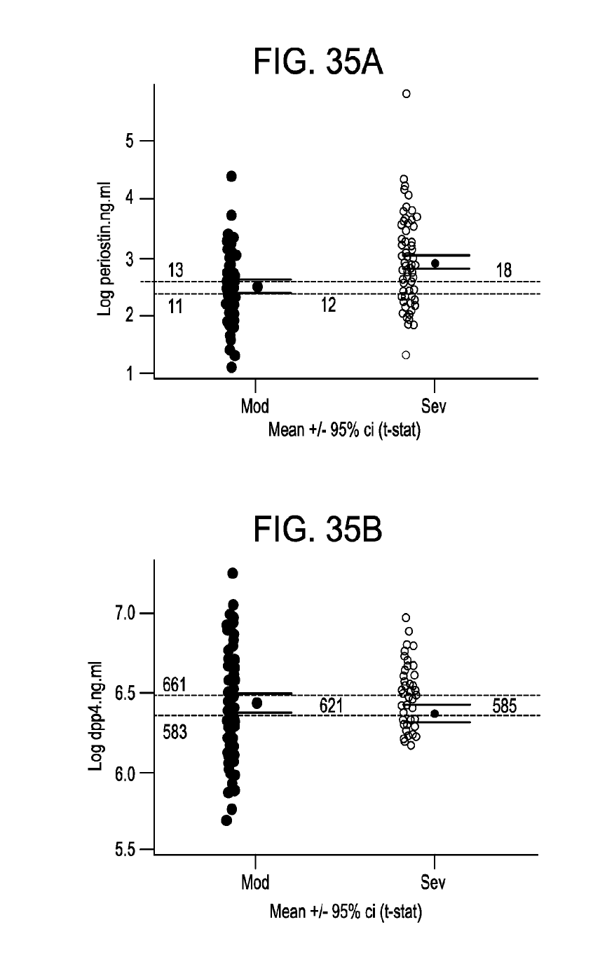

Periostin was expressed in the sera of severe atopic dermatitis patients at higher levels when compared to moderate atopic dermatitis patients. See FIG. 35, panel A. DPP4 expression was also elevated in the sera of atopic dermatitis patients, but expression levels were not related to disease severity. See FIG. 35, panel B. The finding that DPP4 and periostin expression levels are increased in the serum of severe and moderate atopic dermatitis patients indicates that DPP4: (1) could be used as a peripheral biomarker of IL-13 pathway activation in atopic dermatitis patients; (2) could be informative in electing potential therapies for atopic dermatitis patients, and (3) could be useful in selecting patients responsive to therapy using an IL-13 antagonist, for example, an anti-IL-13 antibody such as tralokinumab or lebrikizumab.

### Example 9

### Periostin and Serum DPP4 in Stable COPD and Acute Exacerbations of COPD

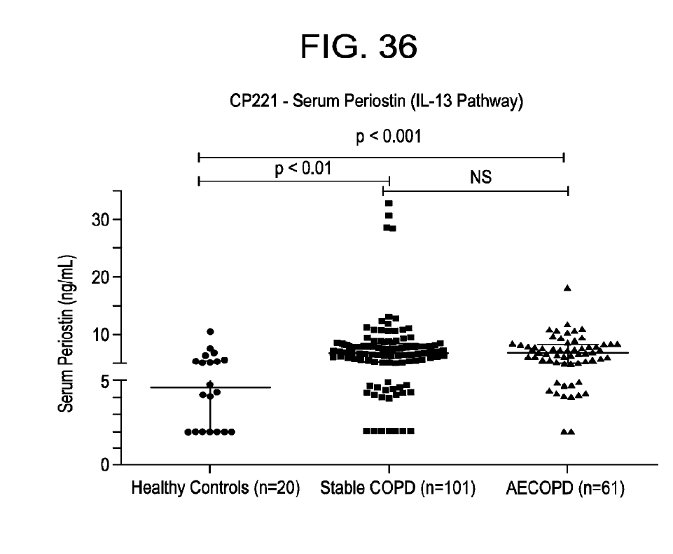

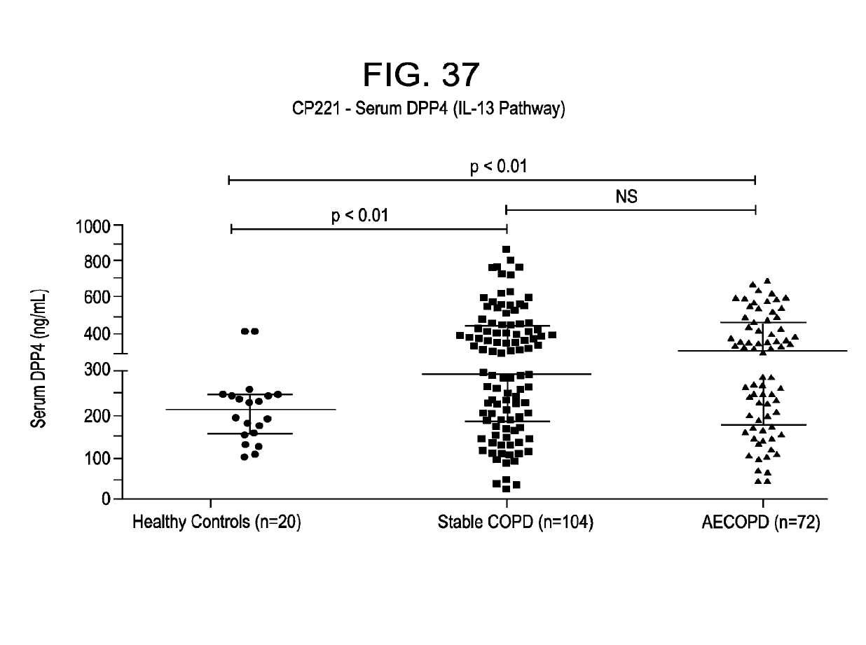

To determine whether there are differences in the expression levels of periostin and/or DPP4 in acute exacerbations of COPD (AECOPD) with respect to the expression levels observed in stable COPD, levels of periostin (FIG. 36) and serum DPP4 (FIG. 37) were measured in healthy controls, stable COPD patients, and patients experiencing acute exacerbations of COPD. Healthy control sera were obtained from Bioreclamation (Baltimore Md., USA) from 20 nonsmoking subjects (10 females and 10 males, ages 17 to 59). COPD and AECOPD sera were from the MI-CP221 clinical biomarker study, sponsored by MedImmune. Periostin and DPP4 levels were measured by immunoassay, as described above. Data showed that periostin and serum DPP4 were increased in stable COPD and also in AECOPD relative to healthy controls. This indicated that DPP4 and/or periostin can be used as biomarkers for both stable COPD and AECOPD and could be useful in selecting COPD patients responsive to therapy using an IL-13 antagonist, for example, an anti-IL-13 antibody such as tralokinumab or lebrikizumab.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. A method of treating a patient having an interleukin-13 (IL-13)-mediated disease or disorder, comprising administering an IL-13 antagonist to the patient if the level of DPP4 (dipeptidyl peptidase-4) in one or more samples taken from the patient is above a predetermined DPP4 threshold level, or is above the DPP4 level in one or more control samples.

2. A method of treating a patient having an interleukin-13 (IL-13)-mediated disease or disorder, comprising administering an IL-13 antagonist to the patient if (a) the level of DPP4 (dipeptidyl peptidase-4) in one or more samples taken from the patient is above a predetermined DPP4 threshold level, or is above the DPP4 level in one or more control samples, and optionally if (b) the patient presents (i) high periostin (≧median serum periostin or about 23 ng/mL), (ii) high eosinophil cell count (blood eosinophil count ≧300 cells/μL), (iii) high Th2 (high Th2 defined as IgE >100 IU/mL and blood eosinophils ≧0.14×10⁹/L), (iv) FEV1 reversibility to a short-acting β2 agonist ≧12%, (v) wall area percentage (WA %) of subsegmental airways from a CT scan of the lungs ≧68%, or (vi) combinations thereof.

3. The method according to claim 1, wherein the patient's DPP4 level is measured in an immunoassay.

4. The method according to claim 3, wherein the immunoassay employs one or more anti-DPP4 antibodies or antigen binding fragments thereof which recognize human DDP4.

5. The method according to claim 1, wherein the IL-13 antagonist comprises one or more of an anti-IL-13 antibody or antigen-binding fragment thereof, an IL-13 mutein, and IL-4 mutein, an anti-IL-13Rα1 antibody or antigen-binding fragment thereof, or an anti-IL-4Rα antibody or antigen-binding fragment thereof.

6. The method according to claim 1, wherein the patient has been treated with one or more additional medications, either before, during, or after administration of an IL-13 antagonist.

7. The method according to claim 6, wherein the one or more additional medications comprises a steroid, and optionally comprises a bronchodilator.

8. The method according to claim 7, wherein the steroid is fluticasone or budesonide, and the bronchodilator is salbutamol or salmeterol.

9. The method according to claim 6, wherein the one or more additional medications are administered by inhalation, by oral administration, by injection, or by a combination thereof.

10. The method according to claim 9, wherein inhalation administration is conducted using a metered dose inhaler (MDI) or a dry powder inhaler (DPI).

11. The method according to claim 7, wherein the steroid is administered at a high dose.

12. The method according to claim 1, wherein the IL-13 antagonist is an anti-IL13 antibody, or antigen-binding fragment thereof, wherein:
(i) the antibody or antigen-binding fragment thereof binds to the same IL-13 epitope as tralokinumab (VH: SEQ ID NO:3; VL:SEQ ID NO:4) or competitively inhibits binding of tralokinumab to IL-13, or both;
(ii) the antibody or antigen-binding fragment thereof comprises tralokinumab (VH: SEQ ID NO:3; VL:SEQ ID NO:4) or an antigen-binding fragment thereof;
(iii) the antibody or antigen-binding fragment thereof consists of tralokinumab (VH: SEQ ID NO:3; VL:SEQ ID NO:4) or an antigen-binding fragment thereof;
(iv) the antibody or antigen-binding fragment thereof binds to the same IL-13 epitope as lebrikizumab (VH: SEQ ID NO:1; VL:SEQ ID:2) or competitively inhibits binding of lebrikizumab to IL-13, or both;
(v) the antibody or antigen-binding fragment thereof comprises lebrikizumab (VH: SEQ ID NO:1; VL:SEQ ID:2) or an antigen-binding fragment thereof;
(vi) the antibody or antigen-binding fragment thereof consists of lebrikizumab (VH: SEQ ID NO:1; VL:SEQ ID:2) or an antigen-binding fragment thereof; or
(vii) the antibody or antigen-binding fragment thereof comprises SEQ ID NO:3, SEQ ID NO:4, or an antigen-binding fragment thereof.

13. The method according to claim 1, wherein the one or more samples taken from the patient and/or the one or more control samples comprises one or more of whole blood, blood serum, plasma, saliva, sputum, bronchoalveolar lavage fluid, lung epithelial cells, urine, skin, nasal polyps, or a combination thereof.

14. The method according to claim 1, wherein the IL-13 antagonist is administered at a fixed dose.

15. The method according to claim 12, wherein the anti-IL13 antibody is tralokinumab, and wherein tralokinumab is administered at a fixed dose of about 300 mg/dose.

16. The method according to claim 1, wherein the IL-13 antagonist is administered in two or more doses.

17. The method according to claim 1, wherein the IL-13 antagonist is administered week, biweekly or monthly.

18. The method according to claim 1, wherein the IL-13 antagonist is administered intravenously, intramuscularly, subcutaneously, or a combination thereof.

19. The method according to claim 1, wherein the predetermined DPP4 threshold level is at least about 250 ng/ml, at least about 350 ng/mL, at least about 375 ng/mL, at least about 400 ng/mL, at least about 450 ng/mL, at least about 500 ng/mL, at least 550 ng/mL, or at least about 600 ng/mL, as measured in serum using an ELISA QUANTIKINE® assay.

20. The method according to claim 1, wherein the predetermined DPP4 threshold level is about 365 ng/mL.

21. The method according to claim 1, wherein the one or more control samples are (i) a sample or samples obtained from normal healthy individuals; (ii) a sample or samples obtained from patients with a non-IL-13-mediated subset of asthma; (iii) a sample or samples obtained from asthma patients naïve for corticosteroid treatment; (iv) a sample or samples obtained from asthma patients treated with corticosteroids; (v) a sample or samples obtained from untreated atopic dermatitis patients; (vi) a sample or samples obtained from treated atopic dermatitis patients; (vii) a pre-determined standard amount of isolated DPP4; or (viii) a combination thereof.

22. The method according to claim 1, wherein administration of the IL-13 antagonist results in:
(a) AER (Acute Exacerbation Rate) reduction, wherein the AER reduction is at least 5%, at least 10%, at least 15%, at least 20%, at least 25%, at least 30%, at least 35%, at least 40%, or at least 45% compared to the AER observed in a population of patients treated with a placebo;
(b) FEV₁ (Forced Expiratory Volume in one second) increase, wherein the FEV₁ increase is at least 3%, at least 5%, at least 7%, at least 9%, at least 11%, at least 13%, at least 15%, least 17%, or at least 19% compared to the FEV₁ observed in a population of patients treated with a placebo;
(c) improved ACQ-6 (Asthma Control Questionnaire, 6-item version) results;
(d) improved AQLQ (Asthma Quality of Life Questionnaire) results; or,
(e) a combination thereof.

23. The method according to claim 1, wherein the IL-13-mediated disease or disorder is a pulmonary disease or disorder, an inflammatory bowel disease or disorder, or a chronic inflammatory skin disease or disorder.

24. The method according to claim 23, wherein the pulmonary disease or disorder is asthma, IPF, COPD, chronic rhinosinusitis, or allergic rhinitis

25. The method according to claim 23, wherein the chronic inflammatory skin disease or disorder is atopic dermatitis, allergic contact dermatitis, eczema or psoriasis.

26. The method according to claim 24, wherein the asthma is allergic asthma, atopic asthma, corticosteroid naive asthma, chronic asthma, corticosteroid resistant asthma, corticosteroid refractory asthma, asthma due to smoking, or asthma uncontrolled on corticosteroids.

27. A method of diagnosing an IL-13 mediated disease or disorder in a patient comprising measuring the level of DPP4 (dipeptidyl peptidase-4) in a sample taken from the patient, wherein the patient is diagnosed with the IL-13 mediated disease or disorder if the level of DPP4 is above a predetermined DPP4 threshold level, or above the DPP4 level in one or more control samples.

28. A method of identifying a patient as a candidate for treatment with an IL-13 antagonist comprising measuring the level of DPP4 (dipeptidyl peptidase-4) in a sample taken from the patient, wherein a level of DPP4 above a predetermined DPP4 threshold level, or above the DPP4 level in one or more control samples identifies the patient as a candidate for treatment with the IL-13 antagonist.


SEQUENCE LISTING (78 sequences)

SEQ ID NO: 1 - protein, 445 aa, Homo sapiens
  misc_feature (1)..(445): Lebrikizumab Heavy chain
QVTLRESGPALVKPTQTLTLTCTVSGFSLSAYSVNWIRQPPGKALEWLAMIWGDGKIVYNSALKSRLTISKDTSKNQVVLTMTNMDPVDTATYYCAGDGYYPYAMDNWGQGSLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 218 aa, Homo sapiens
  misc_feature (1)..(218): Lebrikizumab Light chain
DIVMTQSPDSLSVSLGERATINCRASKSVDSYGNSFMHWYQQKPGQPPKLLIYLASNLESGVPDRFSGSGSGTDFTLTISSLQAEDVAVYYCQQNNEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 122 aa, Homo sapiens
  misc_feature (1)..(122): Tralokinumab Heavy chain
QVQLVQSGAEVKKPGASVKVSCKASGYTFTNYGLSWVRQAPGQGLEWMGWISANNGDTNYGQEFQGRVTMTTDTSTSTAYMELRSLRSDDTAVYYCARDSSSSWARWFFDLWGRGTLVTVSS

SEQ ID NO: 4 - protein, 108 aa, Homo sapiens
  misc_feature (1)..(108): Tralokinumab Light chain
SYVLTQPPSVSVAPGKTARITCGGNIIGSKLVHWYQQKPGQAPVLVIYDDGDRPSGIPERFSGSNSGNTATLTISRVEAGDEADYYCQVWDTGSDPVVFGGGTKLTVL

SEQ ID NO: 5 - protein, 766 aa, Homo sapiens
  misc_feature (1)..(766): DPP4 membrane bound form
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 6 - protein, 728 aa, Homo sapiens
  misc_feature (1)..(728): DPP4 soluble form
SRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 7 - DNA, 3913 bp, Homo sapiens
  misc_feature (1)..(766): DPP4 membrane bound form (full sequence), cDNA
ctttcactggcaagagacggagtcctgggtttcagttccagttgcctgcggtgggctgtgtgagtttgccaaagtcccctgccctctctgggtctcggttccctcgcctgtccacgtgaggttggaggagctgaacgccgacgtcatttttagctaagagggagcagggtccccgagtcgccggcccagggtctgcgcatccgaggccgcgcgccctttcccctcccccacggctcctccgggccccgcactctgcgccccggctgccgcccagcgccctacaccgccctcagggggccctcgcgggctccccccggccgggatgccagtgccccgcgccacgcgcgcctgctcccgcgccgcctgccctgcagcctgcccgcggcgcctttatacccagcgggctcggcgctcactaatgtttaactcggggccgaaacttgccagcggcgagtgactccaccgcccggagcagcggtgcaggacgcgcgtctccgccgcccgcggtgacttctgcctgcgctccttctctgaacgctcacttccgaggagacgccgacgatgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttagcacctcaaaataccatgccatttaaagcttattaaaactcatttttgttttcattatctcaaaactgcactgtcaagatgatgatgatctttaaaatacacactcaaatcaagaaacttaaggttacctttgttcccaaatttcatacctatcatcttaagtagggacttctgtcttcacaacagattattaccttacagaagtttgaattatccggtcgggttttattgtttaaaatcatttctgcatcagctgctgaaacaacaaataggaattgtttttatggaggctttgcatagattccctgagcaggattttaatctttttctaactggactggttcaaatgttgttctcttctttaaagggatggcaagatgtgggcagtgatgtcactagggcagggacaggataagagggattagggagagaagatagcagggcatggctgggaacccaagtccaagcataccaacacgagcaggctactgtcagctcccctcggagaagagctgttcacagccagactggcacagttttctgagaaagactattcaaacagtctcaggaaatcaaatatgcaaagcactgacttctaagtaaaaccacagcagttgaaaagactccaaagaaatgtaagggaaactgccagcaacgcaggcccccaggtgccagttatggctataggtgctacaaaaacacagcaagggtgatgggaaagcattgtaaatgtgcttttaaaaaaaaatactgatgttcctagtgaaagaggcagcttgaaactgagatgtgaacacatcagcttgccctgttaaaagatgaaaatatttgtatcacaaatcttaacttgaaggagtccttgcatcaatttttcttatttcatttctttgagtgtcttaattaaaagaatattttaacttccttggactcattttaaaaaatggaacataaaatacaatgttatgtattattattcccattctacatactatggaatttctcccagtcatttaataaatgtgccttcattttttcagaaaaaaaaaaaaaaa

SEQ ID NO: 8 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 9 - protein, 141 aa, Homo sapiens
MAQHLSTLLLLLATLAVALAWSPKEEDRIIPGGIYNADLNDEWVQRALHFAISEYNKATKDDYYRRPLRVLRARQQTVGGVNYFFDVEVGRTICTKSQPNLDTCAFHEQPELQKKQLCSFEIYEVPWENRRSLVKSRCQES

SEQ ID NO: 10 - protein, 94 aa, Homo sapiens
MMGLSLASAVLLASLLSLHLGTATRGSDISKTCCFQYSHKPLPWTWVRSYEFTSNSCSQRAVIFTTKRGKKVCTHPRKKWVQKYISLLKTPKQL

SEQ ID NO: 11 - protein, 914 aa, Homo sapiens
MGPFKSSVFILILHLLEGALSNSLIQLNNNGYEGIVVAIDPNVPEDETLIQQIKDMVTQASLYLFEATGKRFYFKNVAILIPETWKTKADYVRPKLETYKNADVLVAESTPPGNDEPYTEQMGNCGEKGERIHLTPDFIAGKKLAEYGPQGKAFVHEWAHLRWGVFDEYNNDEKFYLSNGRIQAVRCSAGITGTNVVKKCQGGSCYTKRCTFNKVTGLYEKGCEFVLQSRQTEKASIMFAQHVDSIVEFCTEQNHNKEAPNKQNQKCNLRSTWEVIRDSEDFKKTTPMTTQPPNPTFSLLQIGQRIVCLVLDKSGSMATGNRLNRLNQAGQLFLLQTVELGSWVGMVTFDSAAHVQSELIQINSGSDRDTLAKRLPAAASGGTSICSGLRSAFTVIRKKYPTDGSEIVLLTDGEDNTISGCFNEVKQSGAIIHTVALGPSAAQELEELSKMTGGLQTYASDQVQNNGLIDAFGALSSGNGAVSQRSIQLESKGLTLQNSQWMNGTVIVDSTVGKDTLFLITWTTQPPQILLWDPSGQKQGGFVVDKNTKMAYLQIPGIAKVGTWKYSLQASSQTLTLTVTSRASNATLPPITVTSKTNKDTSKFPSPLVVYANIRQGASPILRASVTALIESVNGKTVTLELLDNGAGADATKDDGVYSRYFTTYDTNGRYSVKVRALGGVNAARRRVIPQQSGALYIPGWIENDEIQWNPPRPEINKDDVQHKQVCFSRTSSGGSFVASDVPNAPIPDLFPPGQITDLKAEIHGGSLINLTWTAPGDDYDHGTAHKYIIRISTSILDLRDKFNESLQVNTTALIPKEANSEEVFLFKPENITFENGTDLFIAIQAVDKVDLKSEISNIARVSLFIPPQTPPETPSPDETSAPCPNIHINSTIPGIHILKIMWKWIGELQLSIA

SEQ ID NO: 12 - protein, 141 aa, Homo sapiens
MAWPLCTLLLLLATQAVALAWSPQEEDRIIEGGIYDADLNDERVQRALHFVISEYNKATEDEYYRRLLRVLRAREQIVGGVNYFFDIEVGRTICTKSQPNLDTCAFHEQPELQKKQLCSFQIYEVPWEDRMSLVNSRCQEA

SEQ ID NO: 13 - protein, 134 aa, Homo sapiens [sequence omitted]

SEQ ID NO: 14 - protein, 415 aa, Homo sapiens
MEDLCVANTLFALNLFKHLAKASPTQNLFLSPWSISSTMAMVYMGSRGSTEDQMAKVLQFNEVGANAVTPMTPENFTSCGFMQQIQKGSYPDAILQAQAADKIHSSFRSLSSAINASTGNYLLESVNKLFGEKSASFREEYIRLCQKYYSSEPQAVDFLECAEEARKKINSWVKTQTKGKIPNLLPEGSVDGDTRMVLVNAVYFKGKWKTPFEKKLNGLYPFRVNSAQRTPVQMMYLREKLNIGYIEDLKAQILELPYAGDVSMFLLLPDEIADVSTGLELLESEITYDKLNKWTSKDKMAEDEVEVYIPQFKLEEHYELRSILRSMGMEDAFNKGRANFSGMSERNDLFLSEVFHQAMVDVNEEGTEAAAGTGGVMTGRTGHGGPQFVADHPFLFLIMHKITNCILFFGRFSSP

SEQ ID NO: 15 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 16 - protein, 1153 aa, Homo sapiens
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 17 - protein, 390 aa, Homo sapiens
MNSLSEANTKFMFDLFQQFRKSKENNIFYSPISITSALGMVLLGAKDNTAQQISKVLHFDQVTENTTEKAATYHVDRSGNVHHQFQKLLTEFNKSTDAYELKIANKLFGEKTYQFLQEYLDAIKKFYQTSVESTDFANAPEESRKKINSWVESQTNEKIKNLFPDGTIGNDTTLVLVNAIYFKGQWENKFKKENTKEEKFWPNKNTYKSVQMMRQYNSFNFALLEDVQAKVLEIPYKGKDLSMIVLLPNEIDGLQKLEEKLTAEKLMEWTSLQNMRETCVDLHLPRFKMEESYDLKDTLRTMGMVNIFNGDADLSGMTWSHGLSVSKVLHKAFVEVTEEGVEAAAATAVVVVELSSPSTNEEFCCNHPFLFFIRQNKTNSILFYGRFSSP

SEQ ID NO: 18 - protein, 141 aa, Homo sapiens
MARPLCTLLLLMATLAGALASSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 19 - protein, 393 aa, Homo sapiens
MLLILLSVALLALSSAQNLNEDVSQEESPSLIAGKPQGPPPQGGNQPQGPPPPPGKPQGPPPQGGNKPQGPPPPGKPQGPPPQGDKSRSPRSPPGKPQGPPPQGGNQPQGPPPPPGKPQGPPPQGGNKPQGPPPPGKPQGPPPQGDNKSRSSRSPPGKPQGPPPQGGNQPQGPPPPPGKPQGPPPQGGNKPQGPPPPGKPQGPPPQGDKSRSPRSPPGKPQGPPPQGGNQPQGPPPPPGKPQGPPPQGGNRPQGPPPPGKPQGPPPQGDKSRSSQSPPGKPQGPPPQGGNQPQGPPPPPGKPQGPPPQGGNKPQGPPPPGKPQGPPAQGGSKSQSARSPPGKPQGPPQQEGNNPQGPPPPAGGNPQQPQAPPAGQPQGPPRPPQGGRPSRPPQ

SEQ ID NO: 20 - protein, 235 aa, Homo sapiens
MLSLLLLALPVLASPAYVAPAPGQALQQTGIVGGQEAPRSKWPWQVSLRVRGPYWMHFCGGSLIHPQWVLTAAHCVEPDIKDLAALRVQLREQHLYYQDQLLPVSRIIVHPQFYIIQTGADIALLELEEPVNISSHIHTVTLPPASETFPPGMPCWVTGWGDVDNNVHLPPPYPLKEVEVPVVENHLCNAEYHTGLHTGHSFQIVRDDMLCAGSENHDSCQGDSGGPLVCKVNGT

SEQ ID NO: 21 - protein, 321 aa, Homo sapiens
MALGACGLLLLLAVPGVSLRTLQPGCGRPQVSDAGGRIVGGHAAPAGAWPWQASLRLRRMHVCGGSLLSPQWVLTAAHCFSGSLNSSDYQVHLGELEITLSPHFSTVRQIILHSSPSGQPGTSGDIALVELSVPVTLSSRILPVCLPEASDDFCPGIRCWVTGWGYTREGEPLPPPYSLREVKVSVVDTETCRRDYPGPGGSILQPDMLCARGPGDACQDDSGGPLVCQVNGAWVQAGIVSWGEGCGRPNRPGVYTRVPAYVNWIRRHITASGGSESGYPRLPLLAGLFLPGLFLLLVSCVLLAKCLLHPSADGTPFPAPD

SEQ ID NO: 22 - protein, 540 aa, Homo sapiens
MAKGEGAESGSAAGLLPTSILQSTERPAQVKKEPKKKKQQLSVCNKLCYALGGAPYQVTGCALGFFLQIYLLDVAQKDEEVVFCFSSFQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELA

SEQ ID NO: 23 - protein, 417 aa, Homo sapiens
MRLILPVGLIATTLAIAPVRFDREKVFRVKPQDEKQADIIKDLAKTNELDFWYPGATHHVAANMMVDFRVSEKESQAIQSALDQNKMHYEILIHDLQEEIEKQFDVKEDIPGRHSYAKYNNWEKIVAWTEKMMDKYPEMVSRIKIGSTVEDNPLYVLKIGEKNERRKAIFMDCGIHAREWVSPAFCQWFVYQATKTYGRNKIMTKLLDRMNFYILPVFNVDGYIWSWTKNRMWRKNRSKNQNSKCIGTDLNRNFNASWNSIPNTNDPCADNYRGSAPESEKETKAVTNFIRSHLNEIKVYITFHSYSQMLLFPYGYTSKLPPNHEDLAKVAKIGTDVLSTRYETRYIYGPIESTIYPISGSSLDWAYDLGIKHTFAFELRDKGKFGFLLPESRIKPTCRETMLAVKFIAKYILKHTS

SEQ ID NO: 24 - protein, 338 aa, Homo sapiens
MINSTSTQPPDESCSQNLLITQQIIPVLYCMVFIAGILLNGVSGWIFFYVPSSKSFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 25 - protein, 852 aa, Homo sapiens
MAMRSGRHPSLLLLLVLLLWLLQVSIIDSVQQETDDLTKQTKEKIYQPLRRSKRRWVITTLELEEEDPGPFPKLIGELFNNMSYNMSLMYLISGPGVDEYPEIGLFSLEDHENGRIYVHRPVDREMTPSFTVYFDVVERSTGKIVDTSLIFNIRISDVNDHAPQFPEKEFNITVQENQSAGQPIFQMLAVDLDEENTPNSQVLYFLISQTPLLKESGFRVDRLSGEIRLSGCLDYETAPQFTLLIRARDCGEPSLSSTTTVHVDVQEGNNHRPAFTQENYKVQIPEGRASQGVLRLLVQDRDSPFTSAWRAKFNILHGNEEGHFDISTDPETNEGILNVIKPLDYETRPAQSLIIVVENEERLVFCERGKLQPPRKAAASATVSVQVTDANDPPAFHPQSFIVNKEEGARPGTLLGTFNAMDPDSQIRYELVHDPANWVSVDKNSGVVITVEPIDRESPHVNNSFYVIIIHAVDDGFPPQTATGTLMLFLSDINDNVPTLRPRSRYMEVCESAVHEPLHIEAEDPDLEPFSDPFTFELDNTWGNAEDTWKLGRNWGQSVELLTLRSLPRGNYLVPLFIGDKQGLSQKQTVHVRICPCASGLTCVELADAEVGLHVGALFPVCAAFVALAVALLFLLRCYFVLEPKRHGCSVSNDEGHQTLVMYNAESKGTSAQTWSDVEGQRPALLICTAAAGPTQGVKAYPDATMHRQLLAPVEGRMAETLNQSKERNRFSLSRGCIIPQGRATAGRGLPQDIYKEMMPRRLTQTGKRKHGALARTPSFKKVVYDHKEDEENKAGRKQRSHLFKVMQLRNEQGGVRVQSAHSPSPLNKKACFPGDYRGESAGGHNCRAVSG

SEQ ID NO: 26 - protein, 782 aa, Homo sapiens
MAPHRPAPALLCALSLALCALSLPVRAATASRGASQAGAPQGRVPEARPNSMVVEHPEFLKAGKEPGLQIWRVEKFDLVPVPTNLYGDFFTGDAYVILKTVQLRNGNLQYDLHYWLGNECSQDESGAAAIFTVQLDDYLNGRAVQHREVQGFESATFLGYFKSGLKYKKGGVASGFKHVVPNEVVVQRLFQVKGRRVVRATEVPVSWESFNNGDCFILDLGNNIHQWCGSNSNRYERLKATQVSKGIRDNERSGRARVHVSEEGTEPEAMLQVLGPKPALPAGTEDTAKEDAANRKLAKLYKVSNGAGTMSVSLVADENPFAQGALKSEDCFILDHGKDGKIFVWKGKQANTEERKAALKTASDFITKMDYPKQTQVSVLPEGGETPLFKQFFKNWRDPDQTDGLGLSYLSSHIANVERVPFDAATLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSREGGQTAPASTRLFQVRANSAGATRAVEVLPKAGALNSNDAFVLKTPSAAYLWVGTGASEAEKTGAQELLRVLRAQPVQVAEGSEPDGFWEALGGKAAYRTSPRLKDKKMDAHPPRLFACSNKIGRFVIEEVPGELMQEDLATDDVMLLDTWDQVFVWVGKDSQEEEKTEALTSAKRYIETDPANRDRRTPITVVKQGFEPPSFVGWFLGWDDDYWSVDPLDRAMAELAA

SEQ ID NO: 27 - protein, 164 aa, Homo sapiens
MGDLPGLVRLSIALRIQPNDGPVFYKVDGQRFGQNRTIKLLTGSSYKVEVKIKPSTLQVENISIGGVLVPLELKSKEPDGDRVVYTGTYDTEGVTPTKSGERQPIQITMPFTDIGTFETVWQVKFYNYHKRDHCQWGSPFSVIEYECKPNETRSLMWVNKESFL

SEQ ID NO: 28 - protein, 295 aa, Homo sapiens
MYRISQLMSTPVASSSRLEREYAGELSPTCIFPSFTCDSLDGYHSFECGSIDPLTGSHYTCRRSPRLLTNGYYIWTEDSFLCDKDGNITLNPSQTSVMYKENLVRIFRKKKRICHSFSSLFNLSTSKSWLHGSIFGDINSSPSEDNWLKGTRRLDTDHCNGNADDLDCSSLTDDWESGKMNAESVITSSSSHIISQPPGGNSHSLSLQSQLTASERFQENSSDHSETRLLQEVFFQAILLAVCLIISACARWFMGEILASVFTCSLMITVAYVKSLFLSLASYFKTTACARFVKI

SEQ ID NO: 29 - protein, 198 aa, Homo sapiens
MDAILNYRSEDTEDYYTLLGCDELSSVEQILAEFKVRALECHPDKHPENPKAVETFQKLQKAKEILTNEESRARYDHWRRSQMSMPFQQWEALNDSVKTSMHWVVRGKKDLMLEESDKTHTTKMENEECNEQRERKKEELASTAEKTEQKEPKPLEKSVSPQNSDSSGFADVNGWHLRFRWSKDAPSELLRKFRNYEI

SEQ ID NO: 30 - protein, 159 aa, Homo sapiens
MSRRNCWICKMCRDESKRPPSNLTLEEVLQWAQSFENLMATKYGPVVYAAYLKMEHSDENIQFWMACETYKKIASRWSRISRAKKLYKIYIQPQSPREINIDSSTRETIIRNIQEPTETCFEEAQKIVYMHMERDSYPRFLKSEMYQKLLKTMQSNNSF

SEQ ID NO: 31 - protein, 514 aa, Homo sapiens
MALSELALVRWLQESRRSRKLILFIVFLALLLDNMLLTVVVPIIPSYLYSIKHEKNATEIQTARPVHTASISDSFQSIFSYYDNSTMVTGNATRDLTLHQTATQHMVTNASAVPSDCPSEDKDLLNENVQVGLLFASKATVQLITNPFIGLLTNRIGYPIPIFAGFCIMFVSTIMFAFSSSYAFLLIARSLQGIGSSCSSVAGMGMLASVYTDDEERGNVMGIALGGLAMGVLVGPPFGSVLYEFVGKTAPFLVLAALVLLDGAIQLFVLQPSRVQPESQKGTPLTTLLKDPYILIAAGSICFANMGIAMLEPALPIWMMETMCSRKWQLGVAFLPASISYLIGTNIFGILAHKMGRWLCALLGMIIVGVSILCIPFAKNIYGLIAPNFGVGFAIGMVDSSMMPIMGYLVDLRHVSVYGSVYAIADVAFCMGYAIGPSAGGAIAKAIGFPWLMTIIGIIDILFAPLCFFLRSPPAKEEKMAILMDHNCPIKTKMYTQNNIQSYPIGEDEESESD

SEQ ID NO: 32 - protein, 397 aa, Homo sapiens
MDSLATSINQFALELSKKLAESAQGKNIFFSSWSISTSLTIVYLGAKGTTAAQMAQVLQFNRDQGVKCDPESEKKRKMEFNLSNSEEIHSDFQTLISEILKPNDDYLLKTANAIYGEKTYAFHNKYLEDMKTYFGAEPQPVNFVEASDQIRKDINSWVERQTEGKIQNLLPDDSVDSTTRMILVNALYFKGIWEHQFLVQNTTEKPFRINETTSKPVQMMFMKKKLHIFHIEKPKAVGLQLYYKSRDLSLLILLPEDINGLEQLEKAITYEKLNEWTSADMMELYEVQLHLPKFKLEDSYDLKSTLSSMGMSDAFSQSKADFSGMSSARNLFLSNVFHKAFVEINEQGTEAAAGSGSEIDIRIRVPSIEFNANHPFLFFIRHNKTNTILFYGRLCSP

SEQ ID NO: 33 - protein, 729 aa, Homo sapiens
MDDLTLLDLLECPVCFEKLDVTAKVLPCQHTFCKPCLQRVFKAHKELRCPECRTPVFSNIEALPANLLLVRLLDGVRSGQSSGRGGSFRRPGTMTLQDGRKSRTNPRRLQASPFRLVPNVRIHMDGVPRAKALCNYRGQNPGDLRFNKGDIILLRRQLDENWYQGEINGISGNFPASSVEVIKQLPQPPPLCRALYNFDLRGKDKSENQDCLTFLKDDIITVISRVDENWAEGKLGDKVGIFPILFVEPNLTARHLLEKNKGRQSSCTKNLSLVSSSSRGNTSTLRRGPGSRRKVPGQFSITTALNTLNRMVHSPSGRHMVEISTPVLISSSNPSVITQPMEKADVPSSCVGQVSTYHPAPVSPGHSTAVVSLPGSQQHLSANMFVALHSYSAHGPDELDLQKGEGVRVLGKCQDGWLRGVSLVTGRVGIFPNNYVIPIFRKTSSFPDSRSPGLYTTWTLSTSSVSSQGSISEGDPRQSRPFKSVFVPTAIVNPVRSTAGPGTLGQGSLRKGRSSMRKNGSLQRPLQSGIPTLVVGSLRRSPTMVLRPQQFQFYQPQGIPSSPSAVVVEMGSKPALTGEPALTCISRGSEARIHSAASSLIMEDKEIPIKSEPLPKPPASAPPSILVKPENSRNGIEKQVKTVRFQNYSPPPTKHYTSHPTSGKPEQPATLKASQPEAASLGPEMTVLFAHRSGCHSGQQTDLRRKSALAKATTLVSTASGTQTVFPSK

SEQ ID NO: 34 - protein, 240 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSP

SEQ ID NO: 35 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 36 - protein, 599 aa, Homo sapiens [sequence omitted]

SEQ ID NO: 37 - protein, 662 aa, Homo sapiens
MGLYRIRVSTGASLYAGSNNQVQLWLVGQHGEAALGKRLWPARGKETELKVEVPEYLGPLLFVKLRKRHLLKDDAWFCNWISVQGPGAGDEVRFPCYRWVEGNGVLSLPEGTGRTVGEDPQGLFQKHREEELEERRKLYRWGNWKDGLILNMAGAKLYDLPVDERFLEDKRVDFEVSLAKGLADLAIKDSLNVLTCWKDLDDFNRIFWCGQSKLAERVRDSWKEDALFGYQFLNGANPVVLRRSAHLPARLVFPPGMEELQAQLEKELEGGTLFEADFSLLDGIKANVILCSQQHLAAPLVMLKLQPDGKLLPMVIQLQLPRTGSPPPPLFLPTDPPMAWLLAKCWVRSSDFQLHELQSHLLRGHLMAEVIVVATMRCLPSIHPIFKLIIPHLRYTLEINVRARTGLVSDMGIFDQIMSTGGGGHVQLLKQAGAFLTYSSFCPPDDLADRGLLGVKSSFYAQDALRLWEIIYRYVEGIVSLHYKTDVAVKDDPELQTWCREITEIGLQGAQDRGFPVSLQARDQVCHFVTMCIFTCTGQHASVHLGQLDWYSWVPNAPCTMRLPPPTTKDATLETVMATLPNFHQASLQMSITWQLGRRQPVMVAVGQHEEEYFSGPEPKAVLKKFREELAALDKEIEIRNAKLDMPYEYLRPSVVENSVAI

SEQ ID NO: 38 - DNA, 530 bp, Homo sapiens
  modified_base (484)..(484): a, c, g, t, unknown or other
aaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatagacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaagacacacccgtgaggaagttgcaagccaacaaaaaagttcaanggatctagaagacgattaagggaaggtcgttctcagtgaaaatcca

SEQ ID NO: 39 - DNA, 413 bp, Homo sapiens
aaattgtggagttagcctcctgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgc

SEQ ID NO: 40 - DNA, 493 bp, Homo sapiens
gcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttatgctctttcgagatctacgaagttccctgggaggacagaatgtccctggtgaattccaggtgtcaagaagcctaggggtctgtgccaggccagtcacaccgaccaccacccactcccaccccctgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccc

SEQ ID NO: 41 - DNA, 365 bp, Homo sapiens
ggaggataggataatcccgggtggcatctataacgcagacctcaatgatgagtgggtacagcgtgcccttcacttcgccatcagcgagtataacaaggccaccaaagatgactactacagacgtccgctgcgggtactaagagccaggcaacagaccgttgggggggtgaattacttcttcgacgtagaggtgggccgaaccatatgtaccaagtcccagcccaacttggacacctgtgccttccatgaacagccagaactgcagaagaaacagttgtgctctttcgagatctacgaagttccctgggagaacagaaggtccctggtgaaatccaggtgtcaagaatcctagggatctgtgccag

SEQ ID NO: 42 - DNA, 410 bp, Homo sapiens
gagaagggcctgatttgcagcatcatgatgggcctctccttggcctctgctgtgctcctggcctccctcctgagtctccaccttggaactgccacacgtgggagtgacatatccaagacctgctgcttccaatacagccacaagccccttccctggacctgggtgcgaagctatgaattcaccagtaacagctgctcccagcgggctgtgatattcactaccaaaagaggcaagaaagtctgtacccatccaaggaaaaaatgggtgcaaaaatacatttctttactgaaaactccgaaacaattgtgactcagctgaattttcatccgaggacgcttggaccccgctcttggctctgcagccctctggggagcctgcggaatcttttctgaaggctacatggacccgct

SEQ ID NO: 43 - DNA, 489 bp, Homo sapiens
ggccaaatcaccgacctgaaggcggaaattcacgggggcagtctcattaatctgacttggacagctcctggggatgattatgaccatggaacagctcacaagtatatcattcgaataagtacaagtattcttgatctcagagacaagttcaatgaatctcttcaagtgaatactactgctctcatcccaaaggaagccaactctgaggaagtctttttgtttaaaccagaaaacattacttttgaaaatggcacagatcttttcattgctattcaggctgttgataaggtcgatctgaaatcagaaatatccaacattgcacgagtatctttgtttattcctccacagactccgccagagacacctagtcctgatgaaacgtctgctccttgtcctaatattcatatcaacagcaccattcctggcattcacattttaaaaattatgtggaagtggataggagaactgcagctgtcaatagcctagggc

SEQ ID NO: 44 - DNA, 324 bp, Homo sapiens
gagcccccaggaggaggacaggataatcgagggtggcatctatgatgcagacctcaatgatgagcgggtacagcgtgcccttcactttgtcatcagcgagtataacaaggccactgaagatgagtactacagacgcctgctgcgggtgctacgagccagggagcagatcgtgggcggggtgaattacttcttcgacatagaggtgggccgaaccatatgtaccaagtcccagcccaacttggacacctgtgccttccatgaacagccagaactgcagaagaaacagttgtgctctttccagatctacgaagttccctgggagga

SEQ ID NO: 45 - DNA, 310 bp, Homo sapiens
aagactttactttcaccataccagatgtagaggactcaagtcagagaccagatcagggaccccagagacctcctcctgaaggactcctacctagaccccctggtgatagtggtaaccaagatgatggtcctcagcagagaccaccaaaaccaggaggccatcaccgccatcctcccccacctccttttcaaaatcagcaacgaccaccccaacgaggacaccgtcaactctctctaccccgatttccttctgtcagcctgcaggaagcatcatcattcttccggagggacagaccagcaagacatcccca

SEQ ID NO: 46 - DNA, 465 bp, Homo sapiens
ttcctcaccctaaaactaagcgtgctgcttctgcaaaagatttttgtagatgagctgtgtgcctcagaattgctatttcaaattgccaaaaatttagagatgttttctacatatttctgctcttctgaacaacttctgctacccactaaataaaaacacagaaataattagacaattgtctattataacatgacaaccctattaatcatttggtcttctaaaatgggatcatgcccatttagattttccttactatcagtttatttttataacattaacttttactttgttatttattattttatataatggtgagtttttaaattattgctcactgcctatttaatgtagctaataaagttatagaagcagatgatctgttaatttcctatctaataaatgcctttaattgttctcataatgaagaataagtaggtaccctccatgcccttctgtaataaatat

SEQ ID NO: 47 - DNA, 323 bp, Homo sapiens
agaagactctgacctgtactcttgaatacaagtttctgataccactgcactgtctgagaatttccaaaactttaatgaactaactgacagcttcatgaaactgtccaccaagatcaagcagagaaaataattaatttcatgggactaaatgaactaatgaggattgctgattctttaaatgtcttgtttcccagatttcaggaaactttttttcttttaagctatccactcttacagcaatttgataaaatatacttttgtgaacaaaaattgagacatttacattttctccctatgtggtcgctccagacttgggaaactat

SEQ ID NO: 48 - DNA, 500 bp, Homo sapiens
tcatcgggcctggcacaggcatcgcgcccttccgcagtttctggcagcaacggctccatgactcccagcacaagggagtgcggggaggccgcatgaccttggtgtttgggtgccgccgcccagatgaggaccacatctaccaggaggagatgctggagatggcccagaagggggtgctgcatgcggtgcacacagcctattcccgcctgcctggcaagcccaaggtctatgttcaggacatcctgcggcagcagctggccagcgaggtgctccgtgtgctccacaaggagccaggccacctctatgtttgcggggatgtgcgcatggcccgggacgtggcccacaccctgaagcagctggtggctgccaagctgaaattgaatgaggagcaggtcgaggactatttctttcagctcaagagccagaagcgctatcacgaagatatctttggtgctgtatttccttacgaggcgaagaaggacagggtggcggtgcagccc

SEQ ID NO: 49 - DNA, 363 bp, Homo sapiens
gtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatgaagagttctgttgtaatcaccctttcctattcttcataaggcaaaataagaccaacagcatcctcttctatggcagattctcatccccatagatgcaattagtctgtcactccatttag

SEQ ID NO: 50 - DNA, 508 bp, Homo sapiens
gatacgacactggttcttgtgaacgcaatctatttcaaagggcagtgggagaataaatttaaaaaagaaaacactaaagaggaaaaattttggccaaacaaggatgtacaggccaaggtcctggaaataccatacaaaggcaaagatctaagcatgattgtgctgctgccaaatgaaatcgatggtctgcagaagcttgaagagaaactcactgctgagaaattgatggaatggacaagtttgcagaatatgagagagacatgtgtcgatttacacttacctcggttcaaaatggaagagagctatgacctcaaggacacgttgagaaccatgggaatggtgaatatcttcaatggggatgcagacctctcaggcatgacctggagccacggtctctcagtatctaaagtcctacacaaggcctttgtggaggtcactgaggagggagtggaagctgcagctgccaccgctgtagtagtagtcgaattatcatctccttcaactaatg

SEQ ID NO: 51 - DNA, 493 bp, Homo sapiens; identical to SEQ ID NO: 40

SEQ ID NO: 52 - DNA, 447 bp, Homo sapiens
ccacctcctccaggaaagccagaaagaccacccccacaaggaggtaaccagtcccaaggtcccccacctcatccaggaaagccagaaggaccacccccacaggaaggaaacaagtcccgaagtgcccgatctcctccaggaaagccacaaggaccaccccaacaagaaggcaacaagcctcaaggtcccccacctcctggaaagccacaaggcccacccccagcaggaggcaatccccagcagcctcaggcacctcctgctggaaagccccaggggccacctccacctcctcaagggggcaggccacccagacctgcccagggacaacagcctccccagtaatctaggattcaatgacaggaagtgaataagaagatatcagtgaattcaaataattcaattgctacaaatgccgtgacattggaacaaggtcatcatagctctaac

SEQ ID NO: 53 - DNA, 304 bp, Homo sapiens
tgacgcaaaataccaccttggcgcctacacgggagacgacgtccgcatcatccgtgacgacatgctgtgtgccgggaacagccagagggactcctgcaagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctgggtgtgccacctgggtcactggaggacca

SEQ ID NO: 54 - DNA, 358 bp, Homo sapiens
ccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaa

SEQ ID NO: 55 - DNA, 542 bp, Homo sapiens
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccctatcctgagcccccttccctgtcctaagcctgacgcctgcaccgggccctccagccctcccctgcccagatagctggtggtgggcgctaatcct

SEQ ID NO: 56 - DNA, 536 bp, Homo sapiens
tgacgcaaaataccaccttggcgcctacacgggagacgacgtccgcatcgtccgtgacgacatgctgtgtgccgggaacacccggagggactcatgccagggcgactccggagggcccctggtgtgcaaggtgaatggcacctggctgcaggcgggcgtggtcagctggggcgagggctgtgcccagcccaaccggcctggcatctacacccgtgtcacctactacttggactggatccaccactatgtccccaaaaagccgtgagtcaggcctgggttggccacctgggtcactggaggaccaacccctgctgtccaaaacaccactgcttcctacccaggtggcgactgccccccacaccttccctgccccgtcctgagtgccccttcctgtcctaagccccctgctctcttctgagccccttcccctgtcctgaggacccttccccatcctgagcccccttccctgtcctaagcctgacgcctgcaccgggccctccggccctcccctgcccaggcagctggtggtgggcgct

SEQ ID NO: 57 - DNA, 538 bp, Homo sapiens
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccggggatgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagggactcatgccagggcgactctggagggcccctggtgtgcaaggtgaatggcacctggctacaggcgggcgtggtcagctgggacgagggctgtgcccagcccaaccggcctggcatctacacccgtgtcacctactacttggactggatccaccactatgtccccaaaaagccgtgagtcaggcctggggtgt

SEQ ID NO: 58 - DNA, 366 bp, Homo sapiens
ccggtcagcaggatcatcgtgcacccacagttctacaccgcccagatcggagcggacatcgccctgctggagctggaggagccggtgaacgtctccagccacgtccacacggtcaccctgccccctgcctcagagaccttccccccggggatgccgtgctgggtcactggctggggcgatgtggacaatgatgagcgcctcccaccgccatttcctctgaagcaggtgaaggtccccataatggaaaaccacatttgtgacgcaaaataccaccttggcgcctacacgggagacgacgtccgcatcgtccgtgacgacatgctgtgtgccgggaacacccggagggactcatgccaggtggcgact

SEQ ID NO: 59 - DNA, 496 bp, Homo sapiens
  modified_base (150)..(151): a, c, g, t, unknown or other
ccggtcagcaggatcatcgtgcacccacagttctacatcatccagactggagcggatatcgccctgctggagctggaggagcccgtgaacatctccagccgcgtccacacggtcatgctgccccctgcctcggagaccttccccccgggnntgccgtgctgggtcactggctggggcgatgtggacaatgatgagcccctcccaccgccatttcccctgaagcaggtgaaggtccccataatggaaaaccacatttgtgacgcaaaataccaccttggcgcctacacgggagacgacgtccgcatcatccgtgacgacatgctgtgtgccgggaacacccggagngnntcatgccagggcgactcnggagggcccctggtgtgcaaggtgaatggcacctggctncaggcgggcgtggtcagctgggncgagggctgtgcccagcccaaccggcctggcatctacacccgtgtcacctactacttggactggatcc

SEQ ID NO: 60 - DNA, 408 bp, Homo sapiens
gtcgtcacggacgatgcggacgtcgtctcccgtgtaggcgccaaggtggtattttgcgtcacaaatgtggttttccattatggggaccttcacctgcttcagaggaaatggcggtgggaggcgctcatcattgtccacatcgccccagccagtgacccagcacggcatccccggggggaaggtctctgaggcagggggcagggtgaccgtgtggacgtggctggagacgttcaccggctcctccagctccagcagggcgatgtccgctccgatctgggcggtgtagaactgtgggtgcacgatgatcctgctgaccggcagcagctggtcctggtagtagaggtgctgctcccgcagttgcaccggtcccacgcagtgcgctgcggtcagcacccactgggggtggat

SEQ ID NO: 61 - DNA, 520 bp, Homo sapiens
ggtgaaagtctccgtggtggacacagagacctgccgccgggactatcccggccccgggggcagcatccttcagcccgacatgctgtgtgcccggggccccggggatgcctgccaggacgactccggggggcctctggtctgccaggtgaacggtgcctgggtgcaggctggcattgtgagctggggtgagggctgcggccgccccaacaggccgggagtctacactcgtgtccctgcctacgtgaactggatccgccgccacatcacagcatcagggggctcagagtctgggtaccccaggctccccctcctggctggcttattcctccccggcctcttccttctgctagtctcctgtgtcctgctggccaagtgcctgctgcacccatctgcggatggtactcccttccccgcccctgactgatggcaggaatccaagtgcatttcttaaataagttactatttattccgctccgccccctccctctcccttgagaagctgagtcttctgcatcagatt

SEQ ID NO: 62 - DNA, 508 bp, Homo sapiens
tgccacaaggtcgctgcttatgagggcgcaaacttcttggcttgtgctcggacccttttctcgtactccactctgttttggcagtaaatcgtgtaggcctctgcttgagctgggtcttggatatttggttcatttagaagttcctgtattcctaataggatctgtttgattgtgatggctggcctccagtccttgtcctcctctaagatggacaggcacactgtccccgaagggtacacattcgggtgaaataatggtggttcgaatttacattttggtggcgaagatggataatcatctttgaaaagcatccgtagtttaaacaagcctccttcccacggagtccctttctttcctggaatggcgcactcccagttcatgaggttcatcgtgccatcgggattttttgttgggacagccacgaaaccaaatgggtggtctttcctccatgctttcctctcctgggcgagtctgctgagggcgatccccgacatgttcaaagtccctc

SEQ ID NO: 63 - DNA, 510 bp, Homo sapiens
agagactttcagggcatacgtgggggccttggccttcctcactcgctcgatggcctcagtgtgctcctcaaggctggtgccaaacacctgctggagatagctgagcagggcctcctcgtcgtccacctggtcagggcccatggtacccgcgcggtaaagcaccgtgtacagggcctcctcgtagagcatctccacctcctctggggccagggctctcaggccgaggctgggatccacaggctccgggggtgctggcgagccactgcgcagggggacctcgaggcacggcaagccctgtctgccttcccccttcttcagcatgaggcgcatgtgggcaaagaactccacgccatccccgggtttccaggcccccgtggcaggctcctgcgggtcggcgctggcactccctgggtcctgctcagtcctgcggcggaaggacgggcacacctgcacctgcctgagcacgctgctcttaatgtccagcaaggtcgacatggcgggtgaccgtgg

SEQ ID NO: 64 - DNA, 407 bp, Homo sapiens
tgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtaga

SEQ ID NO: 65 - DNA, 565 bp, Homo sapiens
tatgaaacccgctacatctatggcccaatagaatcaacaatttacccgatatcaggttcttctttagactgggcttatgacctgggcatcaaacacacatttgcctttgagctccgagataaaggcaaatttggttttctccttccagaatcccggataaagccaacgtgcagagagaccatgctagctgtcaaatttattgccaagtatatcctcaagcatacttcctaaagaactgccctctgtttggaataagccaattaatccttttttgtgcctttcatcagaaagtcaatcttcagttatccccaaatgcagcttctatttcacctgaatccttctcttgctcatttaagtcccatgttactgctgtttgcttttacttactttcagtagcaccataacgaagtagctttaagtgaaaccttttaactacctttctttgctccaagtgaagtttggacccagcagaaagcattattttgaaaggtgatatacagtggggcacagaaaacaaatgaaaaccctcagtttctcacagattttcaccatgtggcttcatcaa

SEQ ID NO: 66 - DNA, 459 bp, Homo sapiens
tgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttact

SEQ ID NO: 67 - DNA, 430 bp, Homo sapiens
  modified_base (343)..(345): a, c, g, t, unknown or other
gggaatcactattcagggatttttcccctttgctcttcttttccctccttaaaagaaaaattaccttctagtcctaggatgaggacacactattagtttgaattaaatgctttgatattctcagatcagccatcttgaaccaaagcaaaaccacaagttacactttcttaaaatttgatttgtcatattttctagagaaacttgaatttaattgtgttattcttagcttccactggcagcctagctttgagggtaaatgaaaatataacccatagattacccagccacttgggaacagcaggtaatactgaagaaaaataaaaatagattttgaaaacgttannnanannnntatgattatgattctgttccatttaagggaaaacttaggtaaatagagaaattttttctataacattgtgtagtcagt

SEQ ID NO: 68 - DNA, 358 bp, Homo sapiens
tgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgt

SEQ ID NO: 69 - DNA, 506 bp, Homo sapiens
  modified_base (86)..(86): a, c, g, t, unknown or other
acttttgaccacttgtgactggagttcagtggccctggcaggcttgtcctgctcttgaccattccactgactaactttggtgtttngtttccaagttaagtgattcctcctttttttngttcaatgttaaatttaaaaataacaatgtgtatgggtcctcccatgtgtaatatggtaacatgtaacttgcagtgtttgccagctttcaaagcaggctttgtgaaaatgtaatacaaacagcagtgaatgggactcaaatgttgtgcttcctataaacagctccgctctttcagggaaggatggtaacaaactagaaggacaaatatgtacgtatttataacgtattaaaactcttttaagtagcttaaggtattgtgcaatggcctagcctagtagaaatgggggaaaagcattgctgtggaccattgttaaagtgacaggagttgtagggttacccctttgacaagcttccatagtcttcagacacgcacattgatggcatccct

SEQ ID NO: 70 - DNA, 496 bp, Homo sapiens
  modified_base (60)..(60): a, c, g, t, unknown or other
ctaactaataccaacctgacaacttgaataacaataaatgcaatttgtacataaaatatnatgctgcaaaagttngtcattcacctcagtggagtgacttgatattaggtggtnaccgtagatgatggttnatatganaantggacaggaaagaagcantttctgaaagttatantcttttgaaccacgttctaaaccaagtntttnatcttcttggggctcgtaattacctttcactttaatgtcacttaaagatataacacagaaaaatgccttgagggcaaaatataggcaaaacaccaatgcgctttcaaatgcatgaaaatggtgcagttgtacccttgagccttgactcaagggctgtagatgttccctttccaccccccacacttggtgcgtgttcacaaagcaaatatggcctgtaattcaaatttgttctatgtgatactctctgagtaaaaactcatacatgcagaaaattgtctttgctcgaaat

SEQ ID NO: 71 - DNA, 560 bp, Homo sapiens
cccaagcccctagagaagtcagtctccccgcaaaattcagattcttcaggttttgcagatgtgaatggttggcaccttcgtttccgctggtccaaggatgctccctcagaactcctgaggaagttcagaaactatgaaatatgaaatatctctgcttcaaaaaatgaggaagagcaagactgtcccctatgctgccaacatgcagtctttgtttatgtcttaaaaatgtcatgtttatgtcatgtctgtgaattgctgagtactaattgattcctccatccttgaatcagttctcataatgctttttaaataagaaaaattcagaagatgaatttcttccaatatttgaataaattaaagctcttagatacagagtagattgtattatatgctttttcctattaatactacttatagaaatccattaaaaagcaatctctgtacagtgtatttaaatatttcattgacatactgtgatctctattagtgatggatgtacaaaaaatgttttcttacccttgacttacaatgaaatgtgaaattacttgtctgaaccccgt

SEQ ID NO: 72 - DNA, 512 bp, Homo sapiens
  modified_base (443)..(443): a, c, g, t, unknown or other
acagcaagcctatgtagttcaattaatatataaggaaaaggaaggtctttcttcatgatacaagcattataaagtttttactgtagtagtcaattaatggatatttccttgttaataaaattttgtgtcataatttacaaattagttctttaaaaattgttgttatatgaattgtgtttctagcatgaatgttctatagagtactctaaataacttgaatttatagacaaatgctactcacagtacaatcaattgtattataccatgagaaaatcaaaaaggtgttcttcagagacattttatctataaaattttcctactattatgttcattaacaaacttctttatcacatgtatcttctacgtgtaaaacatttctgatgattttttaacaaaaaatatatgaatttcttcatttgctcttgcatctacattgctataanggatataaaatgtggtttctatattttgagatgttttttccttacaatgtgaactcatcgtgatcttgg

SEQ ID NO: 73 - DNA, 551 bp, Homo sapiens
  modified_base (114)..(114): a, c, g, t, unknown or other
ctgctactttggaagatggctctggaggaaactctcatatggctaaaaaggcaggctagtttcttacttctacaggggtagagccttaaaaaagaacgtgctacaaattggttntcttnnagggttncnggttctccctgcccccaatnccnatatactttantgcnnttttatttttgcctttacggnctctgtgtctttctgcaagaaggcctggcaaaggtatgcctgctgttggtcccntcgggataagataaaatataaataaaaccttcagaactgttttggagcaaaagatagcttgtacttggggaaaaaaattctaagttcttttatatgactaatattcttggttagcaagactggaaagaggtgtttttttaaaatgtacataccagaacaaagaacatacagctctctgaacatttattttttgaacagaggtggtttttatgtttggacctggtaatacagatacaaaaactttaatgaggtagcaatgaatattcaactgtttgactgctaagtgtatctgtccatattttagcaag

SEQ ID NO: 74 - DNA, 474 bp, Homo sapiens
  modified_base (365)..(365): a, c, g, t, unknown or other
tactacaaaagccgtgacctcagcctgcttatactactgccagaagacattaatgggctggaacagctggaaaaggccatcacctatgagaagctgaatgagtggaccagtgcagacatgatggagttgtatgaagtgcagctacaccttcccaagttcaagctggaagacagttatgatctcaagtcaaccctgagcagtatggggatgagtgatgccttcagccaaagcaaagctgatttctcaggaatgtcttcagcaagaaacctatttttgtccaatgttttccataaggcttttgtggaaataaatgaacaaggtactgaagctgcagctggcagtgggagtgagatagatatacgaantagagtcccatccattgaattcaatgcaaatcacccattcctcttcttcatcaggcacaataanaaccaacaccattcttttttatggaagattatgctccccctaatc

SEQ ID NO: 75 - DNA, 287 bp, Homo sapiens
gattctgtggtagactcagtgctttcagagtccagagcttgacttgggttagtggccttaatgaagtgctaaatttgctctttaccgcgagactgatcagaagaagcaaaaggggaaagggggctagaggtccactcgcaccttttacatcagacaagaggaggactgtgccagaaatctgtgcatgaaacaccatctgctcttcatgcagggaggggtcaaccgtgtgaacgtgcagagattactcgagccttctttgccaaaaatatgcattcttcccagctgta

SEQ ID NO: 76 - DNA, 545 bp, Homo sapiens
taatcacatcacttccatggcatggatgttcacatacagactcttaaccctggtttaccaggacctctaggagtggatccaatctatatctttacagttgtatagtatatgatatctcttttatttcactcaatttatattttcatcattgactacatatttcttatacacaacacacaatttatgaattttttctcaagatcattctgagagttgccccaccctacctgccttttatagtacgcccacctcaggcagacacagagcacaatgctggggttctcttcacactatcactgccccaaattgtctttctaaatttcaacttcaatgtcatcttctccatgaagaccactgaatgaacaccttttcatccagccttaatttcttgctccataactactctatcccacgatgcagtattgtatcattaattattagtgtgcttgtgacctccttatgtattctcaattacctgtatttgtgcaataaattggaataatgtaacttgatttcttatctgtgtttgtgttggcatgcaagat

SEQ ID NO: 77 - DNA, 420 bp, Homo sapiens
aagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgc

SEQ ID NO: 78 - DNA, 534 bp, Homo sapiens
  modified_base (486)..(493): a, c, g, t, unknown or other
agtattgacaactgcacatgaaagttttgcaaagggaaacaggctaaatgcaccaagaaagcttcttcagagtgaagaatcttaatgcttgtaatttaaacatttgttcctggagttttgatttggtggatgtgatggttggttttatttgtcagtttggttgggctatagcacacagttatttaatcaaacagtaatctaggtgtggctgtgaaggtattttgtagatgtgattaacatctacaatcagttgactttaagtgaaagagattacttaaataatttgggtgagctgcacctgattagttgaaaggcctcaagaacaaacactgcagtttcctggaaaagaagaaactttgcctcaagactatagccatcgactcctgcctgagtttccagcctgctagtctgccctatggatttgaagtttgccaaccccaacaattgtgtgaattaatttctaaaaataaagctatatacagccannnnnnnntatttgtgggggatttgtttcaggatctctacagataccaa